(19) United States
(12) Patent Application Publication
(10) Pub. No.: US 2010/0279327 A1
(43) Pub. Date: Nov. 4, 2010

(54) METHOD OF TREATING DISEASES WITH PARP INHIBITORS

(75) Inventors: Valeria S. OSSOVSKAYA, San Francisco, CA (US); Barry M. SHERMAN, Hillsborough, CA (US)

Correspondence Address: BiPar Sciences Inc. c/o Morrison Foerster, 755 Page Mill Road, Palo Alto, CA, 94304, US

(73) Assignee: BiPar Sciences, Inc., South San Francisco, CA (US)

(21) Appl. No.: 12/748,209

(22) Filed: Mar. 26, 2010

Related U.S. Application Data
(63) Continuation-in-part of application No. 11/818,210, filed on Jun. 12, 2007.
Application No. 11/818,210 is a continuation-in-part of application No. 11/940,307, filed on Nov. 14, 2007.
(60) Provisional application No. 60/804,563, filed on Jun. 12, 2006.
(60) Provisional application No. 60/866,602, filed on Nov. 20, 2006.
(60) Provisional application No. 60/866,602, filed on Nov. 20, 2006.

(51) Int. Cl. C12Q 1/48; A61K 31/166; A61P 35/00; A61P 37/00; A61P 7/12; A61P 3/04; A61P 25/16; A61P 25/26; A61P 25/28; A61P 13/00; A61P 11/00; A61P 15/00

(52) U.S. Cl. 435/15; 514/617; 514/619; 514/622

(57) ABSTRACT

The present invention relates to methods of identifying a disease treatable with PARP modulators by identifying a level of PARP in a sample of a subject, making a decision regarding identifying the disease treatable by the PARP modulators wherein the decision is made based on the level of PARP. The method further comprises of treating the disease in the subject with the PARP modulators. The methods relate to identifying up-regulated PARP in a disease and making a decision regarding the treatment of the disease with PARP inhibitors. The extent of PARP up-regulation in a disease can also help in determining the efficacy of the treatment with PARP inhibitors. The present invention also relates to methods of identifying a disease treatable with PARP modulators by identifying a level of PARP in a plurality of samples from a population, making a decision regarding identifying the disease treatable by the PARP modulators wherein the decision is made based on the level of PARP. The method further comprises of treating the disease in a subject population with the PARP modulators. The methods relate to identifying up-regulated PARP in a disease and making a decision regarding the treatment of the disease with PARP inhibitors. The extent of PARP up-regulation in a disease can also help in determining the efficacy of the treatment with PARP inhibitors.

The present invention discloses various diseases that have up-regulated or down-regulated PARP and can be treated with PARP inhibitors or PARP activators, respectively. The examples of the diseases include cancer, inflammation, metabolic disease, CVS disease, CNS disease, disorder of hematolymphoid system, disorder of endocrine and neuroendocrine, disorder of urinary tract, disorder of respiratory system, disorder of female reproductive system, and disorder of male reproductive system.

### RELATED APPLICATIONS

This application is a continuation-in-part of U.S. application Ser. No. 11/818,210, filed Jun. 12, 2007, which claims priority to U.S. Provisional Application No. 60/804,563, filed Jun. 12, 2006 and U.S. Provisional Application No. 60/866,602, filed Nov. 20, 2006, and this application is also a continuation-in-part of U.S. application Ser. No. 11/940,307, filed Nov. 14, 2007, which claims priority to U.S. Provisional Application No. 60/866,602, filed Nov. 20, 2006, the contents of each of which are hereby incorporated by reference in their entirety.

### BACKGROUND OF THE INVENTION

PARP (poly-ADP ribose polymerase) participates in a variety of DNA-related functions including cell proliferation, differentiation, apoptosis, DNA repair and also effects on telomere length and chromosome stability (d'Adda di Fagagna et al, 1999, Nature Gen., 23(1): 76-80). Oxidative stress-induced overactivation of PARP consumes NAD+ and consequently ATP, culminating in cell dysfunction or necrosis. This cellular suicide mechanism has been implicated in the pathomechanism of cancer, stroke, myocardial ischemia, diabetes, diabetes-associated cardiovascular dysfunction, shock, traumatic central nervous system injury, arthritis, colitis, allergic encephalomyelitis, and various other forms of inflammation. PARP has also been shown to associate with and regulate the function of several transcription factors. The multiple functions of PARP make it a target for a variety of serious conditions including various types of cancer and neurodegenerative diseases.

Breast cancer is a malignant tumor that develops from cells in the breast. It is the most common cancer among women, other than skin cancer, and it is the second leading cause of cancer-related death in women. The incidence of breast cancer in women rose from 100.5 cases per 100,000 population in 1991 to 117.2 cases per 100,000 population in 2001; an average increase of 1.4% per annum. In 2004, an estimated 215,990 new cases of invasive breast cancer are anticipated among women in the United States; about 1,450 in men. An additional 59,390 new cases of in situ breast cancer are expected during that period—about 85 percent of which are ductal carcinoma in situ (DCIS).

Node-positive breast cancers often overexpressed the HER2/neu oncogene, meaning there were more copies than normal of the HER2 protein on the cell surface. Women whose breast cancers have more copies of the HER2 gene spread the fastest and had a worse prognosis. This subset of breast cancers is typically treated with Her-2 antibody called Trastuzumab.

Women carrying non-functional BRCA1 and BRCA2 genes and their molecular pathways have up to an 85% chance of developing breast cancer by the age of 70. According to the conclusions of the Breast Cancer Linkage Consortium (1997), the histology of breast cancers in women predisposed by reason of carrying BRCA1 and BRCA2 (600185) mutations differs from that in sporadic cases, and there are differences between breast cancers in carriers of BRCA1 and BRCA2 mutations.

PARP inhibitors may be effective in killing tumor cells in people who have faults in BRCA 1 and BRCA2 (Byrant, et al., 2005, Nature, 434(7035): 913-7 and Farmer, et al., 2005, Nature, 434(7035): 917-21). PARP inhibitors have the potential to help the specific subset of patients who have mutations in these genes. These mutations predispose patients to early-onset of cancer and have been found in breast, ovarian, prostate and pancreatic cancers.

Approximately 148,000 new cases of colorectal cancer are expected in the US and of this 60-70% are expected to be in advanced stages. Colorectal cancer is the second-leading cause of cancer-related death, accounting for more than 50,000 deaths per year. Although the aetiology of colorectal cancer is largely unknown, genetics as well as lifestyle factors, including diet and sedentary lifestyle, may play a significant role in the development of colorectal cancer.

Today's early detection strategies mean that health professionals are catching cancers, including colorectal cancer, in their very early stages, when they are highly treatable. For example, simple screening procedure called a colonoscopy can find polyps before they ever have a chance to become cancerous. Such screening procedures are not as readily available for other cancers, including breast cancer. However, even where screening procedures are available, more efficient and robust strategies for early diagnosis of cancer can be extremely beneficial for prevention and more efficient treatment of cancers.

### SUMMARY OF THE INVENTION

In one aspect, the present invention provides methods to identify diseases treatable by PARP inhibitor in a subject by measuring the level of PARP in the subject and if PARP is up-regulated in the subject further providing treatment of the subject with PARP inhibitors itself or in a combination with other agents or treatments.

In one aspect, the present invention provides methods to identify diseases treatable by PARP inhibitor in a subject by measuring the level of PARP in a plurality of samples from a population and if PARP is up-regulated in the plurality of samples, the disease is treatable by a PARP inhibitor itself or in combination with other agents or treatments.

One aspect of the invention relates to a method of identifying a disease or a stage of a disease treatable by PARP modulator comprising identifying a level of PARP in a sample of a subject, making a decision regarding identifying the disease treatable by the PARP modulators wherein the decision is made based on the level of expression of PARP. In some preferred embodiments, the level of PARP is up-regulated. One aspect of the invention relates to a method of identifying a disease or a stage of a disease treatable by PARP modulator in a combination with other agents comprising identifying a level of PARP in a sample of a subject, making a decision regarding identifying the disease treatable by the PARP modulators in a combination with other agents wherein the decision is made based on the level of expression of PARP. In some preferred embodiments, the level of PARP is up-regulated.

One aspect of the invention relates to a method of identifying a disease or a stage of a disease treatable by PARP modulator comprising identifying a level of PARP in a plurality of samples from a population, making a decision regarding identifying the disease treatable by the PARP modulators wherein the decision is made based on the level of expression of PARP. In some preferred embodiments, the level of PARP is up-regulated. One aspect of the invention relates to a method of identifying a disease or a stage of a disease treatable by PARP modulator in a combination with other agents comprising identifying a level of PARP in a plurality of samples from a population, making a decision regarding identifying the disease treatable by the PARP modulators in combination with other agents wherein the decision is made based on the level of expression of PARP. In some preferred embodiments, the level of PARP is up-regulated.

Another aspect of the invention relates to a method of treating a disease by PARP modulators in a subject comprising identifying a level of PARP in a sample of the subject, making a decision based on the level of PARP regarding identifying the disease treatable by the PARP modulators, and treating the disease in the subject by the PARP modulators. In some preferred embodiments, the level of PARP is up-regulated.

Another aspect of the invention relates to a method of treating a disease by PARP modulators in a subject comprising identifying a level of PARP in a plurality of samples of a population with the disease, making a decision based on the level of PARP regarding identifying the disease treatable by the PARP modulators, comparing the level of PARP in the subject to the level of PARP in the plurality of samples of the population with the disease, and treating the disease in the subject by the PARP modulators. In some preferred embodiments, the level of PARP is up-regulated.

In some embodiments, the disease is selected from the group consisting of cancer, inflammation, metabolic disease, CVS disease, CNS disease, disorder of hematolymphoid system, disorder of endocrine and neuroendocrine, disorder of urinary tract, disorder of respiratory system, disorder of female reproductive system, and disorder of male reproductive system. In some preferred embodiments, the cancer is selected from the group consisting of colon adenocarcinoma, esophagus adenocarcinoma, liver hepatocellular carcinoma, squamous cell carcinoma, pancreas adenocarcinoma, islet cell tumor, rectum adenocarcinoma, gastrointestinal stromal tumor, stomach adenocarcinoma, adrenal cortical carcinoma, follicular carcinoma, papillary carcinoma, breast cancer, ductal carcinoma, lobular carcinoma, intraductal carcinoma, mucinous carcinoma, phyllodes tumor, ovarian adenocarcinoma, endometrium adenocarcinoma, granulose cell tumor, mucinous cystadenocarcinoma, cervix adenocarcinoma, vulva squamous cell carcinoma, basal cell carcinoma, prostate adenocarcinoma, giant cell tumor of bone, bone osteosarcoma, larynx carcinoma, lung adenocarcinoma, kidney carcinoma, urinary bladder carcinoma, Wilm's tumor, and lymphoma.

In some preferred embodiments, the inflammation is selected from the group consisting of Wegener's granulomatosis, Hashimoto's thyroiditis, hepatocellular carcinoma, chronic pancreatitis, rheumatoid arthritis, reactive lymphoid hyperplasia, osteoarthritis, ulcerative colitis, and papillary carcinoma. In some preferred embodiments, the metabolic disease is diabetes or obesity. In some preferred embodiments, the CVS disease is selected from the group consisting of atherosclerosis, coronary artery disease, granulomatous myocarditis, chronic myocarditis, myocardial infarction, and primary hypertrophic cardiomyopathy. In some preferred embodiments, the CNS disease is selected from the group consisting of Alzheimer's disease, cocaine abuse, schizophrenia, and Parkinson's disease. In some preferred embodiments, the disorder of hematolymphoid system is selected from the group consisting of Non-Hodgkin's lymphoma, chronic lymphocyte leukemia, and reactive lymphoid hyperplasia.

In some preferred embodiments, the disorder of endocrine and neuroendocrine is selected from the group consisting of nodular hyperplasia, Hashimoto's thyroiditis, islet cell tumor, and papillary carcinoma. In some preferred embodiments, the disorder of urinary tract is selected from the group consisting of renal cell carcinoma, transitional cell carcinoma, and Wilm's tumor. In some preferred embodiments, the disorder of respiratory system is selected from the group consisting of adenocarcinoma, adenosquamous carcinoma, squamous cell carcinoma, and large cell carcinoma. In some preferred embodiments, the disorder of female reproductive system is selected from the group consisting of adenocarcinoma, leiomyoma, mucinous cystadenocarcinoma, and serous cystadenocarcinoma. In some preferred embodiments, the disorder of male reproductive system is selected from the group consisting of prostate cancer, benign nodular hyperplasia, and seminoma.

In some embodiments, the identification of the level of PARP comprises assay technique. In some preferred embodiments, the assay technique measures expression of PARP gene. In some embodiments, the sample is selected from the group consisting of human normal sample, tumor sample, hair, blood, cell, tissue, organ, brain tissue, blood, serum, sputum, saliva, plasma, nipple aspirant, synovial fluid, cerebrospinal fluid, sweat, urine, fecal matter, pancreatic fluid, trabecular fluid, cerebrospinal fluid, tears, bronchial lavage, swabbing, bronchial aspirant, semen, prostatic fluid, precervicular fluid, vaginal fluids, and pre-ejaculate. In some preferred embodiments, the level of PARP is up-regulated. In some embodiments, the level of PARP is down-regulated. In some embodiments, the PARP modulator is PARP inhibitor or antagonist. In some embodiments, the PARP inhibitor or antagonist is selected from the group consisting of benzamide, quinolone, isoquinolone, benzopyrone, methyl 3,5-diiodo-4-(4′-methoxyphenoxy)benzoate, and methyl-3,5-diiodo-4-(4′-methoxy-3′,5′-diiodo-phenoxy)benzoate, cyclic benzamide, benzimidazole and indole.

In some embodiments, the method further comprises of providing a conclusion regarding the disease to a patient, a health care provider or a health care manager, the conclusion being based on the decision. In some embodiments, the treatment is selected from the group consisting of oral administration, transmucosal administration, buccal administration, nasal administration, inhalation, parental administration, intravenous, subcutaneous, intramuscular, sublingual, transdermal administration, and rectal administration.

Another aspect of the invention relates to a computer-readable medium suitable for transmission of a result of an analysis of a sample wherein the medium comprises of an information regarding a disease in a subject treatable by PARP modulators, the information being derived by identifying a level of PARP in the sample of the subject, and making a decision based on the level of PARP regarding treating the disease by the PARP modulators. In some embodiments, at least one step in the methods is implemented with a computer.

Another aspect of the invention relates to a computer-readable medium suitable for transmission of a result of an analysis of a sample wherein the medium comprises of an information regarding a disease in a subject treatable by PARP modulators, the information being derived by identifying a level of PARP in a plurality of samples of a population, and making a decision based on the level of PARP regarding treating the disease by the PARP modulators. In some embodiments, at least one step in the methods is implemented with a computer.

Another aspect of the invention relates to a selection of patients who are triple-negative (lack receptors for the hormones estrogen (ER-negative) and progesterone (PR-negative), and for the protein HER2) for treatment with a PARP inhibitor. In one embodiment, the cancer type treated with a PARP inhibitor lacks receptors for the hormone estrogen (ER-negative). In another embodiment, the cancer type treated with a PARP inhibitor lacks receptors for the hormone progesterone (PR-negative). In yet another embodiment, the cancer type treated with a PARP inhibitor lacks the protein HER2.

Another aspect of the invention relates to a selection of a group of patients with deficiency of BRCA-dependent pathways and their treatment with PARP inhibitors.

Yet another aspect of the invention relates to a method of identifying a breast cancer treatable by PARP inhibitor or PARP antagonist comprising identifying a level of PARP in a sample of a subject, and making a decision based on the level of PARP regarding identifying the breast cancer treatable by the PARP inhibitor or PARP antagonist. Another aspect of the present invention relates to a method of treating a breast cancer in a subject by PARP inhibitor or PARP antagonist comprising identifying a level of PARP in a sample of the subject, making a decision based on the level of PARP regarding identifying the breast cancer treatable by the PARP modulators, and treating the breast cancer by the PARP inhibitor or PARP antagonist. In some embodiments, the level of PARP is up-regulated. In some embodiments, the subject is deficient in BRCA gene. In some embodiments, the subject has down-regulated BRCA gene. In some methods, increase in PARP levels is an indication of BRCA1 and/or BRACA2 deficiency.

One aspect is methods of diagnosing and/or treating breast cancers. One embodiment is a method of identifying a breast cancer treatable with a PARP inhibitor comprising identifying a level of PARP in a sample from a subject and making a decision based on said level of PARP regarding whether said breast cancer is treatable with said PARP inhibitor. Another embodiment is a method of treating a breast cancer in a subject with a PARP inhibitor comprising identifying a level of PARP in a sample from said subject; making a decision based on said level of PARP regarding whether said breast cancer is treatable with said PARP inhibitor; and treating said breast cancer with said PARP inhibitor. Yet another embodiment is method of classifying a breast tumor in a subject comprising identifying a level of PARP in a tumor sample from said subject and making a decision regarding treating said tumor with a PARP modulator, wherein said decision is made based on said level of PARP. Another embodiment is a method of treating a breast tumor in a subject comprising identifying a level of PARP in a sample from said subject; making a decision based on said level of PARP regarding treating said tumor with a PARP modulator; and treating said tumor in said subject with said PARP modulator. Preferably, the breast tumor is an infiltrating duct carcinoma. In some embodiments, the cancers are negative for ER, Her2-neu, and/or PR. Another embodiment is a method of treating a cancer in a subject comprising identifying a presence or absence of ER, Her2-neu, and PR in a cancer sample from said subject and treating said cancer with a PARP inhibitor, wherein said treatment is performed if said cancer sample is negative for ER, Her2-neu, and/or PR.

One aspect is methods of diagnosing and/or treating breast cancers. One embodiment is a method of identifying a breast cancer treatable with a PARP inhibitor comprising identifying a level of PARP in a plurality of samples from a subject and making a decision based on said level of PARP regarding whether said breast cancer is treatable with said PARP inhibitor. Another embodiment is a method of treating a breast cancer with a PARP inhibitor comprising identifying a level of PARP in a plurality of samples from a population; making a decision based on said level of PARP regarding whether said breast cancer is treatable with said PARP inhibitor; and treating said breast cancer with said PARP inhibitor. Yet another embodiment is method of classifying a breast tumor comprising identifying a level of PARP in a plurality of tumor samples from a population with breast cancer and making a decision regarding treating said tumor with a PARP modulator, wherein said decision is made based on said level of PARP. Another embodiment is a method of treating a breast tumor comprising identifying a level of PARP in a plurality of samples from a population; making a decision based on said level of PARP regarding treating said tumor with a PARP modulator; determining the level of PARP in a subject and comparing said level with the level PARP in the plurality of samples from the population, and treating said tumor in said subject with said PARP modulator if the level of PARP in the subject is above a predetermined value. Preferably, the breast tumor is an infiltrating duct carcinoma. In some embodiments, the cancers are negative for ER, Her2-neu, and/or PR. Another embodiment is a method of treating a cancer in a subject comprising identifying a presence or absence of ER, Her2-neu, and PR in a cancer sample from said subject and treating said cancer with a PARP inhibitor, wherein said treatment is performed if said cancer sample is negative for ER, Her2-neu, and/or PR.

In another aspect the methods of diagnosing and/or treating breast cancers involve comparison of a level of PARP from a subject in need of diagnosis or treatment to a pre-determined level of PARP. One embodiment is a method of identifying a breast cancer treatable with a PARP inhibitor comprising identifying a level of PARP in a sample from a subject; and determining whether said level of PARP is above a predetermined level thereby determining that said breast cancer is treatable with a PARP modulator. Another embodiment is a method of treating a breast cancer in a patient with a PARP inhibitor comprising identifying a level of PARP in a sample from said subject; determining whether said level of PARP is above a predetermined level thereby determining that said breast cancer is treatable with a PARP inhibitor; and treating said breast cancer by administering said PARP inhibitor to said patient. Typically the subject is also BRCA1 or BRCA2 deficient. Some subjects have decreased level of expression of a BRCA gene. Another embodiment is a method of classifying a breast tumor in a patient comprising identifying a level of PARP in a tumor sample from said patient and determining whether said level of PARP is above a predetermined level thereby classifying said breast tumor as treatable with a PARP modulator. One method is a method of treating a breast tumor in a subject comprising identifying a level of PARP in a sample from said subject; determining whether said level of PARP is above a predetermined level thereby determining that said breast tumor is treatable with a PARP modulator and treating said tumor in said patient with said PARP modulator. Yet another method is a method of identifying a breast tumor treatable with a PARP inhibitor comprising identifying a level of PARP in a sample from a patient; determining whether said level of PARP is above a predetermined level thereby identifying said breast tumor as treatable with a PARP inhibitor. Another method is a method of treating a breast tumor in a patient with a PARP inhibitor comprising identifying a level of PARP in a sample from said patient; determining whether said level of PARP is above a predetermined level thereby determining that said breast tumor is treatable with a PARP inhibitor and treating said breast tumor by administering said PARP inhibitor to said patient. Typically the breast tumor is an infiltrating duct carcinoma. Some of the infiltrating duct carcinoma is negative for ER, Her2-neu, and/or PR. A preferred method is a method of treating a cancer in a patient comprising determining whether ER, Her2-neu, and/or PR are present in a cancer sample from said patient and treating said cancer with a PARP inhibitor when ER, Her2-neu, and/or PR are not present in said sample from said patient.

In another aspect the methods of diagnosing and/or treating breast cancers involve comparison of a level of PARP from a subject in need of diagnosis or treatment to a pre-determined level of PARP. One embodiment is a method of identifying a breast cancer treatable with a PARP inhibitor comprising identifying a level of PARP in a sample from a subject; and determining whether said level of PARP is above a predetermined level thereby determining that said breast cancer is treatable with a PARP modulator, wherein the predetermined level is derived from a PARP level of each of a plurality of samples from a population with said breast cancer. Another embodiment is a method of treating a breast cancer in a patient with a PARP inhibitor comprising identifying a level of PARP in a sample from said subject; determining whether said level of PARP is above a predetermined level thereby, determining that said breast cancer is treatable with a PARP inhibitor; and treating said breast cancer by administering said PARP inhibitor to said patient, wherein the predetermined level is derived from a PARP level of each of a plurality of samples from a population with said breast cancer. In some aspects, the subject is also BRCA1 or BRCA2 deficient. Some subjects have decreased level of expression of a BRCA gene. Another embodiment is a method of classifying a breast tumor in a patient comprising identifying a level of PARP in a tumor sample from said patient and determining whether said level of PARP is above a predetermined level thereby classifying said breast tumor as treatable with a PARP modulator, wherein the predetermined level is derived from a PARP level of each of a plurality of samples from a population with said breast cancer. One method is a method of treating a breast tumor in a subject comprising identifying a level of PARP in a sample from said subject; determining whether said level of PARP is above a predetermined level thereby determining that said breast tumor is treatable with a PARP modulator and treating said tumor in said patient with said PARP modulator, wherein the predetermined level is derived from a PARP level of each of a plurality of samples from a population with said breast cancer. Yet another method is a method of identifying a breast tumor treatable with a PARP inhibitor comprising identifying a level of PARP in a sample from a patient; determining whether said level of PARP is above a predetermined level thereby identifying said breast tumor as treatable with a PARP inhibitor, wherein the predetermined level is derived from a PARP level of each of a plurality of samples from a population with said breast cancer. Another method is a method of treating a breast tumor in a patient with a PARP inhibitor comprising identifying a level of PARP in a sample from said patient; determining whether said level of PARP is above a predetermined level thereby determining that said breast tumor is treatable with a PARP inhibitor and treating said breast tumor by administering said PARP inhibitor to said patient, wherein the predetermined level is derived from a PARP level of each of a plurality of samples from a population with said breast cancer. Typically the breast tumor is an infiltrating duct carcinoma. Some of the infiltrating duct carcinoma is negative for ER, Her2-neu, and/or PR. A preferred method is a method of treating a cancer in a patient comprising determining whether ER, Her2-neu, and/or PR are present in a cancer sample from said patient and treating said cancer with a PARP inhibitor when ER, Her2-neu, and/or PR are not present in said sample from said patient, wherein the predetermined level is derived from a PARP level of each of a plurality of samples from a population with said breast cancer.

One embodiment is a method of identifying a PARP mediated disease or a stage of a PARP mediated disease treatable with a PARP modulator comprising identifying a level of PARP in a sample from a subject and determining whether said level of PARP is above a predetermined level thereby determining that said PARP mediated disease is to be treated with a PARP modulator. Another embodiment is a method of treating a disease by administration of a PARP modulator to a patient comprising identifying a level of PARP in a sample from said patient; determining whether said level of PARP is above a predetermined level thereby determining that said PARP mediated disease is to be treated with a PARP modulator and treating said disease in said subject by administering said PARP modulator to said patient.

One embodiment is a method of identifying a PARP mediated disease or a stage of a PARP mediated disease treatable with a PARP modulator comprising identifying a level of PARP in a plurality of samples from a population and determining whether said level of PARP is above a predetermined level thereby determining that said PARP mediated disease is to be treated with a PARP modulator. Another embodiment is a method of treating a disease by administration of a PARP modulator to a patient comprising identifying a level of PARP in a sample from said patient; determining whether said level of PARP is above a predetermined level thereby determining that said PARP mediated disease is to be treated with a PARP modulator and treating said disease in said subject by administering said PARP modulator to said patient, wherein the predetermined level is derived from a PARP level of each of a plurality of samples from a population with the disease.

Yet another aspect of the invention relates to a method of identifying a breast cancer treatable by PARP inhibitor or PARP antagonist comprising identifying a level of PARP in a sample of a subject, and making a decision based on the level of PARP regarding identifying the breast cancer treatable by the PARP inhibitor or PARP antagonist. Another aspect of the present invention relates to a method of treating a breast cancer in a subject by PARP inhibitor or PARP antagonist comprising identifying a level of PARP in a sample of the subject, making a decision based on the level of PARP regarding identifying the breast cancer treatable by the PARP modulators, and treating the breast cancer by the PARP inhibitor or PARP antagonist. In some embodiments, the level of PARP is up-regulated. In some embodiments, the subject is deficient in BRCA gene. In some embodiments, the subject has down-regulated BRCA gene. In some methods, increase in PARP levels is an indication of BRCA1 and/or BRACA2 deficiency.

One aspect is methods of diagnosing and/or treating breast cancers. One embodiment is a method of identifying a breast cancer treatable with a PARP inhibitor comprising identifying a level of PARP in a plurality of samples from a subject and making a decision based on said level of PARP regarding whether said breast cancer is treatable with said PARP inhibitor. Another embodiment is a method of treating a breast cancer with a PARP inhibitor comprising identifying a level of PARP in a plurality of samples from a population; making a decision based on said level of PARP regarding whether said breast cancer is treatable with said PARP inhibitor; and treating said breast cancer with said PARP inhibitor. Yet another embodiment is method of classifying a breast tumor comprising identifying a level of PARP in a plurality of tumor samples from a population with breast cancer and making a decision regarding treating said tumor with a PARP modulator, wherein said decision is made based on said level of PARP. Another embodiment is a method of treating a breast tumor comprising identifying a level of PARP in a plurality of samples from a population; making a decision based on said level of PARP regarding treating said tumor with a PARP modulator; determining the level of PARP in a subject and comparing said level with the level PARP in the plurality of samples from the population, and treating said tumor in said subject with said PARP modulator if the level of PARP in the subject is above a predetermined value. Preferably, the breast tumor is an infiltrating duct carcinoma. In some embodiments, the cancers are negative for ER, Her2-neu, and/or PR. Another embodiment is a method of treating a cancer in a subject comprising identifying a presence or absence of ER, Her2-neu, and PR in a cancer sample from said subject and treating said cancer with a PARP inhibitor, wherein said treatment is performed if said cancer sample is negative for ER, Her2-neu, and/or PR.

In another aspect the methods of diagnosing and/or treating breast cancers involve comparison of a level of PARP from a subject in need of diagnosis or treatment to a pre-determined level of PARP. One embodiment is a method of identifying a breast cancer treatable with a PARP inhibitor comprising identifying a level of PARP in a sample from a subject; and determining whether said level of PARP is above a predetermined level thereby determining that said breast cancer is treatable with a PARP modulator, wherein the predetermined level is derived from a PARP level of each of a plurality of samples from a population with said breast cancer. Another embodiment is a method of treating a breast cancer in a patient with a PARP inhibitor comprising identifying a level of PARP in a sample from said subject; determining whether said level of PARP is above a predetermined level thereby determining that said breast cancer is treatable with a PARP inhibitor; and treating said breast cancer by administering said PARP inhibitor to said patient, wherein the predetermined level is derived from a PARP level of each of a plurality of samples from a population with said breast cancer. In some aspects, the subject is also BRCA 1 or BRCA2 deficient. Some subjects have decreased level of expression of a BRCA gene. Another embodiment is a method of classifying a breast tumor in a patient comprising identifying a level of PARP in a tumor sample from said patient and determining whether said level of PARP is above a predetermined level thereby classifying said breast tumor as treatable with a PARP modulator, wherein the predetermined level is derived from a PARP level of each of a plurality of samples from a population with said breast cancer. One method is a method of treating a breast tumor in a subject comprising identifying a level of PARP in a sample from said subject; determining whether said level of PARP is above a predetermined level thereby determining that said breast tumor is treatable with a PARP modulator and treating said tumor in said patient with said PARP modulator, wherein the predetermined level is derived from a PARP level of each of a plurality of samples from a population with said breast cancer. Yet another method is a method of identifying a breast tumor treatable with a PARP inhibitor comprising identifying a level of PARP in a sample from a patient; determining whether said level of PARP is above a predetermined level thereby identifying said breast tumor as treatable with a PARP inhibitor, wherein the predetermined level is derived from a PARP level of each of a plurality of samples from a population with said breast cancer. Another method is a method of treating a breast tumor in a patient with a PARP inhibitor comprising identifying a level of PARP in a sample from said patient; determining whether said level of PARP is above a predetermined level thereby determining that said breast tumor is treatable with a PARP inhibitor and treating said breast tumor by administering said PARP inhibitor to said patient, wherein the predetermined level is derived from a PARP level of each of a plurality of samples from a population with said breast cancer. Typically the breast tumor is an infiltrating duct carcinoma. Some of the infiltrating duct carcinoma is negative for ER, Her2-neu, and/or PR. A preferred method is a method of treating a cancer in a patient comprising determining whether ER, Her2-neu, and/or PR are present in a cancer sample from said patient and treating said cancer with a PARP inhibitor when ER, Her2-neu, and/or PR are not present in said sample from said patient, wherein the predetermined level is derived from a PARP level of each of a plurality of samples from a population with said breast cancer.

One embodiment is a method of identifying a PARP mediated disease or a stage of a PARP mediated disease treatable with a PARP modulator comprising identifying a level of PARP in a plurality of samples from a population and determining whether said level of PARP is above a predetermined level thereby determining that said PARP mediated disease is to be treated with a PARP modulator. Another embodiment is a method of treating a disease by administration of a PARP modulator to a patient comprising identifying a level of PARP in a sample from said patient; determining whether said level of PARP is above a predetermined level thereby determining that said PARP mediated disease is to be treated with a PARP modulator and treating said disease in said subject by administering said PARP modulator to said patient, wherein the predetermined level is derived from a PARP level of each of a plurality of samples from a population with the disease.

Yet another aspect of the invention relates to a method of identifying a lung cancer treatable by PARP inhibitor or PARP antagonist comprising identifying a level of PARP in a sample of a subject, and making a decision based on the level of PARP regarding identifying the lung cancer treatable by the PARP inhibitor or PARP antagonist. Another aspect of the present invention relates to a method of treating a lung cancer in a subject by PARP inhibitor or PARP antagonist comprising identifying a level of PARP in a sample of the subject, making a decision based on the level of PARP regarding identifying the lung cancer treatable by the PARP modulators, and treating the lung cancer by the PARP inhibitor or PARP antagonist. In some embodiments, the level of PARP is up-regulated.

One aspect is methods of diagnosing and/or treating lung cancers. One embodiment is a method of identifying a lung cancer treatable with a PARP inhibitor comprising identifying a level of PARP in a plurality of samples from a subject and making a decision based on said level of PARP regarding whether said lung cancer is treatable with said PARP inhibitor. Another embodiment is a method of treating a lung cancer with a PARP inhibitor comprising identifying a level of PARP in a plurality of samples from a population; making a decision based on said level of PARP regarding whether said lung cancer is treatable with said PARP inhibitor; and treating said lung cancer with said PARP inhibitor. Yet another embodiment is method of classifying a lung tumor comprising identifying a level of PARP in a plurality of tumor samples from a population with a lung tumor and making a decision regarding treating said tumor with a PARP modulator, wherein said decision is made based on said level of PARP. Another embodiment is a method of treating a lung tumor comprising identifying a level of PARP in a plurality of samples from a population; making a decision based on said level of PARP regarding treating said tumor with a PARP modulator; determining the level of PARP in a subject and comparing said level with the level PARP in the plurality of samples from the population, and treating said tumor in said subject with said PARP modulator if the level of PARP in the subject is above a predetermined value.

In another aspect the methods of diagnosing and/or treating lung cancers involve comparison of a level of PARP from a subject in need of diagnosis or treatment to a pre-determined level of PARP. One embodiment is a method of identifying a lung cancer treatable with a PARP inhibitor comprising identifying a level of PARP in a sample from a subject; and determining whether said level of PARP is above a predetermined level thereby determining that said lung cancer is treatable with a PARP modulator, wherein the predetermined level is derived from a PARP level of each of a plurality of samples from a population with said lung cancer. Another embodiment is a method of treating a lung cancer in a patient with a PARP inhibitor comprising identifying a level of PARP in a sample from said subject; determining whether said level of PARP is above a predetermined level thereby determining that said lung cancer is treatable with a PARP inhibitor; and treating said lung cancer by administering said PARP inhibitor to said patient, wherein the predetermined level is derived from a PARP level of each of a plurality of samples from a population with said lung cancer. Another embodiment is a method of classifying a lung tumor in a patient comprising identifying a level of PARP in a tumor sample from said patient and determining whether said level of PARP is above a predetermined level thereby classifying said lung tumor as treatable with a PARP modulator, wherein the predetermined level is derived from a PARP level of each of a plurality of samples from a population with said lung cancer. One method is a method of treating a lung tumor in a subject comprising identifying a level of PARP in a sample from said subject; determining whether said level of PARP is above a predetermined level thereby determining that said lung tumor is treatable with a PARP modulator and treating said tumor in said patient with said PARP modulator, wherein the predetermined level is derived from a PARP level of each of a plurality of samples from a population with said lung cancer. Yet another method is a method of identifying a lung tumor treatable with a PARP inhibitor comprising identifying a level of PARP in a sample from a patient; determining whether said level of PARP is above a predetermined level thereby identifying said lung tumor as treatable with a PARP inhibitor, wherein the predetermined level is derived from a PARP level of each of a plurality of samples from a population with said lung cancer. Another method is a method of treating a lung tumor in a patient with a PARP inhibitor comprising identifying a level of PARP in a sample from said patient; determining whether said level of PARP is above a predetermined level thereby determining that said lung tumor is treatable with a PARP inhibitor and treating said lung tumor by administering said PARP inhibitor to said patient, wherein the predetermined level is derived from a PARP level of each of a plurality of samples from a population with said lung cancer.

Yet another aspect of the invention relates to a method of identifying an ovarian cancer treatable by PARP inhibitor or PARP antagonist comprising identifying a level of PARP in a sample of a subject, and making a decision based on the level of PARP regarding identifying the ovarian cancer treatable by the PARP inhibitor or PARP antagonist. Another aspect of the present invention relates to a method of treating an ovarian cancer in a subject by PARP inhibitor or PARP antagonist comprising identifying a level of PARP in a sample of the subject, making a decision based on the level of PARP regarding identifying the ovarian cancer treatable by the PARP modulators, and treating the ovarian cancer by the PARP inhibitor or PARP antagonist.

One aspect is methods of diagnosing and/or treating ovarian cancers. One embodiment is a method of identifying an ovarian cancer treatable with a PARP inhibitor comprising identifying a level of PARP in a plurality of samples from a subject and making a decision based on said level of PARP regarding whether said ovarian cancer is treatable with said PARP inhibitor. Another embodiment is a method of treating an ovarian cancer with a PARP inhibitor comprising identifying a level of PARP in a plurality of samples from a population; making a decision based on said level of PARP regarding whether said ovarian cancer is treatable with said PARP inhibitor; and treating said ovarian cancer with said PARP inhibitor. Yet another embodiment is method of classifying a ovarian tumor comprising identifying a level of PARP in a plurality of tumor samples from a population with ovarian cancer and making a decision regarding treating said tumor with a PARP modulator, wherein said decision is made based on said level of PARP. Another embodiment is a method of treating a ovarian tumor comprising identifying a level of PARP in a plurality of samples from a population; making a decision based on said level of PARP regarding treating said tumor with a PARP modulator; determining the level of PARP in a subject and comparing said level with the level PARP in the plurality of samples from the population, and treating said tumor in said subject with said PARP modulator if the level of PARP in the subject is above a predetermined value.

In another aspect the methods of diagnosing and/or treating ovarian cancers involve comparison of a level of PARP from a subject in need of diagnosis or treatment to a pre-determined level of PARP. One embodiment is a method of identifying an ovarian cancer treatable with a PARP inhibitor comprising identifying a level of PARP in a sample from a subject; and determining whether said level of PARP is above a predetermined level thereby determining that said ovarian cancer is treatable with a PARP modulator, wherein the predetermined level is derived from a PARP level of each of a plurality of samples from a population with said ovarian cancer. Another embodiment is a method of treating an ovarian cancer in a patient with a PARP inhibitor comprising identifying a level of PARP in a sample from said subject; determining whether said level of PARP is above a predetermined level thereby determining that said ovarian cancer is treatable with a PARP inhibitor; and treating said ovarian cancer by administering said PARP inhibitor to said patient, wherein the predetermined level is derived from a PARP level of each of a plurality of samples from a population with said ovarian cancer. Another embodiment is a method of classifying an ovarian tumor in a patient comprising identifying a level of PARP in a tumor sample from said patient and determining whether said level of PARP is above a predetermined level thereby classifying said ovarian tumor as treatable with a PARP modulator, wherein the predetermined level is derived from a PARP level of each of a plurality of samples from a population with said ovarian cancer. One method is a method of treating a ovarian tumor in a subject comprising identifying a level of PARP in a sample from said subject; determining whether said level of PARP is above a predetermined level thereby determining that said ovarian tumor is treatable with a PARP modulator and treating said tumor in said patient with said PARP modulator, wherein the predetermined level is derived from a PARP level of each of a plurality of samples from a population with said ovarian cancer. Yet another method is a method of identifying an ovarian tumor treatable with a PARP inhibitor comprising identifying a level of PARP in a sample from a patient; determining whether said level of PARP is above a predetermined level thereby identifying said ovarian tumor as treatable with a PARP inhibitor, wherein the predetermined level is derived from a PARP level of each of a plurality of samples from a population with said ovarian cancer. Another method is a method of treating an ovarian tumor in a patient with a PARP inhibitor comprising identifying a level of PARP in a sample from said patient; determining whether said level of PARP is above a predetermined level thereby determining that said ovarian tumor is treatable with a PARP inhibitor and treating said ovarian tumor by administering said PARP inhibitor to said patient, wherein the predetermined level is derived from a PARP level of each of a plurality of samples from a population with said ovarian cancer.

Yet another aspect of the invention relates to a method of identifying an endometrial cancer treatable by PARP inhibitor or PARP antagonist comprising identifying a level of PARP in a sample of a subject, and making a decision based on the level of PARP regarding identifying the endometrial cancer treatable by the PARP inhibitor or PARP antagonist. Another aspect of the present invention relates to a method of treating an endometrial cancer in a subject by PARP inhibitor or PARP antagonist comprising identifying a level of PARP in a sample of the subject, making a decision based on the level of PARP regarding identifying the endometrial cancer treatable by the PARP modulators, and treating the endometrial cancer by the PARP inhibitor or PARP antagonist.

One aspect is methods of diagnosing and/or treating endometrial cancers. One embodiment is a method of identifying an endometrial cancer treatable with a PARP inhibitor comprising identifying a level of PARP in a plurality of samples from a subject and making a decision based on said level of PARP regarding whether said endometrial cancer is treatable with said PARP inhibitor. Another embodiment is a method of treating an endometrial cancer with a PARP inhibitor comprising identifying a level of PARP in a plurality of samples from a population; making a decision based on said level of PARP regarding whether said endometrial cancer is treatable with said PARP inhibitor; and treating said endometrial cancer with said PARP inhibitor. Yet another embodiment is method of classifying a endometrial tumor comprising identifying a level of PARP in a plurality of tumor samples from a population with endometrial cancer and making a decision regarding treating said tumor with a PARP modulator, wherein said decision is made based on said level of PARP. Another embodiment is a method of treating a endometrial tumor comprising identifying a level of PARP in a plurality of samples from a population; making a decision based on said level of PARP regarding treating said tumor with a PARP modulator; determining the level of PARP in a subject and comparing said level with the level PARP in the plurality of samples from the population, and treating said tumor in said subject with said PARP modulator if the level of PARP in the subject is above a predetermined value.

In another aspect the methods of diagnosing and/or treating endometrial cancers involve comparison of a level of PARP from a subject in need of diagnosis or treatment to a pre-determined level of PARP. One embodiment is a method of identifying an endometrial cancer treatable with a PARP inhibitor comprising identifying a level of PARP in a sample from a subject; and determining whether said level of PARP is above a predetermined level thereby determining that said endometrial cancer is treatable with a PARP modulator, wherein the predetermined level is derived from a PARP level of each of a plurality of samples from a population with said endometrial cancer. Another embodiment is a method of treating an endometrial cancer in a patient with a PARP inhibitor comprising identifying a level of PARP in a sample from said subject; determining whether said level of PARP is above a predetermined level thereby determining that said endometrial cancer is treatable with a PARP inhibitor; and treating said endometrial cancer by administering said PARP inhibitor to said patient, wherein the predetermined level is derived from a PARP level of each of a plurality of samples from a population with said endometrial cancer. Another embodiment is a method of classifying an endometrial tumor in a patient comprising identifying a level of PARP in a tumor sample from said patient and determining whether said level of PARP is above a predetermined level thereby classifying said endometrial tumor as treatable with a PARP modulator, wherein the predetermined level is derived from a PARP level of each of a plurality of samples from a population with said endometrial cancer. One method is a method of treating a endometrial tumor in a subject comprising identifying a level of PARP in a sample from said subject; determining whether said level of PARP is above a predetermined level thereby determining that said endometrial tumor is treatable with a PARP modulator and treating said tumor in said patient with said PARP modulator, wherein the predetermined level is derived from a PARP level of each of a plurality of samples from a population with said endometrial cancer. Yet another method is a method of identifying an endometrial tumor treatable with a PARP inhibitor comprising identifying a level of PARP in a sample from a patient; determining whether said level of PARP is above a predetermined level thereby identifying said endometrial tumor as treatable with a PARP inhibitor, wherein the predetermined level is derived from a PARP level of each of a plurality of samples from a population with said endometrial cancer. Another method is a method of treating an endometrial tumor in a patient with a PARP inhibitor comprising identifying a level of PARP in a sample from said patient; determining whether said level of PARP is above a predetermined level thereby determining that said endometrial tumor is treatable with a PARP inhibitor and treating said endometrial tumor by administering said PARP inhibitor to said patient, wherein the predetermined level is derived from a PARP level of each of a plurality of samples from a population with said endometrial cancer.

One aspect of the invention is a computer-readable medium suitable for transmission of a result of an analysis of a sample wherein the medium comprises an information regarding a disease in a subject treatable with a PARP modulator; said information being derived by identifying a level of PARP in said sample from said subject; and determining whether said level of PARP is above a predetermined level thereby determining that said PARP mediated disease is to be treated with a PARP modulator.

One aspect of the invention is a computer-readable medium suitable for transmission of a result of an analysis of a sample wherein the medium comprises an information regarding a disease in a subject treatable with a PARP modulator; said information being derived by identifying a level of PARP in said sample from said subject; and determining whether said level of PARP is above a predetermined level thereby determining that said PARP mediated disease is to be treated with a PARP modulator, wherein the predetermined level is derived from a PARP level of each of a plurality of samples from a population with said disease.

Yet another aspect of the present invention is classification of patient populations and assessing responses to PARP treatment. One embodiment is a method of selecting a subject for therapy with the PARP inhibitor comprising measuring a level of PARP in a biological sample collected from the subject prior to administration of the PARP inhibitor, determining that the PARP level in the sample is higher than a predetermined value and selecting the subject for therapy with the PARP inhibitor. Yet another embodiment is a method of treating a subject with a PARP inhibitor comprising measuring a level of PARP in a biological sample collected from the subject prior to administration of the PARP inhibitor, determining that the PARP level in the sample is higher than a predetermined value and administering to the subject the PARP inhibitor. Another embodiment is a method of assessing response to treatment in a subject undergoing therapy with a PARP inhibitor the method comprising: measuring the PARP level in the subject at least a first and a second point in time to produce at least a first level of PARP and a second level of PARP, wherein a decrease in the second level of PARP compared to the first level of PARP is indicative of positive response to treatment. Typically, the first time point is before the start of treatment with a PARP inhibitor and the second time point is after start of treatment with a PARP inhibitor. In some embodiments, the first time point after start of treatment with a PARP inhibitor and the second time point is at later time after the first time point, such as a few days, weeks, or months later. Another embodiment is a method for treating a patient whose condition results in an elevated PARP level, wherein a PARP level of a patient sample is higher than a pre-determined PARP level, the method comprising, administering a therapeutically effective amount of a PARP inhibitor.

Yet another aspect of the present invention is the classification of patient populations and, assessing responses to PARP treatment. One embodiment is a method of selecting a subject for therapy with the PARP inhibitor comprising measuring a level of PARP in a biological sample collected from the subject prior to administration of the PARP inhibitor, determining that the PARP level in the sample is higher than a predetermined value and selecting the subject for therapy with the PARP inhibitor, wherein the predetermined level is derived from a PARP level of each of a plurality of samples from a population. Yet another embodiment is a method of treating a subject with a PARP inhibitor comprising measuring a level of PARP in a biological sample collected from the subject prior to administration of the PARP inhibitor, determining that the PARP level in the sample is higher than a predetermined value and administering to the subject the PARP inhibitor, wherein the predetermined level is derived from a PARP level of each of a plurality of samples from a population. Another embodiment is a method of assessing response to treatment in a subject undergoing therapy with a PARP inhibitor the method comprising: measuring the PARP level in the subject at least a first and a second point in time to produce at least a first level of PARP and a second level of PARP, wherein a decrease in the second level of PARP compared to the first level of PARP is indicative of positive response to treatment. Typically, the first time point is before the start of treatment with a PARP inhibitor and the second time point is after start of treatment with a PARP inhibitor. In some embodiments, the first time point after start of treatment with a PARP inhibitor and the second time point is at later time after the first time point, such as a few days, weeks, or months later. Another embodiment is a method for treating a patient whose condition results in an elevated PARP level, wherein a PARP level of a patient sample is higher than a pre-determined PARP level, the method comprising, administering a therapeutically effective amount of a PARP inhibitor.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a flow chart showing the steps of the methods disclosed herein.

FIG. 2 illustrates a computer for implementing selected operations associated with the methods disclosed herein.

FIG. 3 depicts correlation of high expression of PARP1 with lower expression of BRCA1 and 2 in primary ovarian tumors.

FIGS. 4 a-b and 5 a-b depict PARP expression in infiltrating duct carcinoma subtypes.

FIG. 6 depict PARP expression in malignant and normal ovarian tissue.

FIG. 7 depicts PARP expression in malignant and normal endometrium tissue.

FIG. 8 depicts PARP expression in malignant and normal lung tissue.

FIG. 9 depicts PARP expression in malignant and normal prostate tissue.

FIG. 10 depicts PARP expression in human healthy tissues.

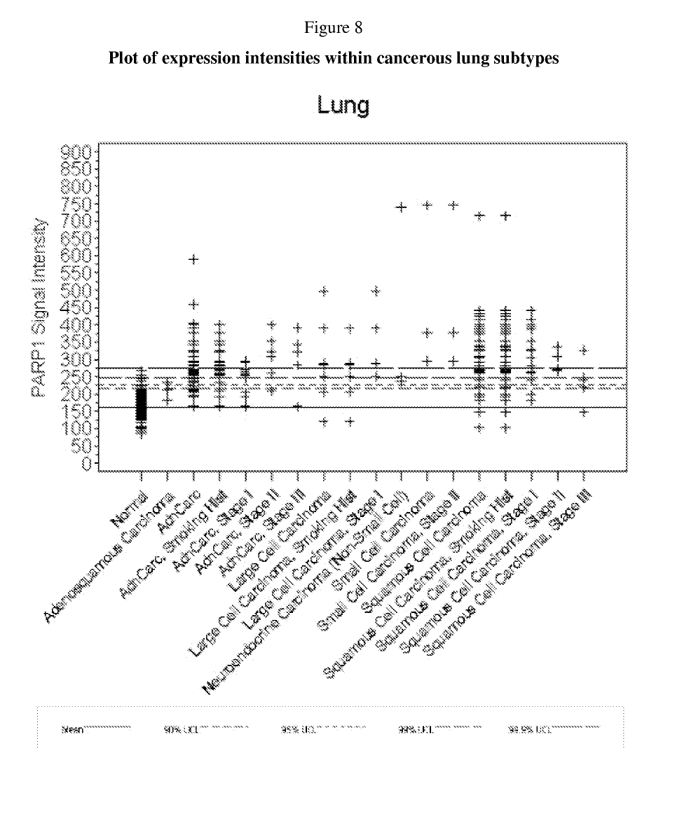

FIG. 11 depicts PARP expression in malignant and normal tissues.

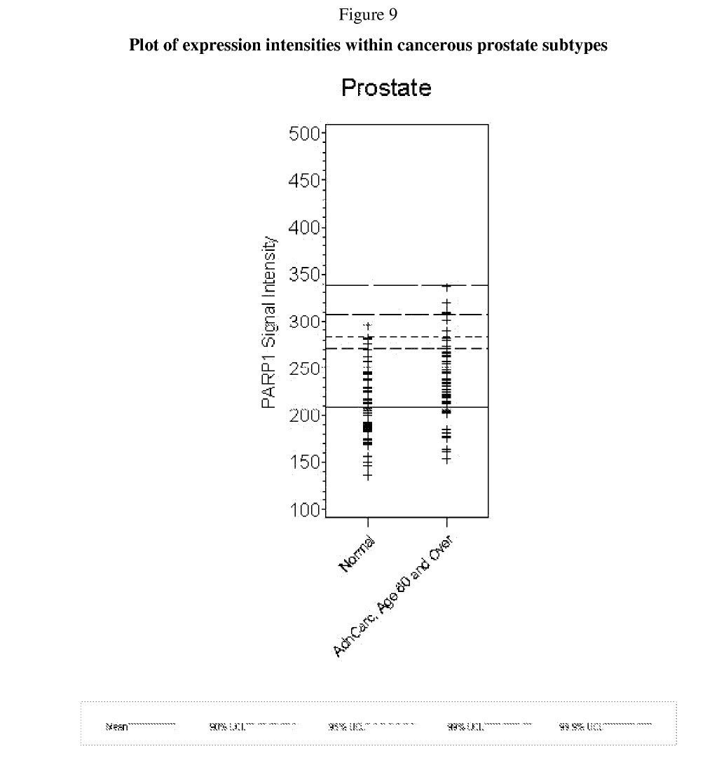

FIG. 12 depicts PARP expression in human primary tumors.

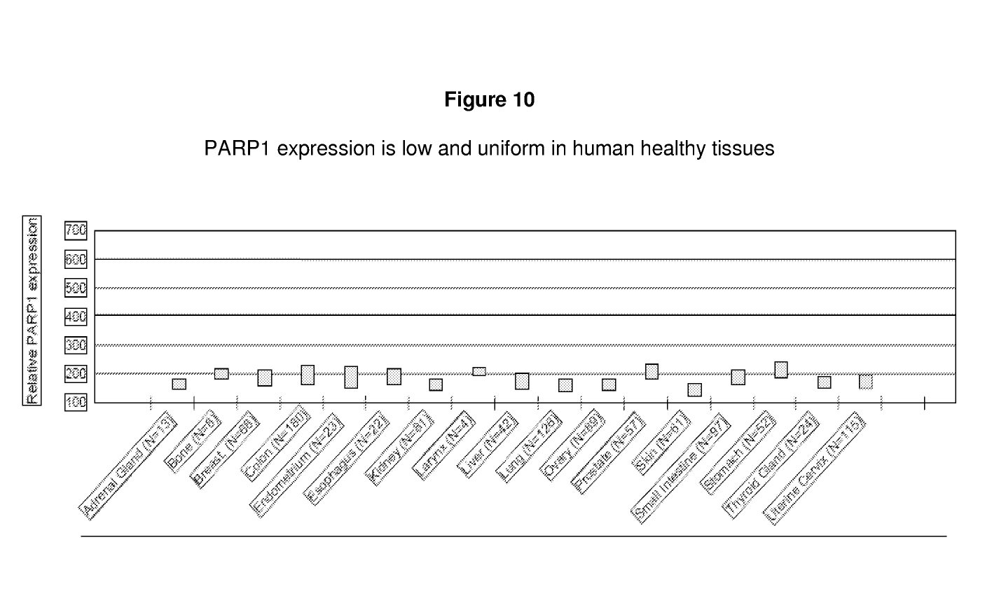

FIG. 13 depicts PARP expression in lung human and tumor syngenic specimens.

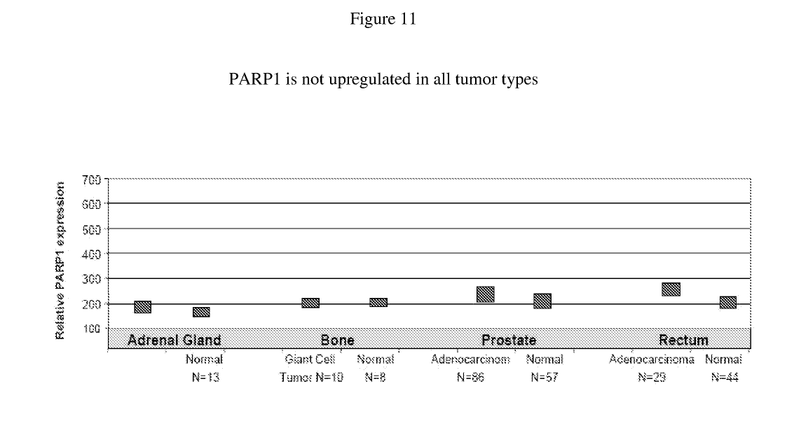

FIG. 14 depicts PARP expression in lung normal and tumor tissues.

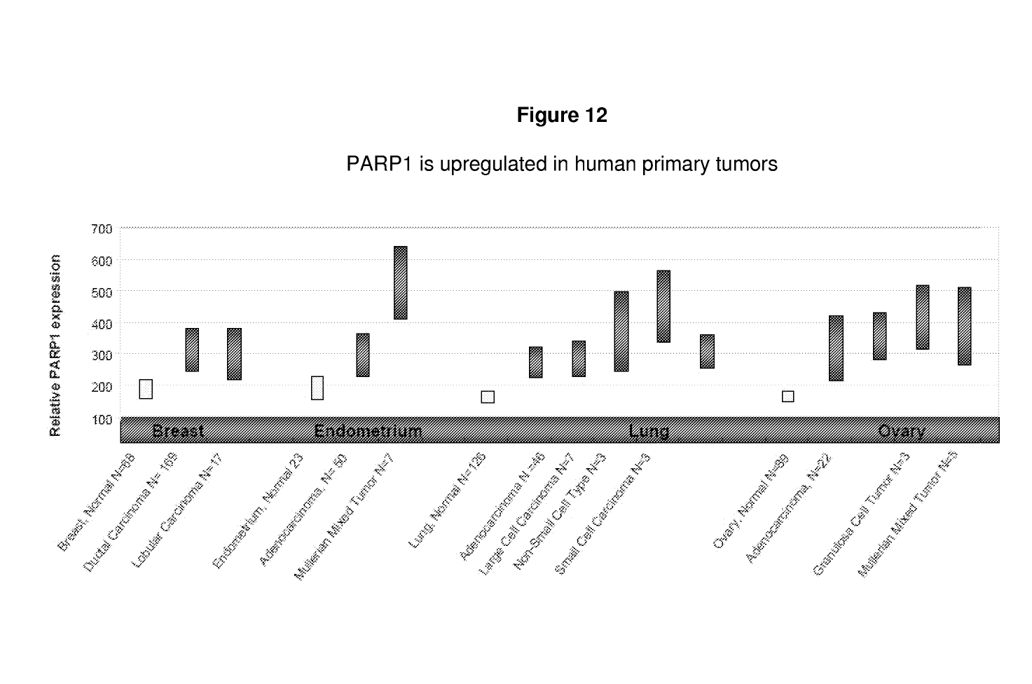

FIG. 15 depicts PARP expression in a lung human normal and tumor syngenic specimen.

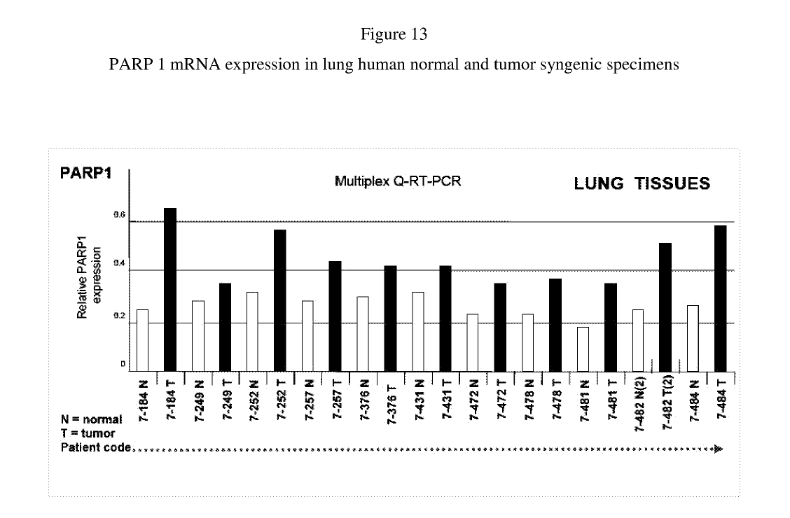

FIG. 16 depicts PARP expression in a lung human normal and tumor syngenic specimen.

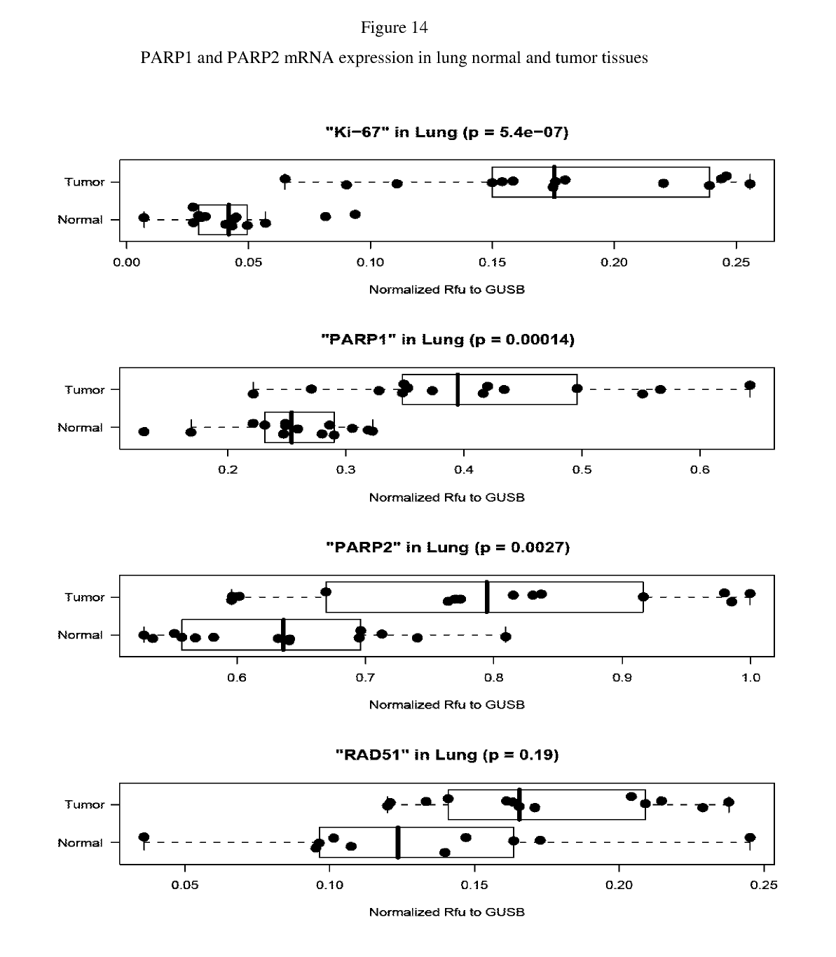

FIG. 17 depicts PARP expression in a lung human normal and tumor syngenic specimen.

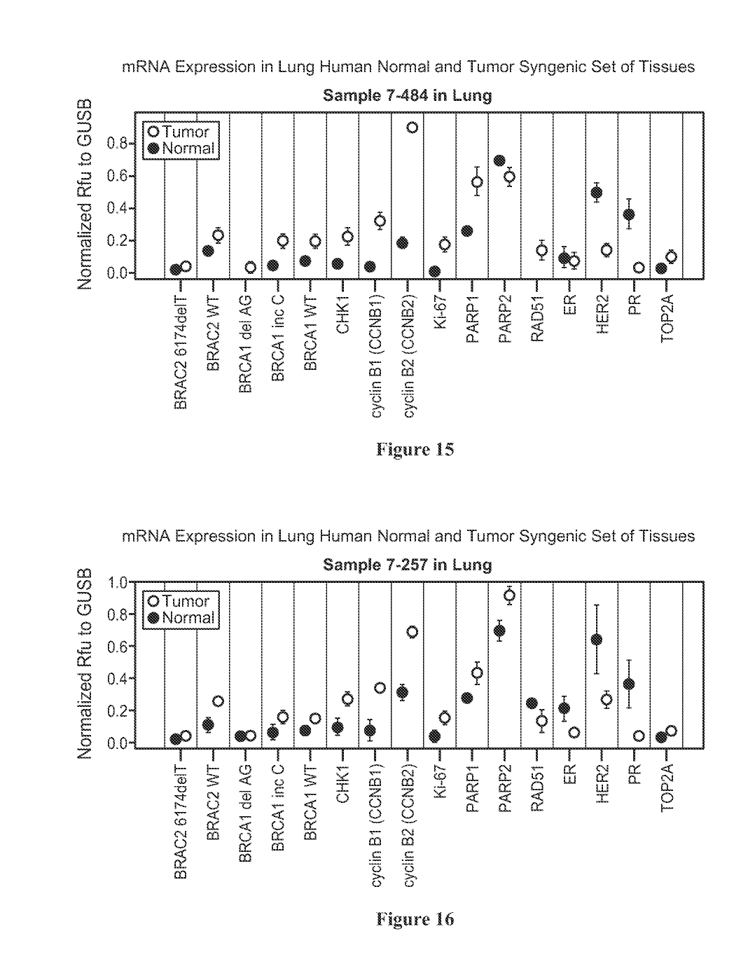

FIG. 18 depicts PARP expression in breast human normal and tumor syngenic specimens.

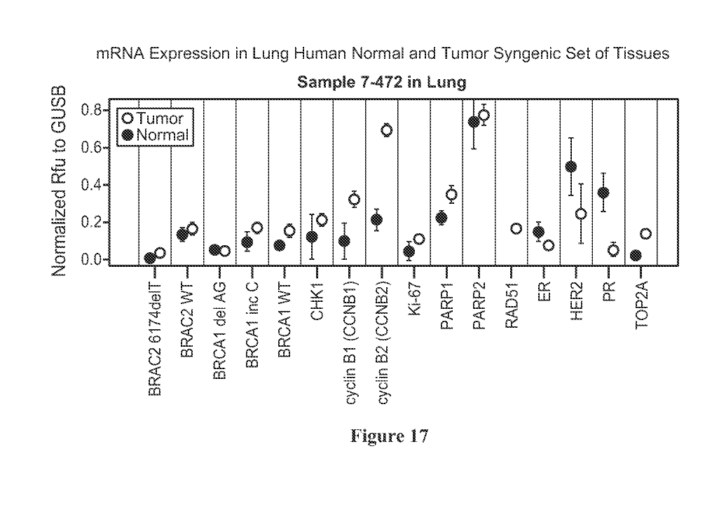

FIG. 19 depicts PARP expression in breast human normal and tumor tissues.

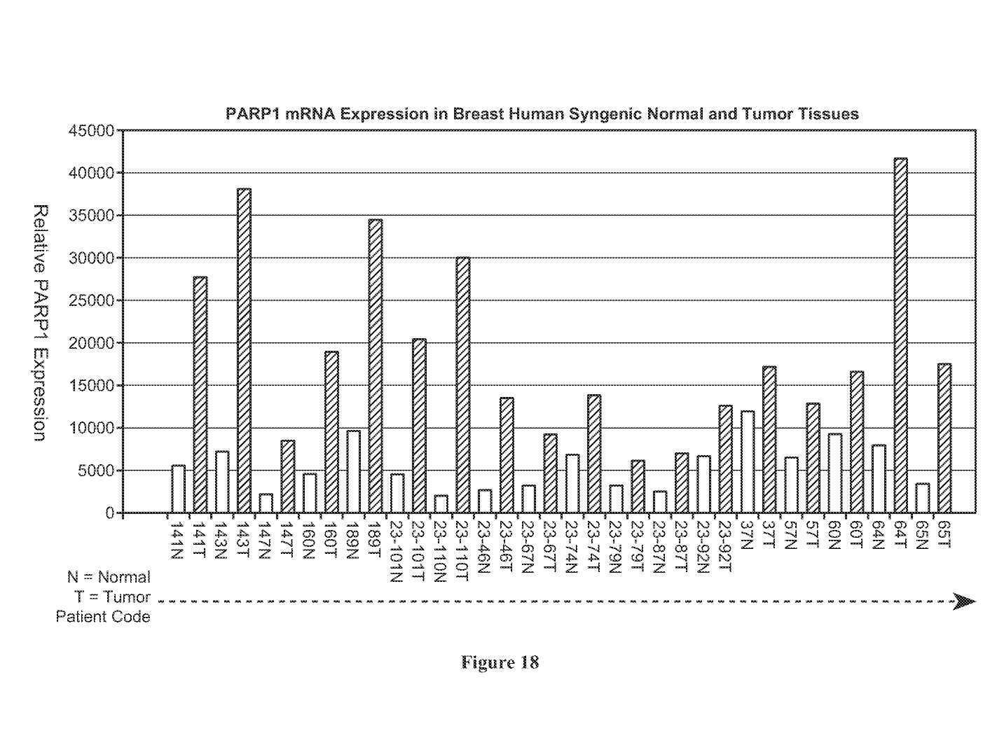

FIG. 20 depicts PARP expression in a breast human normal and tumor syngenic specimen.

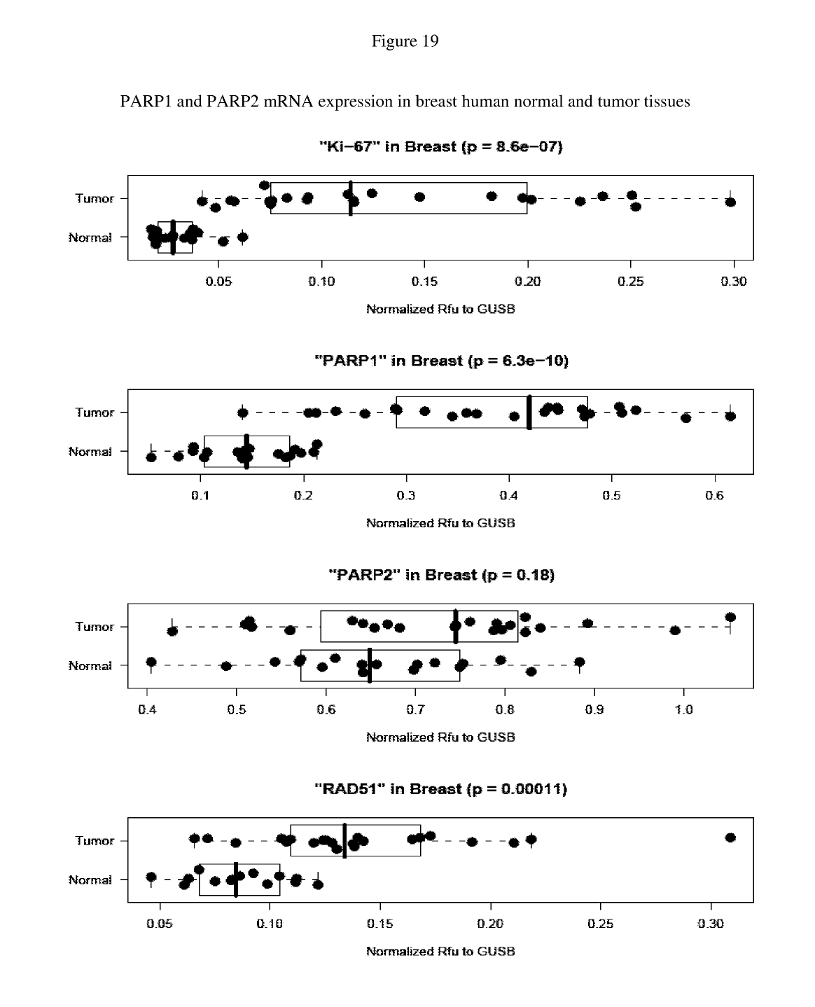

FIG. 21 depicts PARP expression in a breast human normal and tumor syngenic specimen.

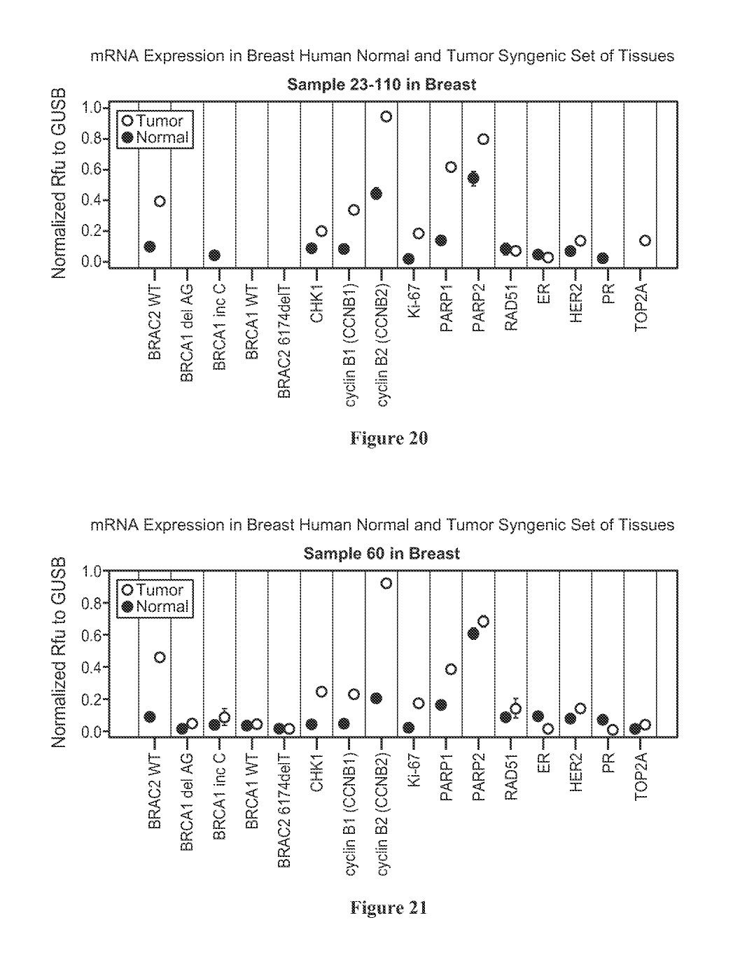

FIG. 22 depicts PARP expression in a breast human normal and tumor syngenic specimen.

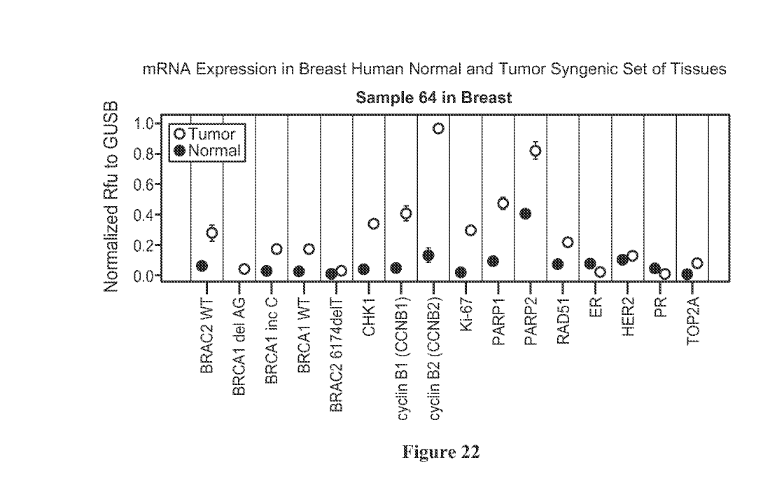

FIG. 23 depicts upregulation of PARP expression in a ER-, PR- and Her-2 negative tissue specimen.

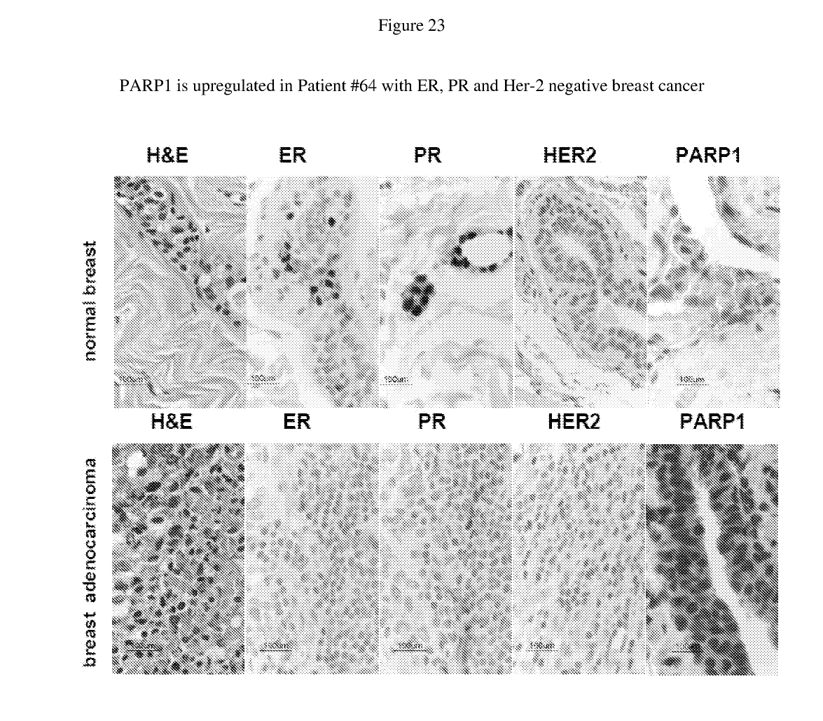

### DETAILED DESCRIPTION OF THE INVENTION

The term “inhibit” or its grammatical equivalent, such as “inhibitory,” is not intended to require complete reduction in PARP activity. Such reduction is preferably by at least about 50%, at least about 75%, at least about 90%, and more preferably by at least about 95% of the activity of the molecule in the absence of the inhibitory effect, e.g., in the absence of an inhibitor, such as PARP inhibitors disclosed in the invention. Most preferably, the term refers to an observable or measurable reduction in activity. In treatment scenarios, preferably the inhibition is sufficient to produce a therapeutic and/or prophylactic benefit in the condition being treated.

The terms “sample”, “biological sample” or its grammatical equivalents, as used herein mean a material known to or suspected of expressing a level of PARP. The test sample can be used directly as obtained from the source or following a pretreatment to modify the character of the sample. The sample can be derived from any biological source, such as tissues or extracts, including cells, and physiological fluids, such as, for example, whole blood, plasma, serum, saliva, ocular lens fluid, cerebrospinal fluid, sweat, urine, milk, ascites fluid, synovial fluid, peritoneal fluid and the like. The sample is obtained from animals or humans, preferably from humans. The sample can be treated prior to use, such as preparing plasma from blood, diluting viscous fluids, and the like. Methods of treating a sample can involve filtration, distillation, extraction, concentration, inactivation of interfering components, the addition of reagents, and the like.

The term “subject” or its grammatical equivalents as used herein refers to a warm-blooded animal such as a mammal who is healthy or is afflicted with, or suspected to be afflicted with a disease. Preferably, “subject” refers to a human.

The term “population” or its grammatical equivalents as used herein refers to a plurality of subjects, preferably warm-blooded animals such as a mammal who is healthy or is afflicted with, or suspected to be afflicted with a disease, most preferably human. A plurality of subjects may consist of at least two or more subjects, at least three or more subjects, at least four or more subjects, at least ten or more subjects, at least twenty or more subjects, at least fifty or more subjects, or at least one hundred or more subjects. In some aspects, a population may consist of only one subject, for example, in cases where the disease is rare or the patient population is difficult to define.

The term “treating” or its grammatical equivalents as used herein, means achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

### Method of Identifying a Disease or Stage of a Disease Treatable by PARP Modulators

In one aspect of the present invention, the methods include identifying a disease treatable by PARP modulators comprising identifying a level of PARP in a sample of a subject, making a decision regarding identifying the disease treatable by the PARP modulators wherein the decision is made based on the level of PARP. In another aspect of the present invention, the methods include treating a disease by PARP modulators in a subject comprising identifying a level of PARP in a sample of the subject, making a decision based on the level of PARP regarding identifying the disease treatable by the PARP modulators, and treating the disease in the subject by the PARP modulators. In another aspect of the present invention, the method further includes providing a conclusion regarding the disease to a patient, a health care provider or a health care manager, where the conclusion is based on the decision. In some preferred embodiments, disease is breast cancer. In some preferred embodiments, the level of PARP is up-regulated. In some preferred embodiments, the level of PARP is detected by measuring expression of PARP gene.

In one aspect of the present invention, the methods include identifying a disease treatable by PARP modulators comprising identifying a level of PARP in a plurality of samples from a population, making a decision regarding identifying the disease treatable by the PARP modulators wherein the decision is made based on the level of PARP. In another aspect of the present invention, the methods include treating a disease by PARP modulators comprising identifying a level of PARP in a plurality of samples of a population, making a decision based on the level of PARP regarding identifying the disease treatable by the PARP modulators, and treating the disease in the subject by the PARP modulators. In another aspect of the present invention, the method further includes providing a conclusion regarding the disease to a patient, a health care provider or a health care manager, where the conclusion is based on the decision. In some preferred embodiments, the disease is breast cancer. In some preferred embodiments, the level of PARP is up-regulated. In some preferred embodiments, the level of PARP is detected by measuring expression of PARP gene.

The present invention relates to identifying a level of PARP in a sample of a subject suffering from a disease where when the level of PARP is up-regulated then the subject is treated with a PARP inhibitor or a PARP antagonist. The present invention identifies diseases such as, cancer, inflammation, metabolic disease, CVS disease, CNS disease, disorder of hematolymphoid system, disorder of endocrine and neuroendocrine, disorder of urinary tract, disorder of respiratory system, disorder of female reproductive system, and disorder of male reproductive system where the level of PARP is up-regulated. Accordingly, the present invention identifies these diseases to be treatable by PARP inhibitors. In a preferred embodiment, the PARP inhibitors used in the methods of the present invention are PARP-1 inhibitors. The PARP inhibitors used in the present invention can act via a direct or indirect interaction with PARP, preferably PARP-1. The PARP inhibitors used herein may modulate PARP or may modulate one or more entities in the PARP pathway. The PARP inhibitors can in some embodiments inhibit PARP activity.

The present invention relates to identifying a level of PARP in a plurality of samples of a population suffering from a disease where when the level of PARP is up-regulated then disease is treatable with a PARP inhibitor or a PARP antagonist. The present invention identifies diseases such as, cancer, inflammation, metabolic disease, CVS disease, CNS disease, disorder of hematolymphoid system, disorder of endocrine and neuroendocrine, disorder of urinary tract, disorder of respiratory system, disorder of female reproductive system, and disorder of male reproductive system where the level of PARP is up-regulated. Accordingly, the present invention identifies these diseases to be treatable by PARP inhibitors. In a preferred embodiment, the PARP inhibitors used in the methods of the present invention are PARP-1 inhibitors. The PARP inhibitors used in the present invention can act via a direct or indirect interaction with PARP, preferably PARP-1. The PARP inhibitors used herein may modulate PARP or may modulate one or more entities in the PARP pathway. The PARP inhibitors can in some embodiments inhibit PARP activity.

The method is particularly useful in treating cancer of female reproductive system. Breast tumours in women who inherit faults in either the BRCA1 or BRCA2 genes occur because the tumour cells have lost a specific mechanism that repair damaged DNA. BRCA1 and BRCA2 are important for DNA double-strand break repair by homologous recombination, and mutations in these genes predispose to breast and other cancers. PARP is involved in base excision repair, a pathway in the repair of DNA single-strand breaks. BRCA1 or BRCA2 dysfunction sensitizes cells to the inhibition of PARP enzymatic activity, resulting in chromosomal instability, cell cycle arrest and subsequent apoptosis.

PARP inhibitors kill cells where this form of DNA repair is absent and so are effective in killing BRCA deficient tumour cells and other similar tumour cells. Normal cells may be unaffected by the drug as they may still possess this DNA repair mechanism. This treatment might also be applicable to other forms of breast cancer that behave like BRCA deficient cancer. Typically, breast cancer patients are treated with drugs that kill tumour cells but also damage normal cells. It is damage to normal cells that can lead to distressing side effects, like nausea and hair loss. In some embodiments, an advantage of treating with PARP inhibitors is that it is targeted; tumour cells are killed while normal cells appear unaffected. This is because PARP inhibitors exploit the specific genetic make-up of some tumour cells.

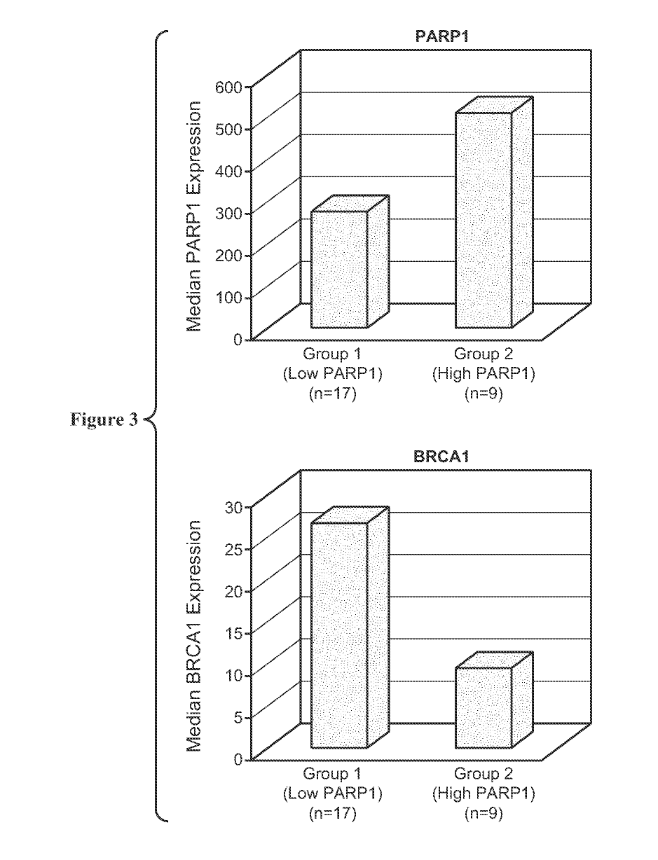

The present invention discloses that the subjects deficient in BRCA genes have up-regulated levels of PARP. FIG. 3 depicts correlation of high expression of PARP-1 with lower expression of BRCA1 and 2 in primary ovarian tumors. PARP up-regulation may be an indicator of other defective DNA-repair pathways and unrecognized BRCA-like genetic defects. Assessment of PARP-1 gene expression is an indicator of tumor sensitivity to PARP inhibitor. Hence, the present invention provides methods to identify early onset of cancer in BRCA deficient patients by measuring the level of PARP. The BRCA deficient patients treatable by PARP inhibitors can be identified if PARP is up-regulated. Further, such BRCA deficient patients can be treated with PARP inhibitors.

The present invention discloses that the subjects deficient in BRCA genes have up-regulated levels of PARP. FIG. 3 depicts correlation of high expression of PARP-1 with lower expression of BRCA 1 and 2 in primary ovarian tumors. PARP up-regulation may be an indicator of other defective DNA-repair pathways and unrecognized BRCA-like genetic defects. Assessment of PARP-1 gene expression is an indicator of tumor sensitivity to PARP inhibitor. Hence, the present invention provides methods to identify early onset of cancer in BRCA deficient patients by measuring the level of PARP in said patients, and comparing the level to a level of PARP from a plurality of samples from a population that is BRCA-deficient. The BRCA deficient patients treatable by PARP inhibitors can be identified if PARP is up-regulated over a predetermined value, wherein the predetermined value is derived from the plurality of samples from the population. Further, such BRCA deficient patients can be treated with PARP inhibitors.

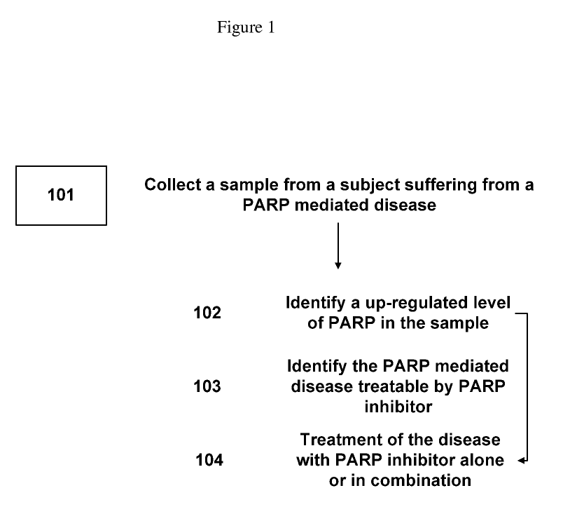

The steps to some of the preferable methods of the present invention are depicted in FIG. 1. Without limiting the scope of the present invention, the steps can be performed independent of each other or one after the other. One or more steps may be skipped in the methods of the present invention. A sample is collected from a subject suffering from a disease at step 101. In a preferred embodiment, the sample is human normal and tumor samples, hair, blood, and other biofluids. A level of the PARP is analyzed at step 102 by techniques well known in the art and based on the level of PARP such as, when PARP is up-regulated identifying the disease treatable by PARP inhibitors at step 103. The level of PARP may be compared to a predetermined value to determine if treatment should be commenced, wherein the predetermined value may be derived from a plurality of samples of a population with the disease. Step 104 comprises treating the subject suffering from the diseases with a PARP inhibitor. It shall be understood that the invention includes other methods not explicitly set forth herein. Without limiting the scope of the present invention, other techniques for collection of sample, analysis of PARP in the sample and treatment of the disease with PARP inhibitors are known in the art and are within the scope of the present invention.

In one embodiment of the present invention, tumors which are homologous recombination deficient are identified by evaluating levels of PARP expression. If upregulation of PARP is observed such tumors can be treated with PARP inhibitors. Another embodiment is a method for treating a homologous recombination deficient cancer comprising evaluating level of PARP expression and if overexpression is observed the cancer is treated with a PARP inhibitor.

### Sample Collection, Preparation and Separation

Biological samples in the present invention can be obtained from individuals with varying phenotypic states, such as various states of cancer or other diseases. Examples of phenotypic states also include phenotypes of normal subjects, which can be used for comparisons to diseased subjects. In some embodiments, subjects with disease are matched with control samples that are obtained from individuals who do not exhibit the disease.

Samples may be collected from a variety of sources from a mammal, preferably a human, including a body fluid sample, or a tissue sample. Samples collected can be human normal and tumor samples, hair, blood, other biofluids, cells, tissues, organs or bodily fluids for example, but not limited to, brain tissue, blood, serum, sputum including saliva, plasma, nipple aspirants, synovial fluids, cerebrospinal fluids, sweat, urine, fecal matter, pancreatic fluid, trabecular fluid, cerebrospinal fluid, tears, bronchial lavage, swabbings, bronchial aspirants, semen, prostatic fluid, precervicular fluid, vaginal fluids, pre-ejaculate, etc. Suitable tissue samples include various types of tumor or cancer tissue, or organ tissue, such as those taken at biopsy.

The samples can be collected from individuals repeatedly over a longitudinal period of time (e.g., about once a day, once a week, once a month, biannually or annually). Obtaining numerous samples from an individual over a period of time can be used to verify results from earlier detections and/or to identify an alteration in biological pattern as a result of, for example, disease progression, drug treatment, etc.

Sample preparation and separation can involve any of the procedures, depending on the type of sample collected and/or analysis of PARP. Such procedures include, by way of example only, concentration, dilution, adjustment of pH, removal of high abundance polypeptides (e.g., albumin, gamma globulin, and transferin, etc.), addition of preservatives and calibrants, addition of protease inhibitors, addition of denaturants, desalting of samples, concentration of sample proteins, extraction and purification of lipids.

The sample preparation can also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

Removal of undesired proteins (e.g., high abundance, uninformative, or undetectable proteins) from a sample can be achieved using high affinity reagents, high molecular weight filters, ultracentrifugation and/or electrodialysis. High affinity reagents include antibodies or other reagents (e.g. aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

Ultracentrifugation is a method for removing undesired polypeptides from a sample. Ultracentrifugation is the centrifugation of a sample at about 15,000-60,000 rpm while monitoring with an optical system the sedimentation (or lack thereof) of particles. Electrodialysis is a procedure which uses an electromembrane or semipermable membrane in a process in which ions are transported through semi-permeable membranes from one solution to another under the influence of a potential gradient. Since the membranes used in electrodialysis may have the ability to selectively transport ions having positive or negative charge, reject ions of the opposite charge, or to allow species to migrate through a semipermable membrane based on size and charge, it renders electrodialysis useful for concentration, removal, or separation of electrolytes.

Separation and purification in the present invention may include any procedure known in the art, such as capillary electrophoresis (e.g., in capillary or on-chip) or chromatography (e.g., in capillary, column or on a chip). Electrophoresis is a method which can be used to separate ionic molecules under the influence of an electric field. Electrophoresis can be conducted in a gel, capillary, or in a microchannel on a chip. Examples of gels used for electrophoresis include starch, acrylamide, polyethylene oxides, agarose, or combinations thereof. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray.

Capillary electrophoresis (CE) is preferred for separating complex hydrophilic molecules and highly charged solutes. CE technology can also be implemented on microfluidic chips. Depending on the types of capillary and buffers used, CE can be further segmented into separation techniques such as capillary zone electrophoresis (CZE), capillary isoelectric focusing (CIEF), capillary isotachophoresis (cITP) and capillary electrochromatography (CEC). An embodiment to couple CE techniques to electrospray ionization involves the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. Capillary zone electrophoresis (CZE), also known as free-solution CE (FSCE), is based on differences in the electrophoretic mobility of the species, determined by the charge on the molecule, and the frictional resistance the molecule encounters during migration which is often directly proportional to the size of the molecule. Capillary isoelectric focusing (CLEF) allows weakly-ionizable amphoteric molecules, to be separated by electrophoresis in a pH gradient. CEC is a hybrid technique between traditional high performance liquid chromatography (HPLC) and CE.

Separation and purification techniques used in the present invention include any chromatography procedures known in the art. Chromatography can be based on the differential adsorption and elution of certain analytes or partitioning of analytes between mobile and stationary phases. Different examples of chromatography include, but not limited to, liquid chromatography (LC), gas chromatography (GC), high performance liquid chromatography (HPLC) etc.

### Identifying Level of PARP

The poly (ADP-ribose) polymerase (PARP) is also known as poly (ADP-ribose) synthase and poly ADP-ribosyltransferase. PARP catalyzes the formation of poly (ADP-ribose) polymers which can attach to nuclear proteins (as well as to itself) and thereby modify the activities of those proteins. The enzyme plays a role in enhancing DNA repair, but it also plays a role in regulating chromatin in the nuclei (for review see: D. D'amours et al. “Poly (ADP-ribosylation reactions in the regulation of nuclear functions,” Biochem. J. 342: 249-268 (1999)).

PARP-1 comprises an N-terminal DNA binding domain, an automodification domain and a C-terminal catalytic domain and various cellular proteins interact with PARP-1. The N-terminal DNA binding domain contains two zinc finger motifs. Transcription enhancer factor-1 (TEF-1), retinoid X receptor α, DNA polymerase α, X-ray repair cross-complementing factor-1 (XRCC1) and PARP-1 itself interact with PARP-1 in this domain. The automodification domain contains a BRCT motif, one of the protein-protein interaction modules. This motif is originally found in the C-terminus of BRCA1 (breast cancer susceptibility protein 1) and is present in various proteins related to DNA repair, recombination and cell-cycle checkpoint control. POU-homeodomain-containing octamer transcription factor-1 (Oct-1), Yin Yang (YY) 1 and ubiquitin-conjugating enzyme 9 (ubc9) could interact with this BRCT motif in PARP-1.

More than 15 members of the PARP family of genes are present in the mammalian genome. PARP family proteins and poly(ADP-ribose) glycohydrolase (PARG), which degrades poly(ADP-ribose) to ADP-ribose, could be involved in a variety of cell regulatory functions including DNA damage response and transcriptional regulation and may be related to carcinogenesis and the biology of cancer in many respects.

Several PARP family proteins have been identified. Tankyrase has been found as an interacting protein of telomere regulatory factor 1 (TRF-1) and is involved in telomere regulation. Vault PARP (VPARP) is a component in the vault complex, which acts as a nuclear-cytoplasmic transporter. PARP-2, PARP-3 and 2,3,7,8-tetrachlorodibenzo-p-dioxin inducible PARP (TiPARP) have also been identified. Therefore, poly (ADP-ribose) metabolism could be related to a variety of cell regulatory functions.

A member of this gene family is PARP-1. The PARP-1 gene product is expressed at high levels in the nuclei of cells and is dependent upon DNA damage for activation. Without being bound by any theory, it is believed that PARP-1 binds to DNA single or double stranded breaks through an amino terminal DNA binding domain. The binding activates the carboxy terminal catalytic domain and results in the formation of polymers of ADP-ribose on target molecules. PARP-1 is itself a target of poly ADP-ribosylation by virtue of a centrally located automodification domain. The ribosylation of PARP-1 causes dissociation of the PARP-1 molecules from the DNA. The entire process of binding, ribosylation, and dissociation occurs very rapidly. It has been suggested that this transient binding of PARP-1 to sites of DNA damage results in the recruitment of DNA repair machinery or may act to suppress the recombination long enough for the recruitment of repair machinery.

The source of ADP-ribose for the PARP reaction is nicotinamide adenosine dinucleotide (NAD). NAD is synthesized in cells from cellular ATP stores and thus high levels of activation of PARP activity can rapidly lead to depletion of cellular energy stores. It has been demonstrated that induction of PARP activity can lead to cell death that is correlated with depletion of cellular NAD and ATP pools. PARP activity is induced in many instances of oxidative stress or during inflammation. For example, during reperfusion of ischemic tissues reactive nitric oxide is generated and nitric oxide results in the generation of additional reactive oxygen species including hydrogen peroxide, peroxynitrate and hydroxyl radical. These latter species can directly damage DNA and the resulting damage induces activation of PARP activity. Frequently, it appears that sufficient activation of PARP activity occurs such that the cellular energy stores are depleted and the cell dies. A similar mechanism is believed to operate during inflammation when endothelial cells and pro-inflammatory cells synthesize nitric oxide which results in oxidative DNA damage in surrounding cells and the subsequent activation of PARP activity. The cell death that results from PARP activation is believed to be a major contributing factor in the extent of tissue damage that results from ischemia-reperfusion injury or from inflammation.

Inhibition of PARP activity can be potentially useful in the treatment of cancer. De-inhibition of the DNAase (by PARP-1 inhibition) may initiate DNA breakdown that is specific for cancer cells and induce apoptosis in cancer cells only. PARP small molecule inhibitors may sensitize treated tumor cell lines to killing by ionizing radiation and by some DNA damaging chemotherapeutic drugs. A monotherapy by PARP inhibitors or a combination therapy with a chemotherapeutic or radiation may be an effective treatment. Combination therapy with a chemotherapeutic can induce tumor regression at concentrations of the chemotherapeutic that are ineffective by themselves. Further, PARP-1 mutant mice and PARP-1 mutant cell lines may be sensitive to radiation and similar types of chemotherapeutic drugs.

One aspect of the invention relates to identifying diseases treatable by PARP modulators such as, PARP inhibitors, where the identification of the disease is based on identifying the level of PARP in a subject. In a preferred embodiment, if PARP expression is up-regulated in a subject, then the subject is treated with PARP inhibitors. A relative level of PARP-1 expression in subjects with prostrate cancer and breast cancer is up-regulated as compared to normal subjects. Similarly, a relative level of PARP-1 expression in subjects with ovarian cancer and endometrium cancer is up-regulated as compared to normal subjects. Within different cancers, each cancer type shows up-regulation to a different extent from each other. For example, different breast cancers show up-regulation to different extent. Similarly, different ovarian cancers show up-regulation to a different extent. It indicates that PARP-1 up-regulation is not only helpful in identifying PARP-1 mediated diseases treatable by PARP-1 inhibitors but it may also be helpful in predicting/determining the efficacy of the treatment with PARP-1 inhibitors depending on the extent of up-regulation of PARP-1 in a subject. Assessment of PARP-1 gene expression can be an indicator of tumor sensitivity to PARP-1 inhibitor. It may also be helpful in personalizing the dose regimen for a subject depending on the level of up-regulated PARP-1.

In some embodiments, the level of PARP in a sample from a patient is compared to predetermined standard sample. The sample from the patient is typically from a diseased tissue, such as cancer cells or tissues. The standard sample can be from the same patient or from a different subject. The standard sample is typically a normal, non-diseased sample. However, in some embodiments, such as for staging of disease or for evaluating the efficacy of treatment, the standard sample is from a diseased tissue. The standard sample can be a combination of samples from several different subjects. In some embodiments, the level of PARP from a patient is compared to a pre-determined level. This pre-determined level is typically obtained from normal samples. As described herein, a “pre-determined PARP level” may be a level of PARP used to, by way of example only, evaluate a patient that may be selected for treatment, evaluate a response to a PARP inhibitor treatment, evaluate a response to a combination of a PARP inhibitor and a second therapeutic agent treatment, and/or diagnose a patient for cancer, inflammation, pain and/or related conditions. A pre-determined PARP level may be determined in populations of patients with or without cancer. The pre-determined PARP level can be a single number, equally applicable to every patient, or the pre-determined PARP level can vary according to specific subpopulations of patients. For example, men might have a different pre-determined PARP level than women; non-smokers may have a different pre-determined PARP level than smokers. Age, weight, and height of a patient may affect the pre-determined PARP level of the individual. Furthermore, the pre-determined PARP level can be a level determined for each patient individually. The pre-determined PARP level can be any suitable standard. For example, the pre-determined PARP level can be obtained from the same or a different human for whom a patient selection is being assessed. In one embodiment, the pre-determined PARP level can be obtained from a previous assessment of the same patient. In such a manner, the progress of the selection of the patient can be monitored over time. In addition, the standard can be obtained from an assessment of another human or multiple humans, e.g., selected groups of humans. In such a manner, the extent of the selection of the human for whom selection is being assessed can be compared to suitable other humans, e.g., other humans who are in a similar situation to the human of interest, such as those suffering from similar or the same condition(s).

In some embodiments, the level of PARP in a sample from a patient is compared to a predetermined standard sample. The sample from the patient is typically from a diseased tissue, such as cancer cells or tissues. The standard sample can be from the same patient or from a different subject. The standard sample is typically a normal, non-diseased sample. However, in some embodiments, such as for staging of disease or for evaluating the efficacy of treatment, the standard sample is from a diseased tissue. The standard sample can be a combination of samples from several different subjects. In some embodiments, the level of PARP from a patient is compared to a pre-determined level. This pre-determined level is typically obtained from normal samples. As described herein, a “pre-determined PARP level” may be a level of PARP used to, by way of example only, evaluate a patient that may be selected for treatment, evaluate a response to a PARP inhibitor treatment, evaluate a response to a combination of a PARP inhibitor and a second therapeutic agent treatment, and/or diagnose a patient for cancer, inflammation, pain and/or related conditions. In other embodiments, a pre-determined PARP level may be determined in populations of patients with or without cancer. The pre-determined PARP level can be a single number, equally applicable to every patient, or the pre-determined PARP level can vary according to specific subpopulations of patients. For example, men might have a different pre-determined PARP level than women; non-smokers may have a different pre-determined PARP level than smokers. Age, weight, and height of a patient may affect the pre-determined PARP level of the individual or of a designated patient population or sub-population. Furthermore, the pre-determined PARP level can be a level determined for each patient individually. The pre-determined PARP level can be any suitable standard. For example, the pre-determined PARP level can be obtained from the same or a different human for whom a patient selection is being assessed. In one embodiment, the pre-determined PARP level can be obtained from a previous assessment of the same patient. In such a manner, the progress of the selection of the patient can be monitored over time. Similarly, the pre-determined PARP level can be from a specific patient population or subpopulations. Accordingly, the standard can be obtained from an assessment of another human or multiple humans, e.g., selected groups of humans. In such a manner, the extent of the selection of the human for whom selection is being assessed can be compared to suitable other humans, e.g., other humans who are in a similar situation to the human of interest, such as those suffering from similar or the same condition(s).

In some embodiments of the present invention the change of PARP from the pre-determined level is about 0.5 fold, about 1.0 fold, about 1.5 fold, about 2.0 fold, about 2.5 fold, about 3.0 fold, about 3.5 fold, about 4.0 fold, about 4.5 fold, or about 5.0 fold. In some embodiments is fold change is less than about 1, less than about 5, less than about 10, less than about 20, less than about 30, less than about 40, or less than about 50. In other embodiments, the changes in PARP level compared to a predetermined level is more than about 1, more than about 5, more than about 10, more than about 20, more than about 30, more than about 40, or more than about 50. Preferred fold changes from a pre-determined level are about 0.5, about 1.0, about 1.5, about 2.0, about 2.5, and about 3.0.

In some embodiments, the level of PARP is measured in a plurality of samples from a patient population, and is compared to a predetermined standard sample or multiple samples. The sample from the patient population is typically from a diseased tissue, such as cancer cells or tissues. The standard sample can be from either the same patient in the patient population, or from a different subject or population of subjects. The standard sample is typically a normal, non-diseased sample. However, in some embodiments, such as for staging of disease or for evaluating the efficacy of treatment, the standard sample may be from a diseased tissue. The standard sample can be a combination of samples from several different subjects. In some embodiments, the level of PARP from a patient population is compared to a pre-determined level, as described above.

In some embodiments of the present invention the change of PARP level from the pre-determined level in the plurality of patient samples from a patient population is about 0.5 fold, about 1.0 fold, about 1.5 fold, about 2.0 fold, about 2.5 fold, about 3.0 fold, about 3.5 fold, about 4.0 fold, about 4.5 fold, or about 5.0 fold. In some embodiments, the fold change is less than about 1, less than about 5, less than about 10, less than about 20, less than about 30, less than about 40, or less than about 50. In other embodiments, the changes in PARP level compared to a predetermined level is more than about 1, more than about 5, more than about 10, more than about 20, more than about 30, more than about 40, or more than about 50. Preferred fold changes from a pre-determined level are about 0.5, about 1.0, about 1.5, about 2.0, about 2.5, and about 3.0.

Tables Ito XXIII as shown below illustrate PARP-1 gene expression data in subjects suffering from cancer, metabolic diseases, endocrine and neoroendocrine system disorders, cardiovascular diseases (CVS), central nervous system diseases (CNS), diseases of male reproductive system, diseases of female reproductive system, respiratory system, disorders of urinary tract, inflammation, hematolymphoid system, and disorders of digestive system. PARP pathways include apoptotic signaling in response to DNA damage, caspase cascade in apoptosis, D4-GDI signaling pathway, FAS signaling pathway (CD95), HIV-I Nef: negative effector of Fas and TNF, opposing roles of AIF in apoptosis and cell survival, and TNFR1 signaling pathway.

In all the tables, C is control, E is experimental samples, SD is standard deviation, and FC is expression level fold change. The expression intensity scale in Table II is 0, 187.0, 374.0, 561.0, and 748. The expression intensity scale in Table IV is 0, 206.0, 412.0, 617.0, and 823. The expression intensity scale in Table VI and Table VII is 0, 97.0, 194.0, 291.0, and 388. The expression intensity scale in Table XV is 0, 139.0, 278.0, 417.0, and 556. The expression intensity scale in Table XVIII is 0, 250.0, 500.0, 750.0, and 999. The expression intensity scale in Table XXII is 0, 132.0, 264.0, 397.0, and 528. The expression intensity scale in Table XXIII is 0, 180.0, 360.0, and 541.0.

Positive value of FC represents up-regulated PARP-1 and negative value of FC represents down-regulated PARP-1. Accordingly, the present invention identifies various diseases with up-regulated PARP-1 which can be treated by PARP-1 inhibitors and the present invention also identifies various diseases with down-regulated PARP-1 which can be treated by PARP-1 activators or agonists. Table I represents various cancers with up-regulated PARP-1 such as, mullerian mixed tumor, Wilm's tumor, serous cystadenocarcinoma etc. Table I also represents cancers with down-regulated PARP-1 such as, Hashimoto's thyroiditis, benign nodular hyperplasia, adenosquamous carcinoma, islet cell tumor, metastatic adenocarcinoma of the stomach etc. Accordingly, the present invention identifies various cancers with up-regulated PARP-1 which can be treated by PARP-1 inhibitors and the present invention also identifies various cancers with down-regulated PARP-1 which can be treated by PARP-1 activators or agonists.

Table III shows up-regulation of PARP-1 for various breast tumors where infiltrating carcinoma of mixed ductal and lobular type shows a down-regulated PARP-1. Table VIII shows the level of PARP-1 for subjects on medications and subjects not on medications. Table X shows various respiratory diseases with up-regulated PARP-1 where adenosquamous carcinoma of primary type shows a down-regulated PARP-1. Table XII shows PARP-1 expression in the control subject and the subjects suffering from inflammations and illustrates the up-regulated and down-regulated PARP-1 in the diseased subjects. Table XVI shows PARP-1 expression in the control subject and the subjects suffering from CNS diseases and illustrates the up-regulated and down-regulated PARP-1 in the diseased subjects. Table XIX shows PARP-1 expression in the control subjects and the subjects suffering from disorders of the hematolymphoid system and illustrates the up-regulated and down-regulated PARP-1 in the diseased subjects. Table XXI shows the PARP-1 expression in the control subjects and the subjects suffering from various disorders of the endocrine and neoruendocrine system and illustrates the up-regulated and down-regulated PARP-1 in the diseased subjects.

The present invention provides a monitoring method in which the level of PARP in cancer patients can be monitored during the course of cancer or anti-neoplastic treatment, and also preferably, prior to and at the start of treatment. The determination of a decrease or increase in the levels of PARP in the cancer patient compared to the levels of PARP in normal individuals without cancer allows the following evaluation related to patient progression and/or outcome: (i) a more severe stage or grade of the cancer; (ii) shorter time to disease progression, and/or (iii) lack of a positive, i.e., effective, response by the patient to the cancer treatment. For example, based on the monitoring of a patient's PARP levels over time relative to normal levels of PARP, as well as to the patient's own prior-determined levels, a determination can be made as to whether a treatment regimen should be changed, i.e., to be more aggressive or less aggressive; to determine if the patient is responding favorably to his or her treatment; and/or to determine disease status, such as advanced stage or phase of the cancer, or a remission, reduction or regression of the cancer or neoplastic disease. The invention allows a determination of clinical benefit, time to progression (TTP), and length of survival time based upon the findings of up-regulated or down-regulated levels of PARP compared to the levels in normal individuals. The present invention also encompasses PARP diagnostics and methods of using the diagnostics.

The present invention provides a monitoring method in which the level of PARP in cancer patients or populations can be monitored during the course of cancer or anti-neoplastic treatment, and also preferably, prior to and at the start of treatment. The determination of a decrease or increase in the levels of PARP in a cancer patient or population compared to the levels of PARP in normal individuals without cancer allows the following evaluation related to patient progression and/or outcome: (i) a more severe stage or grade of the cancer; (ii) shorter time to disease progression, and/or (iii) lack of a positive, i.e., effective, response by the patient to the cancer treatment. For example, based on the monitoring of a patient's PARP levels over time relative to normal levels of PARP, as well as to the patient's own prior-determined levels, a determination can be made as to whether a treatment regimen should be changed, i.e., to be more aggressive or less aggressive; to determine if the patient is responding favorably to his or her treatment; and/or to determine disease status, such as advanced stage or phase of the cancer, or a remission, reduction or regression of the cancer or neoplastic disease. The invention allows a determination of clinical benefit, time to progression (TTP), and length of survival time based upon the findings of up-regulated or down-regulated levels of PARP compared to the levels in normal individuals. The present invention also encompasses PARP diagnostics and methods of using the diagnostics.

The analysis of PARP levels in patients is particularly valuable and informative, as it allows the physician to more effectively select the best treatments, as well as to utilize more aggressive treatments and therapy regimens based on the up-regulated or down-regulated level of PARP. More aggressive treatment, or combination treatments and regimens, can serve to counteract poor patient prognosis and overall survival time. Armed with this information, the medical practitioner can choose to provide certain types of treatment such as treatment with PARP inhibitors, and/or more aggressive therapy.

The analysis of PARP levels in individual patients or patient populations is particularly valuable and informative, as it allows the physician to more effectively select the best treatments, as well as to utilize more aggressive treatments and therapy regimens based on the up-regulated or down-regulated level of PARP. More aggressive treatment, or combination treatments and regimens, can serve to counteract poor patient prognosis and overall survival time. Armed with this information, the medical practitioner can choose to provide certain types of treatment such as treatment with PARP inhibitors, and/or more aggressive therapy.

In monitoring a patient's PARP levels, over a period of time, which may be days, weeks, months, and in some cases, years, or various intervals thereof, the patient's body fluid sample, e.g., serum or plasma, can be collected at intervals, as determined by the practitioner, such as a physician or clinician, to determine the levels of PARP, and compared to the levels in normal individuals over the course or treatment or disease. For example, patient samples can be taken and monitored every month, every two months, or combinations of one, two, or three month intervals according to the invention. In addition, the PARP levels of the patient obtained over time can be conveniently compared with each other, as well as with the PARP values, of normal controls, during the monitoring period, thereby providing the patient's own PARP values, as an internal, or personal, control for long-term PARP monitoring.

In monitoring an individual patient or patient population's PARP levels, over a period of time, which may be days, weeks, months, and in some cases, years, or various intervals thereof, the patient or patient population's body fluid samples, e.g., serum or plasma, can be collected at intervals, as determined by the practitioner, such as a physician or clinician, to determine the levels of PARP, and compared to the levels in normal individuals or a plurality of individuals in a population over the course or treatment or disease. For example, patient samples can be taken and monitored every month, every two months, or combinations of one, two, or three month intervals according to the invention. In addition, the PARP levels of the patient obtained over time can be conveniently compared with each other, as well as with the PARP values, of normal controls, during the monitoring period, thereby providing the patient's own PARP values, as an internal, or personal, control for long-term PARP monitoring. Similarly, PARP levels from a patient population may also be compared with other populations, including a normal control population, providing a convenient means to compare the patient population results over the course of the monitoring period.

TABLE I
PARP1 - Diff/X (Human)
Name: poly (ADP-ribose) polymerase family, member 1
Sort By: FC Up Minimum Fold Change: 1.2 p-Value Range: 0.00-0.05
89 differential expression events found.
Oncology

| | | | | | Control | | Exper- | Exper- | | |
|---|---|---|---|---|---|---|---|---|---|---|
| | | | | Control | number | Exper- | iment | iment | Fold | |
| | | | Control | Standard | of | iment | Standard | number of | change | p- |
| Fragment | Control | Experiment | Mean | Deviation | samples | Mean | Deviation | samples | (FC) | value |
| 208644_at | Endometrium, | Endometrium, Mullerian | 201.21 | 62.21 | 23 | 517.86 | 185.55 | 7 | 2.57 | 0.004 |
| | Normal | Mixed Tumor, Primary | | | | | | | | |
| 208644_at | Breast, | Breast, Infiltrating Carcinoma | 188.81 | 59.90 | 20 | 448.35 | 167.32 | 8 | 2.37 | 0.003 |
| | Fibrocystic | of Mixed Ductal and Lobular | | | | | | | | |
| | Disease | Type, Primary | | | | | | | | |
| 208644_at | Kidney, Normal | Kidney, Wilm's Tumor, | 165.78 | 27.21 | 81 | 385.07 | 125.19 | 8 | 2.32 | 0.002 |
| | | Primary | | | | | | | | |
| 208644_at | Ovary, Normal | Ovary, Mullerian Mixed | 163.31 | 30.51 | 89 | 371.40 | 144.27 | 5 | 2.27 | 0.032 |
| | | Tumor, Primary | | | | | | | | |
| 208644_at | Ovary, Normal | Ovary, Serous | 163.31 | 30.51 | 89 | 371.23 | 104.08 | 8 | 2.27 | 0.001 |
| | | Cystadenocarcinoma, Primary | | | | | | | | |
| 208644_at | Breast, Normal | Breast, Infiltrating Carcinoma | 201.78 | 81.64 | 68 | 448.35 | 167.32 | 8 | 2.22 | 0.004 |
| | | of Mixed Ductal and Lobular | | | | | | | | |
| | | Type, Primary | | | | | | | | |
| 208644_at | Ovary, Normal | Ovary, Adenocarcinoma, | 163.31 | 30.51 | 89 | 361.56 | 153.46 | 36 | 2.21 | 0.000 |
| | | Papillary Serous Type, | | | | | | | | |
| | | Primary | | | | | | | | |
| 208644_at | Ovary, Normal | Ovary, Adenocarcinoma, | 163.31 | 30.51 | 89 | 331.23 | 140.37 | 22 | 2.03 | 0.000 |
| | | Endometrioid Type, Primary | | | | | | | | |
| 208644_at | Breast, | Metastatic Infiltrating Lobular | 244.89 | 87.72 | 3 | 475.11 | 56.80 | 3 | 1.94 | 0.025 |
| | Infiltrating | Carcinoma of Breast, All | | | | | | | | |
| | Lobular | Secondary Sites | | | | | | | | |
| | Carcinoma, | | | | | | | | | |
| | Primary; Stage I | | | | | | | | | |
| 208644_at | Ovary, | Ovary, Serous | 191.45 | 47.99 | 7 | 371.23 | 104.08 | 8 | 1.94 | 0.001 |
| | Mucinous | Cystadenocarcinoma, Primary | | | | | | | | |
| | Cystadenocarcinoma, | | | | | | | | | |
| | Primary | | | | | | | | | |
| 208644_at | Ovary, | Ovary, Adenocarcinoma, | 191.45 | 47.99 | 7 | 361.56 | 153.46 | 36 | 1.89 | 0.000 |
| | Mucinous | Papillary Serous Type, | | | | | | | | |
| | Cystadenocarcinoma, | Primary | | | | | | | | |
| | Primary | | | | | | | | | |
| 208644_at | Testis, Normal | Testis, Seminoma, Primary | 333.35 | 78.19 | 7 | 622.56 | 164.78 | 8 | 1.87 | 0.001 |
| 208644_at | Lung, | Lung, Squamous Cell | 167.99 | 19.89 | 39 | 309.53 | 103.71 | 39 | 1.84 | 0.000 |
| | Pulmonary | Carcinoma, Primary | | | | | | | | |
| | Emphysema, not | | | | | | | | | |
| | Associated with | | | | | | | | | |
| | A1AT | | | | | | | | | |
| | Deficiency | | | | | | | | | |
| 208644_at | Lung, Normal | Lung, Squamous Cell | 170.58 | 56.25 | 126 | 309.53 | 103.71 | 39 | 1.81 | 0.000 |
| | | Carcinoma, Primary | | | | | | | | |
| 208644_at | Endometrium, | Endometrium, Mullerian | 297.42 | 98.78 | 50 | 517.86 | 185.55 | 7 | 1.74 | 0.020 |
| | Adenocarcinoma, | Mixed Tumor, Primary | | | | | | | | |
| | Endometrioid | | | | | | | | | |
| | Type, Primary | | | | | | | | | |
| 208644_at | Breast, | Breast, Infiltrating Ductal | 188.81 | 59.90 | 20 | 328.49 | 135.69 | 169 | 1.74 | 0.000 |
| | Fibrocystic | Carcinoma, Primary | | | | | | | | |
| | Disease | | | | | | | | | |
| 208644_at | Lung, | Lung, Large Cell Carcinoma, | 167.99 | 19.89 | 39 | 291.08 | 122.74 | 7 | 1.73 | 0.038 |
| | Pulmonary | Primary | | | | | | | | |
| | Emphysema, not | | | | | | | | | |
| | Associated with | | | | | | | | | |
| | A1AT | | | | | | | | | |
| | Deficiency | | | | | | | | | |
| 208644_at | Lung, Normal | Lung, Large Cell Carcinoma, | 170.58 | 56.25 | 126 | 291.08 | 122.74 | 7 | 1.71 | 0.041 |
| | | Primary | | | | | | | | |
| 208644_at | Lung, | Lung, Adenocarcinoma, | 167.99 | 19.89 | 39 | 284.99 | 92.24 | 46 | 1.70 | 0.000 |
| | Pulmonary | Primary | | | | | | | | |
| | Emphysema, not | | | | | | | | | |
| | Associated with | | | | | | | | | |
| | A1AT | | | | | | | | | |
| | Deficiency | | | | | | | | | |
| 208644_at | Ovary, | Ovary, Serous | 220.76 | 45.99 | 6 | 371.23 | 104.08 | 8 | 1.68 | 0.004 |
| | Adenocarcinoma, | Cystadenocarcinoma, Primary | | | | | | | | |
| | Clear Cell | | | | | | | | | |
| | Type, Primary | | | | | | | | | |
| 208644_at | Breast, | Breast, Infiltrating Lobular | 188.81 | 59.90 | 20 | 317.43 | 123.81 | 17 | 1.68 | 0.001 |
| | Fibrocystic | Carcinoma, Primary | | | | | | | | |
| | Disease | | | | | | | | | |
| 208644_at | Lung, Normal | Lung, Adenocarcinoma, | 170.58 | 56.25 | 126 | 284.99 | 92.24 | 46 | 1.67 | 0.000 |
| | | Primary | | | | | | | | |
| 208644_at | Endometrium, | Endometrium, | 202.89 | 75.38 | 10 | 336.79 | 71.19 | 6 | 1.66 | 0.004 |
| | Normal; | Adenocarcinoma, | | | | | | | | |
| | Smoking History | Endometrioid Type, Primary; | | | | | | | | |
| | | Smoking History | | | | | | | | |
| 208644_at | Breast, Normal; | Breast, Infiltrating Ductal | 192.72 | 41.09 | 30 | 319.17 | 114.21 | 89 | 1.66 | 0.000 |
| | No Smoking | Carcinoma, Primary; No | | | | | | | | |
| | History | Smoking History | | | | | | | | |
| 208644_at | Skin, Normal | Skin, Basal Cell Carcinoma, | 154.29 | 67.12 | 61 | 255.43 | 62.26 | 4 | 1.66 | 0.043 |
| | | Primary | | | | | | | | |
| 208644_at | Ovary, | Ovary, Adenocarcinoma, | 220.76 | 45.99 | 6 | 361.56 | 153.46 | 36 | 1.64 | 0.000 |
| | Adenocarcinoma, | Papillary Serous Type, | | | | | | | | |
| | Clear Cell | Primary | | | | | | | | |
| | Type, Primary | | | | | | | | | |
| 208644_at | Breast, Normal; | Breast, Infiltrating Lobular | 192.72 | 41.09 | 30 | 313.80 | 134.84 | 10 | 1.63 | 0.020 |
| | No Smoking | Carcinoma, Primary; No | | | | | | | | |
| | History | Smoking History | | | | | | | | |
| 208644_at | Breast, Normal | Breast, Infiltrating Ductal | 201.78 | 81.64 | 68 | 328.49 | 135.69 | 169 | 1.63 | 0.000 |
| | | Carcinoma, Primary | | | | | | | | |
| 208644_at | Breast, | Metastatic Infiltrating Ductal | 266.60 | 67.10 | 18 | 433.92 | 146.92 | 10 | 1.63 | 0.006 |
| | Infiltrating | Carcinoma of Breast, All | | | | | | | | |
| | Ductal | Secondary Sites | | | | | | | | |
| | Carcinoma, | | | | | | | | | |
| | Primary; Stage I | | | | | | | | | |
| 208644_at | Liver, Focal | Liver, Hepatocellular | 151.17 | 14.70 | 8 | 241.43 | 87.63 | 16 | 1.60 | 0.001 |
| | Nodular | Carcinoma | | | | | | | | |
| | Hyperplasia | | | | | | | | | |
| 208644_at | Breast, Normal | Breast, Infiltrating Lobular | 201.78 | 81.64 | 68 | 317.43 | 123.81 | 17 | 1.57 | 0.002 |
| | | Carcinoma, Primary | | | | | | | | |
| 208644_at | Breast, | Breast, Mucinous Carcinoma, | 188.81 | 59.90 | 20 | 293.74 | 61.35 | 4 | 1.56 | 0.032 |
| | Fibrocystic | Primary | | | | | | | | |
| | Disease | | | | | | | | | |
| 208644_at | Soft Tissues | Soft Tissues (Any Body Site), | 164.70 | 15.96 | 5 | 255.19 | 55.84 | 9 | 1.55 | 0.001 |
| | (Any Body Site), | Schwannoma | | | | | | | | |
| | Neurofibroma | | | | | | | | | |
| 208644_at | Kidney, Normal | Kidney, Transitional Cell | 165.78 | 27.21 | 81 | 256.62 | 24.30 | 4 | 1.55 | 0.004 |
| | | Carcinoma, Primary | | | | | | | | |
| 208644_at | Thyroid Gland, | Thyroid Gland, Hashimoto's | 171.03 | 45.31 | 13 | 264.17 | 52.94 | 4 | 1.54 | 0.029 |
| | Normal; Primary | Thyroiditis; Primary | | | | | | | | |
| | Malignancy | Malignancy Elsewhere in | | | | | | | | |
| | Elsewhere in | Thyroid | | | | | | | | |
| | Thyroid | | | | | | | | | |
| 208644_at | Breast, | Metastatic Infiltrating Ductal | 282.58 | 55.53 | 5 | 433.92 | 146.92 | 10 | 1.54 | 0.013 |
| | Infiltrating | Carcinoma of Breast, All | | | | | | | | |
| | Ductal | Secondary Sites | | | | | | | | |
| | Carcinoma, | | | | | | | | | |
| | Primary; Stage | | | | | | | | | |
| | IV | | | | | | | | | |
| 208644_at | Thyroid Gland, | Thyroid Gland, Hashimoto's | 173.82 | 34.78 | 24 | 264.17 | 52.94 | 4 | 1.52 | 0.037 |
| | Normal | Thyroiditis; Primary | | | | | | | | |
| | | Malignancy Elsewhere in | | | | | | | | |
| | | Thyroid | | | | | | | | |
| 208644_at | Esophagus, | Esophagus, Adenocarcinoma, | 191.78 | 40.67 | 22 | 290.09 | 5.61 | 3 | 1.51 | 0.000 |
| | Normal | Primary | | | | | | | | |
| 208644_at | Ovary, | Ovary, Adenocarcinoma, | 220.76 | 45.99 | 6 | 331.23 | 140.37 | 22 | 1.50 | 0.004 |
| | Adenocarcinoma, | Endometrioid Type, Primary | | | | | | | | |
| | Clear Cell | | | | | | | | | |
| | Type, Primary | | | | | | | | | |
| 208644_at | Breast, | Metastatic Infiltrating Lobular | 317.43 | 123.81 | 17 | 475.11 | 56.80 | 3 | 1.50 | 0.011 |
| | Infiltrating | Carcinoma of Breast, All | | | | | | | | |
| | Lobular | Secondary Sites | | | | | | | | |
| | Carcinoma, | | | | | | | | | |
| | Primary | | | | | | | | | |
| 208644_at | Endometrium, | Endometrium, | 201.21 | 62.21 | 23 | 297.42 | 98.78 | 50 | 1.48 | 0.000 |
| | Normal | Adenocarcinoma, | | | | | | | | |
| | | Endometrioid Type, Primary | | | | | | | | |
| 208644_at | Lung, | Lung, Squamous Cell | 209.41 | 25.19 | 3 | 309.53 | 103.71 | 39 | 1.48 | 0.001 |
| | Adenosquamous | Carcinoma, Primary | | | | | | | | |
| | Carcinoma, | | | | | | | | | |
| | Primary | | | | | | | | | |
| 208644_at | Liver, Cirrhosis | Liver, Hepatocellular | 168.31 | 20.68 | 25 | 241.43 | 87.63 | 16 | 1.43 | 0.005 |
| | Secondary to | Carcinoma | | | | | | | | |
| | Chronic | | | | | | | | | |
| | Hepatitis C | | | | | | | | | |
| 208644_at | Endometrium, | Endometrium, | 200.26 | 56.94 | 10 | 286.55 | 91.55 | 40 | 1.43 | 0.001 |
| | Normal; No | Adenocarcinoma, | | | | | | | | |
| | Smoking History | Endometrioid Type, Primary; | | | | | | | | |
| | | No Smoking History | | | | | | | | |
| 208644_at | Liver, Cirrhosis, | Liver, Hepatocellular | 169.21 | 36.75 | 61 | 241.43 | 87.63 | 16 | 1.43 | 0.005 |
| | All Causes | Carcinoma | | | | | | | | |
| 208644_at | Thymus, Normal | Thymus, Thymoma, | 263.23 | 48.02 | 62 | 371.94 | 25.92 | 3 | 1.41 | 0.009 |
| | | Malignant, Primary | | | | | | | | |
| 208644_at | Breast, | Breast, Phyllodes Tumor | 189.96 | 40.20 | 10 | 267.66 | 38.27 | 5 | 1.41 | 0.006 |
| | Fibroadenoma | (Cystosarcoma Phyllodes), | | | | | | | | |
| | | Primary | | | | | | | | |
| 208644_at | Breast, | Metastatic Infiltrating Ductal | 312.55 | 101.26 | 26 | 433.92 | 146.92 | 10 | 1.39 | 0.033 |
| | Infiltrating | Carcinoma of Breast, All | | | | | | | | |
| | Ductal | Secondary Sites | | | | | | | | |
| | Carcinoma, | | | | | | | | | |
| | Primary; PR + | | | | | | | | | |
| 208644_at | Breast, | Metastatic Infiltrating Ductal | 312.55 | 101.26 | 26 | 433.92 | 146.92 | 10 | 1.39 | 0.033 |
| | Infiltrating | Carcinoma of Breast, All | | | | | | | | |
| | Ductal | Secondary Sites | | | | | | | | |
| | Carcinoma, | | | | | | | | | |
| | Primary; ER+ | | | | | | | | | |
| | PR+ | | | | | | | | | |
| 208644_at | Rectum, Normal; | Rectum, Adenocarcinoma | 195.57 | 30.49 | 10 | 269.75 | 45.67 | 5 | 1.38 | 0.017 |
| | No Smoking | (Excluding Mucinous Type), | | | | | | | | |
| | History | Primary; No Smoking History | | | | | | | | |
| 208644_at | Rectum, Normal, | Rectum, Adenocarcinoma | 191.19 | 31.91 | 3 | 262.78 | 62.38 | 29 | 1.37 | 0.032 |
| | No Primary | (Excluding Mucinous Type), | | | | | | | | |
| | Colorectal | Primary | | | | | | | | |
| | Malignancy | | | | | | | | | |
| 208644_at | Bone, Normal | Bone, Osteosarcoma, Primary | 196.06 | 25.06 | 8 | 269.40 | 19.03 | 4 | 1.37 | 0.001 |
| 208644_at | Breast, | Metastatic Infiltrating Ductal | 315.93 | 99.83 | 35 | 433.92 | 146.92 | 10 | 1.37 | 0.035 |
| | Infiltrating | Carcinoma of Breast, All | | | | | | | | |
| | Ductal | Secondary Sites | | | | | | | | |
| | Carcinoma, | | | | | | | | | |
| | Primary; ER + | | | | | | | | | |
| 208644_at | Thyroid Gland, | Thyroid Gland, Hashimoto's | 172.59 | 37.49 | 7 | 236.80 | 77.06 | 10 | 1.37 | 0.039 |
| | Normal; No | Thyroiditis; No Primary | | | | | | | | |
| | Primary Thyroid | Thyroid Malignancy | | | | | | | | |
| | Malignancy | | | | | | | | | |
| 208644_at | Breast, | Metastatic Infiltrating Lobular | 347.91 | 99.95 | 7 | 475.11 | 56.80 | 3 | 1.37 | 0.039 |
| | Infiltrating | Carcinoma of Breast, All | | | | | | | | |
| | Lobular | Secondary Sites | | | | | | | | |
| | Carcinoma, | | | | | | | | | |
| | Primary; ER + | | | | | | | | | |
| 208644_at | Thyroid Gland, | Thyroid Gland, Hashimoto's | 173.82 | 34.78 | 24 | 236.80 | 77.06 | 10 | 1.36 | 0.031 |
| | Normal | Thyroiditis; No Primary | | | | | | | | |
| | | Thyroid Malignancy | | | | | | | | |
| 208644_at | Ovary, Normal | Ovary, Adenocarcinoma, | 163.31 | 30.51 | 89 | 220.76 | 45.99 | 6 | 1.35 | 0.027 |
| | | Clear Cell Type, Primary | | | | | | | | |
| 208644_at | Breast, | Metastatic Infiltrating Ductal | 322.14 | 96.83 | 70 | 433.92 | 146.92 | 10 | 1.35 | 0.041 |
| | Infiltrating | Carcinoma of Breast, All | | | | | | | | |
| | Ductal | Secondary Sites | | | | | | | | |
| | Carcinoma, | | | | | | | | | |
| | Primary; Stage II | | | | | | | | | |
| 208644_at | Breast, Normal | Breast, Phyllodes Tumor | 201.78 | 81.64 | 68 | 267.66 | 38.27 | 5 | 1.33 | 0.012 |
| | | (Cystosarcoma Phyllodes), | | | | | | | | |
| | | Primary | | | | | | | | |
| 208644_at | Colon, Adenoma | Colon, Adenocarcinoma | 201.66 | 38.31 | 19 | 266.64 | 46.55 | 11 | 1.32 | 0.001 |
| | | (Excluding Mucinous Type), | | | | | | | | |
| | | Primary; Stage I | | | | | | | | |
| 208644_at | Thyroid Gland, | Thyroid Gland, Papillary | 173.82 | 34.78 | 24 | 225.17 | 46.13 | 8 | 1.30 | 0.017 |
| | Normal | Carcinoma, Follicular Variant, | | | | | | | | |
| | | Primary | | | | | | | | |
| 208644_at | Thyroid Gland, | Thyroid Gland, Papillary | 173.96 | 29.46 | 58 | 225.17 | 46.13 | 8 | 1.29 | 0.016 |
| | Nodular | Carcinoma, Follicular Variant, | | | | | | | | |
| | Hyperplasia | Primary | | | | | | | | |
| 208644_at | Rectum, Normal | Rectum, Adenocarcinoma | 206.94 | 31.16 | 44 | 262.78 | 62.38 | 29 | 1.27 | 0.000 |
| | | (Excluding Mucinous Type), | | | | | | | | |
| | | Primary | | | | | | | | |
| 208644_at | Breast, Normal; | Breast, Normal; Primary | 169.20 | 34.98 | 18 | 213.73 | 91.93 | 48 | 1.26 | 0.006 |
| | No Disease | Malignancy Elsewhere in | | | | | | | | |
| | Elsewhere in | Breast | | | | | | | | |
| | Breast | | | | | | | | | |
| 208644_at | Rectum, Normal | Rectum, Adenocarcinoma | 206.94 | 31.16 | 44 | 260.98 | 63.95 | 26 | 1.26 | 0.000 |
| | | (Excluding Mucinous Type), | | | | | | | | |
| | | Primary; Age 45 and Over | | | | | | | | |
| 208644_at | Bone, Giant Cell | Bone, Osteosarcoma, Primary | 214.10 | 47.88 | 10 | 269.40 | 19.03 | 4 | 1.26 | 0.009 |
| | Tumor of Bone, | | | | | | | | | |
| | Primary | | | | | | | | | |
| 208644_at | Rectum, Normal, | Rectum, Adenocarcinoma | 209.76 | 32.14 | 35 | 262.78 | 62.38 | 29 | 1.25 | 0.000 |
| | Primary | (Excluding Mucinous Type), | | | | | | | | |
| | Malignancy | Primary | | | | | | | | |
| | Elsewhere in | | | | | | | | | |
| | Colon or Rectum | | | | | | | | | |
| 208644_at | Colon, Normal; | Colon, Adenocarcinoma | 197.05 | 44.62 | 62 | 244.35 | 59.56 | 26 | 1.24 | 0.001 |
| | Smoking History | (Excluding Mucinous Type), | | | | | | | | |
| | | Primary; Smoking History | | | | | | | | |
| 208644_at | Endometrium, | Endometrium, | 250.09 | 12.48 | 3 | 308.83 | 97.96 | 35 | 1.23 | 0.003 |
| | Adenocarcinoma, | Adenocarcinoma, | | | | | | | | |
| | Endometrioid | Endometrioid Type, Primary; | | | | | | | | |
| | Type, Primary; | Postmenopausal | | | | | | | | |
| | Premenopausal | | | | | | | | | |
| 208644_at | Colon, Normal; | Colon, Adenocarcinoma | 199.20 | 44.27 | 56 | 244.35 | 59.56 | 26 | 1.23 | 0.001 |
| | No History of | (Excluding Mucinous Type), | | | | | | | | |
| | Inflammatory | Primary; Smoking History | | | | | | | | |
| | Bowel Disease; | | | | | | | | | |
| | Smoking History | | | | | | | | | |
| 208644_at | Myometrium, | Myometrium, Leiomyoma | 176.66 | 30.91 | 122 | 213.73 | 61.61 | 46 | 1.21 | 0.000 |
| | Normal | | | | | | | | | |
| 208644_at | Breast, | Breast, Infiltrating Ductal | 266.60 | 67.10 | 18 | 322.14 | 96.83 | 70 | 1.21 | 0.007 |
| | Infiltrating | Carcinoma, Primary; Stage II | | | | | | | | |
| | Ductal | | | | | | | | | |
| | Carcinoma, | | | | | | | | | |
| | Primary; Stage I | | | | | | | | | |
| 208644_at | Stomach, | Stomach, Adenocarcinoma | 221.41 | 45.66 | 52 | 267.48 | 108.98 | 27 | 1.21 | 0.044 |
| | Normal | (Excluding Signet Ring Cell | | | | | | | | |
| | | Type), Primary | | | | | | | | |
| 208644_at | Thyroid Gland, | Thyroid Gland, Papillary | 250.53 | 67.58 | 19 | 206.04 | 51.25 | 15 | −1.22 | 0.037 |
| | Hashimoto's | Carcinoma (Excluding | | | | | | | | |
| | Thyroiditis | Follicular Variant), Primary | | | | | | | | |
| 208644_at | Colon, | Colon, Adenocarcinoma | 266.64 | 46.55 | 11 | 219.19 | 49.79 | 10 | −1.22 | 0.037 |
| | Adenocarcinoma | (Excluding Mucinous Type), | | | | | | | | |
| | (Excluding | Primary; Stage IV | | | | | | | | |
| | Mucinous Type), | | | | | | | | | |
| | Primary; Stage I | | | | | | | | | |
| 208644_at | Prostate, Benign | Prostate, Benign Nodular | 224.33 | 42.52 | 10 | 183.10 | 32.34 | 10 | −1.23 | 0.026 |
| | Nodular | Hyperplasia; Primary | | | | | | | | |
| | Hyperplasia; No | Malignancy Elsewhere in | | | | | | | | |
| | Primary | Prostate | | | | | | | | |
| | Prostatic | | | | | | | | | |
| | Malignancy | | | | | | | | | |
| 208644_at | Colon, | Metastatic Adenocarcinoma of | 266.64 | 46.55 | 11 | 217.45 | 61.79 | 22 | −1.23 | 0.017 |
| | Adenocarcinoma | Colon, All Secondary Sites | | | | | | | | |
| | (Excluding | | | | | | | | | |
| | Mucinous Type), | | | | | | | | | |
| | Primary; Stage I | | | | | | | | | |
| 208644_at | Lung, | Lung, Adenosquamous | 284.99 | 92.24 | 46 | 209.41 | 25.19 | 3 | −1.36 | 0.007 |
| | Adenocarcinoma, | Carcinoma, Primary | | | | | | | | |
| | Primary | | | | | | | | | |
| 208644_at | Kidney, Renal | Kidney, Carcinoma, | 178.49 | 58.38 | 15 | 127.65 | 22.77 | 3 | −1.40 | 0.033 |
| | Cell Carcinoma, | Chromophobe Type, Primary | | | | | | | | |
| | Non-Clear Cell | | | | | | | | | |
| | Type, Primary | | | | | | | | | |
| 208644_at | Pancreas, | Pancreas, Islet Cell Tumor, | 321.84 | 69.04 | 46 | 212.69 | 83.22 | 7 | −1.51 | 0.012 |
| | Normal | Malignant, Primary | | | | | | | | |
| 208644_at | Breast, | Breast, Mucinous Carcinoma, | 448.35 | 167.32 | 8 | 293.74 | 61.35 | 4 | −1.53 | 0.044 |
| | Infiltrating | Primary | | | | | | | | |
| | Carcinoma of | | | | | | | | | |
| | Mixed Ductal | | | | | | | | | |
| | and Lobular | | | | | | | | | |
| | Type, Primary | | | | | | | | | |
| 208644_at | Stomach, | Metastatic Adenocarcinoma | 248.25 | 57.84 | 8 | 159.57 | 34.93 | 3 | −1.56 | 0.020 |
| | Adenocarcinoma | (Excluding Signet Ring Cell | | | | | | | | |
| | (Excluding | Type) of Stomach, All | | | | | | | | |
| | Signet Ring Cell | Secondary Sites | | | | | | | | |
| | Type), Primary; | | | | | | | | | |
| | Stage III | | | | | | | | | |
| 208644_at | Kidney, Renal | Kidney, Carcinoma, | 201.00 | 134.98 | 45 | 127.65 | 22.77 | 3 | −1.57 | 0.007 |
| | Cell Carcinoma, | Chromophobe Type, Primary | | | | | | | | |
| | Clear Cell Type, | | | | | | | | | |
| | Primary | | | | | | | | | |
| 208644_at | Pancreas, | Pancreas, Adenocarcinoma, | 305.07 | 61.48 | 11 | 184.74 | 54.40 | 3 | −1.65 | 0.036 |
| | Normal; No | Primary; No Smoking History | | | | | | | | |
| | Smoking History | | | | | | | | | |
| 208644_at | Stomach, | Metastatic Adenocarcinoma | 267.48 | 108.98 | 27 | 159.57 | 34.93 | 3 | −1.68 | 0.006 |
| | Adenocarcinoma | (Excluding Signet Ring Cell | | | | | | | | |
| | (Excluding | Type) of Stomach, All | | | | | | | | |
| | Signet Ring Cell | Secondary Sites | | | | | | | | |
| | Type), Primary | | | | | | | | | |
| 208644_at | Pancreas, | Pancreas, Adenocarcinoma, | 321.84 | 69.04 | 46 | 191.82 | 53.50 | 23 | −1.68 | 0.000 |
| | Normal | Primary | | | | | | | | |
| 208644_at | Ovary, | Ovary, Mucinous | 331.23 | 140.37 | 22 | 191.45 | 47.99 | 7 | −1.73 | 0.000 |
| | Adenocarcinoma, | Cystadenocarcinoma, Primary | | | | | | | | |
| | Endometrioid | | | | | | | | | |
| | Type, Primary | | | | | | | | | |
| 208644_at | Pancreas, | Pancreas, Adenocarcinoma, | 313.58 | 74.61 | 23 | 166.22 | 27.30 | 5 | −1.89 | 0.000 |
| | Normal; | Primary; Smoking History | | | | | | | | |
| | Smoking History | | | | | | | | | |
| 208644_at | Stomach, | Metastatic Adenocarcinoma | 324.58 | 46.07 | 5 | 159.57 | 34.93 | 3 | −2.03 | 0.002 |
| | Adenocarcinoma | (Excluding Signet Ring Cell | | | | | | | | |
| | (Excluding | Type) of Stomach, All | | | | | | | | |
| | Signet Ring Cell | Secondary Sites | | | | | | | | |
| | Type), Primary; | | | | | | | | | |
| | Stage II | | | | | | | | | |

TABLE II
PARP1 - e-Northern (Human)
Name: poly (ADP-ribose) polymerase family, member 1
Sort By: Organ System
View: Primary Malignancy
Fragment Legend: 208644_at

| | | | | Number of | Lower | 25% | | | 75% | Upper |
|---|---|---|---|---|---|---|---|---|---|---|
| Category | Fragment | Freq. | % Present | Samples | Limit | Quan. | Median | Mean | Quan. | Limit |
| Human, Primary Malignancies, Digestive | | | | | | | | | | |
| System | | | | | | | | | | |
| Colon, Adenocarcinoma (Excluding Mucinous | 208644_at | 1.00 | 1.00 | 77 | 119.72 | 192.45 | 218.94 | 234.82 | 269.65 | 385.43 |
| Type), Primary | | | | | | | | | | |
| Colon, Adenocarcinoma, Mucinous Type, | 208644_at | 1.00 | 1.00 | 7 | 183.56 | 204.70 | 215.80 | 233.90 | 244.74 | 304.80 |
| Primary | | | | | | | | | | |
| Colon, Normal | 208644_at | 1.00 | 1.00 | 180 | 88.25 | 166.74 | 191.91 | 198.00 | 229.97 | 324.80 |
| Esophagus, Adenocarcinoma, Primary | 208644_at | 1.00 | 1.00 | 3 | 283.68 | 288.08 | 292.48 | 290.09 | 293.30 | 294.11 |
| Esophagus, Normal | 208644_at | 1.00 | 1.00 | 22 | 132.91 | 162.68 | 187.02 | 191.78 | 219.85 | 291.45 |
| Liver, Hepatocellular Carcinoma | 208644_at | 1.00 | 1.00 | 16 | 140.66 | 177.59 | 231.86 | 241.43 | 272.87 | 415.79 |
| Liver, Normal | 208644_at | 1.00 | 1.00 | 42 | 85.71 | 149.27 | 172.85 | 195.15 | 201.08 | 278.79 |
| Oral Cavity, Squamous Cell Carcinoma, | 208644_at | 1.00 | 1.00 | 3 | 218.13 | 275.93 | 333.73 | 301.56 | 343.27 | 352.81 |
| Primary | | | | | | | | | | |
| Pancreas, Adenocarcinoma, Primary | 208644_at | 1.00 | 1.00 | 23 | 118.87 | 161.59 | 180.11 | 191.82 | 214.60 | 294.12 |
| Pancreas, Islet Cell Tumor, Malignant, | 208644_at | 1.00 | 1.00 | 7 | 138.92 | 147.93 | 164.86 | 212.69 | 272.06 | 345.11 |
| Primary | | | | | | | | | | |
| Pancreas, Normal | 208644_at | 1.00 | 1.00 | 46 | 131.80 | 276.35 | 319.04 | 321.84 | 372.71 | 469.39 |
| Rectum, Adenocarcinoma (Excluding | 208644_at | 1.00 | 1.00 | 29 | 160.75 | 225.65 | 255.76 | 262.78 | 280.49 | 362.74 |
| Mucinous Type), Primary | | | | | | | | | | |
| Rectum, Adenocarcinoma, Mucinous Type, | 208644_at | 1.00 | 1.00 | 3 | 206.09 | 211.90 | 217.71 | 219.41 | 226.07 | 234.44 |
| Primary | | | | | | | | | | |
| Rectum, Normal | 208644_at | 1.00 | 1.00 | 44 | 154.22 | 180.56 | 204.22 | 206.94 | 225.30 | 285.55 |
| Small Intestine, Gastrointestinal Stromal | 208644_at | 1.00 | 1.00 | 4 | 211.81 | 231.96 | 254.27 | 295.77 | 318.07 | 447.24 |
| Tumor (GIST), Primary | | | | | | | | | | |
| Small Intestine, Normal | 208644_at | 1.00 | 1.00 | 97 | 90.87 | 167.17 | 186.89 | 193.57 | 218.03 | 294.33 |
| Stomach, Adenocarcinoma (Excluding Signet | 208644_at | 1.00 | 1.00 | 27 | 126.81 | 208.59 | 249.23 | 267.48 | 317.59 | 481.09 |
| Ring Cell Type), Primary | | | | | | | | | | |
| Stomach, Adenocarcinoma, Signet Ring Cell | 208644_at | 1.00 | 1.00 | 9 | 165.84 | 224.99 | 248.18 | 251.51 | 264.42 | 323.56 |
| Type, Primary | | | | | | | | | | |
| Stomach, Gastrointestinal Stromal Tumor | 208644_at | 1.00 | 1.00 | 9 | 178.05 | 198.43 | 213.69 | 229.85 | 274.87 | 285.79 |
| (GIST), Primary | | | | | | | | | | |
| Stomach, Normal | 208644_at | 1.00 | 0.98 | 52 | 116.33 | 190.63 | 215.46 | 221.41 | 244.02 | 324.10 |
| Human, Primary Malignancies, Endocrine | | | | | | | | | | |
| System | | | | | | | | | | |
| Adrenal Gland, Adrenal Cortical Carcinoma, | 208644_at | 1.00 | 1.00 | 3 | 139.51 | 164.22 | 188.92 | 184.60 | 207.14 | 225.37 |
| Primary | | | | | | | | | | |
| Adrenal Gland, Normal | 208644_at | 1.00 | 0.92 | 13 | 118.84 | 149.84 | 169.92 | 179.87 | 183.69 | 234.47 |
| Thyroid Gland, Follicular Carcinoma, Primary | 208644_at | 1.00 | 1.00 | 3 | 184.37 | 197.50 | 210.63 | 247.16 | 278.56 | 346.49 |
| Thyroid Gland, Normal | 208644_at | 1.00 | 1.00 | 24 | 125.34 | 150.78 | 171.69 | 173.82 | 187.96 | 243.72 |
| Thyroid Gland, Papillary Carcinoma, Primary; | 208644_at | 1.00 | 1.00 | 29 | 131.77 | 174.45 | 207.12 | 209.42 | 250.96 | 310.14 |
| All Variants | | | | | | | | | | |
| Human, Primary Malignancies, Female | | | | | | | | | | |
| Reproductive System | | | | | | | | | | |
| Breast, Infiltrating Carcinoma of Mixed | 208644_at | 1.00 | 1.00 | 8 | 230.17 | 342.70 | 424.60 | 448.35 | 538.87 | 748.60 |
| Ductal and Lobular Type, Primary | | | | | | | | | | |
| Breast, Infiltrating Ductal Carcinoma, Primary | 208644_at | 1.00 | 0.99 | 169 | 97.65 | 241.86 | 312.34 | 328.49 | 378.43 | 583.27 |
| Breast, Infiltrating Lobular Carcinoma, | 208644_at | 1.00 | 1.00 | 17 | 168.65 | 217.68 | 291.47 | 317.43 | 381.66 | 557.68 |
| Primary | | | | | | | | | | |
| Breast, Intraductal Carcinoma | 208644_at | 1.00 | 1.00 | 3 | 151.50 | 260.13 | 368.75 | 306.24 | 383.61 | 398.46 |
| Breast, Mucinous Carcinoma, Primary | 208644_at | 1.00 | 1.00 | 4 | 230.10 | 246.84 | 297.53 | 293.74 | 344.43 | 349.80 |
| Breast, Normal | 208644_at | 1.00 | 1.00 | 68 | 123.07 | 161.29 | 178.83 | 201.78 | 217.55 | 301.94 |
| Breast, Phyllodes Tumor (Cystosarcoma | 208644_at | 1.00 | 1.00 | 5 | 210.75 | 249.68 | 279.85 | 267.66 | 289.54 | 308.51 |
| Phyllodes), Primary | | | | | | | | | | |
| Endometrium, Adenocarcinoma, | 208644_at | 1.00 | 1.00 | 50 | 129.74 | 226.47 | 300.82 | 297.42 | 362.76 | 527.87 |
| Endometrioid Type, Primary | | | | | | | | | | |
| Endometrium, Mullerian Mixed Tumor, | 208644_at | 1.00 | 1.00 | 7 | 213.14 | 408.97 | 597.14 | 517.86 | 637.77 | 721.26 |
| Primary | | | | | | | | | | |
| Endometrium, Normal | 208644_at | 1.00 | 1.00 | 23 | 105.71 | 154.36 | 199.06 | 201.21 | 227.26 | 336.60 |
| Ovary, Adenocarcinoma, Clear Cell Type, | 208644_at | 1.00 | 1.00 | 6 | 183.37 | 184.40 | 201.67 | 220.76 | 253.54 | 288.35 |
| Primary | | | | | | | | | | |
| Ovary, Adenocarcinoma, Endometrioid Type, | 208644_at | 1.00 | 1.00 | 22 | 130.39 | 213.39 | 312.44 | 331.23 | 419.91 | 728.19 |
| Primary | | | | | | | | | | |
| Ovary, Adenocarcinoma, Papillary Serous | 208644_at | 1.00 | 1.00 | 36 | 132.09 | 279.55 | 311.57 | 361.56 | 430.83 | 657.74 |
| Type, Primary | | | | | | | | | | |
| Ovary, Granulosa Cell Tumor, Primary | 208644_at | 1.00 | 1.00 | 3 | 233.86 | 314.88 | 395.91 | 422.98 | 517.54 | 639.17 |
| Ovary, Mucinous Cystadenocarcinoma, | 208644_at | 1.00 | 1.00 | 7 | 133.94 | 174.24 | 182.88 | 191.45 | 201.11 | 241.42 |
| Primary | | | | | | | | | | |
| Ovary, Mullerian Mixed Tumor, Primary | 208644_at | 1.00 | 1.00 | 5 | 217.65 | 263.58 | 329.77 | 371.40 | 511.03 | 534.99 |
| Ovary, Normal | 208644_at | 1.00 | 1.00 | 89 | 98.25 | 147.72 | 161.49 | 163.31 | 180.70 | 224.40 |
| Uterine Cervix, Adenocarcinoma, Primary | 208644_at | 1.00 | 1.00 | 3 | 181.52 | 316.73 | 451.93 | 365.94 | 458.16 | 464.38 |
| Uterine Cervix, Normal | 208644_at | 1.00 | 0.98 | 115 | 77.91 | 150.20 | 168.54 | 178.85 | 198.40 | 270.70 |
| Vulva, Normal | 208644_at | 1.00 | 1.00 | 4 | 115.16 | 149.62 | 168.27 | 160.95 | 179.59 | 192.10 |
| Vulva, Squamous Cell Carcinoma, Primary | 208644_at | 1.00 | 1.00 | 5 | 185.36 | 191.87 | 194.17 | 193.80 | 198.78 | 198.80 |
| Human, Primary Malignancies, | | | | | | | | | | |
| Integumentary System | | | | | | | | | | |
| Skin, Basal Cell Carcinoma, Primary | 208644_at | 1.00 | 1.00 | 4 | 184.31 | 220.72 | 253.10 | 255.43 | 287.80 | 331.20 |
| Skin, Malignant Melanoma, Primary | 208644_at | 1.00 | 1.00 | 7 | 115.32 | 176.26 | 258.73 | 325.86 | 382.59 | 692.09 |
| Skin, Normal | 208644_at | 1.00 | 1.00 | 61 | 73.17 | 123.09 | 149.86 | 154.29 | 167.66 | 234.50 |
| Skin, Squamous Cell Carcinoma, Primary | 208644_at | 1.00 | 1.00 | 4 | 170.28 | 176.91 | 232.43 | 276.90 | 332.42 | 472.46 |
| Human, Primary Malignancies, Male | | | | | | | | | | |
| Reproductive System | | | | | | | | | | |
| Prostate, Adenocarcinoma, Primary | 208644_at | 1.00 | 1.00 | 86 | 153.83 | 207.07 | 232.00 | 236.59 | 265.72 | 341.98 |
| Prostate, Normal | 208644_at | 1.00 | 1.00 | 57 | 135.98 | 184.58 | 201.84 | 209.09 | 236.64 | 294.97 |
| Human, Primary Malignancies, | | | | | | | | | | |
| Musculoskeletal System | | | | | | | | | | |
| Bone, Giant Cell Tumor of Bone, Primary | 208644_at | 1.00 | 1.00 | 10 | 159.53 | 185.87 | 199.58 | 214.10 | 221.59 | 275.16 |
| Bone, Normal | 208644_at | 1.00 | 1.00 | 8 | 154.45 | 183.30 | 194.26 | 196.06 | 218.32 | 225.33 |
| Bone, Osteosarcoma, Primary | 208644_at | 1.00 | 1.00 | 4 | 250.09 | 260.05 | 266.04 | 269.40 | 275.38 | 295.42 |
| Human, Primary Malignancies, | | | | | | | | | | |
| Respiratory System | | | | | | | | | | |
| Larynx, Normal | 208644_at | 1.00 | 1.00 | 4 | 180.40 | 191.48 | 208.13 | 208.76 | 225.41 | 238.37 |
| Larynx, Squamous Cell Carcinoma, Primary | 208644_at | 1.00 | 1.00 | 4 | 218.03 | 225.62 | 233.87 | 236.98 | 245.22 | 262.16 |
| Lung, Adenocarcinoma, Primary | 208644_at | 1.00 | 1.00 | 46 | 164.80 | 224.78 | 263.52 | 284.99 | 317.65 | 456.96 |
| Lung, Adenosquamous Carcinoma, Primary | 208644_at | 1.00 | 1.00 | 3 | 183.37 | 197.28 | 211.18 | 209.41 | 222.42 | 233.67 |
| Lung, Large Cell Carcinoma, Primary | 208644_at | 1.00 | 1.00 | 7 | 119.93 | 228.44 | 284.45 | 291.08 | 339.50 | 497.30 |
| Lung, Neuroendocrine Carcinoma (Non-Small | 208644_at | 1.00 | 1.00 | 3 | 236.03 | 242.86 | 249.69 | 408.91 | 495.35 | 741.01 |
| Cell Type), Primary | | | | | | | | | | |
| Lung, Normal | 208644_at | 1.00 | 1.00 | 126 | 87.04 | 143.56 | 161.44 | 170.58 | 181.24 | 237.76 |
| Lung, Small Cell Carcinoma, Primary | 208644_at | 1.00 | 1.00 | 3 | 295.68 | 336.78 | 377.88 | 473.23 | 562.00 | 746.12 |
| Lung, Squamous Cell Carcinoma, Primary | 208644_at | 1.00 | 1.00 | 39 | 101.75 | 253.38 | 304.52 | 309.53 | 362.02 | 524.99 |
| Human, Primary Malignancies, Urinary | | | | | | | | | | |
| Tract | | | | | | | | | | |
| Kidney, Carcinoma, Chromophobe Type, | 208644_at | 1.00 | 1.00 | 3 | 102.04 | 118.66 | 135.27 | 127.65 | 140.45 | 145.63 |
| Primary | | | | | | | | | | |
| Kidney, Normal | 208644_at | 1.00 | 0.99 | 81 | 115.79 | 143.82 | 165.98 | 165.78 | 184.14 | 244.62 |
| Kidney, Renal Cell Carcinoma, Clear Cell | 208644_at | 1.00 | 0.98 | 45 | 61.86 | 141.71 | 161.47 | 201.00 | 217.16 | 330.33 |
| Type, Primary | | | | | | | | | | |
| Kidney, Renal Cell Carcinoma, Non-Clear | 208644_at | 1.00 | 1.00 | 15 | 85.88 | 143.09 | 175.85 | 178.49 | 191.95 | 265.25 |
| Cell Type, Primary | | | | | | | | | | |
| Kidney, Transitional Cell Carcinoma, Primary | 208644_at | 1.00 | 1.00 | 4 | 236.27 | 242.33 | 249.46 | 256.62 | 263.75 | 291.30 |
| Kidney, Wilm's Tumor, Primary | 208644_at | 1.00 | 1.00 | 8 | 187.26 | 328.40 | 392.63 | 385.07 | 465.48 | 562.67 |
| Urinary Bladder, Normal | 208644_at | 1.00 | 1.00 | 9 | 128.23 | 162.66 | 184.14 | 223.63 | 267.52 | 382.66 |
| Urinary Bladder, Transitional Cell Carcinoma, | 208644_at | 1.00 | 1.00 | 4 | 184.05 | 245.81 | 281.28 | 269.09 | 304.55 | 329.75 |
| Primary | | | | | | | | | | |

TABLE III
PARP1 - Diff/X (Human)
Name: poly (ADP-ribose) polymerase family, member 1
Minimum Fold Change: 1.2
p-Value Range: 0.00-0.05
Sort By: p-value
23 differential expression events found.
Breast

| | | | | Control | | Exper- | Experiment | | Fold | |
|---|---|---|---|---|---|---|---|---|---|---|
| | | | Control | Standard | | iment | Standard | Experiment | change | p- |
| Fragment | Control | Experiment | Mean | Deviation | Control # | Mean | Deviation | # | (FC) | value |
| 208644_at | Breast, Normal | Breast, Infiltrating | 201.78 | 81.64 | 68 | 328.49 | 135.69 | 169 | 1.63 | 0.000 |
| | | Ductal Carcinoma, | | | | | | | | |
| | | Primary | | | | | | | | |
| 208644_at | Breast, Normal; No | Breast, Infiltrating | 192.72 | 41.09 | 30 | 319.17 | 114.21 | 89 | 1.66 | 0.000 |
| | Smoking History | Ductal Carcinoma, | | | | | | | | |
| | | Primary; No Smoking | | | | | | | | |
| | | History | | | | | | | | |
| 208644_at | Breast, Fibrocystic | Breast, Infiltrating | 188.81 | 59.90 | 20 | 328.49 | 135.69 | 169 | 1.74 | 0.000 |
| | Disease | Ductal Carcinoma, | | | | | | | | |
| | | Primary | | | | | | | | |
| 208644_at | Breast, Fibrocystic | Breast, Infiltrating | 188.81 | 59.90 | 20 | 317.43 | 123.81 | 17 | 1.68 | 0.001 |
| | Disease | Lobular Carcinoma, | | | | | | | | |
| | | Primary | | | | | | | | |
| 208644_at | Breast, Normal | Breast, Infiltrating | 201.78 | 81.64 | 68 | 317.43 | 123.81 | 17 | 1.57 | 0.002 |
| | | Lobular Carcinoma, | | | | | | | | |
| | | Primary | | | | | | | | |
| 208644_at | Breast, Fibrocystic | Breast, Infiltrating | 188.81 | 59.90 | 20 | 448.35 | 167.32 | 8 | 2.37 | 0.003 |
| | Disease | Carcinoma of Mixed | | | | | | | | |
| | | Ductal and Lobular | | | | | | | | |
| | | Type, Primary | | | | | | | | |
| 208644_at | Breast, Normal | Breast, Infiltrating | 201.78 | 81.64 | 68 | 448.35 | 167.32 | 8 | 2.22 | 0.004 |
| | | Carcinoma of Mixed | | | | | | | | |
| | | Ductal and Lobular | | | | | | | | |
| | | Type, Primary | | | | | | | | |
| 208644_at | Breast, Infiltrating | Metastatic Infiltrating | 266.60 | 67.10 | 18 | 433.92 | 146.92 | 10 | 1.63 | 0.006 |
| | Ductal Carcinoma, | Ductal Carcinoma of | | | | | | | | |
| | Primary; Stage I | Breast, All Secondary | | | | | | | | |
| | | Sites | | | | | | | | |
| 208644_at | Breast, Normal; No | Breast, Normal; Primary | 169.20 | 34.98 | 18 | 213.73 | 91.93 | 48 | 1.26 | 0.006 |
| | Disease Elsewhere | Malignancy Elsewhere | | | | | | | | |
| | in Breast | in Breast | | | | | | | | |
| 208644_at | Breast, | Breast, Phyllodes | 189.96 | 40.20 | 10 | 267.66 | 38.27 | 5 | 1.41 | 0.006 |
| | Fibroadenoma | Tumor (Cystosarcoma | | | | | | | | |
| | | Phyllodes), Primary | | | | | | | | |
| 208644_at | Breast, Infiltrating | Breast, Infiltrating | 266.60 | 67.10 | 18 | 322.14 | 96.83 | 70 | 1.21 | 0.007 |
| | Ductal Carcinoma, | Ductal Carcinoma, | | | | | | | | |
| | Primary; Stage I | Primary; Stage II | | | | | | | | |
| 208644_at | Breast, Infiltrating | Metastatic Infiltrating | 317.43 | 123.81 | 17 | 475.11 | 56.80 | 3 | 1.50 | 0.011 |
| | Lobular Carcinoma, | Lobular Carcinoma of | | | | | | | | |
| | Primary | Breast, All Secondary | | | | | | | | |
| | | Sites | | | | | | | | |
| 208644_at | Breast, Normal | Breast, Phyllodes | 201.78 | 81.64 | 68 | 267.66 | 38.27 | 5 | 1.33 | 0.012 |
| | | Tumor (Cystosarcoma | | | | | | | | |
| | | Phyllodes), Primary | | | | | | | | |
| 208644_at | Breast, Infiltrating | Metastatic Infiltrating | 282.58 | 55.53 | 5 | 433.92 | 146.92 | 10 | 1.54 | 0.013 |
| | Ductal Carcinoma, | Ductal Carcinoma of | | | | | | | | |
| | Primary; Stage IV | Breast, All Secondary | | | | | | | | |
| | | Sites | | | | | | | | |
| 208644_at | Breast, Normal; No | Breast, Infiltrating | 192.72 | 41.09 | 30 | 313.80 | 134.84 | 10 | 1.63 | 0.020 |
| | Smoking History | Lobular Carcinoma, | | | | | | | | |
| | | Primary; No Smoking | | | | | | | | |
| | | History | | | | | | | | |
| 208644_at | Breast, Infiltrating | Metastatic Infiltrating | 244.89 | 87.72 | 3 | 475.11 | 56.80 | 3 | 1.94 | 0.025 |
| | Lobular Carcinoma, | Lobular Carcinoma of | | | | | | | | |
| | Primary; Stage I | Breast, All Secondary | | | | | | | | |
| | | Sites | | | | | | | | |
| 208644_at | Breast, Fibrocystic | Breast, Mucinous | 188.81 | 59.90 | 20 | 293.74 | 61.35 | 4 | 1.56 | 0.032 |
| | Disease | Carcinoma, Primary | | | | | | | | |
| 208644_at | Breast, Infiltrating | Metastatic Infiltrating | 312.55 | 101.26 | 26 | 433.92 | 146.92 | 10 | 1.39 | 0.033 |
| | Ductal Carcinoma, | Ductal Carcinoma of | | | | | | | | |
| | Primary; ER+ PR+ | Breast, All Secondary | | | | | | | | |
| | | Sites | | | | | | | | |
| 208644_at | Breast, Infiltrating | Metastatic Infiltrating | 312.55 | 101.26 | 26 | 433.92 | 146.92 | 10 | 1.39 | 0.033 |
| | Ductal Carcinoma, | Ductal Carcinoma of | | | | | | | | |
| | Primary; PR + | Breast, All Secondary | | | | | | | | |
| | | Sites | | | | | | | | |
| 208644_at | Breast, Infiltrating | Metastatic Infiltrating | 315.93 | 99.83 | 35 | 433.92 | 146.92 | 10 | 1.37 | 0.035 |
| | Ductal Carcinoma, | Ductal Carcinoma of | | | | | | | | |
| | Primary; ER + | Breast, All Secondary | | | | | | | | |
| | | Sites | | | | | | | | |
| 208644_at | Breast, Infiltrating | Metastatic Infiltrating | 347.91 | 99.95 | 7 | 475.11 | 56.80 | 3 | 1.37 | 0.039 |
| | Lobular Carcinoma, | Lobular Carcinoma of | | | | | | | | |
| | Primary; ER + | Breast, All Secondary | | | | | | | | |
| | | Sites | | | | | | | | |
| 208644_at | Breast, Infiltrating | Metastatic Infiltrating | 322.14 | 96.83 | 70 | 433.92 | 146.92 | 10 | 1.35 | 0.041 |
| | Ductal Carcinoma, | Ductal Carcinoma of | | | | | | | | |
| | Primary; Stage II | Breast, All Secondary | | | | | | | | |
| | | Sites | | | | | | | | |
| 208644_at | Breast, Infiltrating | Breast, Mucinous | 448.35 | 167.32 | 8 | 293.74 | 61.35 | 4 | −1.53 | 0.044 |
| | Carcinoma of Mixed | Carcinoma, Primary | | | | | | | | |
| | Ductal and Lobular | | | | | | | | | |
| | Type, Primary | | | | | | | | | |

TABLE IV
PARP1 - e-Northern (Human)
Name: poly (ADP-ribose) polymerase family, member 1
Sort By: Organ System
View: NCI 60 Cell Lines
Fragment Legend: 208644_at

| | | | % | | Lower | 25% | | | 75% | Upper |
|---|---|---|---|---|---|---|---|---|---|---|
| Category | Fragment | Freq. | Present | Count | Limit | Quan. | Median | Mean | Quan. | Limit |
| Breast Cell Lines, NCI 60 | | | | | | | | | | |
| BT-549 Human Breast Cancer Cell Line | 208644_at | 1.00 | 1.00 | 1 | 426.69 | 426.69 | 426.69 | 426.69 | 426.69 | 426.69 |
| HS 578T Human Breast Cancer Cell Line | 208644_at | 1.00 | 1.00 | 1 | 268.52 | 268.52 | 268.52 | 268.52 | 268.52 | 268.52 |
| MCF7 Human Breast Cancer Cell Line | 208644_at | 1.00 | 1.00 | 1 | 301.34 | 301.34 | 301.34 | 301.34 | 301.34 | 301.34 |
| MDA-MB-231 Human Breast Cancer Cell Line | 208644_at | 1.00 | 1.00 | 1 | 358.90 | 358.90 | 358.90 | 358.90 | 358.90 | 358.90 |
| MDA-MB-435 Human Breast Cancer Cell Line | 208644_at | 1.00 | 1.00 | 1 | 325.61 | 325.61 | 325.61 | 325.61 | 325.61 | 325.61 |
| MDA-N Human Breast Cancer Cell Line; Derivative | 208644_at | 1.00 | 1.00 | 1 | 328.85 | 328.85 | 328.85 | 328.85 | 328.85 | 328.85 |
| of MDA-MB-435 | | | | | | | | | | |
| Mcf-adr-res Multi-drug Resistant Derivative of | 208644_at | 1.00 | 1.00 | 1 | 323.58 | 323.58 | 323.58 | 323.58 | 323.58 | 323.58 |
| Human Breast Cancer Cell Line | | | | | | | | | | |
| T47D Human Breast Cancer Cell Line | 208644_at | 1.00 | 1.00 | 1 | 409.61 | 409.61 | 409.61 | 409.61 | 409.61 | 409.61 |
| Central Nervous System Cell Lines, NCI 60 | | | | | | | | | | |
| SF-268 Human Glioma Cell Line | 208644_at | 1.00 | 1.00 | 1 | 446.56 | 446.56 | 446.56 | 446.56 | 446.56 | 446.56 |
| SF-295 Human Glioblastoma Cell Line | 208644_at | 1.00 | 1.00 | 1 | 280.85 | 280.85 | 280.85 | 280.85 | 280.85 | 280.85 |
| SF-539 Human Glioblastoma Cell Line | 208644_at | 1.00 | 1.00 | 1 | 212.98 | 212.98 | 212.98 | 212.98 | 212.98 | 212.98 |
| SNB-19 Human Glioblastoma Cell Line | 208644_at | 1.00 | 1.00 | 1 | 353.92 | 353.92 | 353.92 | 353.92 | 353.92 | 353.92 |
| SNB-75 Human Glioblastoma Cell Line | 208644_at | 1.00 | 1.00 | 1 | 376.08 | 376.08 | 376.08 | 376.08 | 376.08 | 376.08 |
| U251 Human Glioblastoma Cell Line | 208644_at | 1.00 | 1.00 | 1 | 367.67 | 367.67 | 367.67 | 367.67 | 367.67 | 367.67 |
| Colon Cell Lines, NCI 60 | | | | | | | | | | |
| COLO 205 Human Colon Cancer Cell Line | 208644_at | 1.00 | 1.00 | 1 | 248.41 | 248.41 | 248.41 | 248.41 | 248.41 | 248.41 |
| HCC-2998 Human Colon Cancer Cell Line | 208644_at | 1.00 | 1.00 | 1 | 294.03 | 294.03 | 294.03 | 294.03 | 294.03 | 294.03 |
| HCT-116 Human Colon Cancer Cell Line | 208644_at | 1.00 | 1.00 | 1 | 556.25 | 556.25 | 556.25 | 556.25 | 556.25 | 556.25 |
| HCT-15 Human Colon Cancer Cell Line | 208644_at | 1.00 | 1.00 | 1 | 359.69 | 359.69 | 359.69 | 359.69 | 359.69 | 359.69 |
| HT29 Colon Cancer Cell Line | 208644_at | 1.00 | 1.00 | 1 | 261.01 | 261.01 | 261.01 | 261.01 | 261.01 | 261.01 |
| KM12 Human Colon Cancer Cell Line | 208644_at | 1.00 | 1.00 | 1 | 375.88 | 375.88 | 375.88 | 375.88 | 375.88 | 375.88 |
| SW-620 Human Colon Cancer Cell Line | 208644_at | 1.00 | 1.00 | 1 | 303.72 | 303.72 | 303.72 | 303.72 | 303.72 | 303.72 |
| Kidney Cell Lines, NCI 60 | | | | | | | | | | |
| 786-O Human Primary Renal Cell Adenocarcinoma | 208644_at | 1.00 | 1.00 | 1 | 288.73 | 288.73 | 288.73 | 288.73 | 288.73 | 288.73 |
| Cell Line | | | | | | | | | | |
| A498 Human Kidney Cancer Cell Line | 208644_at | 1.00 | 1.00 | 1 | 331.74 | 331.74 | 331.74 | 331.74 | 331.74 | 331.74 |
| ACHN Human Kidney Cancer Cell Line | 208644_at | 1.00 | 1.00 | 1 | 282.59 | 282.59 | 282.59 | 282.59 | 282.59 | 282.59 |
| CAKI-1 Human Kidney Cancer Cell Line | 208644_at | 1.00 | 1.00 | 1 | 394.35 | 394.35 | 394.35 | 394.35 | 394.35 | 394.35 |
| RXF-393 Human Kidney Cancer Cell Line | 208644_at | 1.00 | 1.00 | 1 | 190.83 | 190.83 | 190.83 | 190.83 | 190.83 | 190.83 |
| SN12C Human Kidney Cancer Cell Line | 208644_at | 1.00 | 1.00 | 1 | 361.50 | 361.50 | 361.50 | 361.50 | 361.50 | 361.50 |
| TK-10 Human Kidney Cancer Cell Line | 208644_at | 1.00 | 1.00 | 1 | 335.59 | 335.59 | 335.59 | 335.59 | 335.59 | 335.59 |
| UO-31 Human Kidney Cancer Cell Line | 208644_at | 1.00 | 1.00 | 1 | 197.90 | 197.90 | 197.90 | 197.90 | 197.90 | 197.90 |
| Leukemia Cell Lines, NCI 60 | | | | | | | | | | |
| CCRF-CEM Human Leukemia Cell Line | 208644_at | 1.00 | 1.00 | 1 | 321.70 | 321.70 | 321.70 | 321.70 | 321.70 | 321.70 |
| HL-60 (Tb) Human Promyelocytic Leukemia Cell | 208644_at | 1.00 | 1.00 | 1 | 408.26 | 408.26 | 408.26 | 408.26 | 408.26 | 408.26 |
| Line | | | | | | | | | | |
| K-562 Human Chronic Myeloid Leukemia (Cml) | 208644_at | 1.00 | 1.00 | 1 | 622.76 | 622.76 | 622.76 | 622.76 | 622.76 | 622.76 |
| Cell Line | | | | | | | | | | |
| MOLT-4 Human T-cell Leukemia Cell Line | 208644_at | 1.00 | 1.00 | 1 | 277.57 | 277.57 | 277.57 | 277.57 | 277.57 | 277.57 |
| RPMI-8226 Human Multiple Myeloma Cell Line | 208644_at | 1.00 | 1.00 | 1 | 677.06 | 677.06 | 677.06 | 677.06 | 677.06 | 677.06 |
| SR Human Lymphoma Cell Line | 208644_at | 1.00 | 1.00 | 1 | 823.03 | 823.03 | 823.03 | 823.03 | 823.03 | 823.03 |
| Lung Cell Lines, NCI 60 | | | | | | | | | | |
| A549 Human Lung Cancer Cell Line | 208644_at | 1.00 | 1.00 | 1 | 281.44 | 281.44 | 281.44 | 281.44 | 281.44 | 281.44 |
| EKVX Human Non-Small Cell Lung Cancer Cell | 208644_at | 1.00 | 1.00 | 1 | 261.21 | 261.21 | 261.21 | 261.21 | 261.21 | 261.21 |
| Line | | | | | | | | | | |
| HOP-62 Human Non-Small Cell Lung Cancer Cell | 208644_at | 1.00 | 1.00 | 1 | 312.45 | 312.45 | 312.45 | 312.45 | 312.45 | 312.45 |
| Line | | | | | | | | | | |
| HOP-92 Human Non-Small Cell Lung Cancer Cell | 208644_at | 1.00 | 1.00 | 1 | 389.05 | 389.05 | 389.05 | 389.05 | 389.05 | 389.05 |
| Line | | | | | | | | | | |
| NCI-H226 Human Lung Cancer Cell Line | 208644_at | 1.00 | 1.00 | 1 | 144.96 | 144.96 | 144.96 | 144.96 | 144.96 | 144.96 |
| NCI-H23 Human Lung Cancer Cell Line | 208644_at | 1.00 | 1.00 | 1 | 402.19 | 402.19 | 402.19 | 402.19 | 402.19 | 402.19 |
| NCI-H322M Human Non-Small Cell Lung Cancer | 208644_at | 1.00 | 1.00 | 1 | 306.25 | 306.25 | 306.25 | 306.25 | 306.25 | 306.25 |
| Cell Line | | | | | | | | | | |
| NCI-H460 Human Lung Cancer Cell Line | 208644_at | 1.00 | 1.00 | 1 | 478.37 | 478.37 | 478.37 | 478.37 | 478.37 | 478.37 |
| NCI-H522 Human Lung Cancer Cell Line | 208644_at | 1.00 | 1.00 | 1 | 383.68 | 383.68 | 383.68 | 383.68 | 383.68 | 383.68 |
| Melanoma Cell Lines, NCI 60 | | | | | | | | | | |
| LOX IMVI Human Melanoma Cell Line | 208644_at | 1.00 | 1.00 | 1 | 207.77 | 207.77 | 207.77 | 207.77 | 207.77 | 207.77 |
| M14 Human Melanoma Cell Line | 208644_at | 1.00 | 1.00 | 1 | 341.84 | 341.84 | 341.84 | 341.84 | 341.84 | 341.84 |
| MALME-3M Human Melanoma Cell Line | 208644_at | 1.00 | 1.00 | 1 | 431.41 | 431.41 | 431.41 | 431.41 | 431.41 | 431.41 |
| SK-MEL-2 Human Melanoma Cell Line | 208644_at | 1.00 | 1.00 | 1 | 438.73 | 438.73 | 438.73 | 438.73 | 438.73 | 438.73 |
| SK-MEL-28 Human Melanoma Cell Line | 208644_at | 1.00 | 1.00 | 1 | 476.83 | 476.83 | 476.83 | 476.83 | 476.83 | 476.83 |
| SK-MEL-5 Human Melanoma Cell Line | 208644_at | 1.00 | 1.00 | 1 | 463.63 | 463.63 | 463.63 | 463.63 | 463.63 | 463.63 |
| UACC-257 Human Melanoma Cell Line | 208644_at | 1.00 | 1.00 | 1 | 444.85 | 444.85 | 444.85 | 444.85 | 444.85 | 444.85 |
| UACC-62 Human Melanoma Cell Line | 208644_at | 1.00 | 1.00 | 1 | 277.94 | 277.94 | 277.94 | 277.94 | 277.94 | 277.94 |
| Ovarian Cell Lines, NCI 60 | | | | | | | | | | |
| IGROV1 Human Ovarian Cancer Cell Line | 208644_at | 1.00 | 1.00 | 1 | 577.51 | 577.51 | 577.51 | 577.51 | 577.51 | 577.51 |
| OVCAR-3 Human Ovarian Cancer Cell Line | 208644_at | 1.00 | 1.00 | 1 | 304.19 | 304.19 | 304.19 | 304.19 | 304.19 | 304.19 |
| OVCAR-4 Human Ovarian Cancer Cell Line | 208644_at | 1.00 | 1.00 | 1 | 182.23 | 182.23 | 182.23 | 182.23 | 182.23 | 182.23 |
| OVCAR-5 Human Ovarian Cancer Cell Line | 208644_at | 1.00 | 1.00 | 1 | 228.97 | 228.97 | 228.97 | 228.97 | 228.97 | 228.97 |
| OVCAR-8 Human Ovarian Cancer Cell Line | 208644_at | 1.00 | 1.00 | 1 | 377.20 | 377.20 | 377.20 | 377.20 | 377.20 | 377.20 |
| SK-OV-3 Human Ovarian Cancer Cell Line | 208644_at | 1.00 | 1.00 | 1 | 340.14 | 340.14 | 340.14 | 340.14 | 340.14 | 340.14 |
| Prostate Cell Lines, NCI 60 | | | | | | | | | | |
| DU-145 Human Prostate Cancer Cell Line | 208644_at | 1.00 | 1.00 | 1 | 470.28 | 470.28 | 470.28 | 470.28 | 470.28 | 470.28 |
| PC-3 Human Prostate Cancer Cell Line | 208644_at | 1.00 | 1.00 | 1 | 412.78 | 412.78 | 412.78 | 412.78 | 412.78 | 412.78 |

TABLE V
PARP1 - Diff/X (Human)
Name: poly (ADP-ribose) polymerase family, member 1
Minimum Fold Change: 1.2 p-Value
Range: 0.00-0.05
Sort By: p-value
One differential expression event found.
Metabolism

| | | | | Control | | | Experiment | | Fold | |
|---|---|---|---|---|---|---|---|---|---|---|
| | | | Control | Standard | | Experiment | Standard | | change | |
| Fragment | Control | Experiment | Mean | Deviation | Control # | Mean | Deviation | Experiment # | (FC) | p-value |
| 208644_at | Liver, | Liver, Steatosis | 195.15 | 85.33 | 42 | 142.95 | 29.82 | 4 | −1.37 | 0.027 |
| | Normal | (Fatty Change) | | | | | | | | |

TABLE VI
PARP1 - e-Northern (Human)
Name: poly (ADP-ribose) polymerase family, member 1
Sort By: Organ System
View: Metabolic Disease
Fragment Legend: 208644_at

| | | | % | | Lower | 25% | | | 75% | Upper |
|---|---|---|---|---|---|---|---|---|---|---|
| Category | Fragment | Freq. | Present | Count | Limit | Quan. | Median | Mean | Quan. | Limit |
| Human, Metabolic Diseases, Digestive System | | | | | | | | | | |
| Liver, Normal | 208644_at | 1.00 | 1.00 | 42 | 85.71 | 149.27 | 172.85 | 195.15 | 201.08 | 278.79 |
| Liver, Normal; Diabetic | 208644_at | 1.00 | 1.00 | 5 | 102.80 | 169.95 | 187.56 | 246.84 | 257.34 | 388.43 |
| Liver, Normal; Nondiabetic | 208644_at | 1.00 | 1.00 | 41 | 85.71 | 153.13 | 175.41 | 195.74 | 200.34 | 271.17 |
| Liver, Normal; Nonobese | 208644_at | 1.00 | 1.00 | 10 | 107.66 | 171.58 | 192.58 | 224.72 | 214.19 | 278.10 |
| Liver, Normal; Nonobese, Nondiabetic | 208644_at | 1.00 | 1.00 | 8 | 161.58 | 174.13 | 192.58 | 218.29 | 202.91 | 246.08 |
| Liver, Normal; Obese | 208644_at | 1.00 | 1.00 | 5 | 128.47 | 141.91 | 194.71 | 204.32 | 257.34 | 299.15 |
| Liver, Normal; Obese, Nondiabetic | 208644_at | 1.00 | 1.00 | 4 | 128.47 | 138.55 | 168.31 | 191.06 | 220.82 | 299.15 |
| Human, Metabolic Diseases, Integumentary System | | | | | | | | | | |
| Adipose Tissue, Normal | 208644_at | 1.00 | 1.00 | 34 | 102.07 | 152.27 | 170.25 | 171.87 | 186.93 | 238.93 |
| Adipose Tissue, Normal, Diabetic | 208644_at | 1.00 | 1.00 | 5 | 137.62 | 141.52 | 170.87 | 161.26 | 172.25 | 184.05 |
| Adipose Tissue, Normal, Nondiabetic | 208644_at | 1.00 | 1.00 | 29 | 102.07 | 154.02 | 172.48 | 175.46 | 190.13 | 240.92 |
| Adipose Tissue, Normal, Nonobese | 208644_at | 1.00 | 1.00 | 7 | 139.06 | 145.65 | 157.10 | 169.13 | 172.09 | 211.76 |
| Adipose Tissue, Normal, Nonobese, | 208644_at | 1.00 | 1.00 | 4 | 143.91 | 153.80 | 165.21 | 181.65 | 193.06 | 251.94 |
| Nondiabetic | | | | | | | | | | |
| Adipose Tissue, Normal, Obese | 208644_at | 1.00 | 1.00 | 18 | 102.07 | 144.12 | 170.94 | 163.81 | 183.85 | 200.44 |
| Adipose Tissue, Normal, Obese, | 208644_at | 1.00 | 1.00 | 4 | 137.62 | 140.54 | 156.89 | 158.86 | 175.20 | 184.05 |
| Diabetic | | | | | | | | | | |
| Adipose Tissue, Normal, Obese, | 208644_at | 1.00 | 1.00 | 15 | 111.91 | 156.35 | 172.48 | 166.89 | 185.97 | 200.44 |
| Nondiabetic | | | | | | | | | | |
| Human, Metabolic Diseases, Musculoskeletal | | | | | | | | | | |
| System | | | | | | | | | | |
| Skeletal Muscle, Normal | 208644_at | 1.00 | 1.00 | 47 | 124.99 | 198.66 | 232.58 | 232.85 | 273.29 | 347.32 |
| Skeletal Muscle, Normal, Diabetic | 208644_at | 1.00 | 1.00 | 5 | 187.24 | 203.45 | 206.20 | 223.82 | 259.99 | 262.24 |
| Skeletal Muscle, Normal, Nondiabetic | 208644_at | 1.00 | 1.00 | 40 | 124.99 | 194.55 | 229.50 | 230.83 | 270.45 | 347.32 |
| Skeletal Muscle, Normal, Nonobese | 208644_at | 1.00 | 1.00 | 17 | 141.60 | 198.18 | 233.67 | 234.09 | 267.61 | 322.92 |
| Skeletal Muscle, Normal, Nonobese, | 208644_at | 1.00 | 1.00 | 15 | 141.60 | 190.91 | 232.58 | 229.04 | 267.18 | 322.92 |
| Nondiabetic | | | | | | | | | | |
| Skeletal Muscle, Normal, Obese | 208644_at | 1.00 | 1.00 | 9 | 150.38 | 166.21 | 203.45 | 221.01 | 279.83 | 325.09 |
| Skeletal Muscle, Normal, Obese, | 208644_at | 1.00 | 1.00 | 7 | 150.38 | 163.21 | 235.93 | 228.34 | 280.28 | 325.09 |
| Nondiabetic | | | | | | | | | | |

TABLE VII
PARP1 - Diff/X (Human)
Name: poly (ADP-ribose) polymerase family, member 1
Sort By: Name (A-Z)
View: Metabolic Disease
Fragment Legend: 208644_at

| | | | % | | Lower | 25% | | | 75% | Upper |
|---|---|---|---|---|---|---|---|---|---|---|
| Sample Set | Fragment | Freq. | Present | Count | Limit | Quan. | Median | Mean | Quan. | Limit |
| Adipose Tissue, Normal | 208644_at | 1.00 | 1.00 | 34 | 102.07 | 152.27 | 170.25 | 171.87 | 186.93 | 238.93 |
| Adipose Tissue, Normal, Diabetic | 208644_at | 1.00 | 1.00 | 5 | 137.62 | 141.52 | 170.87 | 161.26 | 172.25 | 184.05 |
| Adipose Tissue, Normal, Nondiabetic | 208644_at | 1.00 | 1.00 | 29 | 102.07 | 154.02 | 172.48 | 175.46 | 190.13 | 240.92 |
| Adipose Tissue, Normal, Nonobese | 208644_at | 1.00 | 1.00 | 7 | 139.06 | 145.65 | 157.10 | 169.13 | 172.09 | 211.76 |
| Adipose Tissue, Normal, Nonobese, | 208644_at | 1.00 | 1.00 | 4 | 143.91 | 153.80 | 165.21 | 181.65 | 193.06 | 251.94 |
| Nondiabetic | | | | | | | | | | |
| Adipose Tissue, Normal, Obese | 208644_at | 1.00 | 1.00 | 18 | 102.07 | 144.12 | 170.94 | 163.81 | 183.85 | 200.44 |
| Adipose Tissue, Normal, Obese, | 208644_at | 1.00 | 1.00 | 4 | 137.62 | 140.54 | 156.89 | 158.86 | 175.20 | 184.05 |
| Diabetic | | | | | | | | | | |
| Adipose Tissue, Normal, Obese, | 208644_at | 1.00 | 1.00 | 15 | 111.91 | 156.35 | 172.48 | 166.89 | 185.97 | 200.44 |
| Nondiabetic | | | | | | | | | | |
| Liver, Normal | 208644_at | 1.00 | 1.00 | 42 | 85.71 | 149.27 | 172.85 | 195.15 | 201.08 | 278.79 |
| Liver, Normal; Diabetic | 208644_at | 1.00 | 1.00 | 5 | 102.80 | 169.95 | 187.56 | 246.84 | 257.34 | 388.43 |
| Liver, Normal; Nondiabetic | 208644_at | 1.00 | 1.00 | 41 | 85.71 | 153.13 | 175.41 | 195.74 | 200.34 | 271.17 |
| Liver, Normal; Nonobese | 208644_at | 1.00 | 1.00 | 10 | 107.66 | 171.58 | 192.58 | 224.72 | 214.19 | 278.10 |
| Liver, Normal; Nonobese, Nondiabetic | 208644_at | 1.00 | 1.00 | 8 | 161.58 | 174.13 | 192.58 | 218.29 | 202.91 | 246.08 |
| Liver, Normal; Obese | 208644_at | 1.00 | 1.00 | 5 | 128.47 | 141.91 | 194.71 | 204.32 | 257.34 | 299.15 |
| Liver, Normal; Obese, Nondiabetic | 208644_at | 1.00 | 1.00 | 4 | 128.47 | 138.55 | 168.31 | 191.06 | 220.82 | 299.15 |
| Skeletal Muscle, Normal | 208644_at | 1.00 | 1.00 | 47 | 124.99 | 198.66 | 232.58 | 232.85 | 273.29 | 347.32 |
| Skeletal Muscle, Normal, Diabetic | 208644_at | 1.00 | 1.00 | 5 | 187.24 | 203.45 | 206.20 | 223.82 | 259.99 | 262.24 |
| Skeletal Muscle, Normal, Nondiabetic | 208644_at | 1.00 | 1.00 | 40 | 124.99 | 194.55 | 229.50 | 230.83 | 270.45 | 347.32 |
| Skeletal Muscle, Normal, Nonobese | 208644_at | 1.00 | 1.00 | 17 | 141.60 | 198.18 | 233.67 | 234.09 | 267.61 | 322.92 |
| Skeletal Muscle, Normal, Nonobese, | 208644_at | 1.00 | 1.00 | 15 | 141.60 | 190.91 | 232.58 | 229.04 | 267.18 | 322.92 |
| Nondiabetic | | | | | | | | | | |
| Skeletal Muscle, Normal, Obese | 208644_at | 1.00 | 1.00 | 9 | 150.38 | 166.21 | 203.45 | 221.01 | 279.83 | 325.09 |
| Skeletal Muscle, Normal, Obese, | 208644_at | 1.00 | 1.00 | 7 | 150.38 | 163.21 | 235.93 | 228.34 | 280.28 | 325.09 |
| Nondiabetic | | | | | | | | | | |

TABLE VIII
PARP1 - Diff/X (Human)
Name: poly (ADP-ribose) polymerase family, member 1
Minimum Fold Change: 1.2
p-Value Range: 0.00-0.05
Sort By: p-value
4 differential expression events found.
Medications

| | | | | Control | | | Experiment | | Fold | |
|---|---|---|---|---|---|---|---|---|---|---|
| | | | Control | Standard | | Experiment | Standard | | change | p- |
| Fragment | Control | Experiment | Mean | Deviation | Control # | Mean | Deviation | Experiment # | (FC) | value |
| 208644_at | Ovary, Normal, | Ovary, Normal, | 168.46 | 28.39 | 69 | 138.57 | 5.06 | 3 | −1.22 | 0.000 |
| | Patients Not | Patients Taking | | | | | | | | |
| | Taking Atenolol | Atenolol | | | | | | | | |
| 208644_at | Stomach, Normal, | Stomach, Normal, | 180.56 | 13.33 | 5 | 228.75 | 22.92 | 5 | 1.27 | 0.006 |
| | Female Patients | Patients Taking | | | | | | | | |
| | Not Taking | Estrogens | | | | | | | | |
| | Estrogens | | | | | | | | | |
| 208644_at | Stomach, Normal, | Stomach, Normal, | 219.86 | 40.17 | 21 | 176.85 | 25.05 | 4 | −1.24 | 0.028 |
| | Patients Not | Patients Taking | | | | | | | | |
| | Taking | Atorvastatin | | | | | | | | |
| | Atorvastatin | | | | | | | | | |
| 208644_at | Superior | Superior Temporal | 226.78 | 23.15 | 4 | 290.49 | 61.79 | 7 | 1.28 | 0.039 |
| | Temporal Gyrus | Gyrus (Brodmann | | | | | | | | |
| | (Brodmann Area | Area 22), Normal, | | | | | | | | |
| | 22), Normal, | Patients Taking | | | | | | | | |
| | Patients Not | Acetaminophen | | | | | | | | |
| | Taking | | | | | | | | | |
| | Acetaminophen | | | | | | | | | |

TABLE IX
PARP1 - Diff/X (Human)
Name: poly (ADP-ribose) polymerase family, member 1
Minimum Fold Change: 1.2
p-Value Range: 0.00-0.05
Sort By: p-value
2 differential expression events found.
Male reproductive system

| | | | | Control | | | Experiment | | Fold | |
|---|---|---|---|---|---|---|---|---|---|---|
| | | | Control | Standard | Control | Experiment | Standard | Experiment | change | p- |
| Fragment | Control | Experiment | Mean | Deviation | # | Mean | Deviation | # | (FC) | value |
| 208644_at | Testis, Normal | Testis, Seminoma, | 333.35 | 78.19 | 7 | 622.56 | 164.78 | 8 | 1.87 | 0.001 |
| | | Primary | | | | | | | | |
| 208644_at | Prostate, Benign | Prostate, Benign Nodular | 224.33 | 42.52 | 10 | 183.10 | 32.34 | 10 | −1.23 | 0.026 |
| | Nodular | Hyperplasia; Primary | | | | | | | | |
| | Hyperplasia; No | Malignancy Elsewhere in | | | | | | | | |
| | Primary Prostatic | Prostate | | | | | | | | |
| | Malignancy | | | | | | | | | |

TABLE X
PARP1 - Diff/X (Human)
Name: poly (ADP-ribose) polymerase family, member 1
Minimum Fold Change: 1.2
p-Value Range: 0.00-0.05
Sort By: FC Up
8 differential expression events found.
Respiratory System

| | | | | Control | | | Experiment | | Fold | |
|---|---|---|---|---|---|---|---|---|---|---|
| | | | Control | Standard | Control | Experiment | Standard | Experiment | change | p- |
| Fragment | Control | Experiment | Mean | Deviation | # | Mean | Deviation | # | (FC) | value |
| 208644_at | Lung, Pulmonary | Lung, Squamous | 167.99 | 19.89 | 39 | 309.53 | 103.71 | 39 | 1.84 | 0.000 |
| | Emphysema, not | Cell Carcinoma, | | | | | | | | |
| | Associated with A1AT | Primary | | | | | | | | |
| | Deficiency | | | | | | | | | |
| 208644_at | Lung, Normal | Lung, Squamous | 170.58 | 56.25 | 126 | 309.53 | 103.71 | 39 | 1.81 | 0.000 |
| | | Cell Carcinoma, | | | | | | | | |
| | | Primary | | | | | | | | |
| 208644_at | Lung, Pulmonary | Lung, Large Cell | 167.99 | 19.89 | 39 | 291.08 | 122.74 | 7 | 1.73 | 0.038 |
| | Emphysema, not | Carcinoma, Primary | | | | | | | | |
| | Associated with A1AT | | | | | | | | | |
| | Deficiency | | | | | | | | | |
| 208644_at | Lung, Normal | Lung, Large Cell | 170.58 | 56.25 | 126 | 291.08 | 122.74 | 7 | 1.71 | 0.041 |
| | | Carcinoma, Primary | | | | | | | | |
| 208644_at | Lung, Pulmonary | Lung, | 167.99 | 19.89 | 39 | 284.99 | 92.24 | 46 | 1.70 | 0.000 |
| | Emphysema, not | Adenocarcinoma, | | | | | | | | |
| | Associated with A1AT | Primary | | | | | | | | |
| | Deficiency | | | | | | | | | |
| 208644_at | Lung, Normal | Lung, | 170.58 | 56.25 | 126 | 284.99 | 92.24 | 46 | 1.67 | 0.000 |
| | | Adenocarcinoma, | | | | | | | | |
| | | Primary | | | | | | | | |
| 208644_at | Lung, Adenosquamous | Lung, Squamous | 209.41 | 25.19 | 3 | 309.53 | 103.71 | 39 | 1.48 | 0.001 |
| | Carcinoma, Primary | Cell Carcinoma, | | | | | | | | |
| | | Primary | | | | | | | | |
| 208644_at | Lung, | Lung, | 284.99 | 92.24 | 46 | 209.41 | 25.19 | 3 | −1.36 | 0.007 |
| | Adenocarcinoma, | Adenosquamous | | | | | | | | |
| | Primary | Carcinoma, Primary | | | | | | | | |

TABLE XI
PARP1 - Diff/X (Human)
Name: poly (ADP-ribose) polymerase family, member 1
Minimum Fold Change: 1.2
p-Value Range: 0.00-0.05
Sort By: FC Up
4 differential expression events found.
Urinary tract

| | | | | Control | | | Experiment | | Fold | |
|---|---|---|---|---|---|---|---|---|---|---|
| | | | Control | Standard | | Experiment | Standard | | change | p- |
| Fragment | Control | Experiment | Mean | Deviation | Control # | Mean | Deviation | Experiment # | (FC) | value |
| 208644_at | Kidney, Normal | Kidney, Wilm's | 165.78 | 27.21 | 81 | 385.07 | 125.19 | 8 | 2.32 | 0.002 |
| | | Tumor, Primary | | | | | | | | |
| 208644_at | Kidney, Normal | Kidney, | 165.78 | 27.21 | 81 | 256.62 | 24.30 | 4 | 1.55 | 0.004 |
| | | Transitional Cell | | | | | | | | |
| | | Carcinoma, | | | | | | | | |
| | | Primary | | | | | | | | |
| 208644_at | Kidney, Renal Cell | Kidney, | 178.49 | 58.38 | 15 | 127.65 | 22.77 | 3 | −1.40 | 0.033 |
| | Carcinoma, Non- | Carcinoma, | | | | | | | | |
| | Clear Cell Type, | Chromophobe | | | | | | | | |
| | Primary | Type, Primary | | | | | | | | |
| 208644_at | Kidney, Renal Cell | Kidney, | 201.00 | 134.98 | 45 | 127.65 | 22.77 | 3 | −1.57 | 0.007 |
| | Carcinoma, Clear | Carcinoma, | | | | | | | | |
| | Cell Type, Primary | Chromophobe | | | | | | | | |
| | | Type, Primary | | | | | | | | |

TABLE XII
PARP1 - Diff/X (Human)
Name: poly (ADP-ribose) polymerase family, member 1
Minimum Fold Change: 1.2
p-Value Range: 0.00-0.05
Sort By: p-value
20 differential expression events found.
Inflammation

| | | | | Control | | | Experiment | Exper- | Fold | |
|---|---|---|---|---|---|---|---|---|---|---|
| | | | Control | Standard | Control | Experiment | Standard | iment | change | p- |
| Fragment | Control | Experiment | Mean | Deviation | # | Mean | Deviation | # | (FC) | value |
| 208644_at | Lymph Node, | Lymph Node, Non- | 475.49 | 123.80 | 9 | 756.79 | 372.23 | 91 | 1.59 | 0.000 |
| | Reactive Lymphoid | Hodgkin's Lymphoma, | | | | | | | | |
| | Hyperplasia | All Types | | | | | | | | |
| 208644_at | White Blood Cells, | White Blood Cells, | 176.91 | 35.10 | 14 | 119.48 | 38.30 | 27 | −1.48 | 0.000 |
| | Normal | Wegener's | | | | | | | | |
| | | Granulomatosis | | | | | | | | |
| 208644_at | Thyroid Gland, | Thyroid Gland, | 173.96 | 29.46 | 58 | 250.53 | 67.58 | 19 | 1.44 | 0.000 |
| | Nodular Hyperplasia | Hashimoto's Thyroiditis | | | | | | | | |
| 208644_at | Thyroid Gland, | Thyroid Gland, | 173.82 | 34.78 | 24 | 250.53 | 67.58 | 19 | 1.44 | 0.000 |
| | Normal | Hashimoto's Thyroiditis | | | | | | | | |
| 208644_at | Liver, Cirrhosis | Liver, Hepatocellular | 168.31 | 20.68 | 25 | 241.43 | 87.63 | 16 | 1.43 | 0.005 |
| | Secondary to Chronic | Carcinoma | | | | | | | | |
| | Hepatitis C | | | | | | | | | |
| 208644_at | Liver, Cirrhosis, All | Liver, Hepatocellular | 169.21 | 36.75 | 61 | 241.43 | 87.63 | 16 | 1.43 | 0.005 |
| | Causes | Carcinoma | | | | | | | | |
| 208644_at | Thymus, Normal | Thymus, Atrophy | 263.23 | 48.02 | 62 | 184.89 | 19.58 | 3 | −1.42 | 0.007 |
| 208644_at | Pancreas, Normal | Pancreas, Chronic | 321.84 | 69.04 | 46 | 245.29 | 70.89 | 10 | −1.31 | 0.008 |
| | | Pancreatitis | | | | | | | | |
| 208644_at | Thyroid Gland, | Thyroid Gland, | 179.21 | 28.92 | 8 | 249.79 | 64.31 | 10 | 1.39 | 0.008 |
| | Normal; No Smoking | Hashimoto's Thyroiditis; | | | | | | | | |
| | History | No Smoking History | | | | | | | | |
| 208644_at | Pancreas, Normal; | Pancreas, Chronic | 313.58 | 74.61 | 23 | 224.88 | 62.47 | 7 | −1.39 | 0.009 |
| | Smoking History | Pancreatitis; Smoking | | | | | | | | |
| | | History | | | | | | | | |
| 208644_at | White Blood Cells, | White Blood Cells, | 176.91 | 35.10 | 14 | 137.82 | 38.04 | 14 | −1.28 | 0.009 |
| | Normal | Rheumatoid Arthritis | | | | | | | | |
| 208644_at | Lymph Node, Normal | Lymph Node, Reactive | 325.59 | 104.66 | 10 | 475.49 | 123.80 | 9 | 1.46 | 0.012 |
| | | Lymphoid Hyperplasia | | | | | | | | |
| 208644_at | Bone, Degenerative | Bone, Degenerative | 179.81 | 26.23 | 27 | 227.29 | 21.64 | 4 | 1.26 | 0.013 |
| | Joint Disease | Joint Disease | | | | | | | | |
| | (Osteoarthritis); Knee | (Osteoarthritis); Hip | | | | | | | | |
| | Joints Only | Joints Only | | | | | | | | |
| 208644_at | Pancreas, Normal | Pancreas, Chronic | 321.84 | 69.04 | 46 | 215.92 | 55.96 | 4 | −1.49 | 0.025 |
| | | Pancreatitis with | | | | | | | | |
| | | Fibrosis | | | | | | | | |
| 208644_at | Thyroid Gland, | Thyroid Gland, | 171.03 | 45.31 | 13 | 264.17 | 52.94 | 4 | 1.54 | 0.029 |
| | Normal; Primary | Hashimoto's Thyroiditis; | | | | | | | | |
| | Malignancy Elsewhere | Primary Malignancy | | | | | | | | |
| | in Thyroid | Elsewhere in Thyroid | | | | | | | | |
| 208644_at | Colon, Normal; No | Colon, Ulcerative | 197.19 | 44.36 | 152 | 237.19 | 57.53 | 13 | 1.20 | 0.029 |
| | History of | Colitis, Active (Acute | | | | | | | | |
| | Inflammatory Bowel | Inflammation) | | | | | | | | |
| | Disease | | | | | | | | | |
| 208644_at | Thyroid Gland, | Thyroid Gland, | 173.82 | 34.78 | 24 | 236.80 | 77.06 | 10 | 1.36 | 0.031 |
| | Normal | Hashimoto's Thyroiditis; | | | | | | | | |
| | | No Primary Thyroid | | | | | | | | |
| | | Malignancy | | | | | | | | |
| 208644_at | Thyroid Gland, | Thyroid Gland, Papillary | 250.53 | 67.58 | 19 | 206.04 | 51.25 | 15 | −1.22 | 0.037 |
| | Hashimoto's | Carcinoma (Excluding | | | | | | | | |
| | Thyroiditis | Follicular Variant), | | | | | | | | |
| | | Primary | | | | | | | | |
| 208644_at | Thyroid Gland, | Thyroid Gland, | 173.82 | 34.78 | 24 | 264.17 | 52.94 | 4 | 1.52 | 0.037 |
| | Normal | Hashimoto's Thyroiditis; | | | | | | | | |
| | | Primary Malignancy | | | | | | | | |
| | | Elsewhere in Thyroid | | | | | | | | |
| 208644_at | Thyroid Gland, | Thyroid Gland, | 172.59 | 37.49 | 7 | 236.80 | 77.06 | 10 | 1.37 | 0.039 |
| | Normal; No Primary | Hashimoto's Thyroiditis; | | | | | | | | |
| | Thyroid Malignancy | No Primary Thyroid | | | | | | | | |
| | | Malignancy | | | | | | | | |

TABLE XIII
PARP1 - Diff/X (Human)
Name: poly (ADP-ribose) polymerase family, member 1
Minimum Fold Change: 1.2
p-Value Range: 0.00-0.05
Sort By: p-value
18 differential expression events found.
Female reproductive system

| | | | | Control | | | Experiment | Experi- | Fold | |
|---|---|---|---|---|---|---|---|---|---|---|
| | | | Control | Standard | | Experiment | Standard | ment | change | p- |
| Fragment | Control | Experiment | Mean | Deviation | Control # | Mean | Deviation | # | (FC) | value |
| 208644_at | Ovary, Normal | Ovary, | 163.31 | 30.51 | 89 | 361.56 | 153.46 | 36 | 2.21 | 0.000 |
| | | Adenocarcinoma, | | | | | | | | |
| | | Papillary Serous Type, | | | | | | | | |
| | | Primary | | | | | | | | |
| 208644_at | Endometrium, Normal | Endometrium, | 201.21 | 62.21 | 23 | 297.42 | 98.78 | 50 | 1.48 | 0.000 |
| | | Adenocarcinoma, | | | | | | | | |
| | | Endometrioid Type, | | | | | | | | |
| | | Primary | | | | | | | | |
| 208644_at | Ovary, Mucinous | Ovary, | 191.45 | 47.99 | 7 | 361.56 | 153.46 | 36 | 1.89 | 0.000 |
| | Cystadenocarcinoma, | Adenocarcinoma, | | | | | | | | |
| | Primary | Papillary Serous Type, | | | | | | | | |
| | | Primary | | | | | | | | |
| 208644_at | Ovary, Normal | Ovary, | 163.31 | 30.51 | 89 | 331.23 | 140.37 | 22 | 2.03 | 0.000 |
| | | Adenocarcinoma, | | | | | | | | |
| | | Endometrioid Type, | | | | | | | | |
| | | Primary | | | | | | | | |
| 208644_at | Ovary, | Ovary, | 220.76 | 45.99 | 6 | 361.56 | 153.46 | 36 | 1.64 | 0.000 |
| | Adenocarcinoma, | Adenocarcinoma, | | | | | | | | |
| | Clear Cell Type, | Papillary Serous Type, | | | | | | | | |
| | Primary | Primary | | | | | | | | |
| 208644_at | Myometrium, Normal | Myometrium, | 176.66 | 30.91 | 122 | 213.73 | 61.61 | 46 | 1.21 | 0.000 |
| | | Leiomyoma | | | | | | | | |
| 208644_at | Ovary, | Ovary, Mucinous | 331.23 | 140.37 | 22 | 191.45 | 47.99 | 7 | −1.73 | 0.000 |
| | Adenocarcinoma, | Cystadenocarcinoma, | | | | | | | | |
| | Endometrioid Type, | Primary | | | | | | | | |
| | Primary | | | | | | | | | |
| 208644_at | Ovary, Normal | Ovary, Serous | 163.31 | 30.51 | 89 | 371.23 | 104.08 | 8 | 2.27 | 0.001 |
| | | Cystadenocarcinoma, | | | | | | | | |
| | | Primary | | | | | | | | |
| 208644_at | Endometrium, Normal; | Endometrium, | 200.26 | 56.94 | 10 | 286.55 | 91.55 | 40 | 1.43 | 0.001 |
| | No Smoking History | Adenocarcinoma, | | | | | | | | |
| | | Endometrioid Type, | | | | | | | | |
| | | Primary; No Smoking | | | | | | | | |
| | | History | | | | | | | | |
| 208644_at | Ovary, Mucinous | Ovary, Serous | 191.45 | 47.99 | 7 | 371.23 | 104.08 | 8 | 1.94 | 0.001 |
| | Cystadenocarcinoma, | Cystadenocarcinoma, | | | | | | | | |
| | Primary | Primary | | | | | | | | |
| 208644_at | Endometrium, | Endometrium, | 250.09 | 12.48 | 3 | 308.83 | 97.96 | 35 | 1.23 | 0.003 |
| | Adenocarcinoma, | Adenocarcinoma, | | | | | | | | |
| | Endometrioid Type, | Endometrioid Type, | | | | | | | | |
| | Primary; | Primary; | | | | | | | | |
| | Premenopausal | Postmenopausal | | | | | | | | |
| 208644_at | Endometrium, Normal | Endometrium, | 201.21 | 62.21 | 23 | 517.86 | 185.55 | 7 | 2.57 | 0.004 |
| | | Mullerian Mixed | | | | | | | | |
| | | Tumor, Primary | | | | | | | | |
| 208644_at | Endometrium, Normal; | Endometrium, | 202.89 | 75.38 | 10 | 336.79 | 71.19 | 6 | 1.66 | 0.004 |
| | Smoking History | Adenocarcinoma, | | | | | | | | |
| | Endometrioid Type, | | | | | | | | | |
| | Primary; Smoking | | | | | | | | | |
| | History | | | | | | | | | |
| 208644_at | Ovary, | Ovary, Serous | 220.76 | 45.99 | 6 | 371.23 | 104.08 | 8 | 1.68 | 0.004 |
| | Adenocarcinoma, | Cystadenocarcinoma, | | | | | | | | |
| | Clear Cell Type, | Primary | | | | | | | | |
| | Primary | | | | | | | | | |
| 208644_at | Ovary, | Ovary, | 220.76 | 45.99 | 6 | 331.23 | 140.37 | 22 | 1.50 | 0.004 |
| | Adenocarcinoma, | Adenocarcinoma, | | | | | | | | |
| | Clear Cell Type, | Endometrioid Type, | | | | | | | | |
| | Primary | Primary | | | | | | | | |
| 208644_at | Endometrium, | Endometrium, | 297.42 | 98.78 | 50 | 517.86 | 185.55 | 7 | 1.74 | 0.020 |
| | Adenocarcinoma, | Mullerian Mixed | | | | | | | | |
| | Endometrioid Type, | Tumor, Primary | | | | | | | | |
| | Primary | | | | | | | | | |
| 208644_at | Ovary, Normal | Ovary, | 163.31 | 30.51 | 89 | 220.76 | 45.99 | 6 | 1.35 | 0.027 |
| | | Adenocarcinoma, | | | | | | | | |
| | | Clear Cell Type, | | | | | | | | |
| | | Primary | | | | | | | | |
| 208644_at | Ovary, Normal | Ovary, Mullerian | 163.31 | 30.51 | 89 | 371.40 | 144.27 | 5 | 2.27 | 0.032 |
| | | Mixed Tumor, Primary | | | | | | | | |

TABLE XIV
PARP1 - Diff/X (Human)
Name: poly (ADP-ribose) polymerase family, member 1
Minimum Fold Change: 1.6
p-Value Range: 0.00-0.05
Sort By: FC Up
35 differential expression events found.
ONCOLOGY

| | | | | Control | | | Experiment | Experi- | Fold | |
|---|---|---|---|---|---|---|---|---|---|---|
| | | | Control | Standard | | Experiment | Standard | ment | change | p- |
| Fragment | Control | Experiment | Mean | Deviation | Control # | Mean | Deviation | # | (FC) | value |
| 208644_at | Endometrium, | Endometrium, | 201.21 | 62.21 | 23 | 517.86 | 185.55 | 7 | 2.57 | 0.004 |
| | Normal | Mullerian Mixed | | | | | | | | |
| | | Tumor, Primary | | | | | | | | |
| 208644_at | Breast, Fibrocystic | Breast, Infiltrating | 188.81 | 59.90 | 20 | 448.35 | 167.32 | 8 | 2.37 | 0.003 |
| | Disease | Carcinoma of Mixed | | | | | | | | |
| | | Ductal and Lobular | | | | | | | | |
| | | Type, Primary | | | | | | | | |
| 208644_at | Kidney, Normal | Kidney, Wilm's | 165.78 | 27.21 | 81 | 385.07 | 125.19 | 8 | 2.32 | 0.002 |
| | | Tumor, Primary | | | | | | | | |
| 208644_at | Ovary, Normal | Ovary, Mullerian | 163.31 | 30.51 | 89 | 371.40 | 144.27 | 5 | 2.27 | 0.032 |
| | | Mixed Tumor, | | | | | | | | |
| | | Primary | | | | | | | | |
| 208644_at | Ovary, Normal | Ovary, Serous | 163.31 | 30.51 | 89 | 371.23 | 104.08 | 8 | 2.27 | 0.001 |
| | | Cystadenocarcinoma, | | | | | | | | |
| | | Primary | | | | | | | | |
| 208644_at | Breast, Normal | Breast, Infiltrating | 201.78 | 81.64 | 68 | 448.35 | 167.32 | 8 | 2.22 | 0.004 |
| | | Carcinoma of Mixed | | | | | | | | |
| | | Ductal and Lobular | | | | | | | | |
| | | Type, Primary | | | | | | | | |
| 208644_at | Ovary, Normal | Ovary, | 163.31 | 30.51 | 89 | 361.56 | 153.46 | 36 | 2.21 | 0.000 |
| | | Adenocarcinoma, | | | | | | | | |
| | | Papillary Serous | | | | | | | | |
| | | Type, Primary | | | | | | | | |
| 208644_at | Ovary, Normal | Ovary, | 163.31 | 30.51 | 89 | 331.23 | 140.37 | 22 | 2.03 | 0.000 |
| | | Adenocarcinoma, | | | | | | | | |
| | | Endometrioid Type, | | | | | | | | |
| | | Primary | | | | | | | | |
| 208644_at | Breast, Infiltrating | Metastatic Infiltrating | 244.89 | 87.72 | 3 | 475.11 | 56.8 | 3 | 1.94 | 0.025 |
| | Lobular | Lobular Carcinoma | | | | | | | | |
| | Carcinoma, | of Breast, All | | | | | | | | |
| | Primary; Stage I | Secondary Sites | | | | | | | | |
| 208644_at | Ovary, Mucinous | Ovary, Serous | 191.45 | 47.99 | 7 | 371.23 | 104.08 | 8 | 1.94 | 0.001 |
| | Cystadenocarcinoma, | Cystadenocarcinoma, | | | | | | | | |
| | Primary | Primary | | | | | | | | |
| 208644_at | Ovary, Mucinous | Ovary, | 191.45 | 47.99 | 7 | 361.56 | 153.46 | 36 | 1.89 | 0.000 |
| | Cystadenocarcinoma, | Adenocarcinoma, | | | | | | | | |
| | Primary | Papillary Serous | | | | | | | | |
| | | Type, Primary | | | | | | | | |
| 208644_at | Testis, Normal | Testis, Seminoma, | 333.35 | 78.19 | 7 | 622.56 | 164.78 | 8 | 1.87 | 0.001 |
| | | Primary | | | | | | | | |
| 208644_at | Lung, Pulmonary | Lung, Squamous Cell | 167.99 | 19.89 | 39 | 309.53 | 103.71 | 39 | 1.84 | 0.000 |
| | Emphysema, not | Carcinoma, Primary | | | | | | | | |
| | Associated with | | | | | | | | | |
| | A1AT Deficiency | | | | | | | | | |
| 208644_at | Lung, Normal | Lung, Squamous Cell | 170.58 | 56.25 | 126 | 309.53 | 103.71 | 39 | 1.81 | 0.000 |
| | | Carcinoma, Primary | | | | | | | | |
| 208644_at | Endometrium, | Endometrium, | 297.42 | 98.78 | 50 | 517.86 | 185.55 | 7 | 1.74 | 0.020 |
| | Adenocarcinoma, | Mullerian Mixed | | | | | | | | |
| | Endometrioid | Tumor, Primary | | | | | | | | |
| | Type, Primary | | | | | | | | | |
| 208644_at | Breast, Fibrocystic | Breast, Infiltrating | 188.81 | 59.9 | 20 | 328.49 | 135.69 | 169 | 1.74 | 0.000 |
| | Disease | Ductal Carcinoma, | | | | | | | | |
| | | Primary | | | | | | | | |
| 208644_at | Lung, Pulmonary | Lung, Large Cell | 167.99 | 19.89 | 39 | 291.08 | 122.74 | 7 | 1.73 | 0.038 |
| | Emphysema, not | Carcinoma, Primary | | | | | | | | |
| | Associated with | | | | | | | | | |
| | A1AT Deficiency | | | | | | | | | |
| 208644_at | Lung, Normal | Lung, Large Cell | 170.58 | 56.25 | 126 | 291.08 | 122.74 | 7 | 1.71 | 0.041 |
| | | Carcinoma, Primary | | | | | | | | |
| 208644_at | Lung, Pulmonary | Lung, | 167.99 | 19.89 | 39 | 284.99 | 92.24 | 46 | 1.70 | 0.000 |
| | Emphysema, not | Adenocarcinoma, | | | | | | | | |
| | Associated with | Primary | | | | | | | | |
| | A1AT Deficiency | | | | | | | | | |
| 208644_at | Ovary, | Ovary, Serous | 220.76 | 45.99 | 6 | 371.23 | 104.08 | 8 | 1.68 | 0.004 |
| | Adenocarcinoma, | Cystadenocarcinoma, | | | | | | | | |
| | Clear Cell Type, | Primary | | | | | | | | |
| | Primary | | | | | | | | | |
| 208644_at | Breast, Fibrocystic | Breast, Infiltrating | 188.81 | 59.90 | 20 | 317.43 | 123.81 | 17 | 1.68 | 0.001 |
| | Disease | Lobular Carcinoma, | | | | | | | | |
| | | Primary | | | | | | | | |
| 208644_at | Lung, Normal | Lung, | 170.58 | 56.25 | 126 | 284.99 | 92.24 | 46 | 1.67 | 0.000 |
| | | Adenocarcinoma, | | | | | | | | |
| | | Primary | | | | | | | | |
| 208644_at | Endometrium, | Endometrium, | 202.89 | 75.38 | 10 | 336.79 | 71.19 | 6 | 1.66 | 0.004 |
| | Normal; Smoking | Adenocarcinoma, | | | | | | | | |
| | History | Endometrioid Type, | | | | | | | | |
| | | Primary; Smoking | | | | | | | | |
| | | History | | | | | | | | |
| 208644_at | Breast, Normal; No | Breast, Infiltrating | 192.72 | 41.09 | 30 | 319.17 | 114.21 | 89 | 1.66 | 0.000 |
| | Smoking History | Ductal Carcinoma, | | | | | | | | |
| | | Primary; No | | | | | | | | |
| | | Smoking History | | | | | | | | |
| 208644_at | Skin, Normal | Skin, Basal Cell | 154.29 | 67.12 | 61 | 255.43 | 62.26 | 4 | 1.66 | 0.043 |
| | | Carcinoma, Primary | | | | | | | | |
| 208644_at | Ovary, | Ovary, | 220.76 | 45.99 | 6 | 361.56 | 153.46 | 36 | 1.64 | 0.000 |
| | Adenocarcinoma, | Adenocarcinoma, | | | | | | | | |
| | Clear Cell Type, | Papillary Serous | | | | | | | | |
| | Primary | Type, Primary | | | | | | | | |
| 208644_at | Breast, Normal; No | Breast, Infiltrating | 192.72 | 41.09 | 30 | 313.8 | 134.84 | 10 | 1.63 | 0.020 |
| | Smoking History | Lobular Carcinoma, | | | | | | | | |
| | | Primary; No | | | | | | | | |
| | | Smoking History | | | | | | | | |
| 208644_at | Breast, Normal | Breast, Infiltrating | 201.78 | 81.64 | 68 | 328.49 | 135.69 | 169 | 1.63 | 0.000 |
| | | Ductal Carcinoma, | | | | | | | | |
| | | Primary | | | | | | | | |
| 208644_at | Breast, Infiltrating | Metastatic Infiltrating | 266.6 | 67.10 | 18 | 433.92 | 146.92 | 10 | 1.63 | 0.006 |
| | Ductal Carcinoma, | Ductal Carcinoma of | | | | | | | | |
| | Primary; Stage I | Breast, All Secondary | | | | | | | | |
| | | Sites | | | | | | | | |
| 208644_at | Pancreas, Normal; | Pancreas, | 305.07 | 61.48 | 11 | 184.74 | 54.4 | 3 | −1.65 | 0.036 |
| | No Smoking | Adenocarcinoma, | | | | | | | | |
| | History | Primary; No | | | | | | | | |
| | | Smoking History | | | | | | | | |
| 208644_at | Stomach, | Metastatic | 267.48 | 108.98 | 27 | 159.57 | 34.93 | 3 | −1.68 | 0.006 |
| | Adenocarcinoma | Adenocarcinoma | | | | | | | | |
| | (Excluding Signet | (Excluding Signet | | | | | | | | |
| | Ring Cell Type), | Ring Cell Type) of | | | | | | | | |
| | Primary | Stomach, All | | | | | | | | |
| | | Secondary Sites | | | | | | | | |
| 208644_at | Pancreas, Normal | Pancreas, | 321.84 | 69.04 | 46 | 191.82 | 53.5 | 23 | −1.68 | 0.000 |
| | | Adenocarcinoma, | | | | | | | | |
| | | Primary | | | | | | | | |
| 208644_at | Ovary, | Ovary, Mucinous | 331.23 | 140.37 | 22 | 191.45 | 47.99 | 7 | −1.73 | 0.000 |
| | Adenocarcinoma, | Cystadenocarcinoma, | | | | | | | | |
| | Endometrioid | Primary | | | | | | | | |
| | Type, Primary | | | | | | | | | |
| 208644_at | Pancreas, Normal; | Pancreas, | 313.58 | 74.61 | 23 | 166.22 | 27.3 | 5 | −1.89 | 0.000 |
| | Smoking History | Adenocarcinoma, | | | | | | | | |
| | | Primary; Smoking | | | | | | | | |
| | | History | | | | | | | | |
| 208644_at | Stomach, | Metastatic | 324.58 | 46.07 | 5 | 159.57 | 34.93 | 3 | −2.03 | 0.002 |
| | Adenocarcinoma | Adenocarcinoma | | | | | | | | |
| | (Excluding Signet | (Excluding Signet | | | | | | | | |
| | Ring Cell Type), | Ring Cell Type) of | | | | | | | | |
| | Primary; Stage II | Stomach, All | | | | | | | | |
| | | Secondary Sites | | | | | | | | |

TABLE XV
PARP1 - e-Northern (Human)
Name: poly (ADP-ribose) polymerase family, member 1
View: CVS Disease
Sort By: Organ System
Fragment Legend: 208644_at

| | | | | | | | | | | |
|---|---|---|---|---|---|---|---|---|---|---|
| | | | % | | Lower | 25% | | | 75% | Upper |
| Category | Fragment | Freq. | Present | Count | Limit | Quan. | Median | Mean | Quan. | Limit |

Human, Cardiovascular System Diseases

| | | | | | | | | | | |
|---|---|---|---|---|---|---|---|---|---|---|
| Artery, Atherosclerosis | 208644_at | 1.00 | 1.00 | 4 | 140.38 | 159.37 | 182.96 | 180.30 | 203.89 | 214.92 |
| Artery, Normal | 208644_at | 1.00 | 1.00 | 4 | 95.96 | 129.13 | 141.33 | 160.15 | 172.35 | 237.17 |
| Cardiac Myocyte from Heart | 208644_at | 1.00 | 1.00 | 1 | 423.41 | 423.41 | 423.41 | 423.41 | 423.41 | 423.41 |
| with Old Myocardial Infarct | | | | | | | | | | |
| Left Atrium, Granulomatous | 208644_at | 1.00 | 1.00 | 3 | 167.79 | 181.71 | 195.63 | 186.36 | 195.65 | 195.67 |
| Myocarditis, | | | | | | | | | | |
| Nonhypersensitivity Type | | | | | | | | | | |
| Left Atrium, Myocardial | 208644_at | 1.00 | 1.00 | 4 | 210.92 | 214.88 | 227.39 | 228.46 | 240.97 | 248.14 |
| Fibrosis Secondary to | | | | | | | | | | |
| Valvular Heart Disease | | | | | | | | | | |
| Left Atrium, Myocardial | 208644_at | 1.00 | 1.00 | 4 | 198.54 | 212.48 | 239.67 | 240.41 | 267.6 | 283.76 |
| Fibrosis without Infarction, | | | | | | | | | | |
| Secondary to Coronary Artery | | | | | | | | | | |
| Disease | | | | | | | | | | |
| Left Atrium, Normal | 208644_at | 1.00 | 1.00 | 18 | 121.96 | 186.33 | 206.52 | 204.39 | 232.62 | 259.32 |
| Left Atrium, Primary | 208644_at | 1.00 | 1.00 | 33 | 141.66 | 199.28 | 231.42 | 229.09 | 256.72 | 339.30 |
| Congestive Dilated | | | | | | | | | | |
| Cardiomyopathy | | | | | | | | | | |
| Left Atrium, Primary | 208644_at | 1.00 | 1.00 | 9 | 150.73 | 183.14 | 245.06 | 228.61 | 260.06 | 304.58 |
| Hypertrophic | | | | | | | | | | |
| Cardiomyopathy | | | | | | | | | | |
| Left Atrium, Viable Tissue; | 208644_at | 1.00 | 1.00 | 63 | 151.04 | 191.73 | 222.30 | 227.62 | 247.43 | 308.77 |
| from Heart with Old (Healed) | | | | | | | | | | |
| Myocardial Infarction | | | | | | | | | | |
| Left Atrium, Viable Tissue; | 208644_at | 1.00 | 1.00 | 8 | 158.54 | 198.27 | 215.44 | 212.22 | 230.31 | 250.68 |
| from Heart with Recent | | | | | | | | | | |
| Myocardial Infarction | | | | | | | | | | |
| Left Ventricle, Chronic | 208644_at | 1.00 | 1.00 | 3 | 269.03 | 279.19 | 289.34 | 293.3 | 305.44 | 321.53 |
| Myocarditis | | | | | | | | | | |
| (Nongranulomatous) | | | | | | | | | | |
| Left Ventricle, | 208644_at | 1.00 | 1.00 | 4 | 261.55 | 299.53 | 349.13 | 368.31 | 417.92 | 513.42 |
| Granulomatous Myocarditis, | | | | | | | | | | |
| Nonhypersensitivity Type | | | | | | | | | | |
| Left Ventricle, Myocardial | 208644_at | 1.00 | 1.00 | 4 | 223.73 | 305.57 | 345.44 | 321.72 | 361.58 | 372.26 |
| Fibrosis Secondary to | | | | | | | | | | |
| Valvular Heart Disease | | | | | | | | | | |
| Left Ventricle, Myocardial | 208644_at | 1.00 | 1.00 | 4 | 204.67 | 310.93 | 363.42 | 352.83 | 405.32 | 479.83 |
| Fibrosis without Infarction, | | | | | | | | | | |
| Secondary to Coronary Artery | | | | | | | | | | |
| Disease | | | | | | | | | | |
| Left Ventricle, Normal | 208644_at | 1.00 | 1.00 | 3 | 256.11 | 281.73 | 307.35 | 305.95 | 330.86 | 354.38 |
| Left Ventricle, Primary | 208644_at | 1.00 | 1.00 | 46 | 201.25 | 288.71 | 335.18 | 339.07 | 374.61 | 503.47 |
| Congestive Dilated | | | | | | | | | | |
| Cardiomyopathy | | | | | | | | | | |
| Left Ventricle, Primary | 208644_at | 1.00 | 1.00 | 24 | 232.78 | 282.3 | 321.7 | 343.74 | 391.91 | 556.32 |
| Hypertrophic | | | | | | | | | | |
| Cardiomyopathy | | | | | | | | | | |
| Left Ventricle, Viable Tissue; | 208644_at | 1.00 | 1.00 | 1.02 | 197.38 | 294.76 | 345.34 | 341 | 380.77 | 509.78 |
| from Heart with Old (Healed) | | | | | | | | | | |
| Myocardial Infarction | | | | | | | | | | |
| Left Ventricle, Viable Tissue; | 208644_at | 1.00 | 1.00 | 31 | 154.54 | 282.14 | 305.69 | 318.18 | 367.2 | 474.83 |
| from Heart with Recent | | | | | | | | | | |
| Myocardial Infarction | | | | | | | | | | |
| Right Atrium, Granulomatous | 208644_at | 1.00 | 1.00 | 3 | 123.50 | 157.81 | 192.11 | 176.41 | 202.86 | 213.61 |
| Myocarditis, | | | | | | | | | | |
| Nonhypersensitivity Type | | | | | | | | | | |
| Right Atrium, Myocardial | 208644_at | 1.00 | 1.00 | 6 | 150.93 | 187.87 | 225.72 | 216.46 | 253.3 | 258.67 |
| Fibrosis Secondary to | | | | | | | | | | |
| Valvular Heart Disease | | | | | | | | | | |
| Right Atrium, Myocardial | 208644_at | 1.00 | 1.00 | 6 | 190.94 | 224.74 | 241.42 | 235.15 | 250.04 | 265.04 |
| Fibrosis without Infarction, | | | | | | | | | | |
| Secondary to Coronary Artery | | | | | | | | | | |
| Disease | | | | | | | | | | |
| Right Atrium, Normal | 208644_at | 1.00 | 1.00 | 4 | 204.76 | 209.20 | 216.10 | 219.89 | 226.79 | 242.61 |
| Right Atrium, Primary | 208644_at | 1.00 | 1.00 | 39 | 167.45 | 201.32 | 235.33 | 234.78 | 257.73 | 305.18 |
| Congestive Dilated | | | | | | | | | | |
| Cardiomyopathy | | | | | | | | | | |
| Right Atrium, Primary | 208644_at | 1.00 | 1.00 | 10 | 131.82 | 204.29 | 223.37 | 229.33 | 256.97 | 318.55 |
| Hypertrophic | | | | | | | | | | |
| Cardiomyopathy | | | | | | | | | | |
| Right Atrium, Viable Tissue; | 208644_at | 1.00 | 1.00 | 69 | 139.82 | 200.04 | 225.71 | 228.58 | 248.48 | 321.13 |
| from Heart with Old (Healed) | | | | | | | | | | |
| Myocardial Infarction | | | | | | | | | | |
| Right Atrium, Viable Tissue; | 208644_at | 1.00 | 1.00 | 10 | 162.05 | 187.38 | 207.95 | 211.96 | 233.69 | 288.07 |
| from Heart with Recent | | | | | | | | | | |
| Myocardial Infarction | | | | | | | | | | |
| Right Ventricle, | 208644_at | 1.00 | 1.00 | 3 | 278.33 | 289.20 | 300.07 | 308.06 | 322.92 | 345.76 |
| Granulomatous Myocarditis, | | | | | | | | | | |
| Nonhypersensitivity Type | | | | | | | | | | |
| Right Ventricle, Myocardial | 208644_at | 1.00 | 1.00 | 7 | 290.75 | 354.32 | 369.35 | 375.44 | 396.7 | 460.27 |
| Fibrosis Secondary to | | | | | | | | | | |
| Valvular Heart Disease | | | | | | | | | | |
| Right Ventricle, Myocardial | 208644_at | 1.00 | 1.00 | 5 | 290.58 | 317.96 | 343.99 | 356.56 | 370.21 | 448.58 |
| Fibrosis without Infarction, | | | | | | | | | | |
| Secondary to Coronary Artery | | | | | | | | | | |
| Disease | | | | | | | | | | |
| Right Ventricle, Normal | 208644_at | 1.00 | 1.00 | 4 | 220.97 | 291.24 | 318.13 | 311.18 | 338.08 | 393.16 |
| Right Ventricle, Primary | 208644_at | 1.00 | 1.00 | 46 | 233.71 | 293.98 | 359.13 | 357.31 | 402.81 | 521.65 |
| Congestive Dilated | | | | | | | | | | |
| Cardiomyopathy | | | | | | | | | | |
| Right Ventricle, Primary | 208644_at | 1.00 | 1.00 | 20 | 271.87 | 310.96 | 348.93 | 350.46 | 377.21 | 476.58 |
| Hypertrophic | | | | | | | | | | |
| Cardiomyopathy | | | | | | | | | | |
| Right Ventricle, Viable | 208644_at | 1.00 | 1.00 | 98 | 182.44 | 298.61 | 339.97 | 345.78 | 381.34 | 505.43 |
| Tissue; from Heart with Old | | | | | | | | | | |
| (Healed) Myocardial | | | | | | | | | | |
| Infarction | | | | | | | | | | |
| Right Ventricle, Viable | 208644_at | 1.00 | 1.00 | 18 | 250.29 | 309.06 | 342.83 | 347.87 | 377.59 | 480.38 |
| Tissue; from Heart with | | | | | | | | | | |
| Recent Myocardial Infarction | | | | | | | | | | |

TABLE XVI
PARP1 - Diff/X (Human)
Name: poly (ADP-ribose) polymerase family, member 1
Sort By: p-value
Minimum Fold Change: 1.2
p-Value Range: 0.00-0.05
One differential expression event found.
Cardiovascular System

| | | | | Control | | | Experiment | | Fold | |
|---|---|---|---|---|---|---|---|---|---|---|
| | | | Control | Standard | | Experiment | Standard | | change | p- |
| Fragment | Control | Experiment | Mean | Deviation | Control # | Mean | Deviation | Experiment # | (FC) | value |
| 208644_at | Left Ventricle, | Left Ventricle, | 277.9 | 30.09 | 3 | 336.04 | 67.61 | 30 | 1.21 | 0.047 |
| | Normal; No | Viable Tissue; from | | | | | | | | |
| | Smoking History | Heart with Old | | | | | | | | |
| | | (Healed) | | | | | | | | |
| | | Myocardial | | | | | | | | |
| | | Infarction; No | | | | | | | | |
| | | Smoking History | | | | | | | | |

TABLE XVII
PARP1 - Diff/X (Human)
Name: poly (ADP-ribose) polymerase family, member 1
Sort By: p-value
Minimum Fold Change: 1.2
p-Value Range: 0.00-0.05
7 differential expression events found.
Central Nervous System

| | | | | Control | | Exper- | Experiment | | Fold | |
|---|---|---|---|---|---|---|---|---|---|---|
| | | | Control | Standard | Control | iment | Standard | Experiment | change | p- |
| Fragment | Control | Experiment | Mean | Deviation | # | Mean | Deviation | # | (FC) | value |
| 208644_at | Dorsal Raphe, Normal, | Dorsal Raphe, | 251.21 | 30.59 | 7 | 308.80 | 28.73 | 5 | 1.23 | 0.009 |
| | Control for Parkinson's | Parkinson's Disease | | | | | | | | |
| | Disease | | | | | | | | | |
| 208644_at | Superior Frontal Gyrus | Superior Frontal | 255.79 | 64.52 | 15 | 313.11 | 46.15 | 10 | 1.22 | 0.016 |
| | (Brodmann Area 8), | Gyrus (Brodmann | | | | | | | | |
| | Normal, Control for | Area 8), Possible | | | | | | | | |
| | Alzheimer's Disease | Alzheimer's Disease | | | | | | | | |
| | | (CERAD-4) | | | | | | | | |
| 208644_at | Insula, Schizophrenia | Insula, Cocaine | 223.61 | 23.43 | 4 | 289.33 | 31.94 | 4 | 1.29 | 0.018 |
| | | Abuse | | | | | | | | |
| 208644_at | Amygdala, Suicide with | Amygdala, | 318.61 | 33.19 | 4 | 252.86 | 51.47 | 10 | −1.26 | 0.020 |
| | No History of | Schizophrenia | | | | | | | | |
| | Depression | | | | | | | | | |
| 208644_at | Insula, Normal, Control | Insula, Cocaine | 225.48 | 40.08 | 5 | 289.33 | 31.94 | 4 | 1.28 | 0.033 |
| | for Cocaine Abuse | Abuse | | | | | | | | |
| 208644_at | Hippocampus, Normal, | Hippocampus, | 266.34 | 38.90 | 10 | 330.07 | 59.96 | 7 | 1.24 | 0.034 |
| | Control for Alzheimer's | Possible Alzheimer's | | | | | | | | |
| | Disease | Disease (CERAD-4) | | | | | | | | |
| 208644_at | Superior Temporal | Superior Temporal | 226.78 | 23.15 | 4 | 290.49 | 61.79 | 7 | 1.28 | 0.039 |
| | Gyrus (Brodmann Area | Gyrus (Brodmann | | | | | | | | |
| | 22), Normal, Patients | Area 22), Normal, | | | | | | | | |
| | Not Taking | Patients Taking | | | | | | | | |
| | Acetaminophen | Acetaminophen | | | | | | | | |

TABLE XVIII
PARP1 - e-Northern (Human)
Name: poly (ADP-ribose) polymerase family, member 1
View: Inflammation
Sort By: Organ System
Fragment Legend: 208644_at

| | | | | | | | | | | |
|---|---|---|---|---|---|---|---|---|---|---|
| | | | % | | Lower | 25% | | | 75% | Upper |
| Category | Fragment | Freq. | Present | Count | Limit | Quan. | Median | Mean | Quan. | Limit |

Human, General Inflammatory Diseases, Digestive System

| | | | | | | | | | | |
|---|---|---|---|---|---|---|---|---|---|---|
| Colon, Crohn's Disease, Active | 208644_at | 1.00 | 1.00 | 5 | 182.84 | 187.32 | 216.79 | 233.13 | 251.28 | 327.43 |
| (Acute Inflammation) | | | | | | | | | | |
| Colon, Crohn's Disease, Active | 208644_at | 1.00 | 1.00 | 4 | 146.50 | 195.56 | 223.82 | 213.86 | 242.12 | 261.28 |
| (Chronic Inflammation) | | | | | | | | | | |
| Colon, Normal | 208644_at | 1.00 | 1.00 | 180 | 88.25 | 166.74 | 191.91 | 198.00 | 229.97 | 324.80 |
| Colon, Ulcerative Colitis, Active | 208644_at | 1.00 | 1.00 | 13 | 169.60 | 202.83 | 220.49 | 237.19 | 297.14 | 324.24 |
| (Acute Inflammation) | | | | | | | | | | |
| Colon, Ulcerative Colitis, Active | 208644_at | 1.00 | 1.00 | 3 | 206.04 | 220.86 | 235.69 | 234.45 | 248.66 | 261.62 |
| (Chronic Inflammation) | | | | | | | | | | |
| Gallbladder, Acute Cholecystitis | 208644_at | 1.00 | 1.00 | 10 | 122.77 | 148.17 | 155.85 | 163.34 | 180.27 | 228.42 |
| Gallbladder, Chronic Cholecystitis | 208644_at | 1.00 | 1.00 | 54 | 102.08 | 142.94 | 160.70 | 165.17 | 184.40 | 246.59 |
| Gallbladder, Normal | 208644_at | 1.00 | 1.00 | 7 | 113.46 | 129.19 | 142.84 | 155.06 | 185.08 | 200.60 |
| Liver, Cirrhosis Secondary to | 208644_at | 1.00 | 1.00 | 25 | 143.33 | 153.78 | 161.77 | 168.31 | 178.32 | 215.12 |
| Chronic Hepatitis C | | | | | | | | | | |
| Liver, Cirrhosis, All Causes | 208644_at | 1.00 | 1.00 | 61 | 107.06 | 150.80 | 160.97 | 169.21 | 179.97 | 223.71 |
| Liver, Normal | 208644_at | 1.00 | 1.00 | 42 | 85.71 | 149.27 | 172.85 | 195.15 | 201.08 | 278.79 |
| Major Salivary Gland (Excluding | 208644_at | 1.00 | 1.00 | 4 | 149.83 | 161.58 | 168.30 | 174.91 | 181.64 | 211.73 |
| Parotid), Chronic Sialadenitis | | | | | | | | | | |
| Major Salivary Gland (Excluding | 208644_at | 1.00 | 1.00 | 8 | 121.57 | 132.84 | 143.81 | 164.83 | 211.27 | 224.94 |
| Parotid), Normal | | | | | | | | | | |
| Omentum, Normal | 208644_at | 1.00 | 1.00 | 15 | 112.36 | 177.28 | 201.36 | 219.15 | 220.56 | 285.48 |
| Omentum, Peritonitis | 208644_at | 1.00 | 1.00 | 3 | 125.18 | 150.29 | 175.41 | 167.92 | 189.30 | 203.19 |
| Pancreas, Acute Pancreatitis | 208644_at | 1.00 | 1.00 | 3 | 214.89 | 271.35 | 327.82 | 297.92 | 339.43 | 351.05 |
| Pancreas, Chronic Pancreatitis | 208644_at | 1.00 | 1.00 | 10 | 155.48 | 208.15 | 214.99 | 245.29 | 279.84 | 371.64 |
| Pancreas, Normal | 208644_at | 1.00 | 1.00 | 46 | 131.80 | 276.35 | 319.04 | 321.84 | 372.71 | 469.39 |
| Small Intestine, Crohn's Disease, | 208644_at | 1.00 | 1.00 | 4 | 152.55 | 181.94 | 195.68 | 197.94 | 211.67 | 247.85 |
| Active (Acute Inflammation) | | | | | | | | | | |
| Small Intestine, Crohn's Disease, | 208644_at | 1.00 | 1.00 | 3 | 150.13 | 168.97 | 187.80 | 198.47 | 222.64 | 257.49 |
| Active (Chronic Inflammation) | | | | | | | | | | |
| Small Intestine, Normal | 208644_at | 1.00 | 1.00 | 97 | 90.87 | 167.17 | 186.89 | 193.57 | 218.03 | 294.33 |
| Stomach, Chronic Gastritis | 208644_at | 1.00 | 1.00 | 40 | 131.34 | 197.49 | 227.83 | 237.00 | 270.87 | 373.57 |
| Stomach, Normal | 208644_at | 1.00 | 0.98 | 52 | 116.33 | 190.63 | 215.46 | 221.41 | 244.02 | 324.10 |

Human, General Inflammatory Diseases, Endocrine System

| | | | | | | | | | | |
|---|---|---|---|---|---|---|---|---|---|---|
| Thyroid Gland, Hashimoto's | 208644_at | 1.00 | 1.00 | 19 | 137.35 | 205.59 | 247.58 | 250.53 | 310.93 | 350.33 |
| Thyroiditis | | | | | | | | | | |
| Thyroid Gland, Nodular | 208644_at | 1.00 | 1.00 | 58 | 110.90 | 155.21 | 174.31 | 173.96 | 192.22 | 240.77 |
| Hyperplasia | | | | | | | | | | |
| Thyroid Gland, Normal | 208644_at | 1.00 | 1.00 | 24 | 125.34 | 150.78 | 171.69 | 173.82 | 187.96 | 243.72 |

Human, General Inflammatory Diseases, Female Reproductive System

| | | | | | | | | | | |
|---|---|---|---|---|---|---|---|---|---|---|
| Myometrium, Adenomyosis | 208644_at | 1.00 | 1.00 | 6 | 175.91 | 190.07 | 192.94 | 178.67 | 199.51 | 208.66 |
| Myometrium, Normal | 208644_at | 1.00 | 1.00 | 122 | 99.43 | 155.41 | 175.44 | 176.66 | 195.83 | 256.47 |
| Ovary, Endometriosis | 208644_at | 1.00 | 1.00 | 7 | 134.48 | 141.61 | 157.72 | 152.26 | 161.13 | 168.14 |
| Ovary, Normal | 208644_at | 1.00 | 1.00 | 89 | 98.25 | 147.72 | 161.49 | 163.31 | 180.70 | 224.40 |
| Uterine Cervix, Acute Cervicitis | 208644_at | 1.00 | 1.00 | 3 | 144.14 | 164.10 | 184.07 | 179.56 | 197.27 | 210.47 |
| Uterine Cervix, Chronic Cervicitis | 208644_at | 1.00 | 1.00 | 11 | 153.01 | 168.18 | 183.09 | 180.14 | 190.99 | 214.65 |
| Uterine Cervix, Normal | 208644_at | 1.00 | 0.98 | 115 | 77.91 | 150.20 | 168.54 | 178.85 | 198.40 | 270.70 |

Human, General Inflammatory Diseases, Hematopoietic System

| | | | | | | | | | | |
|---|---|---|---|---|---|---|---|---|---|---|
| Adenoids, Lymphoid Hyperplasia | 208644_at | 1.00 | 1.00 | 3 | 481.35 | 485.17 | 488.98 | 523.08 | 543.95 | 598.92 |
| Lymph Node, Normal | 208644_at | 1.00 | 1.00 | 10 | 217.06 | 316.25 | 355.00 | 325.59 | 382.39 | 452.30 |
| Lymph Node, Reactive Lymphoid | 208644_at | 1.00 | 1.00 | 9 | 274.24 | 429.78 | 485.93 | 475.49 | 533.48 | 656.69 |
| Hyperplasia | | | | | | | | | | |
| Monocytes, Normal, CD14+ | 208644_at | 1.00 | 1.00 | 8 | 117.24 | 126.21 | 133.50 | 138.79 | 148.85 | 172.61 |
| Mononuclear White Blood Cells, | 208644_at | 1.00 | 1.00 | 9 | 166.29 | 193.67 | 225.79 | 223.53 | 246.91 | 304.52 |
| Multiple Sclerosis, All Types | | | | | | | | | | |
| Mononuclear White Blood Cells, | 208644_at | 1.00 | 1.00 | 8 | 219.71 | 243.66 | 263.41 | 259.16 | 277.35 | 296.62 |
| Normal | | | | | | | | | | |
| Natural Killer Cells, Normal, | 208644_at | 1.00 | 1.00 | 4 | 229.11 | 234.35 | 252.60 | 259.27 | 277.52 | 302.76 |
| CD56+ | | | | | | | | | | |
| Neutrophils, Normal | 208644_at | 1.00 | 0.67 | 3 | 28.89 | 33.76 | 38.62 | 41.87 | 48.36 | 58.10 |
| Spleen, Hypertrophy Secondary to | 208644_at | 1.00 | 1.00 | 5 | 184.41 | 253.24 | 278.41 | 258.07 | 299.12 | 330.22 |
| Idiopathic Thrombocytopenic | | | | | | | | | | |
| Purpura | | | | | | | | | | |
| Spleen, Lymphoid Hyperplasia | 208644_at | 1.00 | 1.00 | 3 | 283.97 | 290.92 | 297.87 | 304.48 | 314.74 | 331.60 |
| Spleen, Normal | 208644_at | 1.00 | 1.00 | 34 | 207.76 | 255.56 | 298.84 | 302.23 | 335.73 | 409.07 |
| T-lymphocytes, Normal, CD4+ | 208644_at | 1.00 | 1.00 | 4 | 212.49 | 284.03 | 322.24 | 304.66 | 342.88 | 361.67 |
| T-lymphocytes, Normal, CD8+ | 208644_at | 1.00 | 1.00 | 6 | 235.18 | 258.74 | 279.61 | 273.01 | 290.19 | 298.15 |
| Thymus, Atrophy | 208644_at | 1.00 | 1.00 | 3 | 166.15 | 174.74 | 183.33 | 184.89 | 194.27 | 205.20 |
| Thymus, Normal | 208644_at | 1.00 | 1.00 | 62 | 177.63 | 226.17 | 261.44 | 263.23 | 292.65 | 392.37 |
| Tonsil, Reactive Lymphoid | 208644_at | 1.00 | 1.00 | 80 | 118.52 | 449.04 | 514.77 | 554.51 | 669.38 | 999.90 |
| Hyperplasia | | | | | | | | | | |
| White Blood Cells, Crohn's | 208644_at | 1.00 | 1.00 | 14 | 83.44 | 114.33 | 142.25 | 145.07 | 152.44 | 209.61 |
| Disease | | | | | | | | | | |
| White Blood Cells, Normal | 208644_at | 1.00 | 1.00 | 14 | 130.23 | 168.79 | 178.89 | 176.91 | 194.50 | 233.06 |
| White Blood Cells, Primary IgA | 208644_at | 1.00 | 1.00 | 7 | 67.30 | 117.71 | 154.49 | 143.04 | 174.92 | 194.22 |
| Nephropathy | | | | | | | | | | |
| White Blood Cells, Rheumatoid | 208644_at | 1.00 | 1.00 | 14 | 63.76 | 115.74 | 136.52 | 137.82 | 159.29 | 199.44 |
| Arthritis | | | | | | | | | | |
| White Blood Cells, Systemic | 208644_at | 1.00 | 1.00 | 15 | 96.91 | 127.35 | 140.46 | 154.57 | 156.95 | 201.34 |
| Lupus Erythematosus | | | | | | | | | | |
| White Blood Cells, Ulcerative | 208644_at | 1.00 | 1.00 | 9 | 102.30 | 110.68 | 123.27 | 144.61 | 158.48 | 230.19 |
| Colitis | | | | | | | | | | |
| White Blood Cells, Wegener's | 208644_at | 1.00 | 0.96 | 27 | 65.21 | 91.05 | 109.17 | 119.48 | 143.14 | 207.13 |
| Granulomatosis | | | | | | | | | | |

Human, General Inflammatory Diseases, Integumentary System

| | | | | | | | | | | |
|---|---|---|---|---|---|---|---|---|---|---|
| Skin, Normal | 208644_at | 1.00 | 1.00 | 61 | 73.17 | 123.09 | 149.86 | 154.29 | 167.66 | 234.50 |
| Skin, Patients With Psoriasis; | 208644_at | 1.00 | 1.00 | 6 | 121.68 | 124.53 | 128.10 | 131.36 | 138.35 | 145.16 |
| Region of Active Inflammation | | | | | | | | | | |
| Skin, Patients With Psoriasis; | 208644_at | 1.00 | 1.00 | 6 | 91.42 | 119.22 | 127.19 | 128.48 | 141.49 | 162.25 |
| Uninvolved Region | | | | | | | | | | |

Human, General Inflammatory Diseases, Musculoskeletal System

| | | | | | | | | | | |
|---|---|---|---|---|---|---|---|---|---|---|
| Bone, Degenerative Joint Disease | 208644_at | 1.00 | 1.00 | 32 | 136.56 | 163.72 | 189.56 | 188.99 | 208.97 | 276.84 |
| (Osteoarthritis) | | | | | | | | | | |
| Bone, Normal | 208644_at | 1.00 | 1.00 | 8 | 154.45 | 183.30 | 194.26 | 196.06 | 218.32 | 225.33 |
| Synovium, Osteoarthritis | 208644_at | 1.00 | 1.00 | 4 | 133.41 | 155.00 | 178.15 | 184.00 | 207.14 | 246.28 |
| (Degenerative Joint Disease) | | | | | | | | | | |
| Synovium, Rheumatoid Arthritis | 208644_at | 1.00 | 1.00 | 3 | 154.38 | 172.23 | 190.08 | 183.21 | 197.63 | 205.17 |

Human, General Inflammatory Diseases, Respiratory System

| | | | | | | | | | | |
|---|---|---|---|---|---|---|---|---|---|---|
| Lung, Normal | 208644_at | 1.00 | 1.00 | 126 | 87.04 | 143.56 | 161.44 | 170.58 | 181.24 | 237.76 |
| Lung, Pulmonary Emphysema, | 208644_at | 1.00 | 1.00 | 3 | 112.06 | 117.61 | 123.16 | 137.16 | 149.70 | 176.25 |
| Associated with A1AT Deficiency | | | | | | | | | | |
| Lung, Pulmonary Emphysema, not | 208644_at | 1.00 | 1.00 | 39 | 127.94 | 153.58 | 163.47 | 167.99 | 182.45 | 217.50 |
| Associated with A1AT Deficiency | | | | | | | | | | |

Human, General Inflammatory Diseases, Urinary Tract

| | | | | | | | | | | |
|---|---|---|---|---|---|---|---|---|---|---|
| Kidney, Chronic Pyelonephritis | 208644_at | 1.00 | 1.00 | 10 | 136.04 | 147.83 | 158.74 | 174.04 | 196.78 | 249.92 |
| Kidney, Normal | 208644_at | 1.00 | 0.99 | 81 | 115.79 | 143.82 | 165.98 | 165.78 | 184.14 | 244.62 |
| Urinary Bladder, Chronic Cystitis | 208644_at | 1.00 | 1.00 | 3 | 205.25 | 246.32 | 287.39 | 296.53 | 342.17 | 396.95 |
| Urinary Bladder, Normal | 208644_at | 1.00 | 1.00 | 9 | 128.23 | 162.66 | 184.14 | 223.63 | 267.52 | 382.66 |

TABLE XIX
PARP1 - Diff/X (Human)
Name: poly (ADP-ribose) polymerase family, member 1
Sort By: p-value
Minimum Fold Change: 1.2
p-Value Range: 0.00-0.05
39 differential expression events found.
Hematolymphoid System

| | | | | Control | | | Experiment | | Fold | |
|---|---|---|---|---|---|---|---|---|---|---|
| | | | Control | Standard | Control | Experiment | Standard | | change | |
| Fragment | Control | Experiment | Mean | Deviation | # | Mean | Deviation | Experiment # | (FC) | p-value |
| 208644_at | Lymph Node, | Lymph Node, Non- | 325.59 | 104.66 | 10 | 756.79 | 372.23 | 91 | 2.32 | 0.000 |
| | Normal | Hodgkin's | | | | | | | | |
| | | Lymphoma, All | | | | | | | | |
| | | Types | | | | | | | | |
| 208644_at | B- | White Blood Cells, | 625.82 | 21.60 | 4 | 307.50 | 62.85 | 12 | −2.04 | 0.000 |
| | Lymphocytes, | Chronic | | | | | | | | |
| | Germinal | Lymphocytic | | | | | | | | |
| | Center, Resting, | Leukemia | | | | | | | | |
| | IgD+ | | | | | | | | | |
| 208644_at | Lymph Node, | Lymph Node, Non- | 325.59 | 104.66 | 10 | 752.45 | 201.14 | 30 | 2.31 | 0.000 |
| | Normal | Hodgkin's | | | | | | | | |
| | | Lymphoma, | | | | | | | | |
| | | Follicular Type | | | | | | | | |
| 208644_at | B | B Lymphocytes, | 583.14 | 112.02 | 14 | 228.18 | 18.72 | 5 | −2.56 | 0.000 |
| | Lymphocytes, | PMA + Ionomycin; | | | | | | | | |
| | Control; 0 hours | 2 hours | | | | | | | | |
| 208644_at | Lymph Node, | Lymph Node, Non- | 325.59 | 104.66 | 10 | 958.33 | 465.47 | 32 | 2.94 | 0.000 |
| | Normal | Hodgkin's | | | | | | | | |
| | | Lymphoma, Diffuse | | | | | | | | |
| | | Large B-Cell Type | | | | | | | | |
| 208644_at | B | B Lymphocytes, | 583.14 | 112.02 | 14 | 272.36 | 57.28 | 6 | −2.14 | 0.000 |
| | Lymphocytes, | LPS; 2 hours | | | | | | | | |
| | Control; 0 hours | | | | | | | | | |
| 208644_at | Lymph Node, | Lymph Node, Non- | 475.49 | 123.80 | 9 | 958.33 | 465.47 | 32 | 2.02 | 0.000 |
| | Reactive | Hodgkin's | | | | | | | | |
| | Lymphoid | Lymphoma, Diffuse | | | | | | | | |
| | Hyperplasia | Large B-Cell Type | | | | | | | | |
| 208644_at | Lymph Node, | Lymph Node, Non- | 958.33 | 465.47 | 32 | 419.03 | 74.52 | 3 | −2.29 | 0.000 |
| | Non-Hodgkin's | Hodgkin's | | | | | | | | |
| | Lymphoma, | Lymphoma, Small | | | | | | | | |
| | Diffuse Large | Lymphocytic Type | | | | | | | | |
| | B-Cell Type | | | | | | | | | |
| 208644_at | Non-Hodgkin's | Non-Hodgkin's | 878.35 | 423.12 | 58 | 442.13 | 87.15 | 4 | −1.99 | 0.000 |
| | Lymphoma, | Lymphoma, Small | | | | | | | | |
| | Diffuse Large | Lymphocytic Type, | | | | | | | | |
| | B-Cell Type, | All Body Sites | | | | | | | | |
| | All Body Sites | | | | | | | | | |
| 208644_at | B | B Lymphocytes, | 583.14 | 112.02 | 14 | 251.12 | 76.97 | 5 | −2.32 | 0.000 |
| | Lymphocytes, | Anti-IgG; 2 hours | | | | | | | | |
| | Control; 0 hours | | | | | | | | | |
| 208644_at | Lymph Node, | Lymph Node, Non- | 475.49 | 123.80 | 9 | 756.79 | 372.23 | 91 | 1.59 | 0.000 |
| | Reactive | Hodgkin's | | | | | | | | |
| | Lymphoid | Lymphoma, All | | | | | | | | |
| | Hyperplasia | Types | | | | | | | | |
| 208644_at | Non-Hodgkin's | Non-Hodgkin's | 878.35 | 423.12 | 58 | 515.31 | 128.29 | 7 | −1.70 | 0.000 |
| | Lymphoma, | Lymphoma, | | | | | | | | |
| | Diffuse Large | Extranodal, | | | | | | | | |
| | B-Cell Type, | Marginal Zone B | | | | | | | | |
| | All Body Sites | Cell MALT Type | | | | | | | | |
| 208644_at | B | B Lymphocytes, | 272.36 | 57.28 | 6 | 497.93 | 55.79 | 6 | 1.83 | 0.000 |
| | Lymphocytes, | LPS; 8 hours | | | | | | | | |
| | LPS; 2 hours | | | | | | | | | |
| 208644_at | Lymph Node, | Lymph Node, Non- | 475.49 | 123.80 | 9 | 752.45 | 201.14 | 30 | 1.58 | 0.000 |
| | Reactive | Hodgkin's | | | | | | | | |
| | Lymphoid | Lymphoma, | | | | | | | | |
| | Hyperplasia | Follicular Type | | | | | | | | |
| 208644_at | B- | White Blood Cells, | 1033.85 | 90.83 | 4 | 307.50 | 62.85 | 12 | −3.36 | 0.000 |
| | Lymphocytes, | Chronic | | | | | | | | |
| | Germinal | Lymphocytic | | | | | | | | |
| | Center, CD38+ | Leukemia | | | | | | | | |
| | CD77− | | | | | | | | | |
| 208644_at | B- | White Blood Cells, | 1164.76 | 222.38 | 6 | 307.50 | 62.85 | 12 | −3.79 | 0.000 |
| | Lymphocytes, | Chronic | | | | | | | | |
| | Germinal | Lymphocytic | | | | | | | | |
| | Center, CD38+ | Leukemia | | | | | | | | |
| 208644_at | Non-Hodgkin's | Non-Hodgkin's | 878.35 | 423.12 | 58 | 531.74 | 170.32 | 9 | −1.65 | 0.000 |
| | Lymphoma, | Lymphoma, Mantle | | | | | | | | |
| | Diffuse Large | Cell Type, All Body | | | | | | | | |
| | B-Cell Type, | Sites | | | | | | | | |
| | All Body Sites | | | | | | | | | |
| 208644_at | Lymph Node, | Lymph Node, Non- | 958.33 | 465.47 | 32 | 528.09 | 181.71 | 8 | −1.81 | 0.000 |
| | Non-Hodgkin's | Hodgkin's | | | | | | | | |
| | Lymphoma, | Lymphoma, Mantle | | | | | | | | |
| | Diffuse Large | Cell Type | | | | | | | | |
| | B-Cell Type | | | | | | | | | |
| 208644_at | Non-Hodgkin's | Non-Hodgkin's | 763.96 | 215.03 | 43 | 442.13 | 87.15 | 4 | −1.73 | 0.001 |
| | Lymphoma, | Lymphoma, Small | | | | | | | | |
| | Follicular Type, | Lymphocytic Type, | | | | | | | | |
| | All Body Sites | All Body Sites | | | | | | | | |
| 208644_at | B | B Lymphocytes, | 228.18 | 18.72 | 5 | 456.35 | 61.55 | 5 | 2.00 | 0.001 |
| | Lymphocytes, | PMA + Ionomycin; | | | | | | | | |
| | PMA + | 8 hours | | | | | | | | |
| | Ionomycin; 2 | | | | | | | | | |
| | hours | | | | | | | | | |
| 208644_at | Non-Hodgkin's | Non-Hodgkin's | 515.31 | 128.29 | 7 | 763.96 | 215.03 | 43 | 1.48 | 0.001 |
| | Lymphoma, | Lymphoma, | | | | | | | | |
| | Extranodal, | Follicular Type, All | | | | | | | | |
| | Marginal Zone | Body Sites | | | | | | | | |
| | B Cell MALT | | | | | | | | | |
| | Type | | | | | | | | | |
| 208644_at | Lymph Node, | Lymph Node, Non- | 752.45 | 201.14 | 30 | 419.03 | 74.52 | 3 | −1.80 | 0.001 |
| | Non-Hodgkin's | Hodgkin's | | | | | | | | |
| | Lymphoma, | Lymphoma, Small | | | | | | | | |
| | Follicular Type | Lymphocytic Type | | | | | | | | |
| 208644_at | B-lymphocytes, | White Blood Cells, | 529.94 | 81.31 | 5 | 307.50 | 62.85 | 12 | −1.72 | 0.001 |
| | Normal, CD19+ | Chronic | | | | | | | | |
| | | Lymphocytic | | | | | | | | |
| | | Leukemia | | | | | | | | |
| 208644_at | B | B Lymphocytes, | 583.14 | 112.02 | 14 | 394.31 | 86.95 | 6 | −1.48 | 0.001 |
| | Lymphocytes, | Anti-IgG; 8 hours | | | | | | | | |
| | Control; 0 hours | | | | | | | | | |
| 208644_at | Non-Hodgkin's | Non-Hodgkin's | 763.96 | 215.03 | 43 | 531.74 | 170.32 | 9 | −1.44 | 0.003 |
| | Lymphoma, | Lymphoma, Mantle | | | | | | | | |
| | Follicular Type, | Cell Type, All Body | | | | | | | | |
| | All Body Sites | Sites | | | | | | | | |
| 208644_at | Lymph Node, | White Blood Cells, | 475.49 | 123.80 | 9 | 307.50 | 62.85 | 12 | −1.55 | 0.003 |
| | Reactive | Chronic | | | | | | | | |
| | Lymphoid | Lymphocytic | | | | | | | | |
| | Hyperplasia | Leukemia | | | | | | | | |
| 208644_at | Thymus, | Thymus, Atrophy | 263.23 | 48.02 | 62 | 184.89 | 19.58 | 3 | −1.42 | 0.007 |
| | Normal | | | | | | | | | |
| 208644_at | B | B Lymphocytes, | 583.14 | 112.02 | 14 | 456.35 | 61.55 | 5 | −1.28 | 0.008 |
| | Lymphocytes, | PMA + Ionomycin; | | | | | | | | |
| | Control; 0 hours | 8 hours | | | | | | | | |
| 208644_at | Spleen, Normal | Spleen, Non- | 302.23 | 58.99 | 34 | 689.94 | 338.39 | 9 | 2.28 | 0.009 |
| | | Hodgkin's | | | | | | | | |
| | | Lymphoma, All | | | | | | | | |
| | | Types | | | | | | | | |
| 208644_at | Lymph Node, | Lymph Node, Non- | 752.45 | 201.14 | 30 | 528.09 | 181.71 | 8 | −1.42 | 0.010 |
| | Non-Hodgkin's | Hodgkin's | | | | | | | | |
| | Lymphoma, | Lymphoma, Mantle | | | | | | | | |
| | Follicular Type | Cell Type | | | | | | | | |
| 208644_at | Lymph Node, | Lymph Node, | 325.59 | 104.66 | 10 | 475.49 | 123.80 | 9 | 1.46 | 0.012 |
| | Normal | Reactive Lymphoid | | | | | | | | |
| | | Hyperplasia | | | | | | | | |
| 208644_at | Lymph Node, | Lymph Node, Non- | 325.59 | 104.66 | 10 | 528.09 | 181.71 | 8 | 1.62 | 0.018 |
| | Normal | Hodgkin's | | | | | | | | |
| | | Lymphoma, Mantle | | | | | | | | |
| | | Cell Type | | | | | | | | |
| 208644_at | B | B Lymphocytes, | 251.12 | 76.97 | 5 | 394.31 | 86.95 | 6 | 1.57 | 0.018 |
| | Lymphocytes, | Anti-IgG; 8 hours | | | | | | | | |
| | Anti-IgG; 2 | | | | | | | | | |
| | hours | | | | | | | | | |
| 208644_at | White Blood | White Blood Cells, | 180.31 | 57.64 | 5 | 87.11 | 38.59 | 4 | −2.07 | 0.024 |
| | Cells, Baseline | PMA + Ionomycin, | | | | | | | | |
| | Control, 0 | 4 Hours | | | | | | | | |
| | Hours | | | | | | | | | |
| 208644_at | Lymph Node, | Lymph Node, Non- | 958.33 | 465.47 | 32 | 752.45 | 201.14 | 30 | −1.27 | 0.027 |
| | Non-Hodgkin's | Hodgkin's | | | | | | | | |
| | Lymphoma, | Lymphoma, | | | | | | | | |
| | Diffuse Large | Follicular Type | | | | | | | | |
| | B-Cell Type | | | | | | | | | |
| 208644_at | Spleen, Non- | Lymph Node, Non- | 517.69 | 106.07 | 3 | 752.45 | 201.14 | 30 | 1.45 | 0.034 |
| | Hodgkin's | Hodgkin's | | | | | | | | |
| | Lymphoma, | Lymphoma, | | | | | | | | |
| | Follicular Type | Follicular Type | | | | | | | | |
| 208644_at | Lymph Node, | Lymph Node, | 325.59 | 14.66 | 10 | 412.41 | 57.47 | 12 | 1.27 | 0.035 |
| | Normal | Hodgkin's Disease, | | | | | | | | |
| | | Nodular Sclerosis | | | | | | | | |
| | | Type | | | | | | | | |
| 208644_at | B | B Lymphocytes, | 394.31 | 86.95 | 6 | 497.93 | 55.79 | 6 | 1.26 | 0.038 |
| | Lymphocytes, | LPS; 8 hours | | | | | | | | |
| | Anti-IgG; 8 | | | | | | | | | |
| | hours | | | | | | | | | |
| 208644_at | White Blood | Non-Hodgkin's | 307.50 | 62.85 | 12 | 442.13 | 87.15 | 4 | 1.44 | 0.045 |
| | Cells, Chronic | Lymphoma, Small | | | | | | | | |
| | Lymphocytic | Lymphocytic Type, | | | | | | | | |
| | Leukemia | All Body Sites | | | | | | | | |

TABLE XX
PARP1 - Diff/X (Human)
Name: poly (ADP-ribose) polymerase family, member 1
Sort By: p-value
Minimum Fold Change: 1.2
p-Value Range: 0.00-0.05
26 differential expression events found.
Digestive

| | | | | Control | | | Experiment | | Fold | |
|---|---|---|---|---|---|---|---|---|---|---|
| | | | Control | Standard | Control | Experiment | Standard | Experi- | change | p- |
| Fragment | Control | Experiment | Mean | Deviation | # | Mean | Deviation | ment # | (FC) | value |
| 208644_at | Pancreas, | Pancreas, Adenocarcinoma, | 321.84 | 69.04 | 46 | 191.82 | 53.50 | 23 | −1.68 | 0.000 |
| | Normal | Primary | | | | | | | | |
| 208644_at | Esophagus, | Esophagus, | 191.78 | 40.67 | 22 | 290.09 | 5.61 | 3 | 1.51 | 0.000 |
| | Normal | Adenocarcinoma, Primary | | | | | | | | |
| 208644_at | Pancreas, | Pancreas, Adenocarcinoma, | 313.58 | 74.61 | 23 | 166.22 | 27.30 | 5 | −1.89 | 0.000 |
| | Normal; | Primary; Smoking History | | | | | | | | |
| | Smoking | | | | | | | | | |
| | History | | | | | | | | | |
| 208644_at | Rectum, | Rectum, Adenocarcinoma | 206.94 | 31.16 | 44 | 262.78 | 62.38 | 29 | 1.27 | 0.000 |
| | Normal | (Excluding Mucinous | | | | | | | | |
| | | Type), Primary | | | | | | | | |
| 208644_at | Rectum, | Rectum, Adenocarcinoma | 209.76 | 32.14 | 35 | 262.78 | 62.38 | 29 | 1.25 | 0.000 |
| | Normal, | (Excluding Mucinous | | | | | | | | |
| | Primary | Type), Primary | | | | | | | | |
| | Malignancy | | | | | | | | | |
| | Elsewhere in | | | | | | | | | |
| | Colon or | | | | | | | | | |
| | Rectum | | | | | | | | | |
| 208644_at | Rectum, | Rectum, Adenocarcinoma | 206.94 | 31.16 | 44 | 260.98 | 63.95 | 26 | 1.26 | 0.000 |
| | Normal | (Excluding Mucinous | | | | | | | | |
| | | Type), Primary; Age 45 and | | | | | | | | |
| | | Over | | | | | | | | |
| 208644_at | Colon, | Colon, Adenocarcinoma | 197.05 | 44.62 | 62 | 244.35 | 59.56 | 26 | 1.24 | 0.001 |
| | Normal; | (Excluding Mucinous | | | | | | | | |
| | Smoking | Type), Primary; Smoking | | | | | | | | |
| | History | History | | | | | | | | |
| 208644_at | Liver, Focal | Liver, Hepatocellular | 151.17 | 14.70 | 8 | 241.43 | 87.63 | 16 | 1.60 | 0.001 |
| | Nodular | Carcinoma | | | | | | | | |
| | Hyperplasia | | | | | | | | | |
| 208644_at | Colon, | Colon, Adenocarcinoma | 199.20 | 44.27 | 56 | 244.35 | 59.56 | 26 | 1.23 | 0.001 |
| | Normal; No | (Excluding Mucinous | | | | | | | | |
| | History of | Type), Primary; Smoking | | | | | | | | |
| | Inflammatory | History | | | | | | | | |
| | Bowel | | | | | | | | | |
| | Disease; | | | | | | | | | |
| | Smoking | | | | | | | | | |
| | History | | | | | | | | | |
| 208644_at | Stomach, | Metastatic Adenocarcinoma | 324.58 | 46.07 | 5 | 159.57 | 34.93 | 3 | −2.03 | 0.002 |
| | Adenocarcinoma | (Excluding Signet Ring Cell | | | | | | | | |
| | (Excluding | Type) of Stomach, All | | | | | | | | |
| | Signet Ring | Secondary Sites | | | | | | | | |
| | Cell Type), | | | | | | | | | |
| | Primary; Stage | | | | | | | | | |
| | II | | | | | | | | | |
| 208644_at | Liver, | Liver, Hepatocellular | 168.31 | 20.68 | 25 | 241.43 | 87.63 | 16 | 1.43 | 0.005 |
| | Cirrhosis | Carcinoma | | | | | | | | |
| | Secondary to | | | | | | | | | |
| | Chronic | | | | | | | | | |
| | Hepatitis C | | | | | | | | | |
| 208644_at | Liver, | Liver, Hepatocellular | 169.21 | 36.75 | 61 | 241.43 | 87.63 | 16 | 1.43 | 0.005 |
| | Cirrhosis, All | Carcinoma | | | | | | | | |
| | Causes | | | | | | | | | |
| 208644_at | Stomach, | Metastatic Adenocarcinoma | 267.48 | 108.98 | 27 | 159.57 | 34.93 | 3 | −1.68 | 0.006 |
| | Adenocarcinoma | (Excluding Signet Ring Cell | | | | | | | | |
| | (Excluding | Type) of Stomach, All | | | | | | | | |
| | Signet Ring | Secondary Sites | | | | | | | | |
| | Cell Type), | | | | | | | | | |
| | Primary | | | | | | | | | |
| 208644_at | Pancreas, | Pancreas, Chronic | 321.84 | 69.04 | 46 | 245.29 | 70.89 | 10 | −1.31 | 0.008 |
| | Normal | Pancreatitis | | | | | | | | |
| 208644_at | Pancreas, | Pancreas, Chronic | 313.58 | 74.61 | 23 | 224.88 | 62.47 | 7 | −1.39 | 0.009 |
| | Normal; | Pancreatitis; Smoking | | | | | | | | |
| | Smoking | History | | | | | | | | |
| | History | | | | | | | | | |
| 208644_at | Pancreas, | Pancreas, Islet Cell Tumor, | 321.84 | 69.04 | 46 | 212.69 | 83.22 | 7 | −1.51 | 0.012 |
| | Normal | Malignant, Primary | | | | | | | | |
| 208644_at | Colon, | Metastatic Adenocarcinoma | 266.64 | 46.55 | 11 | 217.45 | 61.79 | 22 | −1.23 | 0.017 |
| | Adenocarcinoma | of Colon, All Secondary | | | | | | | | |
| | (Excluding | Sites | | | | | | | | |
| | Mucinous | | | | | | | | | |
| | Type), | | | | | | | | | |
| | Primary; Stage I | | | | | | | | | |
| 208644_at | Rectum, | Rectum, Adenocarcinoma | 195.57 | 30.49 | 10 | 269.75 | 45.67 | 5 | 1.38 | 0.017 |
| | Normal; No | (Excluding Mucinous | | | | | | | | |
| | Smoking | Type), Primary; No | | | | | | | | |
| | History | Smoking History | | | | | | | | |
| 208644_at | Stomach, | Metastatic Adenocarcinoma | 248.25 | 57.84 | 8 | 159.57 | 34.93 | 3 | −1.56 | 0.020 |
| | Adenocarcinoma | (Excluding Signet Ring Cell | | | | | | | | |
| | (Excluding | Type) of Stomach, All | | | | | | | | |
| | Signet Ring | Secondary Sites | | | | | | | | |
| | Cell Type), | | | | | | | | | |
| | Primary; Stage | | | | | | | | | |
| | III | | | | | | | | | |
| 208644_at | Pancreas, | Pancreas, Chronic | 321.84 | 69.04 | 46 | 215.92 | 55.96 | 4 | −1.49 | 0.025 |
| | Normal | Pancreatitis with Fibrosis | | | | | | | | |
| 208644_at | Liver, Normal | Liver, Steatosis (Fatty | 195.15 | 85.33 | 42 | 142.95 | 29.82 | 4 | −1.37 | 0.027 |
| | | Change) | | | | | | | | |
| 208644_at | Colon, | Colon, Ulcerative Colitis, | 197.19 | 44.36 | 152 | 237.19 | 57.53 | 13 | 1.20 | 0.029 |
| | Normal; No | Active (Acute | | | | | | | | |
| | History of | Inflammation) | | | | | | | | |
| | Inflammatory | | | | | | | | | |
| | Bowel Disease | | | | | | | | | |
| 208644_at | Rectum, | Rectum, Adenocarcinoma | 191.19 | 31.91 | 3 | 262.78 | 62.38 | 29 | 1.37 | 0.032 |
| | Normal, No | (Excluding Mucinous | | | | | | | | |
| | Primary | Type), Primary | | | | | | | | |
| | Colorectal | | | | | | | | | |
| | Malignancy | | | | | | | | | |
| 208644_at | Pancreas, | Pancreas, Adenocarcinoma, | 305.07 | 61.48 | 11 | 184.74 | 54.40 | 3 | −1.65 | 0.036 |
| | Normal; No | Primary; No Smoking | | | | | | | | |
| | Smoking | History | | | | | | | | |
| | History | | | | | | | | | |
| 208644_at | Colon, | Colon, Adenocarcinoma | 266.64 | 46.55 | 11 | 219.19 | 49.79 | 10 | −1.22 | 0.037 |
| | Adenocarcinoma | (Excluding Mucinous | | | | | | | | |
| | (Excluding | Type), Primary; Stage IV | | | | | | | | |
| | Mucinous | | | | | | | | | |
| | Type), | | | | | | | | | |
| | Primary; Stage I | | | | | | | | | |
| 208644_at | Stomach, | Stomach, Adenocarcinoma | 221.41 | 45.66 | 52 | 267.48 | 108.98 | 27 | 1.21 | 0.044 |
| | Normal | (Excluding Signet Ring Cell | | | | | | | | |
| | | Type), Primary | | | | | | | | |

TABLE XXI
PARP1 - Diff/X (Human)
Name: poly (ADP-ribose) polymerase family, member 1
Sort By: p-value
Minimum Fold Change: 1.2
p-Value Range: 0.00-0.05
10 differential expression events found.
Endocrine and neuroendocrine

| | | | | Control | | | Experiment | | Fold | |
|---|---|---|---|---|---|---|---|---|---|---|
| | | | Control | Standard | | Experiment | Standard | Experi- | change | p- |
| Fragment | Control | Experiment | Mean | Deviation | Control # | Mean | Deviation | ment # | (FC) | value |
| 208644_at | Thyroid Gland, | Thyroid Gland, Hashimoto's | 173.96 | 29.46 | 58 | 250.53 | 67.58 | 19 | 1.44 | 0.000 |
| | Nodular | Thyroiditis | | | | | | | | |
| | Hyperplasia | | | | | | | | | |
| 208644_at | Thyroid Gland, | Thyroid Gland, Hashimoto's | 173.82 | 34.78 | 24 | 250.53 | 67.58 | 19 | 1.44 | 0.000 |
| | Normal | Thyroiditis | | | | | | | | |
| 208644_at | Pancreas, | Pancreas, Islet Cell Tumor, | 321.84 | 69.04 | 46 | 212.69 | 83.22 | 7 | −1.51 | 0.012 |
| | Normal | Malignant, Primary | | | | | | | | |
| 208644_at | Thyroid Gland, | Thyroid Gland, Papillary | 173.96 | 29.46 | 58 | 225.17 | 46.13 | 8 | 1.29 | 0.016 |
| | Nodular | Carcinoma, Follicular Variant, | | | | | | | | |
| | Hyperplasia | Primary | | | | | | | | |
| 208644_at | Thyroid Gland, | Thyroid Gland, Papillary | 173.82 | 34.78 | 24 | 225.17 | 46.13 | 8 | 1.30 | 0.017 |
| | Normal | Carcinoma, Follicular Variant, | | | | | | | | |
| | | Primary | | | | | | | | |
| 208644_at | Thyroid Gland, | Thyroid Gland, Hashimoto's | 171.03 | 45.31 | 13 | 264.17 | 52.94 | 4 | 1.54 | 0.029 |
| | Normal; | Thyroiditis; Primary | | | | | | | | |
| | Primary | Malignancy Elsewhere in | | | | | | | | |
| | Malignancy | Thyroid | | | | | | | | |
| | Elsewhere in | | | | | | | | | |
| | Thyroid | | | | | | | | | |
| 208644_at | Thyroid Gland, | Thyroid Gland, Hashimoto's | 173.82 | 34.78 | 24 | 236.80 | 77.06 | 10 | 1.36 | 0.031 |
| | Normal | Thyroiditis; No Primary | | | | | | | | |
| | | Thyroid Malignancy | | | | | | | | |
| 208644_at | Thyroid Gland, | Thyroid Gland, Papillary | 250.53 | 67.58 | 19 | 206.04 | 51.25 | 15 | −1.22 | 0.037 |
| | Hashimoto's | Carcinoma (Excluding | | | | | | | | |
| | Thyroiditis | Follicular Variant), Primary | | | | | | | | |
| 208644_at | Thyroid Gland, | Thyroid Gland, Hashimoto's | 173.82 | 34.78 | 24 | 264.17 | 52.94 | 4 | 1.52 | 0.037 |
| | Normal | Thyroiditis; Primary | | | | | | | | |
| | | Malignancy Elsewhere in | | | | | | | | |
| | | Thyroid | | | | | | | | |
| 208644_at | Thyroid Gland, | Thyroid Gland, Hashimoto's | 172.59 | 37.49 | 7 | 236.80 | 77.06 | 10 | 1.37 | 0.039 |
| | Normal; No | Thyroiditis; No Primary | | | | | | | | |
| | Primary | Thyroid Malignancy | | | | | | | | |
| | Thyroid | | | | | | | | | |
| | Malignancy | | | | | | | | | |

TABLE XXII
PARP1 - e-Northern (Human)
Name: poly (ADP-ribose) polymerase family, member 1
View: CNS Disease
Sort By: Organ System
Fragment Legend: 208644_at

| | | | | | | | | | | |
|---|---|---|---|---|---|---|---|---|---|---|
| | | | % | | Lower | 25% | | | 75% | Upper |
| Category | Fragment | Freq. | Present | Count | Limit | Quan. | Median | Mean | Quan. | Limit |

Human, Central Nervous System Diseases

| | | | | | | | | | | |
|---|---|---|---|---|---|---|---|---|---|---|
| Frontal Pole (Brodmann Area 10), | 208644_at | 1.00 | 1.00 | 28 | 181.05 | 213.24 | 242.76 | 246.63 | 268.30 | 329.01 |
| Alzheimer's Disease | | | | | | | | | | |
| Frontal Pole (Brodmann Area 10), | 208644_at | 1.00 | 1.00 | 5 | 237.30 | 253.72 | 263.31 | 267.76 | 269.86 | 294.07 |
| Cocaine Abuse | | | | | | | | | | |
| Frontal Pole (Brodmann Area 10), | 208644_at | 1.00 | 1.00 | 13 | 172.51 | 217.43 | 246.28 | 239.09 | 252.87 | 306.02 |
| Normal, Control for Alzheimer's | | | | | | | | | | |
| Disease | | | | | | | | | | |
| Frontal Pole (Brodmann Area 10), | 208644_at | 1.00 | 1.00 | 5 | 229.04 | 231.23 | 252.08 | 254.81 | 255.54 | 292.01 |
| Normal, Control for Cocaine Abuse | | | | | | | | | | |
| Frontal Pole (Brodmann Area 10), | 208644_at | 1.00 | 1.00 | 5 | 210.43 | 221.60 | 257.34 | 257.33 | 271.98 | 325.28 |
| Normal, Control for Schizophrenia | | | | | | | | | | |
| Frontal Pole (Brodmann Area 10), | 208644_at | 1.00 | 0.90 | 10 | 175.85 | 233.19 | 254.10 | 247.91 | 271.41 | 296.18 |
| Normal, Control for Suicide | | | | | | | | | | |
| Frontal Pole (Brodmann Area 10), | 208644_at | 1.00 | 1.00 | 4 | 226.08 | 232.78 | 245.47 | 244.26 | 256.96 | 260.04 |
| Schizophrenia | | | | | | | | | | |
| Frontal Pole (Brodmann Area 10), | 208644_at | 1.00 | 1.00 | 13 | 191.56 | 228.93 | 243.90 | 247.91 | 253.83 | 291.20 |
| Suicide with History of Depression | | | | | | | | | | |
| Frontal Pole (Brodmann Area 10), | 208644_at | 1.00 | 1.00 | 7 | 202.41 | 220.38 | 241.85 | 238.98 | 248.03 | 289.49 |
| Suicide with No History of | | | | | | | | | | |
| Depression | | | | | | | | | | |
| Globus Pallidus, Cocaine Abuse | 208644_at | 1.00 | 1.00 | 3 | 302.66 | 309.12 | 315.58 | 355.59 | 382.06 | 448.55 |
| Globus Pallidus, Normal, Control for | 208644_at | 1.00 | 1.00 | 5 | 298.23 | 335.54 | 367.85 | 368.05 | 399.65 | 439.00 |
| Cocaine Abuse | | | | | | | | | | |
| Globus Pallidus, Normal, Control for | 208644_at | 1.00 | 1.00 | 7 | 211.05 | 244.52 | 266.29 | 267.82 | 286.46 | 335.47 |
| Parkinson's Disease | | | | | | | | | | |
| Globus Pallidus, Parkinson's Disease | 208644_at | 1.00 | 1.00 | 3 | 283.65 | 299.37 | 315.09 | 310.93 | 324.57 | 334.06 |
| Hippocampus, Alzheimer's Disease | 208644_at | 1.00 | 1.00 | 24 | 228.60 | 281.28 | 302.35 | 316.71 | 351.95 | 457.95 |
| Hippocampus, Cocaine Abuse | 208644_at | 1.00 | 1.00 | 3 | 234.84 | 249.25 | 263.67 | 271.61 | 289.99 | 316.32 |
| Hippocampus, Normal, Control for | 208644_at | 1.00 | 1.00 | 10 | 200.23 | 246.25 | 261.12 | 266.34 | 286.51 | 327.95 |
| Alzheimer's Disease | | | | | | | | | | |
| Hippocampus, Normal, Control for | 208644_at | 1.00 | 1.00 | 4 | 215.03 | 240.17 | 264.13 | 258.83 | 282.79 | 292.05 |
| Cocaine Abuse | | | | | | | | | | |
| Hippocampus, Normal, Control for | 208644_at | 1.00 | 1.00 | 5 | 194.29 | 227.61 | 228.76 | 234.31 | 252.09 | 268.81 |
| Schizophrenia | | | | | | | | | | |
| Hippocampus, Normal, Control for | 208644_at | 1.00 | 1.00 | 9 | 210.03 | 273.68 | 275.59 | 301.54 | 324.12 | 399.78 |
| Suicide | | | | | | | | | | |
| Hippocampus, Schizophrenia | 208644_at | 1.00 | 1.00 | 3 | 205.30 | 256.21 | 307.12 | 274.77 | 309.51 | 311.91 |
| Hippocampus, Suicide with History | 208644_at | 1.00 | 1.00 | 13 | 211.94 | 243.55 | 260.10 | 265.15 | 285.48 | 335.41 |
| of Depression | | | | | | | | | | |
| Hippocampus, Suicide with No | 208644_at | 1.00 | 1.00 | 4 | 253.63 | 262.43 | 273.73 | 271.74 | 283.05 | 285.90 |
| History of Depression | | | | | | | | | | |
| Hypothalamus, Cocaine Abuse | 208644_at | 1.00 | 1.00 | 3 | 211.56 | 225.09 | 238.63 | 236.11 | 248.39 | 258.15 |
| Hypothalamus, Normal, Control for | 208644_at | 1.00 | 1.00 | 5 | 216.98 | 230.52 | 232.27 | 233.92 | 239.55 | 253.10 |
| Cocaine Abuse | | | | | | | | | | |
| Hypothalamus, Normal, Control for | 208644_at | 1.00 | 1.00 | 6 | 178.60 | 210.99 | 238.71 | 237.73 | 260.16 | 301.31 |
| Schizophrenia | | | | | | | | | | |
| Hypothalamus, Normal, Control for | 208644_at | 1.00 | 1.00 | 10 | 211.21 | 245.46 | 259.94 | 297.36 | 335.96 | 471.71 |
| Suicide | | | | | | | | | | |
| Hypothalamus, Schizophrenia | 208644_at | 1.00 | 1.00 | 4 | 209.25 | 217.69 | 246.61 | 247.98 | 276.90 | 289.45 |
| Hypothalamus, Suicide with History | 208644_at | 1.00 | 1.00 | 10 | 166.61 | 241.15 | 275.50 | 283.51 | 331.57 | 382.80 |
| of Depression | | | | | | | | | | |
| Hypothalamus, Suicide with No | 208644_at | 1.00 | 1.00 | 6 | 173.38 | 231.70 | 262.26 | 287.21 | 322.00 | 457.46 |
| History of Depression | | | | | | | | | | |
| Middle Temporal Gyrus (Brodmann | 208644_at | 1.00 | 1.00 | 23 | 186.15 | 201.48 | 239.67 | 249.07 | 287.57 | 333.80 |
| Area 21), Alzheimer's Disease | | | | | | | | | | |
| Middle Temporal Gyrus (Brodmann | 208644_at | 1.00 | 1.00 | 5 | 228.42 | 245.60 | 251.65 | 259.35 | 283.86 | 287.21 |
| Area 21), Cocaine Abuse | | | | | | | | | | |
| Middle Temporal Gyrus (Brodmann | 208644_at | 1.00 | 1.00 | 13 | 166.94 | 229.83 | 270.81 | 261.79 | 286.11 | 329.08 |
| Area 21), Normal, Control for | | | | | | | | | | |
| Alzheimer's Disease | | | | | | | | | | |
| Middle Temporal Gyrus (Brodmann | 208644_at | 1.00 | 1.00 | 4 | 222.13 | 236.64 | 242.46 | 240.49 | 246.31 | 257.74 |
| Area 21), Normal, Control for | | | | | | | | | | |
| Cocaine Abuse | | | | | | | | | | |
| Middle Temporal Gyrus (Brodmann | 208644_at | 1.00 | 1.00 | 5 | 226.88 | 251.89 | 251.92 | 264.68 | 268.57 | 293.58 |
| Area 21), Normal, Control for | | | | | | | | | | |
| Schizophrenia | | | | | | | | | | |
| Middle Temporal Gyrus (Brodmann | 208644_at | 1.00 | 1.00 | 9 | 213.87 | 227.38 | 242.69 | 246.71 | 254.72 | 295.73 |
| Area 21), Normal, Control for | | | | | | | | | | |
| Suicide | | | | | | | | | | |
| Middle Temporal Gyrus (Brodmann | 208644_at | 1.00 | 1.00 | 3 | 243.88 | 245.99 | 248.09 | 260.43 | 268.71 | 289.33 |
| Area 21), Schizophrenia | | | | | | | | | | |
| Middle Temporal Gyrus (Brodmann | 208644_at | 1.00 | 1.00 | 13 | 187.01 | 218.56 | 226.29 | 235.20 | 258.42 | 297.74 |
| Area 21), Suicide with History of | | | | | | | | | | |
| Depression | | | | | | | | | | |
| Middle Temporal Gyrus (Brodmann | 208644_at | 1.00 | 1.00 | 5 | 208.01 | 213.06 | 218.72 | 240.08 | 274.17 | 286.43 |
| Area 21), Suicide with No History of | | | | | | | | | | |
| Depression | | | | | | | | | | |
| Orbital Gyri (Brodmann Area 11), | 208644_at | 1.00 | 1.00 | 6 | 223.10 | 270.25 | 276.05 | 282.87 | 301.68 | 348.84 |
| Cocaine Abuse | | | | | | | | | | |
| Orbital Gyri (Brodmann Area 11), | 208644_at | 1.00 | 1.00 | 4 | 162.55 | 201.93 | 241.10 | 238.16 | 277.33 | 307.89 |
| Normal, Control for Cocaine Abuse | | | | | | | | | | |
| Orbital Gyri (Brodmann Area 11), | 208644_at | 1.00 | 1.00 | 7 | 198.59 | 218.74 | 272.96 | 255.36 | 284.60 | 309.28 |
| Normal, Control for Schizophrenia | | | | | | | | | | |
| Orbital Gyri (Brodmann Area 11), | 208644_at | 1.00 | 1.00 | 11 | 164.98 | 207.28 | 266.77 | 253.97 | 303.79 | 315.25 |
| Normal, Control for Suicide | | | | | | | | | | |
| Orbital Gyri (Brodmann Area 11), | 208644_at | 1.00 | 1.00 | 3 | 198.10 | 228.48 | 258.86 | 290.58 | 336.82 | 414.78 |
| Schizophrenia | | | | | | | | | | |
| Orbital Gyri (Brodmann Area 11), | 208644_at | 1.00 | 1.00 | 13 | 216.58 | 228.95 | 241.10 | 258.04 | 255.79 | 296.05 |
| Suicide with History of Depression | | | | | | | | | | |
| Orbital Gyri (Brodmann Area 11), | 208644_at | 1.00 | 1.00 | 7 | 202.12 | 230.70 | 268.53 | 261.28 | 279.44 | 338.03 |
| Suicide with No History of | | | | | | | | | | |
| Depression | | | | | | | | | | |
| Substantia Nigra, Cocaine Abuse | 208644_at | 1.00 | 1.00 | 4 | 356.39 | 362.40 | 372.59 | 418.80 | 428.99 | 528.87 |
| Substantia Nigra, Normal, Control | 208644_at | 1.00 | 1.00 | 7 | 207.11 | 236.60 | 271.47 | 278.05 | 310.90 | 372.76 |
| for Cocaine Abuse | | | | | | | | | | |
| Substantia Nigra, Normal, Control | 208644_at | 1.00 | 1.00 | 7 | 209.02 | 251.99 | 284.95 | 281.51 | 302.75 | 367.08 |
| for Parkinson's Disease | | | | | | | | | | |
| Substantia Nigra, Normal, Control | 208644_at | 1.00 | 1.00 | 7 | 209.02 | 251.99 | 284.95 | 281.51 | 302.75 | 367.08 |
| for Schizophrenia | | | | | | | | | | |
| Substantia Nigra, Parkinson's | 208644_at | 1.00 | 1.00 | 11 | 226.36 | 302.00 | 335.16 | 342.26 | 384.38 | 474.81 |
| Disease | | | | | | | | | | |
| Substantia Nigra, Schizophrenia | 208644_at | 1.00 | 1.00 | 4 | 248.14 | 267.98 | 308.80 | 305.65 | 346.47 | 356.86 |
| Superior Temporal Gyrus | 208644_at | 1.00 | 1.00 | 31 | 179.60 | 247.51 | 273.23 | 271.68 | 292.78 | 360.69 |
| (Brodmann Area 22), Alzheimer's | | | | | | | | | | |
| Disease | | | | | | | | | | |
| Superior Temporal Gyrus | 208644_at | 1.00 | 1.00 | 3 | 218.45 | 234.49 | 250.53 | 251.64 | 268.24 | 285.94 |
| (Brodmann Area 22), Cocaine Abuse | | | | | | | | | | |
| Superior Temporal Gyrus | 208644_at | 1.00 | 1.00 | 11 | 213.70 | 251.89 | 290.15 | 295.56 | 334.00 | 390.34 |
| (Brodmann Area 22), Normal, | | | | | | | | | | |
| Control for Alzheimer's Disease | | | | | | | | | | |
| Superior Temporal Gyrus | 208644_at | 1.00 | 1.00 | 5 | 203.91 | 230.71 | 241.65 | 237.45 | 248.58 | 275.38 |
| (Brodmann Area 22), Normal, | | | | | | | | | | |
| Control for Cocaine Abuse | | | | | | | | | | |
| Superior Temporal Gyrus | 208644_at | 1.00 | 1.00 | 4 | 200.91 | 225.92 | 238.62 | 229.90 | 242.60 | 246.96 |
| (Brodmann Area 22), Normal, | | | | | | | | | | |
| Control for Schizophrenia | | | | | | | | | | |
| Superior Temporal Gyrus | 208644_at | 1.00 | 1.00 | 3 | 224.12 | 250.20 | 276.27 | 263.57 | 283.29 | 290.32 |
| (Brodmann Area 22), Schizophrenia | | | | | | | | | | |

TABLE XXIII
PARP1 - e-Northern (Human)
Name: poly (ADP-ribose) polymerase family, member 1
View: Normal Tissues
Fragment Legend: 208644_at
Sort By: Organ System

| | | | % | | Lower | 25% | | | | Upper |
|---|---|---|---|---|---|---|---|---|---|---|
| Category | Fragment | Freq. | Present | Count | Limit | Quan. | Median | Mean | 75% Quan. | Limit |
| Human, Cardiovascular System | | | | | | | | | | |
| Artery, Normal | 208644_at | 1.00 | 1.00 | 4 | 95.96 | 129.13 | 141.33 | 160.15 | 172.35 | 237.17 |
| Left Atrium, Normal | 208644_at | 1.00 | 1.00 | 18 | 121.96 | 186.33 | 206.52 | 204.39 | 232.62 | 259.32 |
| Left Ventricle, Normal | 208644_at | 1.00 | 1.00 | 3 | 256.11 | 281.73 | 307.35 | 305.95 | 330.86 | 354.38 |
| Right Atrium, Normal | 208644_at | 1.00 | 1.00 | 4 | 204.76 | 209.20 | 216.10 | 219.89 | 226.79 | 242.61 |
| Right Ventricle, Normal | 208644_at | 1.00 | 1.00 | 4 | 220.97 | 291.24 | 318.13 | 311.18 | 338.08 | 393.16 |
| Human, Central Nervous System | | | | | | | | | | |
| Frontal Pole (Brodmann Area 10) Normal | 208644_at | 1.00 | 0.97 | 33 | 170.12 | 221.60 | 247.07 | 246.91 | 264.26 | 325.28 |
| Globus Pallidus, Normal | 208644_at | 1.00 | 1.00 | 13 | 211.05 | 266.29 | 298.23 | 311.27 | 335.54 | 439.00 |
| Hippocampus, Normal | 208644_at | 1.00 | 1.00 | 28 | 194.29 | 241.49 | 270.01 | 270.86 | 288.02 | 357.82 |
| Hypothalamus, Normal | 208644_at | 1.00 | 1.00 | 21 | 178.60 | 232.27 | 245.24 | 265.22 | 286.90 | 368.85 |
| Inferior Temporal Gyrus (Brodmann, | 208644_at | 1.00 | 1.00 | 29 | 163.86 | 206.45 | 235.52 | 243.86 | 280.06 | 320.23 |
| Area 20), Normal | | | | | | | | | | |
| Middle Temporal Gyrus (Brodmann Area 21) | 208644_at | 1.00 | 1.00 | 32 | 166.94 | 229.22 | 251.90 | 255.80 | 277.85 | 330.43 |
| Normal | | | | | | | | | | |
| Motor Cortex (Brodmann Area 4), | 208644_at | 1.00 | 1.00 | 22 | 165.95 | 214.75 | 253.98 | 247.82 | 270.52 | 354.17 |
| Normal | | | | | | | | | | |
| Orbital Gyri (Broadmann Area 11), | 208644_at | 1.00 | 1.00 | 23 | 162.55 | 210.27 | 266.77 | 249.59 | 303.58 | 315.25 |
| Normal | | | | | | | | | | |
| Substantia Nigra, Normal | 208644_at | 1.00 | 1.00 | 14 | 207.11 | 234.99 | 278.87 | 279.78 | 312.01 | 372.76 |
| Superior Frontas Gyrus (Broadmann Area | | | | 15 | 160.65 | 223.87 | 245.57 | 255.79 | 266.94 | 331.56 |
| 8) Normal | | | | | | | | | | |
| Superior Temporal Gyrus (Broadmann | 208644_at | 1.00 | 1.00 | 21 | 188.97 | 229.84 | 246.96 | 265.75 | 290.15 | 380.62 |
| Area 22) Normal | | | | | | | | | | |
| Temporal Pole (Broadmann Area 38), | 208644_at | 1.00 | 1.00 | 10 | 174.34 | 229.92 | 256.75 | 251.35 | 272.51 | 333.98 |
| Normal | | | | | | | | | | |
| Human, Digestive System | | | | | | | | | | |
| Appendix, Normal | 208644_at | 1.00 | 1.00 | 3 | 232.12 | 327.87 | 432.62 | 458.83 | 576.69 | 720.76 |
| Colon, Normal | | | | 180 | 88.25 | 166.74 | 191.91 | 198.00 | 229.97 | 324.80 |
| Duodenum, Normal | 208644_at | 1.00 | 1.00 | 77 | 125.77 | 168.39 | 183.76 | 186.90 | 202.06 | 252.56 |
| Esophagus, Normal | 208644_at | 1.00 | 1.00 | 22 | 132.91 | 162.68 | 187.02 | 191.78 | 219.85 | 291.45 |
| Gallbladder, Normal | 208644_at | 1.00 | 1.00 | 7 | 113.46 | 129.19 | 142.84 | 155.06 | 185.08 | 200.60 |
| Liver, Normal | 208644_at | 1.00 | 1.00 | 42 | 85.71 | 149.27 | 172.85 | 195.15 | 201.08 | 278.79 |
| Pancreas, Normal | 208644_at | 1.00 | 1.00 | 46 | 131.80 | 276.35 | 319.04 | 321.84 | 372.71 | 469.39 |
| Rectum, Normal | 208644_at | | 1.00 | 44 | 154.22 | 180.56 | 204.22 | 206.94 | 225.30 | 285.55 |
| Small Intestine, Normal | 208644_at | 1.00 | 1.00 | 97 | 90.87 | 167.17 | 186.89 | 193.57 | 218.03 | 294.33 |
| Stomach, Normal | 208644_at | 1.00 | 0.98 | 52 | 116.33 | 190.63 | 215.46 | 221.41 | 244.02 | 324.10 |
| Human, Endocrine System | | | | | | | | | | |
| Adrenal Gland, Normal | 208644_at | 1.00 | 0.92 | 13 | 118.84 | 149.84 | 169.92 | 179.87 | 183.69 | 234.47 |
| Thyroid Gland, Normal | 208644_at | 1.00 | 1.00 | 24 | 125.34 | 150.78 | 171.69 | 173.82 | 187.96 | 243.72 |
| Human, Female Reproductive System | | | | | | | | | | |
| Breast, Normal | 208644_at | 1.00 | 1.00 | 68 | 123.07 | 161.29 | 178.83 | 201.78 | 217.55 | 301.94 |
| Endometrium, Normal | 208644_at | 1.00 | 1.00 | 23 | 105.71 | 154.36 | 199.06 | 201.21 | 227.26 | 336.60 |
| Fallopian Tube, Normal | 208644_at | 1.00 | 1.00 | 49 | 102.55 | 161.59 | 181.65 | 188.16 | 206.60 | 274.11 |
| Myometrium, Normal | 208644_at | 1.00 | 1.00 | 122 | 99.43 | 155.41 | 175.44 | 176.66 | 195.83 | 256.47 |
| Ovary, Normal | 208644_at | 1.00 | 1.00 | 89 | 98.25 | 147.72 | 161.49 | 163.31 | 180.70 | 224.40 |
| Uterine Cervix, Normal | 208644_at | 1.00 | 0.98 | 115 | 77.91 | 150.20 | 168.54 | 178.85 | 198.40 | 270.70 |
| Uterus (Endometrium + Myometrium), | 208644_at | 1.00 | 1.00 | 58 | 109.78 | 153.82 | 179.77 | 186.55 | 209.66 | 293.42 |
| Normal | | | | | | | | | | |
| Human, Immune System | | | | | | | | | | |
| B-lymphocytes, Normal, CD19+ | 208644_at | 1.00 | 1.00 | 5 | 407.27 | 508.54 | 529.66 | 529.94 | 586.12 | 618.14 |
| Lymph Node, Normal | 208644_at | 1.00 | 1.00 | 10 | 217.06 | 316.25 | 355.00 | 325.59 | 382.39 | 452.30 |
| Monocytes, Normal, CD14+ | 208644_at | 1.00 | 1.00 | 8 | 117.24 | 126.21 | 133.50 | 138.79 | 148.85 | 172.61 |
| Mononuclear White Blood Cells, Normal | 208644_at | 1.00 | 1.00 | 8 | 219.71 | 243.66 | 263.41 | 259.16 | 277.35 | 296.62 |
| Natural Killer Cells, Normal, CD56+ | 208644_at | 1.00 | 1.00 | 4 | 229.11 | 234.35 | 252.60 | 259.27 | 277.52 | 302.76 |
| Neutrophils, Normal | 208644_at | 1.00 | 0.67 | 3 | 28.89 | 33.76 | 38.62 | 41.87 | 48.36 | 58.10 |
| Spleen, Normal | 208644_at | 1.00 | 1.00 | 34 | 207.76 | 255.56 | 298.84 | 302.23 | 335.73 | 409.07 |
| T-lymphocytes, Normal, CD4+ | 208644_at | 1.00 | 1.00 | 4 | 212.49 | 284.03 | 322.24 | 304.66 | 342.88 | 361.67 |
| T-lymphocytes, Normal, CD8+ | 208644_at | 1.00 | 1.00 | 6 | 235.18 | 258.74 | 279.61 | 273.01 | 290.19 | 298.15 |
| Thymus, Normal | 208644_at | 1.00 | 1.00 | 62 | 177.63 | 226.17 | 261.44 | 263.23 | 292.65 | 392.37 |
| White Blood Cells, Normal | 208644_at | 1.00 | 1.00 | 14 | 130.23 | 168.79 | 178.89 | 176.91 | 194.50 | 233.06 |
| Human, Integumentary and | | | | | | | | | | |
| Musculoskeletal System | | | | | | | | | | |
| Adipose Tissue, Normal | 208644_at | 1.00 | 1.00 | 34 | 102.07 | 152.27 | 170.25 | 171.87 | 186.93 | 238.93 |
| Bone, Normal | 208644_at | 1.00 | 1.00 | 8 | 154.45 | 183.30 | 194.26 | 196.06 | 218.32 | 225.33 |
| Omentum, Normal | 208644_at | 1.00 | 1.00 | 15 | 112.36 | 177.28 | 201.36 | 219.15 | 220.56 | 285.48 |
| Skeletal Muscle, Normal | 208644_at | 1.00 | 1.00 | 47 | 124.99 | 198.66 | 232.58 | 232.85 | 273.29 | 347.32 |
| Skin, Normal | 208644_at | 1.00 | 1.00 | 61 | 73.17 | 123.09 | 149.86 | 154.29 | 167.66 | 234.50 |
| Human, Male Reproductive System | | | | | | | | | | |
| Prostate, Normal | 208644_at | 1.00 | 1.00 | 57 | 135.98 | 184.58 | 201.84 | 209.09 | 236.64 | 294.97 |
| Testis, Normal | 208644_at | 1.00 | 1.00 | 7 | 246.00 | 277.07 | 325.94 | 333.35 | 368.24 | 470.90 |
| Human, Respiratory System | | | | | | | | | | |
| Larynx, Normal | 208644_at | 1.00 | 1.00 | 4 | 180.40 | 191.48 | 208.13 | 208.76 | 225.41 | 238.37 |
| Lung, Normal | 208644_at | 1.00 | 1.00 | 126 | 87.04 | 143.56 | 161.44 | 170.58 | 181.24 | 237.76 |
| Human, Urinary Tract | | | | | | | | | | |
| Kidney, Normal | 208644_at | 1.00 | 0.99 | 81 | 115.79 | 143.82 | 165.98 | 165.78 | 184.14 | 244.62 |
| Urinary Bladder, Normal | 208644_at | 1.00 | 1.00 | 9 | 128.23 | 162.66 | 184.14 | 223.63 | 267.52 | 382.66 |

### Techniques for Analysis of PARP

The analysis of the PARP may include analysis of PARP gene expression, including an analysis of DNA, RNA, analysis of the level of PARP and/or analysis of the activity of PARP including a level of mono- and poly-ADP-ribozylation. Without limiting the scope of the present invention, any number of techniques known in the art can be employed for the analysis of PARP and they are all within the scope of the present invention. Some of the examples of such detection technique are given below but these examples are in no way limiting to the various detection techniques that can be used in the present invention.

Gene Expression Profiling: Methods of gene expression profiling include methods based on hybridization analysis of polynucleotides, polyribonucleotides methods based on sequencing of polynucleotides, polyribonucleotides and proteomics-based methods. The most commonly used methods known in the art for the quantification of mRNA expression in a sample include northern blotting and in situ hybridization (Parker & Barnes, Methods in Molecular Biology 106:247-283 (1999)); RNAse protection assays (Hod, Biotechniques 13:852-854 (1992)); and PCR-based methods, such as reverse transcription polymerase chain reaction (RT-PCR) (Weis et al., Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS), Comparative Genome Hybridisation (CGH), Chromatin Immunoprecipitation (ChIP), Single nucleotide polymorphism (SNP) and SNP arrays, Fluorescent in situ Hybridization (FISH), Protein binding arrays and DNA microarray (also commonly known as gene or genome chip, DNA chip, or gene array), RNA microarrays.

Reverse Transcriptase PCR (RT-PCR): One of the most sensitive and most flexible quantitative PCR-based gene expression profiling methods is RT-PCR, which can be used to compare mRNA levels in different sample populations, in normal and tumor tissues, with or without drug treatment, to characterize patterns of gene expression, to discriminate between closely related mRNAs, and to analyze RNA structure.

The first step is the isolation of mRNA from a target sample. For example, the starting material can be typically total RNA isolated from human tumors or tumor cell lines, and corresponding normal tissues or cell lines, respectively. Thus RNA can be isolated from a variety of normal and diseased cells and tissues, for example tumors, including breast, lung, colorectal, prostate, brain, liver, kidney, pancreas, spleen, thymus, testis, ovary, uterus, etc., or tumor cell lines. If the source of mRNA is a primary tumor, mRNA can be extracted, for example, from frozen or archived fixed tissues, for example paraffin-embedded and fixed (e.g. formalin-fixed) tissue samples. General methods for mRNA extraction are well known in the art and are disclosed in standard textbooks of molecular biology, including Ausubel et al., Current Protocols of Molecular Biology, John Wiley and Sons (1997).

In particular, RNA isolation can be performed using purification kit, buffer set and protease from commercial manufacturers, according to the manufacturer's instructions. RNA prepared from tumor can be isolated, for example, by cesium chloride density gradient centrifugation. As RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. The derived cDNA can then be used as a template in the subsequent PCR reaction.

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

A more recent variation of the RT-PCR technique is the real time quantitative PCR, which measures PCR product accumulation through a dual-labeled fluorigenic probe. Real time PCR is compatible both with quantitative competitive PCR, where internal competitor for each target sequence is used for normalization, and with quantitative comparative PCR using a normalization gene contained within the sample, or a housekeeping gene for RT-PCR.

Fluorescence Microscopy: Some embodiments of the invention include fluorescence microscopy for analysis of PARP. Fluorescence microscopy enables the molecular composition of the structures being observed to be identified through the use of fluorescently-labeled probes of high chemical specificity such as antibodies. It can be done by directly conjugating a fluorophore to a protein and introducing this back into a cell. Fluorescent analogue may behave like the native protein and can therefore serve to reveal the distribution and behavior of this protein in the cell. Along with NMR, infrared spectroscopy, circular dichroism and other techniques, protein intrinsic fluorescence decay and its associated observation of fluorescence anisotropy, collisional quenching and resonance energy transfer are techniques for protein detection. The naturally fluorescent proteins can be used as fluorescent probes. The jellyfish aequorea victoria produces a naturally fluorescent protein known as green fluorescent protein (GFP). The fusion of these fluorescent probes to a target protein enables visualization by fluorescence microscopy and quantification by flow cytometry.

By way of example only, some of the probes are labels such as, fluorescein and its derivatives, carboxyfluoresceins, rhodamines and their derivatives, atto labels, fluorescent red and fluorescent orange: cy3/cy5 alternatives, lanthanide complexes with long lifetimes, long wavelength labels—up to 800 nm, DY cyanine labels, and phycobili proteins. By way of example only, some of the probes are conjugates such as, isothiocyanate conjugates, streptavidin conjugates, and biotin conjugates. By way of example only, some of the probes are enzyme substrates such as, fluorogenic and chromogenic substrates. By way of example only, some of the probes are fluorochromes such as, FITC (green fluorescence, excitation/emission=506/529 nm), rhodamine B (orange fluorescence, excitation/emission=560/584 nm), and nile blue A (red fluorescence, excitation/emission=636/686 nm). Fluorescent nanoparticles can be used for various types of immunoassays. Fluorescent nanoparticles are based on different materials, such as, polyacrylonitrile, and polystyrene etc. Fluorescent molecular rotors are sensors of microenvironmental restriction that become fluorescent when their rotation is constrained. Few examples of molecular constraint include increased dye (aggregation), binding to antibodies, or being trapped in the polymerization of actin. IEF (isoelectric focusing) is an analytical tool for the separation of ampholytes, mainly proteins. An advantage for IEF-gel electrophoresis with fluorescent IEF-marker is the possibility to directly observe the formation of gradient. Fluorescent IEF-marker can also be detected by UV-absorption at 280 nm (20° C.).

A peptide library can be synthesized on solid supports and, by using coloring receptors, subsequent dyed solid supports can be selected one by one. If receptors cannot indicate any color, their binding antibodies can be dyed. The method can not only be used on protein receptors, but also on screening binding ligands of synthesized artificial receptors and screening new metal binding ligands as well. Automated methods for HTS and FACS (fluorescence activated cell sorter) can also be used. A FACS machine originally runs cells through a capillary tube and separate cells by detecting their fluorescent intensities.

Immunoassays: Some embodiments of the invention include immunoassay for the analysis of PARP. In immunoblotting like the western blot of electrophoretically separated proteins a single protein can be identified by its antibody. Immunoassay can be competitive binding immunoassay where analyte competes with a labeled antigen for a limited pool of antibody molecules (e.g. radioimmunoassay, EMIT). Immunoassay can be non-competitive where antibody is present in excess and is labeled. As analyte antigen complex is increased, the amount of labeled antibody-antigen complex may also increase (e.g. ELISA). Antibodies can be polyclonal if produced by antigen injection into an experimental animal, or monoclonal if produced by cell fusion and cell culture techniques. In immunoassay, the antibody may serve as a specific reagent for the analyte antigen.

Without limiting the scope and content of the present invention, some of the types of immunoassays are, by way of example only, RIAs (radioimmunoassay), enzyme immunoassays like ELISA (enzyme-linked immunosorbent assay), EMIT (enzyme multiplied immunoassay technique), microparticle enzyme immunoassay (MEIA), LIA (luminescent immunoassay), and FIA (fluorescent immunoassay). These techniques can be used to detect biological substances in the nasal specimen. The antibodies—either used as primary or secondary ones—can be labeled with radioisotopes (e.g. 125I), fluorescent dyes (e.g. FITC) or enzymes (e.g. HRP or AP) which may catalyse fluorogenic or luminogenic reactions.

Biotin, or vitamin H is a co-enzyme which inherits a specific affinity towards avidin and streptavidin. This interaction makes biotinylated peptides a useful tool in various biotechnology assays for quality and quantity testing. To improve biotin/streptavidin recognition by minimizing steric hindrances, it can be necessary to enlarge the distance between biotin and the peptide itself. This can be achieved by coupling a spacer molecule (e.g., 6-aminohexanoic acid) between biotin and the peptide.

The biotin quantitation assay for biotinylated proteins provides a sensitive fluorometric assay for accurately determining the number of biotin labels on a protein. Biotinylated peptides are widely used in a variety of biomedical screening systems requiring immobilization of at least one of the interaction partners onto streptavidin coated beads, membranes, glass slides or microtiter plates. The assay is based on the displacement of a ligand tagged with a quencher dye from the biotin binding sites of a reagent. To expose any biotin groups in a multiply labeled protein that are sterically restricted and inaccessible to the reagent, the protein can be treated with protease for digesting the protein.

EMIT is a competitive binding immunoassay that avoids the usual separation step. A type of immunoassay in which the protein is labeled with an enzyme, and the enzyme-protein-antibody complex is enzymatically inactive, allowing quantitation of unlabelled protein. Some embodiments of the invention include ELISA to analyze PARP. ELISA is based on selective antibodies attached to solid supports combined with enzyme reactions to produce systems capable of detecting low levels of proteins. It is also known as enzyme immunoassay or EIA. The protein is detected by antibodies that have been made against it, that is, for which it is the antigen. Monoclonal antibodies are often used.

The test may require the antibodies to be fixed to a solid surface, such as the inner surface of a test tube, and a preparation of the same antibodies coupled to an enzyme. The enzyme may be one (e.g., β-galactosidase) that produces a colored product from a colorless substrate. The test, for example, may be performed by filling the tube with the antigen solution (e.g., protein) to be assayed. Any antigen molecule present may bind to the immobilized antibody molecules. The antibody-enzyme conjugate may be added to the reaction mixture. The antibody part of the conjugate binds to any antigen molecules that were bound previously, creating an antibody-antigen-antibody “sandwich”. After washing away any unbound conjugate, the substrate solution may be added. After a set interval, the reaction is stopped (e.g., by adding 1 N NaOH) and the concentration of colored product formed is measured in a spectrophotometer. The intensity of color is proportional to the concentration of bound antigen.

ELISA can also be adapted to measure the concentration of antibodies, in which case, the wells are coated with the appropriate antigen. The solution (e.g., serum) containing antibody may be added. After it has had time to bind to the immobilized antigen, an enzyme-conjugated anti-immunoglobulin may be added, consisting of an antibody against the antibodies being tested for. After washing away unreacted reagent, the substrate may be added. The intensity of the color produced is proportional to the amount of enzyme-labeled antibodies bound (and thus to the concentration of the antibodies being assayed).

Some embodiments of the invention include radioimmunoassays to analyze PARP. Radioactive isotopes can be used to study in vivo metabolism, distribution, and binding of small amount of compounds. Radioactive isotopes of ¹H, ¹²C, ³¹P, ³²S, and ¹²⁷I in body are used such as ³H, ¹⁴C, ³²P, ³⁵S, and ¹²⁵I. In receptor fixation method in 96 well plates, receptors may be fixed in each well by using antibody or chemical methods and radioactive labeled ligands may be added to each well to induce binding: Unbound ligands may be washed out and then the standard can be determined by quantitative analysis of radioactivity of bound ligands or that of washed-out ligands. Then, addition of screening target compounds may induce competitive binding reaction with receptors. If the compounds show higher affinity to receptors than standard radioactive ligands, most of radioactive ligands would not bind to receptors and may be left in solution. Therefore, by analyzing quantity of bound radioactive ligands (or washed-out ligands), testing compounds' affinity to receptors can be indicated.

The filter membrane method may be needed when receptors cannot be fixed to 96 well plates or when ligand binding needs to be done in solution phase. In other words, after ligand-receptor binding reaction in solution, if the reaction solution is filtered through nitrocellulose filter paper, small molecules including ligands may go through it and only protein receptors may be left on the paper. Only ligands that strongly bound to receptors may stay on the filter paper and the relative affinity of added compounds can be identified by quantitative analysis of the standard radioactive ligands.

Some embodiments of the invention include fluorescence immunoassays for the analysis of PARP. Fluorescence based immunological methods are based upon the competitive binding of labeled ligands versus unlabeled ones on highly specific receptor sites. The fluorescence technique can be used for immunoassays based on changes in fluorescence lifetime with changing analyte concentration. This technique may work with short lifetime dyes like fluorescein isothiocyanate (FITC) (the donor) whose fluorescence may be quenched by energy transfer to eosin (the acceptor). A number of photoluminescent compounds may be used, such as cyanines, oxazines, thiazines, porphyrins, phthalocyanines, fluorescent infrared-emitting polynuclear aromatic hydrocarbons, phycobiliproteins, squaraines and organo-metallic complexes, hydrocarbons and azo dyes.

Fluorescence based immunological methods can be, for example, heterogenous or homogenous. Heterogenous immunoassays comprise physical separation of bound from free labeled analyte. The analyte or antibody may be attached to a solid surface. The technique can be competitive (for a higher selectivity) or noncompetitive (for a higher sensitivity). Detection can be direct (only one type of antibody used) or indirect (a second type of antibody is used). Homogenous immunoassays comprise no physical separation. Double-antibody fluorophore-labeled antigen participates in an equilibrium reaction with antibodies directed against both the antigen and the fluorophore. Labeled and unlabeled antigen may compete for a limited number of anti-antigen antibodies.

Some of the fluorescence immunoassay methods include simple fluorescence labeling method, fluorescence resonance energy transfer (FRET), time resolved fluorescence (TRF), and scanning probe microscopy (SPM). The simple fluorescence labeling method can be used for receptor-ligand binding, enzymatic activity by using pertinent fluorescence, and as a fluorescent indicator of various in vivo physiological changes such as pH, ion concentration, and electric pressure. TRF is a method that selectively measures fluorescence of the lanthanide series after the emission of other fluorescent molecules is finished. TRF can be used with FRET and the lanthanide series can become donors or acceptors. In scanning probe microscopy, in the capture phase, for example, at least one monoclonal antibody is adhered to a solid phase and a scanning probe microscope is utilized to detect antigen/antibody complexes which may be present on the surface of the solid phase. The use of scanning tunneling microscopy eliminates the need for labels which normally is utilized in many immunoassay systems to detect antigen/antibody complexes.

Protein identification methods: By way of example only, protein identification methods include low-throughput sequencing through Edman degradation, mass spectrometry techniques, peptide mass fingerprinting, de novo sequencing, and antibody-based assays. The protein quantification assays include fluorescent dye gel staining, tagging or chemical modification methods (i.e. isotope-coded affinity tags (ICATS), combined fractional diagonal chromatography (COFRADIC)). The purified protein may also be used for determination of three-dimensional crystal structure, which can be used for modeling intermolecular interactions. Common methods for determining three-dimensional crystal structure include x-ray crystallography and NMR spectroscopy. Characteristics indicative of the three-dimensional structure of proteins can be probed with mass spectrometry. By using chemical crosslinking to couple parts of the protein that are close in space, but far apart in sequence, information about the overall structure can be inferred. By following the exchange of amide protons with deuterium from the solvent, it is possible to probe the solvent accessibility of various parts of the protein.

In one embodiment, fluorescence-activated cell-sorting (FACS) is used to identify PARP expressing cells. FACS is a specialised type of flow cytometry. It provides a method for sorting a heterogenous mixture of biological cells into two or more containers, one cell at a time, based upon the specific light scattering and fluorescent characteristics of each cell. It provides quantitative recording of fluorescent signals from individual cells as well as physical separation of cells of particular interest. In yet another embodiment, microfluidic based devices are used to evaluate PARP expression.

Mass spectrometry can also be used to characterize PARP from patient samples. The two methods for ionization of whole proteins are electrospray ionization (ESI) and matrix-assisted laser desorption/ionization (MALDI). In the first, intact proteins are ionized by either of the two techniques described above, and then introduced to a mass analyser. In the second, proteins are enzymatically digested into smaller peptides using an agent such as trypsin or pepsin. Other proteolytic digest agents are also used. The collection of peptide products are then introduced to the mass analyser. This is often referred to as the “bottom-up” approach of protein analysis.

Whole protein mass analysis is conducted using either time-of-flight (TOF) MS, or Fourier transform ion cyclotron resonance (FT-ICR). The instrument used for peptide mass analysis is the quadrupole ion trap. Multiple stage quadrupole-time-of-flight and MALDI time-of-flight instruments also find use in this application.

Two methods used to fractionate proteins, or their peptide products from an enzymatic digestion. The first method fractionates whole proteins and is called two-dimensional gel electrophoresis. The second method, high performance liquid chromatography is used to fractionate peptides after enzymatic digestion. In some situations, it may be necessary to combine both of these techniques.

There are two ways mass spectroscopy can be used to identify proteins. Peptide mass uses the masses of proteolytic peptides as input to a search of a database of predicted masses that would arise from digestion of a list of known proteins. If a protein sequence in the reference list gives rise to a significant number of predicted masses that match the experimental values, there is some evidence that this protein was present in the original sample.

Tandem MS is also a method for identifying proteins. Collision-induced dissociation is used in mainstream applications to generate a set of fragments from a specific peptide ion. The fragmentation process primarily gives rise to cleavage products that break along peptide bonds.

A number of different algorithmic approaches have been described to identify peptides and proteins from tandem mass spectrometry (MS/MS), peptide de novo sequencing and sequence tag based searching. One option that combines a comprehensive range of data analysis features is PEAKS. Other existing mass spec analysis software include: Peptide fragment fingerprinting SEQUEST, Mascot, OMSSA and X!Tandem).

Proteins can also be quantified by mass spectrometry. Typically, stable (e.g. non-radioactive) heavier isotopes of carbon (C13) or nitrogen (N15) are incorporated into one sample while the other one is labelled with corresponding light isotopes (e.g. C12 and N14). The two samples are mixed before the analysis. Peptides derived from the different samples can be distinguished due to their mass difference. The ratio of their peak intensities corresponds to the relative abundance ratio of the peptides (and proteins). The methods for isotope labelling are SILAC (stable isotope labelling with amino acids in cell culture), trypsin-catalyzed 018 labeling, ICAT (isotope coded affinity tagging), ITRAQ (isotope tags for relative and absolute quantitation). “Semi-quantitative” mass spectrometry can be performed without labeling of samples. Typically, this is done with MALDI analysis (in linear mode). The peak intensity, or the peak area, from individual molecules (typically proteins) is here correlated to the amount of protein in the sample. However, the individual signal depends on the primary structure of the protein, on the complexity of the sample, and on the settings of the instrument.

N-terminal sequencing aids in the identification of unknown proteins, confirm recombinant protein identity and fidelity (reading frame, translation start point, etc.), aid the interpretation of NMR and crystallographic data, demonstrate degrees of identity between proteins, or provide data for the design of synthetic peptides for antibody generation, etc. N-terminal sequencing utilises the Edman degradative chemistry, sequentially removing amino acid residues from the N-terminus of the protein and identifying them by reverse-phase HPLC. Sensitivity can be at the level of 100 s femtomoles and long sequence reads (20-40 residues) can often be obtained from a few 10 s picomoles of starting material. Pure proteins (>90%) can generate easily interpreted data, but insufficiently purified protein mixtures may also provide useful data, subject to rigorous data interpretation. N-terminally modified (especially acetylated) proteins cannot be sequenced directly, as the absence of a free primary amino-group prevents the Edman chemistry. However, limited proteolysis of the blocked protein (e.g. using cyanogen bromide) may allow a mixture of amino acids to be generated in each cycle of the instrument, which can be subjected to database analysis in order to interpret meaningful sequence information. C-terminal sequencing is a post-translational modification, affecting the structure and activity of a protein. Various disease situations can be associated with impaired protein processing and C-terminal sequencing provides an additional tool for the investigation of protein structure and processing mechanisms.

### Identifying Diseases Treatable by PARP Inhibitors

Some embodiments of the present invention relate to identifying a disease treatable by PARP modulators comprising identifying a level of PARP in a sample of a subject, making a decision regarding identifying the disease treatable by the PARP modulators wherein the decision is made based on the level of PARP. The identification of the level of PARP may include analysis of RNA, analysis of level of PARP and/or analysis of PARP activity. When the level of PARP is up-regulated in a disease, the disease may be treated with PARP inhibitors. In some embodiments, PARP levels are used to identify angiogenesis related diseases.

In other embodiments of the present invention, the level of PARP is determined in samples from a patient population and compared with samples from a normal population in order to correlate any changes in PARP levels with the existence of a disease. The identification and analysis of the level of PARP may also include analysis of RNA, analysis of the level of PARP as well as analysis of PARP activity. When the level of PARP is increased in a number of samples from a patient population in comparison to samples from a normal population, the disease may be treated with PARP inhibitors. In some embodiments, a change of about 0.5 fold, about 1.0 fold, about 1.5 fold, about 2.0 fold, about 2.5 fold, about 3.0 fold, about 3.5 fold, about 4.0 fold, about 4.5 fold, or about 5.0 fold or more may indicate sufficient correlation of a change in PARP expression for a specific disease or group of diseases. In some embodiments the fold change is less than about 1, less than about 5, less than about 10, less than about 20, less than about 30, less than about 40, or less than about 50. In other embodiments, the changes in PARP level compared to a predetermined level is more than about 1, more than about 5, more than about 10, more than about 20, more than about 30, more than about 40, or more than about 50. Preferred fold changes from a pre-determined level are about 0.5, about 1.0, about 1.5, about 2.0, about 2.5, and about 3.0.

In some embodiments, the level of PARP from the plurality of samples may be averaged over the entire population in order to derive a predetermined value from which to compare the PARP levels from individual subjects. Such data manipulation is well known to those of ordinary skill in the art, and may take into account variances in the population, including geographical location of the population or individuals within the population, age, race, diet, financial status or other sociological factors that may impact the results. The plurality of samples may be pooled from a group of disease states, including by way of example only, breast cancer, or only one individual disease state, including by way of example only, intraductal carcinoma breast cancer. Moreover, the plurality of samples may consist of at least two subjects, at least five subjects, at least ten subjects, at least twenty subjects, at least fifty subjects, at least one hundred subjects, at least one thousand subjects. Alternatively, the population may consist of only one subject individual, including by way of example only, for rare diseases or for a disease where the patient population may be difficult to define.

In one embodiment, PARP upregulation is used as an embodiment of BRCA deficient cancer and PARP upregulation can be used to identify a BRCA mediated cancer treatable by PARP modulators. In another embodiment, the identification of a level of PARP is used as a marker of changes in regulation of DNA-repair of double-strand breaks by homologous recombination (HR) and the level of PARP is used to make a decision regarding identifying a disease treatable by the PARP modulators. The identification of a level of PARP may involve one or more comparisons with reference samples. The reference samples may be obtained from the same subject or from a different subject who is either not affected with the disease (such as, normal subject) or is a patient. The reference sample could be obtained from one subject, multiple subjects or is synthetically generated. The identification may also involve the comparison of the identification data with the databases. One embodiment of the invention relates to identifying the level of PARP in a subject afflicted with disease and correlating it with the PARP level of the normal subjects. In some embodiments, the step of correlating the level of PARP is performed by a software algorithm. Preferably, the data generated is transformed into computer readable form; and an algorithm is executed that classifies the data according to user input parameters, for detecting signals that represent level of PARP in diseased patients and PARP levels in normal subjects.

In one embodiment, PARP upregulation is used as an embodiment of BRCA deficient cancer and PARP upregulation can be used to identify a BRCA mediated cancer treatable by PARP modulators. In another embodiment, the identification of a level of PARP is used as a marker of changes in regulation of DNA-repair of double-strand breaks by homologous recombination (HR) and the level of PARP is used to make a decision regarding identifying a disease treatable by the PARP modulators. The identification of a level of PARP in each of the plurality of samples may involve one or more comparisons with reference samples. The reference samples may be obtained from the same subject or from a different subject who is either not affected with the disease (such as, normal subject) or is a patient. The reference sample could be obtained from one subject, multiple subjects or is synthetically generated. The identification may also involve the comparison of the identification data with the databases. One embodiment of the invention relates to identifying the level of PARP in a subject or a patient population afflicted with disease and correlating it with the PARP level of normal subjects and/or a normal population. In some embodiments, the step of correlating the level of PARP is performed by a software algorithm. Preferably, the data generated is transformed into computer readable form; and an algorithm is executed that classifies the data according to user input parameters, for detecting signals that represent level of PARP in diseased patients or patient populations, and correspondingly PARP levels in normal subjects or populations.

The identification and analysis of the level of PARP have numerous therapeutic and diagnostic applications. Clinical applications include, for example, detection of disease, distinguishing disease states to inform prognosis, selection of therapy such as, treatment with PARP inhibitors, and/or prediction of therapeutic response, disease staging, identification of disease processes, prediction of efficacy of therapy, monitoring of patients trajectories (e.g., prior to onset of disease), prediction of adverse response, monitoring of therapy associated efficacy and toxicity, and detection of recurrence.

The identification of the level of PARP and the subsequent identification of a disease in a subject treatable by PARP inhibitors, as disclosed in the present invention can be used to enable or assist in the pharmaceutical drug development process for therapeutic agents. The identification of the level of PARP can be used to diagnose disease for patients enrolling in a clinical trial. The identification of the level of PARP can indicate the state of the disease of patients undergoing treatment in clinical trials, and show changes in the state during the treatment. The identification of the level of PARP can demonstrate the efficacy of treatment with PARP inhibitors, and can be used to stratify patients according to their responses to various therapies.

The identification of the level of PARP and the subsequent identification of a disease in a subject or subject population treatable by PARP inhibitors, as disclosed in the present invention can be used to enable or assist in the pharmaceutical drug development process for therapeutic agents. The identification of the level of PARP can be used to diagnose disease for patients enrolling in a clinical trial, for example in a patient population. The identification of the level of PARP can indicate the state of the disease of patients undergoing treatment in clinical trials, and show changes in the state during the treatment. The identification of the level of PARP can demonstrate the efficacy of treatment with PARP inhibitors, and can be used to stratify patients according to their responses to various therapies.

The methods described herein can be used to identify the state of a disease in a patient. In one embodiment, the methods are used to detect the earliest stages of disease. In other embodiments, the methods are used to grade the identified disease. In certain embodiments, patients, health care providers, such as doctors and nurses, or health care managers, use the level of PARP in a subject to make a diagnosis, prognosis, and/or select treatment options, such as treatment with PARP inhibitors.

The methods described herein can be used to identify the state of a disease in either an individual patient or a patient population. In one embodiment, the methods are used to detect the earliest stages of disease. In other embodiments, the methods are used to grade the identified disease. In certain embodiments, patients, health care providers, such as doctors and nurses, or health care managers, use the level of PARP in a subject to make a diagnosis, prognosis, and/or select treatment options, such as treatment with PARP inhibitors. In other embodiments, health care providers and patients may use the level of PARP obtained in a patient population to also make a diagnosis, prognosis, and/or select treatment options, such as treatment with PARP inhibitors.

In other embodiments, the methods described herein can be used to predict the likelihood of response for any individual to a particular treatment (such as treatment with PARP inhibitors), select a treatment, or to preempt the possible adverse effects of treatments on a particular individual. Also, the methods can be used to evaluate the efficacy of treatments over time. For example, biological samples can be obtained from a patient over a period of time as the patient is undergoing treatment. The level of PARP in the different samples can be compared to each other to determine the efficacy of the treatment. Also, the methods described herein can be used to compare the efficacies of different disease therapies and/or responses to one or more treatments in different populations (e.g., ethnicities, family histories, etc.).

In other embodiments, the methods described herein can be used to predict the likelihood of response for any individual or patient population to a particular treatment (such as treatment with PARP inhibitors), select a treatment, or to preempt the possible adverse effects of treatments on a particular individual. Also, the methods can be used to evaluate the efficacy of treatments over time. For example, biological samples can be obtained from a patient or from a plurality of patients in a population over a period of time as each patient is undergoing treatment. The level of PARP in the different samples can be compared to each other to determine the efficacy of the treatment. Also, the methods described herein can be used to compare the efficacies of different disease therapies and/or responses to one or more treatments in different populations (e.g., ethnicities, family histories, etc.).

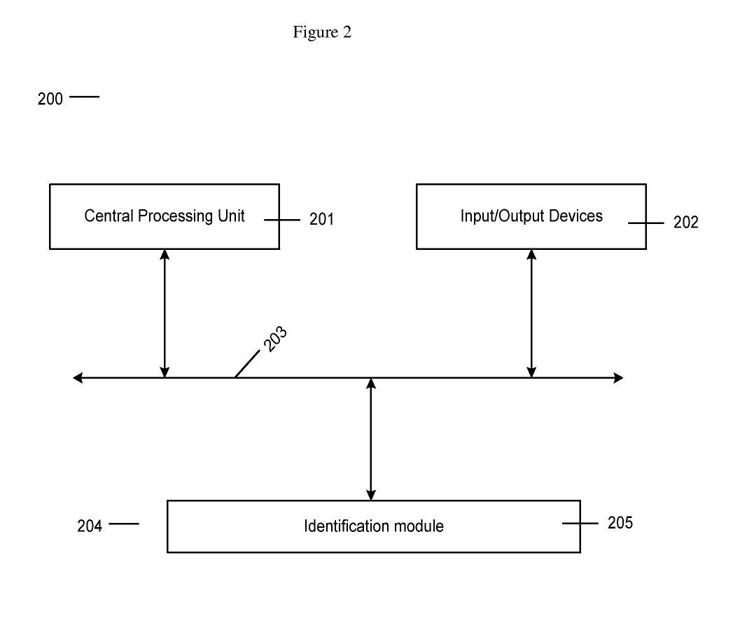

In some preferred embodiments, at least one step of the methods of the present invention is performed using a computer as depicted in FIG. 2. FIG. 2 illustrates a computer for implementing selected operations associated with the methods of the present invention. The computer 200 includes a central processing unit 201 connected to a set of input/output devices 202 via a system bus 203. The input/output devices 202 may include a keyboard, mouse, scanner, data port, video monitor, liquid crystal display, printer, and the like. A memory 204 in the form of primary and/or secondary memory is also connected to the system bus 203. These components of FIG. 2 characterize a standard computer. This standard computer is programmed in accordance with the invention. In particular, the computer 200 can be programmed to perform various operations of the methods of the present invention.

The memory 204 of the computer 200 may store an identification module 205. In other words, the identification module 205 can perform the operations associated with step 102, 103, and 104 of FIG. 1. The term “identification module” used herein includes, but is not limited to, analyzing PARP in a sample of a subject; optionally comparing the PARP level data of the test sample with the reference sample; identifying the level of PARP in the sample; identifying the disease; and further identifying the disease treatable by PARP inhibitors. The identification module may also include a decision module where the decision module includes executable instructions to make a decision regarding identifying the disease treatable by PARP inhibitors and/or provide a conclusion regarding the disease to a patient, a health care provider or a health care manager. The executable code of the identification module 205 may utilize any number of numerical techniques to perform the comparisons and diagnosis.

Some embodiments of the present invention include a computer readable medium with information regarding a disease in a subject treatable by PARP modulators, the information being derived by identifying a level of PARP in the sample of the subject, and making a decision based on the level of PARP regarding treating the disease by the PARP modulators. The medium may contain a reference pattern of one or more of levels of PARP in a sample. This reference pattern can be used to compare the pattern obtained from a test subject and an analysis of the disease can be made based on this comparison. This reference pattern can be from normal subjects, i.e., subjects with no disease, subjects with different levels of disease, subjects with disease of varying severity. These reference patterns can be used for diagnosis, prognosis, evaluating efficacy of treatment, and/or determining the severity of the disease state of a subject. The methods of the present invention also include sending information regarding levels of PARP in a sample in a subject and/or decision regarding identifying the disease treatable by PARP inhibitors of the present invention, between one or more computers, for example with the use of the internet.

Some embodiments of the present invention include a computer readable medium with information regarding a disease treatable by PARP modulators, the information being derived by identifying a level of PARP in plurality of samples from a population, and making a decision based on the level of PARP regarding treating the disease by the PARP modulators. The medium may contain a reference pattern of one or more of levels of PARP in a sample. This reference pattern can be used to compare the pattern obtained from a test subject and an analysis of the disease can be made based on this comparison. This reference pattern can be from normal subjects, i.e., subjects with no disease, subjects with different levels of disease, subjects with disease of varying severity. These reference patterns can be used for diagnosis, prognosis, evaluating efficacy of treatment, and/or determining the severity of the disease state of a subject. The methods of the present invention also include sending information regarding levels of PARP in a sample in a subject or patient population and/or decision regarding identifying the disease treatable by PARP inhibitors of the present invention, between one or more computers, for example with the use of the internet.

### Diseases

Various disease include, but are not limited to, cancer types including adrenal cortical cancer, anal cancer, aplastic anemia, bile duct cancer, bladder cancer, bone cancer, bone metastasis, adult CNS brain tumors, children CNS brain tumors, breast cancer, castleman disease, cervical cancer, childhood Non-Hodgkin's lymphoma, colon and rectum cancer, endometrial cancer, esophagus cancer, Ewing's family of tumors, eye cancer, gallbladder cancer, gastrointestianl carcinoid tumors, gastrointestinal stromal tumors, gestational trophoblastic disease, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, nonmelanoma skin cancer, stomach cancer, testicular cancer, thymus cancer, thyroid cancer, uterine sarcoma, vaginal cancer, vulvar cancer, Waldenstrom's macroglobulinemia, chronic lymphocyte leukemia, and reactive lymphoid hyperplasia.

Diseases include angiogenesis in cancers, inflammation, degenerative diseases, CNS diseases, autoimmune diseases, and viral diseases, including HIV. The compounds described herein are also useful in the modulation of cellular response to pathogens. The invention also provides methods to treat other diseases, such as, viral diseases. Some of the viral diseases are, but not limited to, human immunodeficiency virus (HIV), herpes simplex virus type-1 and 2 and cytomegalovirus (CMV), a dangerous co-infection of HIV.

Some examples of the diseases are set forth herein, but without limiting the scope of the present invention, there may be other diseases known in the art and are within the scope of the present invention.

Examples of Cancer

Examples of cancers include, but are not limited to, lymphomas, carcinomas and hormone-dependent tumors (e.g., breast, prostate or ovarian cancer). Abnormal cellular proliferation conditions or cancers that may be treated in either adults or children include solid phase tumors/malignancies, locally advanced tumors, human soft tissue sarcomas, metastatic cancer, including lymphatic metastases, blood cell malignancies including multiple myeloma, acute and chronic leukemias, and lymphomas, head and neck cancers including mouth cancer, larynx cancer and thyroid cancer, lung cancers including small cell carcinoma and non-small cell cancers, breast cancers including small cell carcinoma and ductal carcinoma, gastrointestinal cancers including esophageal cancer, stomach cancer, colon cancer, colorectal cancer and polyps associated with colorectal neoplasia, pancreatic cancers, liver cancer, urologic cancers including bladder cancer and prostate cancer, malignancies of the female reproductive tract including ovarian carcinoma, uterine (including endometrial) cancers, and solid tumor in the ovarian follicle, kidney cancers including renal cell carcinoma, brain cancers including intrinsic brain tumors, neuroblastoma, astrocytic brain tumors, gliomas, metastatic tumor cell invasion in the central nervous system, bone cancers including osteomas, skin cancers including malignant melanoma, tumor progression of human skin keratinocytes, squamous cell carcinoma, basal cell carcinoma, hemangiopericytoma and Karposi's sarcoma.

In some preferred embodiments of the present invention, cancer includes colon adenocarcinoma, esophagus adenocarcinoma, liver hepatocellular carcinoma, squamous cell carcinoma, pancreas adenocarcinoma, islet cell tumor, rectum adenocarcinoma, gastrointestinal stromal tumor, stomach adenocarcinoma, adrenal cortical carcinoma, follicular carcinoma, papillary carcinoma, breast cancer, ductal carcinoma, lobular carcinoma, intraductal carcinoma, mucinous carcinoma, phyllodes tumor, ovarian adenocarcinoma, endometrium adenocarcinoma, granulose cell tumor, mucinous cystadenocarcinoma, cervix adenocarcinoma, vulva squamous cell carcinoma, basal cell carcinoma, prostate adenocarcinoma, giant cell tumor of bone, bone osteosarcoma, larynx carcinoma, lung adenocarcinoma, kidney carcinoma, urinary bladder carcinoma, and Wilm's tumor.

In still further preferred embodiments of the present invention, cancer includes mullerian mixed tumor of the endometrium, infiltrating carcinoma of mixed ductal and lobular type, Wilm's tumor, mullerian mixed tumor of the ovary, serous cystadenocarcinoma, ovary adenocarcinoma (papillary serous type), ovary adenocarcinoma (endometrioid type), metastatic infiltrating lobular carcinoma of breast, testis seminoma, prostate benign nodular hyperplasia, lung squamous cell carcinoma, lung large cell carcinoma, lung adenocarcinoma, endometrium adenocarcinoma (endometrioid type), infiltrating ductal carcinoma, skin basal cell carcinoma, breast infiltrating lobular carcinoma, fibrocystic disease, fibroadenoma, glioma, chronic myeloid leukemia, liver hepatocellular carcinoma, mucinous carcinoma, schwannoma, kidney transitional cell carcinoma, Hashimoto's thyroiditis, metastatic infiltrating ductal carcinoma of breast, esophagus adenocarcinoma, thymoma, phyllodes tumor, rectum adenocarcinoma, osteosarcoma, colon adenocarcinoma, thyroid gland papillary carcinoma, leiomyoma, and stomach adenocarcinoma.

Infiltrating Duct Carcinoma:

The expression of PARP1 in infiltrating duct carcinoma (IDC) of the breast was elevated compared to normals. In more than two-thirds of IDC cases PARP1 expression was above the 95% upper confidence limit of the normal population (“over-expression”). Estrogen receptor (ER)-negative and Her2-neu-negative subgroups of IDC had an incidence of PARP1 over-expression in approximately 90% of tumors.

In one aspect of the invention, IDC is treated with PARP inhibitors. In one embodiment, PARP expression and ER and/or progesterone receptor (PR) and/or Her2-neu status is evaluated, prior to administration of a PARP inhibitor. Preferably, PARP inhibitors are used to treat estrogen receptor-negative and Her2-neu-negative subgroups of IDC. Even more preferably, PARP inhibitors are used to treat cancers that do not qualify for anti-estrogen or anti-Her2-neu therapies. In a preferred embodiment, PARP inhibitors are used to treat triple negative breast cancers, such as triple negative infiltrating duct carcinomas.

Triple Negative Cancers:

In one embodiment, triple negative cancers are treated with PARP inhibitors. Preferably, the level of PARP is evaluated in the triple negative cancer and if an over expression of PARP is observed, the cancer is treated with a PARP inhibitor. “Triple negative” breast cancer, means the tumors lack receptors for the hormones estrogen (ER-negative) and progesterone (PR-negative), and for the protein HER2. This makes them resistant to several powerful cancer-fighting drugs like tamoxifen, aromatase inhibitors, and Herceptin. Surgery and chemotherapy are standard treatment options for most forms of triple-negative cancer. In a preferred embodiment, the standard of care for triple negative cancers is combined with PARP inhibitors to treat these cancers.

Examples of Inflammation

Examples of inflammation include, but are not limited to, systemic inflammatory conditions and conditions associated locally with migration and attraction of monocytes, leukocytes and/or neutrophils. Inflammation may result from infection with pathogenic organisms (including gram-positive bacteria, gram-negative bacteria, viruses, fungi, and parasites such as protozoa and helminths), transplant rejection (including rejection of solid organs such as kidney, liver, heart, lung or cornea, as well as rejection of bone marrow transplants including graft-versus-host disease (GVHD)), or from localized chronic or acute autoimmune or allergic reactions. Autoimmune diseases include acute glomerulonephritis; rheumatoid or reactive arthritis; chronic glomerulonephritis; inflammatory bowel diseases such as Crohn's disease, ulcerative colitis and necrotizing enterocolitis; granulocyte transfusion associated syndromes; inflammatory dermatoses such as contact dermatitis, atopic dermatitis, psoriasis; systemic lupus erythematosus (SLE), autoimmune thyroiditis, multiple sclerosis, and some forms of diabetes, or any other autoimmune state where attack by the subject's own immune system results in pathologic tissue destruction. Allergic reactions include allergic asthma, chronic bronchitis, acute and delayed hypersensitivity. Systemic inflammatory disease states include inflammation associated with trauma, burns, reperfusion following ischemic events (e.g. thrombotic events in heart, brain, intestines or peripheral vasculature, including myocardial infarction and stroke), sepsis, ARDS or multiple organ dysfunction syndrome. Inflammatory cell recruitment also occurs in atherosclerotic plaques.

In some preferred embodiments, the inflammation includes Non-Hodgkin's lymphoma, Wegener's granulomatosis, Hashimoto's thyroiditis, hepatocellular carcinoma, thymus atrophy, chronic pancreatitis, rheumatoid arthritis, reactive lymphoid hyperplasia, osteoarthritis, ulcerative colitis, papillary carcinoma, Crohn's disease, ulcerative colitis, acute cholecystitis, chronic cholecystitis, cirrhosis, chronic sialadenitis, peritonitis, acute pancreatitis, chronic pancreatitis, chronic Gastritis, adenomyosis, endometriosis, acute cervicitis, chronic cervicitis, lymphoid hyperplasia, multiple sclerosis, hypertrophy secondary to idiopathic thrombocytopenic purpura, primary IgA nephropathy, systemic lupus erythematosus, psoriasis, pulmonary emphysema, chronic pyelonephritis, and chronic cystitis.

Examples of Endocrine and Neuroendocrine Disorders

Examples of endocrine disorders include disorders of adrenal, breast, gonads, pancreas, parathyroid, pituitary, thyroid, dwarfism etc. The adrenal disorders include, but are not limited to, Addison's disease, hirsutism, cancer, multiple endocrine neoplasia, congenital adrenal hyperplasia, and pheochromocytoma. The breast disorders include, but are not limited to, breast cancer, fibrocystic breast disease, and gynecomastia. The gonad disorders include, but are not limited to, congenital adrenal hyperplasia, polycystic ovarian syndrome, and turner syndrome. The pancreas disorders include, but are not limited to, diabetes (type I and type II), hypoglycemia, and insulin resistance. The parathyroid disorders include, but are not limited to, hyperparathyroidism, and hypoparathyroidism. The pituitary disorders include, but are not limited to, acromegaly, Cushing's syndrome, diabetes insipidus, empty sella syndrome, hypopituitarism, and prolactinoma. The thyroid disorders include, but are not limited to, cancer, goiter, hyperthyroid, hypothyroid, nodules, thyroiditis, and Wilson's syndrome. The examples of neuroendocrine disorders include, but are not limited to, depression and anxiety disorders related to a hormonal imbalance, catamenial epilepsy, menopause, menstrual migraine, reproductive endocrine disorders, gastrointestinal disorders such as, gut endocrine tumors including carcinoid, gastrinoma, and somatostatinoma, achalasia, and Hirschsprung's disease. In some embodiments, the endocrine and neuroendocrine disorders include nodular hyperplasia, Hashimoto's thyroiditis, islet cell tumor, and papillary carcinoma.

The endocrine and neuroendocrine disorders in children include endocrinologic conditions of growth disorder and diabetes insipidus. Growth delay may be observed with congenita ectopic location or aplasia/hypoplasia of the pituitary gland, as in holoprosencephaly, septo-optic dysplasia and basal encephalocele. Acquired conditions, such as craniopharyngioma, optic/hypothalamic glioma may be present with clinical short stature and diencephalic syndrome. Precocious puberty and growth excess may be seen in the following conditions: arachnoid cyst, hydrocephalus, hypothalamic hamartoma and germinoma. Hypersecretion of growth hormone and adrenocorticotropic hormone by a pituitary adenoma may result in pathologically tall stature and truncal obesity in children. Diabetes insipidus may occur secondary to infiltrative processes such as langerhans cell of histiocytosis, tuberculosis, germinoma, post traumatic/surgical injury of the pituitary stalk and hypoxic ischemic encephalopathy.

Examples of Nutritional and Metabolic Disorders

The examples of nutritional and metabolic disorders include, but are not limited to, aspartylglusomarinuria, biotinidase deficiency, carbohydrate deficient glycoprotein syndrome (CDGS), Crigler-Najjar syndrome, cystinosis, diabetes insipidus, fabry, fatty acid metabolism disorders, galactosemia, gaucher, glucose-6-phosphate dehydrogenase (G6PD), glutaric aciduria, hurler, hurler-scheie, hunter, hypophosphatemia, I-cell, krabbe, lactic acidosis, long chain 3 hydroxyacyl CoA dehydrogenase deficiency (LCHAD), lysosomal storage diseases, mannosidosis, maple syrup urine, maroteaux-lamy, metachromatic leukodystrophy, mitochondrial, morquio, mucopolysaccharidosis, neuro-metabolic, niemann-pick, organic acidemias, purine, phenylketonuria (PKU), pompe, pseudo-hurler, pyruvate dehydrogenase deficiency, sandhoff, sanfilippo, scheie, sly, tay-sachs, trimethylaminuria (fish-malodor syndrome), urea cycle conditions, vitamin D deficiency rickets, metabolic disease of muscle, inherited metabolic disorders, acid-base imbalance, acidosis, alkalosis, alkaptonuria, alpha-mannosidosis, amyloidosis, anemia, iron-deficiency, ascorbic acid deficiency, avitaminosis, beriberi, biotimidase deficiency, deficient glycoprotein syndrome, carnitine disorders, cystinosis, cystinuria, fabry disease, fatty acid oxidation disorders, fucosidosis, galactosemias, gaucher disease, gilbert disease, glucosephosphate dehydrogenase deficiency, glutaric academia, glycogen storage disease, hartnup disease, hemochromatosis, hemosiderosis, hepatolenticular degeneration, histidinemia, homocystinuria, hyperbilirubinemia, hypercalcemia, hyperinsulinism, hyperkalemia, hyperlipidemia, hyperoxaluria, hypervitaminosis A, hypocalcemia, hypoglycemia, hypokalemia, hyponatremia, hypophosphotasia, insulin resistance, iodine deficiency, iron overload, jaundice, chronic idiopathic, leigh disease, Lesch-Nyhan syndrome, leucine metabolism disorders, lysosomal storage diseases, magnesium deficiency, maple syrup urine disease, MELAS syndrome, menkes kinky hair syndrome, metabolic syndrome X, mucolipidosis, mucopolysacchabridosis, Niemann-Pick disease, obesity, ornithine carbamoyltransferase deficiency disease, osteomalacia, pellagra, peroxisomal disorders, porphyria, erythropoietic, porphyries, progeria, pseudo-gaucher disease, refsum disease, reye syndrome, rickets, sandhoff disease, tangier disease, Tay-sachs disease, tetrahydrobiopterin deficiency, trimethylaminuria (fish odor syndrome), tyrosinemias, urea cycle disorders, water-electrolyte imbalance, wernicke encephalopathy, vitamin A deficiency, vitamin B12 deficiency, vitamin B deficiency, wolman disease, and zellweger syndrome.

In some preferred embodiments, the metabolic diseases include diabetes and obesity.

Examples of Hematolymphoid System

A hematolymphoid system includes hemic and lymphatic diseases. A “hematological disorder” includes a disease, disorder, or condition which affects a hematopoietic cell or tissue. Hematological disorders include diseases, disorders, or conditions associated with aberrant hematological content or function. Examples of hematological disorders include disorders resulting from bone marrow irradiation or chemotherapy treatments for cancer, disorders such as pernicious anemia, hemorrhagic anemia, hemolytic anemia, aplastic anemia, sickle cell anemia, sideroblastic anemia, anemia associated with chronic infections such as malaria, trypanosomiasis, HIV, hepatitis virus or other viruses, myelophthisic anemias caused by marrow deficiencies, renal failure resulting from anemia, anemia, polycethemia, infectious mononucleosis (IM), acute non-lymphocytic leukemia (ANLL), acute Myeloid Leukemia (AML), acute promyelocytic leukemia (APL), acute myelomonocytic leukemia (AMMoL), polycethemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalessemia, antibody-mediated disorders such as transfusion reactions and erythroblastosis, mechanical trauma to red blood cells such as micro-angiopathic hemolytic anemias, thrombotic thrombocytopenic purpura and disseminated intravascular coagulation, infections by parasites such as plasmodium, chemical injuries from, e.g., lead poisoning, and hypersplenism.

Lymphatic diseases include, but are not limited to, lymphadenitis, lymphagiectasis, lymphangitis, lymphedema, lymphocele, lymphoproliferative disorders, mucocutaneous lymph node syndrome, reticuloendotheliosis, splenic diseases, thymus hyperplasia, thymus neoplasms, tuberculosis, lymph node, pseudolymphoma, and lymphatic abnormalities.

In some preferred embodiments, the disorders of hematolymphoid system include, non-Hodgkin's lymphoma, chronic lymphocytic leukemia, and reactive lymphoid hyperplasia.

Examples of CNS Diseases

The examples of CNS diseases include, but are not limited to, neurodegenerative diseases, drug abuse such as, cocaine abuse, multiple sclerosis, schizophrenia, acute disseminated encephalomyelitis, transverse myelitis, demyelinating genetic diseases, spinal cord injury, virus-induced demyelination, progressive multifocal leucoencephalopathy, human lymphotrophic T-cell virus I (HTLVI)-associated myelopathy, and nutritional metabolic disorders.

In some preferred embodiments, the CNS diseases include Parkinson disease, Alzheimer's disease, cocaine abuse, and schizophrenia.

Examples of Neurodegenerative Diseases

Neurodegenerative diseases in the methods of the present invention include, but are not limited to, Alzheimer's disease, Pick's disease, diffuse lewy body disease, progressive supranuclear palsy (Steel-Richardson syndrome), multisystem degeneration (Shy-Drager syndrome), motor neuron diseases including amyotrophic lateral sclerosis, degenerative ataxias, cortical basal degeneration, ALS-Parkinson's-dementia complex of guam, subacute sclerosing panencephalitis, Huntington's disease, Parkinson's disease, synucleinopathies, primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, and prion diseases (including Creutzfeldt-Jakob, Gerstmann-Straussler-Scheinker disease, kuru and fatal familial insomnia), Alexander disease, alper's disease, amyotrophic lateral sclerosis, ataxia telangiectasia, batten disease, canavan disease, cockayne syndrome, corticobasal degeneration, Creutzfeldt-Jakob disease, Huntington disease, Kennedy's disease, Krabbe disease, lewy body dementia, Machado-Joseph disease, spinocerebellar ataxia type 3, multiple sclerosis, multiple system atrophy, Parkinson disease, Pelizaeus-Merzbacher Disease, Refsum's disease, Schilder's disease, Spielmeyer-Vogt-Sjogren-Batten disease, Steele-Richardson-Olszewski disease, and tabes dorsalis.

Examples of Disorders of Urinary Tract

Disorders of urinary tract in the methods of the present invention include, but are not limited to, disorders of kidney, ureters, bladder, and urethera. For example, urethritis, cystitis, pyelonephritis, renal agenesis, hydronephrosis, polycystic kidney disease, multicystic kidneys, low urinary tract obstruction, bladder exstrophy and epispadias, hypospadias, bacteriuria, prostatitis, intrarenal and peripheral abscess, benign prostate hypertrophy, renal cell carcinoma, transitional cell carcinoma, Wilm's tumor, uremia, and glomerolonephritis.

Examples of Respiratory Diseases

The respiratory diseases and conditions include, but are not limited to, asthma, chronic obstructive pulmonary disease (COPD), adenocarcinoma, adenosquamous carcinoma, squamous cell carcinoma, large cell carcinoma, cystic fibrosis (CF), dispnea, emphysema, wheezing, pulmonary hypertension, pulmonary fibrosis, hyper-responsive airways, increased adenosine or adenosine receptor levels, pulmonary bronchoconstriction, lung inflammation and allergies, and surfactant depletion, chronic bronchitis, bronchoconstriction, difficult breathing, impeded and obstructed lung airways, adenosine test for cardiac function, pulmonary vasoconstriction, impeded respiration, acute respiratory distress syndrome (ARDS), administration of certain drugs, such as adenosine and adenosine level increasing drugs, and other drugs for, e.g. treating supraventricular tachycardia (SVT), and the administration of adenosine stress tests, infantile respiratory distress syndrome (infantile RDS), pain, allergic rhinitis, decreased lung surfactant, decreased ubiquinone levels, or chronic bronchitis, among others.

Examples of Disorders of Female Reproductive System

The disorders of the female reproductive system include diseases of the vulva, vagina, cervix uteri, corpus uteri, fallopian tube, and ovary. Some of the examples include, adnexal diseases such as, fallopian tube disease, ovarian disease, leiomyoma, mucinous cystadenocarcinoma, serous cystadenocarcinoma, parovarian cyst, and pelvic inflammatory disease; endometriosis; reproductive neoplasms such as, fallopian tube neoplasms, uterine neoplasms, vaginal neoplasms, vulvar neoplasms, and ovarian neoplasms; gynatresia; reproductive herpes; infertility; sexual dysfunction such as, dyspareunia, and impotence; tuberculosis; uterine diseases such as, cervix disease, endometrial hyperplasia, endometritis, hematometra, uterine hemorrhage, uterine neoplasms, uterine prolapse, uterine rupture, and uterine inversion; vaginal diseases such as, dyspareunia, hematocolpos, vaginal fistula, vaginal neoplasms, vaginitis, vaginal discharge, and candidiasis or vulvovaginal; vulvar diseases such as, kraurosis vulvae, pruritus, vulvar neoplasm, vulvitis, and candidiasis; and urogenital diseases such as urogenital abnormalities and urogenital neoplasms.

Examples of Disorders of Male Reproductive System

The disorders of the male reproductive system include, but are not limited to, epididymitis; reproductive neoplasms such as, penile neoplasms, prostatic neoplasms, and testicular neoplasms; hematocele; reproductive herpes; hydrocele; infertility; penile diseases such as, balanitis, hypospadias, peyronie disease, penile neoplasms, phimosis, and priapism; prostatic diseases such as, prostatic hyperplasia, prostatic neoplasms, and prostatitis; organic sexual dysfunction such as, dyspareunia, and impotence; spermatic cord torsion; spermatocele; testicular diseases such as, cryptorchidism, orchitis, and testicular neoplasms; tuberculosis; varicocele; urogenital diseases such as, urogenital abnormalities, and urogenital neoplasms; and fournier gangrene.

Examples of Cardiovascular Disorders (CVS)

The cardiovascular disorders include those disorders that can either cause ischemia or are caused by reperfusion of the heart. Examples include, but are not limited to, atherosclerosis, coronary artery disease, granulomatous myocarditis, chronic myocarditis (non-granulomatous), primary hypertrophic cardiomyopathy, peripheral artery disease (PAD), stroke, angina pectoris, myocardial infarction, cardiovascular tissue damage caused by cardiac arrest, cardiovascular tissue damage caused by cardiac bypass, cardiogenic shock, and related conditions that would be known by those of ordinary skill in the art or which involve dysfunction of or tissue damage to the heart or vasculature, especially, but not limited to, tissue damage related to PARP activation.

In some preferred embodiments of the present invention, CVS diseases include, atherosclerosis, granulomatous myocarditis, myocardial infarction, myocardial fibrosis secondary to valvular heart disease, myocardial fibrosis without infarction, primary hypertrophic cardiomyopathy, and chronic myocarditis (non-granulomatous).

Method of Treatment with PARP Inhibitors

PARP inhibitors have potential therapeutic benefit when used independently in the treatment of various diseases such as, myocardial ischemia, stroke, head trauma, and neurodegenerative disease, and as an adjunct therapy with other agents including chemotherapeutic agents, radiation, oligonucleotides, or antibodies in cancer therapy. Without limiting the scope of the present invention, it shall be understood that various PARP inhibitors are known in the art and are all within the scope of the present invention. Some of the examples of PARP inhibitors are disclosed herein but they are not in any way limiting to the scope of the present invention.

A great preponderance of PARP inhibitors have been designed as analogs of benzamides, which bind competitively with the natural substrate NAD in the catalytic site of PARP. The PARP inhibitors include, but are not limited to, benzamides, quinolones and isoquinolones, benzopyrones, methyl 3,5-diiodo-4-(4′-methoxyphenoxy)benzoate, and methyl-3,5-diiodo-4-(4′-methoxy-3′,5′-diiodo-phenoxy)benzoate (U.S. Pat. No. 5,464,871, U.S. Pat. No. 5,670,518, U.S. Pat. No. 6,004,978, U.S. Pat. No. 6,169,104, U.S. Pat. No. 5,922,775, U.S. Pat. No. 6,017,958, U.S. Pat. No. 5,736,576, and U.S. Pat. No. 5,484,951, all incorporated herein in their entirety). The PARP inhibitors include a variety of cyclic benzamide analogs (i.e. lactams) which are potent inhibitors at the NAD site. Other PARP inhibitors include, but are not limited to, benzimidazoles and indoles (EP841924, EP1127052, U.S. Pat. No. 6,100,283, U.S. Pat. No. 6,310,082, US2002/156050, US2005/054631, WO05/012305, WO99/11628, and US2002/028815). A number of low-molecular-weight inhibitors of PARP have been used to elucidate the functional role of poly ADP-ribosylation in DNA repair. In cells treated with alkylating agents, the inhibition of PARP leads to a marked increase in DNA-strand breakage and cell killing (Durkacz et al, 1980, Nature 283: 593-596; and Berger, N. A., 1985, Radiation Research, 101: 4-14). Subsequently, such inhibitors have been shown to enhance the effects of radiation response by suppressing the repair of potentially lethal damage (Ben-Hur et al, 1984, British Journal of Cancer, 49 (Suppl. VI): 34-42; and Schlicker et al, 1999, Int. J. Radiat. Bioi., 75: 91-100). PARP inhibitors have been reported to be effective in radio sensitising hypoxic tumour cells (U.S. Pat. Nos. 5,032,617, 5,215,738 and 5,041,653). Furthermore, PARP knockout (PARP −/−) animals exhibit genomic instability in response to alkylating agents and γ-irradiation (Wang et al, 1995, Genes Dev., 9: 509-520; and Menissier de Murcia et al, 1997, Proc. Natl. Acad. Sci. USA, 94: 7303-7307).

Oxygen radical DNA damage that leads to strand breaks in DNA, which are subsequently recognised by PARP, is a major contributing factor to such disease states as shown by PARP inhibitor studies (Cosi et al, 1994, J. Neurosci. Res., 39: 38-46; and Said et al, 1996, Proc. Natl. Acad. Sci. U.S.A., 93: 4688-4692). It has also been demonstrated that efficient retroviral infection of mammalian cells is blocked by the inhibition of PARP activity. Such inhibition of recombinant retroviral vector infections was shown to occur in various different cell types (Gaken et al, 1996, J. Virology, 70(6): 3992-4000). Inhibitors of PARP have thus been developed for the use in anti-viral therapies and in cancer treatment (WO91/18591). Moreover, PARP inhibition has been speculated to delay the onset of aging characteristics in human fibroblasts (Rattan and Clark, 1994, Biochem. Biophys. Res. Comm., 201 (2): 665-672). This may be related to the role that PARP plays in controlling telomere function (d'Adda di Fagagna et al, 1999, Nature Gen., 23(1): 76-80).

PARP inhibitors may possess following structural characteristics: 1) amide or lactam functionality; 2) an NH proton of this amide or lactam functionality could be conserved for effective bonding; 3) an amide group attached to an aromatic ring or a lactam group fused to an aromatic ring; 4) optimal cis-configuration of the amide in the aromatic plane; and 5) constraining mono-aryl carboxamide into heteropolycyclic lactams (Costantino et al., 2001, J Med. Chem., 44:3786-3794). Virag et al., 2002, Pharmacol Rev., 54:375-429, 2002 summarizes various PARP inhibitors. Some of the examples of PARP inhibitors include, but are not limited to, isoquinolinone and dihydrolisoquinolinone (for example, U.S. Pat. No. 6,664,269, and WO 99/11624), nicotinamide, 3-aminobenzamide, monoaryl amides and bi-, tri-, or tetracyclic lactams, phenanthridinones (Perkins et al., 2001, Cancer Res., 61:4175-4183), 3,4-dihydro-5-methyl-isoquinolin-1(2H)-one and benzoxazole-4-carboxamide (Griffin et al., 1995, Anticancer Drug Des, 10:507-514; Griffin et al., 1998, J Med Chem, 41:5247-5256; and Griffin et al., 1996, Pharm Sci, 2:43-48), dihydroisoquinolin-1(2H)-nones, 1,6-naphthyridine-5(6H)-ones, quinazolin-4(3H)-ones, thieno[3,4-c]pyridin-4(5H) ones and thieno[3,4-d]pyrimidin-4(3H) ones, 1,5-dihydroxyisoquinoline, and 2-methyl-quinazolin-4[3H]-one (Yoshida et al., 1991, J Antibiot (Tokyo,) 44:111-112; Watson et al., 1998, Bioorg Med. Chem., 6:721-734; and White et al., 2000, J Med Chem., 43:4084-4097), 1,8-Napthalimide derivatives and (5H)phenanthridin-6-ones (Banasik et al., 1992, J Biol Chem, 267:1569-1575; Watson et al., 1998, Bioorg Med Chem., 6:721-734; Soriano et al., 2001, Nat Med., 7:108-113; Li et al., 2001, Bioorg Med Chem Lett., 11:1687-1690; and Jagtap et al., 2002, Crit Care Med., 30:1071-1082), tetracyclic lactams, 1,11b-dihydro-[2H]benzopyrano[4,3,2-de]isoquinolin-3-one, 1-methyl-4-phenyl-1,2,3,6-tetrahydropyridine (MPTP) (Zhang et al., 2000, Biochem Biophys Res Commun., 278:590-598; and Mazzon et al., 2001, Eur J Pharmacol, 415:85-94). Other examples of PARP inhibitors include, but are not limited to, those detailed in the patents: U.S. Pat. No. 5,719,151, U.S. Pat. No. 5,756,510, U.S. Pat. No. 6,015,827, U.S. Pat. No. 6,100,283, U.S. Pat. No. 6,156,739, U.S. Pat. No. 6,310,082, U.S. Pat. No. 6,316,455, U.S. Pat. No. 6,121,278, U.S. Pat. No. 6,201,020, U.S. Pat. Nos. 6,235,748, 6,306,889, U.S. Pat. No. 6,346,536, U.S. Pat. No. 6,380,193, U.S. Pat. No. 6,387,902, U.S. Pat. No. 6,395,749, U.S. Pat. No. 6,426,415, U.S. Pat. No. 6,514,983, U.S. Pat. No. 6,723,733, U.S. Pat. No. 6,448,271, U.S. Pat. No. 6,495,541, U.S. Pat. No. 6,548,494, U.S. Pat. No. 6,500,823, U.S. Pat. No. 6,664,269, U.S. Pat. No. 6,677,333, U.S. Pat. No. 6,903,098, U.S. Pat. No. 6,924,284, U.S. Pat. No. 6,989,388, U.S. Pat. No. 6,277,990, U.S. Pat. No. 6,476,048, and U.S. Pat. No. 6,531,464. Additional examples of PARP inhibitors include, but are not limited to, those detailed in the patent application publications: US 2004198693A1, US 2004034078A1, US 2004248879A1, US 2004249841A1, US 2006074073A1, US 2006100198A1, US 2004077667A1, US 2005080096A1, US 2005171101A1, US 2005054631A1, WO 05054201A1, WO 05054209A1, WO 05054210A1, WO 05058843A1, WO 06003146A1, WO 06003147A1, WO 06003148A1, WO 06003150A1, and WO 05097750A1.

In one embodiment of the present invention, the PARP inhibitors are compounds of Formula (Ia)

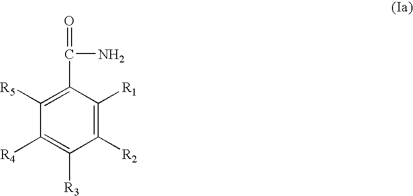

wherein R₁, R₂, R₃, R₄, and R₅ are, independently selected from the group consisting of hydrogen, hydroxy, amino, nitro, iodo, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen, at least one of the five substituents are always nitro, and at least one substituent positioned adjacent to a nitro is always iodo, and pharmaceutically acceptable salts, solvates, isomers, tautomers, metabolites, analogs, or prodrugs thereof. R₁, R₂, R₃, R₄, and R₅ can also be a halide such as chloro, fluoro, or bromo. Further details regarding compounds of formula Ia are provided in U.S. Pat. No. 5,464,871.

A preferred compound of formula Ia is a compound according to the formula Ia

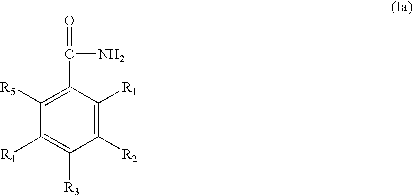

wherein R₂, R₃, R₄, and R₅ are, independent of one another, selected from the group consisting of hydrogen, hydroxy, amino, nitro, iodo, (C₁-C₆) alkyl, (C₁-C₆) alkoxy, (C₃-C₇) cycloalkyl, and phenyl and pharmaceutically acceptable salts thereof, wherein at least two of the five R₁, R₂, R₃, R₄, and R₅ substituents are always hydrogen and at least one of the five substituents are always nitro.

A preferred compound of formula Ia is

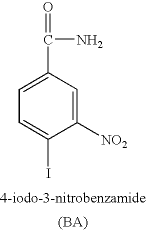

In some embodiments, benzopyrone compounds of formula II are used in the methods of the present invention. The benzopyrone compounds of formula II are,

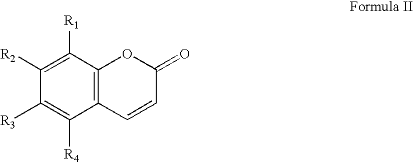

wherein R₁, R₂, R₃ and R₄ are independently selected from the group consisting of H, halogen, optionally substituted hydroxy, optionally substituted amine, optionally substituted lower alkyl, optionally substituted phenyl, optionally substituted C₄-C₁₀ heteroaryl and optionally substituted C₃-C₈ cycloalkyl or a salt, solvate, isomer, tautomers, metabolite, or prodrug thereof (U.S. Pat. No. 5,484,951 is incorporated herein by reference in its entirety).

Some embodiments employ a compound having the chemical formula:

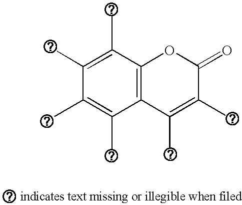

wherein R₁, R₂, R₃, or R₄ are each independently selected from the group consisting of hydrogen, hydroxy, amino, (C₁-C₆) alkyl, (C₁-C₆) alkoxy, (C₃-C₇) cycloalkyl, halo and phenyl and pharmaceutically acceptable salts thereof, wherein at least three of the four R₁, R₂, R₃, or R₄ substituents are always hydrogen.

Some embodiments employ a compound having the chemical formula:

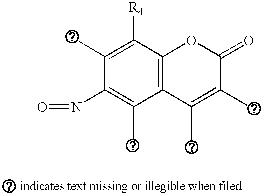

wherein R₁, R₂, R₃, or R₄ are each independently selected from the group consisting of hydrogen, hydroxy, amino, (C₁-C₆) alkyl, (C₁-C₆) alkoxy, (C₃-C₇) cycloalkyl, halo and phenyl and pharmaceutically acceptable salts thereof, wherein at least three of the four R₁, R₂, R₃, or R₄ substituents are always hydrogen.

Some embodiments employ a compound of the chemical formula:

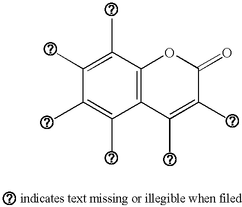

wherein R₁, R₂, R₃, or R₄, are each independently selected from the group consisting of hydrogen, hydroxy, amino, (C₁-C₆) alkyl, (C₁-C₆) alkoxy, (C₃-C₇) cycloalkyl, halo and phenyl, wherein at least three of the four R₁, R₂, R₃, or R₄ substituents are always hydrogen.

In a preferred embodiment, the invention relates to the following benzopyrone compound of formula II

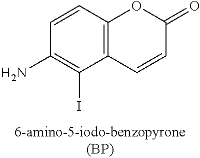

In yet another embodiment the compound used in the methods described herein is

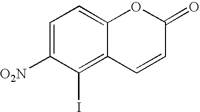

Further details regarding the benzopyrone compounds are in U.S. Pat. No. 5,484,951, which is herein incorporated by reference in its entirety.

It is likely that the most potent and effective PARP inhibitors (i.e., the likely candidates for drug development) are not yet available in the scientific literature but rather are undergoing clinical trials or may ultimately emerge in the various databases of published patents and pending patent applications. All such PARP inhibitors are within the scope of the present invention. In addition to selective, potent enzymatic inhibition of PARP, several additional approaches may be employed to inhibit the cellular activity of PARP in cells or in experimental animals. The inhibition of intracellular calcium mobilization protects against oxidant-induced PARP activation, NAD+depletion, and cell necrosis, as demonstrated in thymocytes (Virag et al., 1999, Mol. Pharmacol., 56:824-833) and in intestinal epithelial cells (Karczewski et al., 1999, Biochem Pharmacol., 57:19-26). Similar to calcium chelators, intracellular zinc chelators have been shown to protect against oxidant-mediated PARP activation and cell necrosis (Virag et al., 1999, Br J Pharmacol., 126:769-777). Intracellular purines (inosine, hypoxanthine), in addition to a variety of effects, may also exert biological actions as inhibitors of PARP (Virag et al., 2001, FASEB J., 15:99-107).

The methods provided by the invention may comprise the administration of PARP inhibitors by itself or in combination with other therapies. The choice of therapy that can be co-administered with the compositions of the invention will depend, in part, on the condition being treated. For example, for treating acute myeloid leukemia, compound of some embodiments of the invention can be used in combination with radiation therapy, monoclonal antibody therapy, chemotherapy, bone marrow transplantation, or a combination thereof.

An effective therapeutic amount of the PARP inhibitors as disclosed herein is administered to a patient, preferably a mammal and more preferably a human, to affect a pharmacological activity involving inhibition of a PARP enzyme or PARP activity. As such, PARP inhibitors of the present invention may be useful in treating or preventing a variety of diseases and illnesses including neural tissue damage resulting from cell damage or death due to necrosis or apoptosis, cerebral ischemia and reperfusion injury or neurodegenerative diseases in an animal. In addition, compounds of the present invention can also be used to treat a cardiovascular disorder in an animal, by administering an effective amount of the PARP inhibitor to the animal. Further still, the compounds of the invention can be used to treat cancer and to radiosensitize or chemosensitize tumor cells.

In some embodiments of the present invention, the PARP inhibitors can be used to modulate damaged neurons, promote neuronal regeneration, prevent neurodegeneration and/or treat a neurological disorder. The PARP inhibitors inhibit PARP activity and, thus, are useful for treating neural tissue damage, particularly damage resulting from cancer, cardiovascular disease, cerebral ischemia and reperfusion injury or neurodegenerative diseases in animals. The PARP inhibitors in the present invention are useful for treating cardiac tissue damage, particularly damage resulting from cardiac ischemia or caused by reperfusion injury in a patient. The compounds of the invention are particularly useful for treating cardiovascular disorders selected from the group consisting of: coronary artery disease, such as atherosclerosis; angina pectoris; myocardial infarction; myocardial ischemia and cardiac arrest; cardiac bypass; and cardiogenic shock.

In another aspect, the PARP inhibitors in the present invention can be used to treat cancer, or in combination with chemotherapeutics, radiotherapeutics, or radiation. The PARP inhibitors of the present invention can be “anti-cancer agents,” which term also encompasses “anti-tumor cell growth agents” and “anti-neoplastic agents.” For example, the PARP inhibitors of the invention are useful for treating cancers, and radiosensitizing and/or chemosensitizing tumor cells in cancers.

Radiosensitizers are known to increase the sensitivity of cancerous cells to the toxic effects of electromagnetic radiation. Many cancer treatment protocols currently employ radiosensitizers activated by the electromagnetic radiation of x-rays. Examples of x-ray activated radiosensitizers include, but are not limited to, the following: metronidazole, misonidazole, desmethylmisonidazole, pimonidazole, etanidazole, nimorazole, mitomycin C, RSU 1069, SR 4233, EO9, RB 6145, nicotinamide, 5-bromodeoxyuridine (BUdR),5-iododeoxyuridine (IUdR), bromodeoxycytidine, fluorodeoxyuridine (FudR), hydroxyurea, cisplatin, and therapeutically effective analogs and derivatives of the same.

Photodynamic therapy (PDT) of cancers employs visible light as the radiation activator of the sensitizing agent. Examples of photodynamic radiosensitizers include the following, but are not limited to: hematoporphyrin derivatives, photofrin, benzoporphyrin derivatives, NPe6, tin etioporphyrin SnET2, pheoborbide-α, bacteriochlorophyll-α, naphthalocyanines, phthalocyanines, zinc phthalocyanine, and therapeutically effective analogs and derivatives of the same.

Radiosensitizers can be administered in conjunction with a therapeutically effective amount of one or more other PARP inhibitors, including but not limited to: PARP inhibitors which promote the incorporation of radiosensitizers to the target cells; PARP inhibitors which control the flow of therapeutics, to nutrients, and/or oxygen to the target calls. Similarly, chemosensitizers are also known to increase the sensitivity of cancerous cells to the toxic effects of chemotherapeutic compounds. Exemplary chemotherapeutic agents that can be used in conjunction with PARP inhibitors include, but are not limited to, adriamycin, camptothecin, dacarbazine, carboplatin, cisplatin, daunorubicin, docetaxel, doxorubicin, interferon (alpha, beta, gamma), interleukin 2, innotecan, paclitaxel, streptozotocin, temozolomide, topotecan, and therapeutically effective analogs and derivatives of the same. In addition, other therapeutic agents which can be used in conjunction with a PARP inhibitors include, but are not limited to, 5-fluorouracil, leucovorin, 5′-amino-5′-deoxythymidine, oxygen, carbogen, red cell transfusions, perfluorocarbons (e.g., Fluosol-DA), 2,3-DPG, BW12C, calcium channel blockers, pentoxyfylline, antiangiogenesis compounds, hydralazine, and L-BSO.

In some embodiments, the therapeutic agents for the treatment include antibodies or reagents that bind to PARP, and thereby lower the level of PARP in a subject. In other embodiments, cellular expression can be modulated in order to affect the level of PARP and/or PARP activity in a subject. Therapeutic and/or prophylactic polynucleotide molecules can be delivered using gene transfer and gene therapy technologies. Still other agents include small molecules that bind to or interact with the PARP and thereby affect the function thereof, and small molecules that bind to or interact with nucleic acid sequences encoding PARP, and thereby affect the level of PARP in the present invention. These agents may be administered alone or in combination with other types of treatments known and available to those skilled in the art for treating diseases. In some embodiment, the PARP inhibitors for the treatment can be used either therapeutically, prophylactically, or both. The PARP inhibitors may either directly act on PARP or modulate other cellular constituents which then have an effect on the level of PARP. In some preferred embodiments, the PARP inhibitors inhibit the activity of PARP.

The methods of treatment as disclosed herein can be via oral administration, transmucosal administration, buccal administration, nasal administration, inhalation, parental administration, intravenous, subcutaneous, intramuscular, sublingual, transdermal administration, ocular administration, and rectal administration.

Pharmaceutical compositions of PARP inhibitors suitable for use in treatment following the identification of a disease treatable by PARP inhibitors in a subject, include compositions wherein the active ingredient is contained in a therapeutically or prophylactically effective amount, i.e., in an amount effective to achieve therapeutic or prophylactic benefit. The actual amount effective for a particular application will depend, inter alia, on the condition being treated and the route of administration. Determination of an effective amount is well within the capabilities of those skilled in the art. The pharmaceutical compositions comprise the PARP inhibitors, one or more pharmaceutically acceptable carriers, diluents or excipients, and optionally additional therapeutic agents. The compositions can be formulated for sustained or delayed release.

The compositions can be administered by injection, topically, orally, transdermally, rectally, or via inhalation. The oral form in which the therapeutic agent is administered can include powder, tablet, capsule, solution, or emulsion. The effective amount can be administered in a single dose or in a series of doses separated by appropriate time intervals, such as hours. Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Suitable techniques for preparing pharmaceutical compositions of the therapeutic agents of the present invention are well known in the art.

A preferred dose for 4-iodo-3-nitrobenzamide is 4 mg/kg IV over one hour twice weekly beginning on day 1 (doses of 4-iodo-3-nitrobenzamide are preferably separated by at least 2 days). 4-iodo-3-nitrobenzamide treatment is preferably given twice weekly as an IV infusion for three consecutive weeks in each 28-day cycle. Other preferred doses include 0.5, 1.0, 1.4, 2.8 and 4 mg/kg either as a monotherapy or a combination therapy.

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular PARP inhibitor, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

### Standard of Care for Cancer Sites

In another aspect of the invention, PARP inhibitors are used in combination with the primary standards of treatment for the cancer being treated. Described herein is the standard of care for certain types of cancers. In some embodiments, the PARP inhibitors are used in combination with the standard of care described herein.

### Endometrial

There are four primary standards of care for treating endometrial cancers including surgery (total hysterectomy, bilateral salpingo-oophorectomy, and radical hysterectomy), radiation, chemotherapy, and hormone therapy. Adjuvant therapies involving said therapies are administered in some cases.

### Breast

Breast cancer treatments currently involve breast-conserving surgery and radiation therapy with or without tamoxifen, total mastectomy with or without tamoxifen, breast-conserving surgery without radiation therapy, bilateral prophylactic total mastectomy without axillary node dissection, delivering tamoxifen to decrease the incidence of subsequent breast cancers, and adjuvant therapies involving said therapies.

### Ovary

If the tumor is well- or moderately well-differentiated, total abdominal hysterectomy and bilateral salpingo-oophorectomy with omentectomy is adequate for patients with early stage disease. Patients diagnosed with stage III and stage IV disease are treated with surgery and chemotherapy.

### Cervix

Methods to treat ectocervical lesions include loop electrosurgical excision procedure (LEEP), laser therapy, conization, and cryotherapy. For stage I and stage II tumors, treatment options include: total hysterectomy, conization, radical hysterectomy, and intracavitary radiation therapy alone, bilateral pelvic lymphadenectomy, postoperative total pelvic radiation therapy plus chemotherapy, and radiation therapy plus chemotherapy with cisplatin or cisplatin/5-FU. For stage III and stage IV tumors, the standard of treatment of cervical cancer is radiation and/or chemotherapy with drugs including cisplatin, ifosfamide, ifosfamide-cisplatin, paclitaxel, irinotecan, paclitaxel/cisplatin, and cisplatin/gemcitabine.

### Testes

The standards of treatment of seminoma are radical inguinal orchiectomy with or without by single-dose carboplatin adjuvant therapy, removal of the testicle via radical inguinal orchiectomy followed by radiation therapy, and radical inguinal orchiectomy followed by combination chemotherapy or by radiation therapy to the abdominal and pelvic lymph nodes. For nonseminoma patients treatments include removal of the testicle through the groin followed by retroperitoneal lymph node dissection, radical inguinal orchiectomy with or without removal of retroperitoneal lymph nodes with or without fertility-preserving retroperitoneal lymph node dissection with or without chemotherapy.

### Lung

In non-small cell lung cancer (NSCLC), results of standard treatment are poor except for the most localized cancers. All newly diagnosed patients with NSCLC are potential candidates for studies evaluating new forms of treatment. Surgery is the most potentially curative therapeutic option for this disease; radiation therapy can produce a cure in a small number of patients and can provide palliation in most patients. Adjuvant chemotherapy may provide an additional benefit to patients with resected NSCLC. In advanced-stage disease, chemotherapy is used.

### Skin

The traditional methods of basal cell carcinoma treatment involve the use of cryosurgery, radiation therapy, electrodesiccation and curettage, and simple excision. Localized squamous cell carcinoma of the skin is a highly curable disease. The traditional methods of treatment involve the use of cryosurgery, radiation therapy, electrodesiccation and curettage, and simple excision.

### Liver

Hepatocellular carcinoma is potentially curable by surgical resection, but surgery is the treatment of choice for only the small fraction of patients with localized disease. Other treatments remain in the clinical study phase including systemic or infusional chemotherapy, hepatic artery ligation or embolization, percutaneous ethanol injection, radiofrequency ablation, cryotherapy, and radiolabeled antibodies, often in conjunction with surgical resection and/or radiation therapy.

### Thyroid

Standard treatment options of thyroid cancers include total thyroidectomy, lobectomy, and combinations of said surgeries with I¹³¹ ablation, external-beam radiation therapy, thyroid-stimulating hormone suppression with thyroxine, and chemotherapy.

### Esophagus

Primary treatment modalities include surgery alone or chemotherapy with radiation therapy. Effective palliation may be obtained in individual cases with various combinations of surgery, chemotherapy, radiation therapy, stents, photodynamic therapy, and endoscopic therapy with Nd: YAG laser.

### Kidney

Surgical resection is the mainstay of treatment of this disease. Even in patients with disseminated tumor, locoregional forms of therapy may play an important role in palliating symptoms of the primary tumor or of ectopic hormone production. Systemic therapy has demonstrated only limited effectiveness.

In one embodiment, PARP inhibitors are combined with other chemotherapeutics such as, irinotecan, topotecan, cisplatin, or temozolomide to improve the treatment of a number of cancers such as colorectal and gastric cancers, and melanoma and glioma, respectively. In another embodiment, PARP inhibitors are combined with irinotecan to treat advanced colorectal cancer or with temozolomide to treat malignant melanoma.

In cancer patients, in one embodiment PARP inhibition is used to increase the therapeutic benefits of radiation and chemotherapy. In another embodiment, targeting PARP is used to prevent tumor cells from repairing DNA themselves and developing drug resistance, which may make them more sensitive to cancer therapies. In yet another embodiment, PARP inhibitors are used to increase the effect of various chemotherapeutic agents (e.g. methylating agents, DNA topoisomerase inhibitors, cisplatin etc.), as well as radiation, against a broad spectrum of tumors (e.g. glioma, melanoma, lymphoma, colorectal cancer, head and neck tumors).

### Kits

In yet another aspect, the invention provides kits for identifying a disease in a subject treatable by PARP modulators, wherein the kits can be used to detect the level of PARP in a sample obtained from a subject. For example, the kits can be used to identify the level and/or activity of PARP in normal and diseased tissue as described herein, where PARP level is differentially present in samples of a diseased patient and normal subjects. In one embodiment, a kit comprises a substrate comprising an adsorbent thereon, wherein the adsorbent is suitable for binding PARP and/or RNA, and instructions to identify PARP and/or level of PARP and/or PAR (monoribose and polyribose) by contacting a sample with the adsorbent and detecting PARP retained by the adsorbent. In another embodiment, a kit comprises (a) a reagent that specifically binds to or interacts with PARP; and (b) a detection reagent. In some embodiments, the kit may further comprise instructions for suitable operation parameters in the form of a label or a separate insert. Optionally, the kit may further comprise a standard or control information so that the test sample can be compared with the control information standard to determine if the test amount of PARP detected in a sample is a diagnostic amount.

In some embodiments, the therapeutic agent can also be provided as separate compositions in separate containers within the kit for the treatment. Suitable packaging and additional articles for use (e.g., measuring cup for liquid preparations, foil wrapping to minimize exposure to air, and the like) are known in the art and may be included in the kit.

### Example 1

GeneChip arrays have been widely used for monitoring mRNA expression in many areas of biomedical research. The high-density oligonucleotide array technology allows researchers to monitor tens of thousands of genes in a single hybridization experiment as they are expressed differently in tissues and cells. The expression profile of a mRNA molecule of a gene is obtained by the combined intensity information from probes in a probe set, which consists of 11-20 probe pairs of oligonucleotides of 25 by in length, interrogating a different part of the sequence of a gene.

The gene expressions were assessed using the Affymetrix human genome genechips (45,000 gene transcripts covering 28,473 UniGene clusters). Approximately 5 μg total RNA from each sample were labeled using high yield transcript labeling kit and labeled RNAs were hybridized, washed, and scanned according to manufacturer's specifications (Affymetrix, Inc., Santa Clara, Calif.). Affymetrix Microarray Suite 5.0 software (MASS) was used to estimate transcript signal levels from scanned images (Affymetrix). The signals on each array were normalized to a trimmed mean value of 500, excluding lowest 2% and highest 2% of the signals. An Affymetrix probe set representing a unique Genbank sequence is referred as a probe or gene hereafter for convenience. To verify any errors in the expressions caused by image defects, the correlation coefficient of each array to an idealized distribution was determined where the idealized distribution is mean of all arrays. The genes are filtered from the remaining arrays using detection P value reported by MASS. The genes having P>0.065 in 95% of the arrays are eliminated and all other signals are included for statistical comparisons of classes.

### Example 2

### Expression of PARP1 mRNA 1n human normal breast and infiltrating duct Carcinoma

### Study Design

Normal breast and infiltrating duct carcinoma samples were identified in the BioExpress® System that were members of the sample sets defined for the ASCENTA® System. Each tumor sample was also assessed for its percent tumor annotation, which is a quantitative determination by the reviewing pathologist of the ratio of malignant to non-malignant nucleated cells present in a microscopic slide from a section taken adjacent to the processed sample.

A total of 237 independent samples were assessed in this study, with numbers of samples relative to each of the IDC subtypes presented in Table A. Table A also presents sample numbers for each IDC subtype based on the percentage of the sample observed as tumor tissue.

TABLE A
Sample Numbers by Pathology Class and Percent Tumor

| | Percent Tumor | | | | |
|---|---|---|---|---|---|
| Group | 25-50 | 50-75 | 75-90 | >90 | All |
| Normal | N/A | N/A | N/A | N/A | 68 |
| IDC | 15 | 36 | 60 | 58 | 169 |
| IDC ER(+) | 10 | 9 | 11 | 5 | 35 |
| IDC ER(+)/PR(+) | 8 | 7 | 8 | 3 | 26 |
| IDC ER(+)/PR(−) | 1 | 2 | 3 | 2 | 8 |
| IDC ER(−) | 3 | 6 | 8 | 1 | 18 |
| IDC ER(−)/PR(−) | | | 7 | 1 | 8 |
| IDC Her2-neu(+) | 8 | 5 | 11 | | 24 |
| IDC Her2-neu(−) | 2 | 3 | 4 | 1 | 10 |
| IDC PR(+) | 8 | 7 | 8 | 3 | 26 |
| IDC PR(−) | 1 | 5 | 11 | 3 | 20 |
| IDC Stage I | | 3 | 9 | 6 | 18 |
| IDC Stage II | | 19 | 21 | 30 | 70 |
| IDC Stage III | | 2 | 8 | 4 | 14 |
| IDC Stage IV | | | 2 | 3 | 5 |
| IDC p53(+) | 2 | 3 | 3 | | 8 |
| IDC p53(−) | 7 | 4 | 5 | | 16 |

Table A indicates that >90% of the IDC samples are composed of 50% or greater tumor tissue and that about two-thirds of all IDC samples are comprised of 75% or greater tumor tissue, indicating a good representation of tumor-rich samples.

It should be noted that any IDC sample may be represented in more than one subtype grouping. An example is shown in Table B for seven selected IDC samples and their presence in multiple, single, or no IDC subtypes. For instance, sample GID 7273 is not classified into any single subtype and is therefore only assessed as a general IDC sample. Sample GID 7287 is classified into only one subtype and would therefore contribute to results for its Stage II class as well as the general IDC class. Sample GID 7387 is classified into two subtypes and would therefore contribute to results for both of these subtypes as well as the general IDC class.

TABLE B
Example of Subtype Classifications for Selected IDC Samples

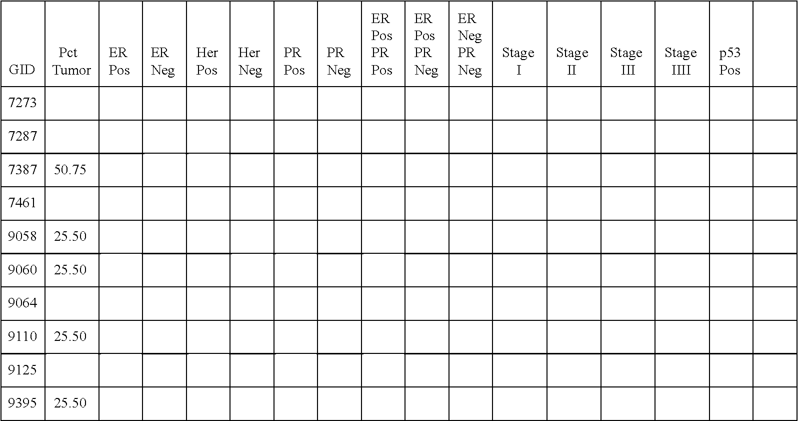

The PARP1 gene is represented on the HG-U133A array by a single probe set with the identifier W208644_at”. All results in this report were generated based on the MASS expression signal intensities for this probe set and will be referred to as “PARP1”.

Full Sample Set Statistical Analysis

Normal and IDC Summary Statistics

The normal and general IDC sample classes were summarized by mean, standard deviation, standard error, and several upper confidence limits based on at distribution. The upper confidence limits (UCL) are similar to standard deviations statistics in that they identify specific regions of probability for observing a value. For instance, a 95% upper confidence limit is akin to a value that would be expected by chance in 5% of samples.

In the case of the breast normal data, the number of samples (n=68) is large enough that the t distribution closely approximates results obtained when a standard deviation only is used to set limits. For instance, the mean+2SD of the normal breast expression intensities is 365.06, which is very similar to the 95% confidence limit of 365.92. This would not be the case for organs where the normal sample numbers are lower.

Table C shows summary statistics for each of the normal breast and general IDC sample sets.

TABLE C
Summary Statistics for the Normal and IDC Breast Sample Sets

| | | | | | 90% | 95% | 99% | 99.9% |
|---|---|---|---|---|---|---|---|---|
| Group | N | Mean | Std Dev | Std Err | UCL | UCL | UCL | UCL |
| Infiltrating duct | 169 | 328.487 | 135.695 | 10.4381 | 553.586 | 597.166 | 683.073 | 784.324 |
| carcinoma | | | | | | | | |
| Normal tissue | 68 | 201.780 | 81.636 | 9.8998 | 338.939 | 365.919 | 419.800 | 484.808 |

IDC mean/Normal mean = 1.63
t-test for (IDC mean = Normal mean) yields p = 6 * 10⁻¹⁶

Therefore, while the fold change is moderate for IDC with respect to normal samples, the change is very highly significant.

### Individual Sample Assessments

Next, individual samples from the general IDC breast sample set and all IDC subtypes were individually tested relative to the normal breast sample distribution. Each was defined as exceeding the 90%, 95%, 99%, and 99.9% upper confidence limits. None of the IDC samples were below the 90% Lower Confidence Limit of 64.6 and so LCL bounds are not presented.

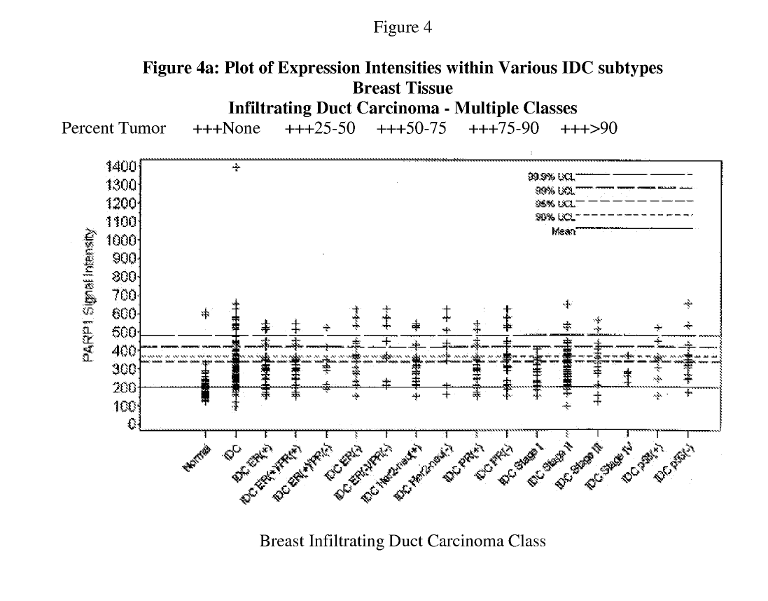

FIG. 4a shows a visual summary of the results for each of the classes of breast samples. Each cross indicates a single sample according to the subtype shown on the x-axis and its expression intensity on the y-axis. In addition, each point is colored by the percent tumor inherent in the sample. FIG. 4b is identical to FIG. 4a except that the highest sample within the IDC grouping has been removed to allow for better scaling.

The results based on FIG. 4 are:
The high degree of expression of PARP1 in IDC breast samples is apparent relative to normal breast samples.
The IDC breast sample expression of PARP1 exhibits a much higher degree of variation (i.e., greater spread) than that of the normal breast samples.
Two normal breast samples have higher PARP1 expression intensities than the other 66 samples and do not seem to be a part of the same underlying distributions.
One IDC breast sample has very high expression intensity and does not seem to be a part of the same underlying distribution.
Percent tumor does not seem to influence expression intensity to a great degree within the breast IDC samples, at least visually.

Table D summarizes the percentage and numbers of samples that exceed predefined upper confidence limits for the IDC class and its subtypes.

TABLE D
Percentage and Numbers of Samples Exceeding UCL for IDC and its Subtypes

| | >90% UCL | | >95% UCL | | >99% UCL | | >99.9% UCL | |
|---|---|---|---|---|---|---|---|---|
| Normal | 2.9% | (2/68) | 2.9% | (2/68) | 2.9% | (2/68) | 2.9% | (2/68) |
| IDC | 39.6% | (67/169) | 30.2% | (51/169) | 16.0% | (27/169) | 8.9% | (15/169) |
| IDC ER(+) | 37.1% | (13/35) | 22.9% | (8/35) | 17.1% | (6/35) | 8.6% | (3/35) |
| IDC ER(+)/PR(+) | 38.5% | (10/26) | 23.1% | (6/26) | 15.4% | (4/26) | 7.7% | (2/26) |
| IDC ER(+)/PR(−) | 37.5% | (3/8) | 25.0% | (2/8) | 25.0% | (2/8) | 12.5% | (1/8) |
| IDC ER(−) | 61.1% | (11/18) | 55.6% | (10/18) | 33.3% | (6/18) | 16.7% | (3/18) |
| IDC ER(−)/PR(−) | 75.0% | (6/8) | 62.5% | (5/8) | 50.0% | (4/8) | 37.5% | (3/8) |
| IDC Her2-neu(+) | 50.0% | (12/24) | 29.2% | (7/24) | 25.0% | (6/24) | 12.5% | (3/24) |
| IDC Her2-neu(−) | 80.0% | (8/10) | 70.0% | (7/10) | 40.0% | (4/10) | 30.0% | (3/10) |
| IDC PR(+) | 38.5% | (10/26) | 23.1% | (6/26) | 15.4% | (4/26) | 7.7% | (2/26) |
| IDC PR(−) | 55.0% | (11/20) | 45.0% | (9/20) | 35.0% | (7/20) | 20.0% | (4/20) |
| IDC Stage I | 16.7% | (3/18) | 5.6% | (1/18) | 0.0% | (0/18) | 0.0% | (0/18) |
| IDC Stage II | 44.3% | (31/70) | 35.7% | (25/70) | 12.9% | (9/70) | 4.3% | (3/70) |
| IDC Stage III | 42.9% | (6/14) | 35.7% | (5/14) | 21.4% | (3/14) | 14.3% | (2/14) |
| IDC Stage IV | 20.0% | (1/5) | 20.0% | (1/5) | 0.0% | (0/5) | 0.0% | (0/5) |
| IDC p53(+) | 62.5% | (5/8) | 37.5% | (3/8) | 25.0% | (2/8) | 12.5% | (1/8) |
| IDC p53(−) | 50.0% | (8/16) | 43.8% | (7/16) | 31.3% | (5/16) | 12.5% | (2/16) |

The results that can be made from the summary table are as follows:
Most subtypes of IDC showed at least 30% of samples above the 95% UCL, there were some notable exceptions:
   All IDC ER+sets
   IDCHer2-neu+
   All IDC PR+sets
   Stages I and IV
Class comparisons of PARP1 expression:
   IDC ER->IDC ER+
   IDC Her2-neu->IDC Her2-neu+
   IDC PR->IDC PR+
   IDC p53-˜=IDC p53−
   IDC Stg II, III>IDC Stg I, IV

### Curated Sample Set Statistical Analysis

### Normal and IDC Summary Statistics

The reason for elevated expression in the two normal samples and the one IDC sample well above the rest of the samples in their groups was not apparent based on what is known about the samples. The quality control methods implemented by Gene Logic in defining samples for ASCENTA™ include outlier assessments on a multivariate level, but utilize the full gene set on the array and do not make specific comparisons to other sample sets. These samples were not originally identified as outliers in the context of the full set of genes measured on the HG-U133A array. To more closely assess the samples in the context of this particular dataset, we performed a quality assessment using a focused set of genes selected to differentiate normal from infiltrating duct carcinoma.

A set of about 1,700 genes was selected which differentiate normal breast tissue from IDC and principal components analysis and correlation analysis were performed. Each of the selected genes exhibited a fold change of at least 2 and had a t-test p-value less than 0.01. The results of the analysis indicated that the two outlier samples appear to be misclassified and should be removed. As part of the investigation of the two outliers identified in FIGS. 4a and 4b, a larger assessment of the set of 237 samples was performed. The results of these analyses indicate that another 3 normal and 5 IDC samples should be removed from the analysis. These samples appear to be misclassified and are not appropriate samples for this analysis. The removal of 10 outlier samples leaves 63 normals and 164 IDC samples. The remaining numbers of samples in each IDC subgroup are detailed in Table E below.

All of the subgroups continue to have at least 5 samples. The one IDC sample that was identified as an outlier for PARP1 expression did not appear to be an outlier in this quality assessment. This sample was left in the analysis.

The 5 normals that were removed tended to be at the higher end of the normal expression range. The removal of these 5 would therefore tend to lower the overall average. In addition, the removal of the two outliers in particular resulted in narrower confidence limits. In the IDC category, the 5 outliers identified tended to be at the lower end of the IDC expression range. Removal of these samples resulted in slightly increased summary statistics. The updated summary statistics are presented in Table F. The change in the IDC group is not as significant as the normals because of the increased number of samples and because none of the 5 samples removed appeared to be outliers for PARP1.

TABLE E
Sample numbers by Percent Tumor and Pathology
(with outliers removed)

| Group | 25-50 | 50-75 | 75-90 | >90 | All |
|---|---|---|---|---|---|
| Normal | N/A | N/A | N/A | N/A | 63 |
| IDC | 14 | 36 | 59 | 55 | 164 |
| IDC ER(+) | 9 | 9 | 11 | 5 | 34 |
| IDC ER(+)/PR(+) | 7 | 7 | 8 | 3 | 25 |
| IDC ER(+)/PR(−) | 1 | 2 | 3 | 2 | 8 |
| IDC ER(−) | 3 | 6 | 7 | 1 | 17 |
| IDC ER(−)/PR(−) | | | 7 | 1 | 8 |
| IDC Her2-neu(+) | 8 | 5 | 10 | | 23 |
| IDC Her2-neu(−) | 1 | 3 | 4 | 1 | 9 |
| IDC PR(+) | 7 | 7 | 8 | 3 | 25 |
| IDC PR(−) | 1 | 5 | 10 | 3 | 19 |
| IDC Stage I | | 3 | 9 | 6 | 18 |
| IDC Stage II | | 19 | 21 | 28 | 68 |
| IDC Stage III | | 2 | 8 | 4 | 14 |
| IDC Stage IV | | | 2 | 3 | 5 |
| IDC p53(+) | 2 | 3 | 2 | | 7 |
| IDC p53(−) | 7 | 4 | 5 | | 16 |

Removal of the outlier samples resulted in an increase in the fold change between IDC and Normal mean intensities. The t-test for significant differences between the two groups resulted in a reduced p-value. Overall, the removal of the outliers results in a larger difference in mean intensity between Normal and IDC and this difference was more significant.

TABLE F
Summary Statistics for the Normal and IDC Breast Sample Sets without Outliers

| | | | | | 90% | 95% | 99% | 99.9% |
|---|---|---|---|---|---|---|---|---|
| Group | N | Mean | Std Dev | Std Err | UCL | UCL | UCL | UCL |
| Infiltrating duct | 164 | 332.819 | 135.360 | 10.5698 | 557.421 | 600.918 | 686.686 | 787.821 |
| carcinoma | | | | | | | | |
| Normal tissue | 63 | 186.413 | 40.367 | 5.0857 | 254.350 | 267.743 | 294.534 | 326.961 |

IDC mean/Normal mean = 1.79
t-test for (IDC mean = Normal mean) yields p = 2 * 10⁻²⁷

### Individual Sample Assessments

As observed in Table C, the upper confidence limits calculated for the normal samples were reduced when the outliers were removed. This resulted in more IDC samples outside the various limits defined. FIGS. 5a and 5b reflect the reduced number of samples and the tighter confidence limits that resulted.

Comparing the results to FIGS. 4a and 4b, the mean of the normals has dropped below 200 and the upper confidence limits are notably closer to the mean than in the analysis of the full 237. There continues to be no apparent difference between the various classes of percent tumor. This is based on the observation that several samples classified as >90% tumor tend to be at the lower end of the infiltrating duct carcinoma range and that samples in the 25%-50% tumor class have higher PARP1 expression. In addition, the 50%-75% and the 75%-90% classes tend to be uniformly distributed across the range of expression for the tumor samples. Overall, more IDC samples are above each of the confidence limits than in the earlier analysis.

As observed in the analysis of all samples, PARP1 expression tends to be slightly higher in the ER(−), PR(−), and Her2-neu(−) classes as compared to their respective (+) classes. This finding is not observed in the p53 classes or in the tumor stage classes. The fact that individual samples are contributing to multiple categories in this analysis could be influencing this conclusion. A review of the supplementary dataset reveals that the highest PARP1 expressor in the ER(−) group is the same high expressor in the PR(−) and Her2-neu(−) groups. The same is true for the lowest expressor in the (+) groups.

As predicted earlier in this section, the numbers of IDC samples above the Normal UCLs is increased with the outliers removed. Table G summarizes the numbers of samples above each confidence limit for the various categories of infiltrating duct carcinoma. For the 164 IDC samples as a whole, 74% and 45% of the samples are above the 90% and 99.9% UCLs, respectively as compared to 39% and 9% previously. The (−) status categories for ER, PR, and Her2-neu remain elevated compared to their respective (+) categories. The difference is most pronounced when comparing groups at the 99.9% UCL level. The difference in PR categories is less pronounced than in the ER and Her2-neu groups.

TABLE G
Percentage and Numbers of Samples Exceeding UCLs for IDC and its Subtypes with
Outliers Removed

| | >90% UCL | | >95% UCL | | >99% UCL | | >99.9% UCL | |
|---|---|---|---|---|---|---|---|---|
| Normal | 7.9% | (5/63) | 4.8% | (3/63) | 1.6% | (1/63) | 0.0% | (0/63) |
| IDC | 74.4% | (122/164) | 70.1% | (115/164) | 58.5% | (96/164) | 45.7% | (75/164) |
| IDC ER(+) | 73.5% | (25/34) | 73.5% | (25/34) | 61.8% | (21/34) | 38.2% | (13/34) |
| IDC ER(+)/PR(+) | 72.0% | (18/25) | 72.0% | (18/25) | 60.0% | (15/25) | 40.0% | (10/25) |
| IDC ER(+)/PR(−) | 75.0% | (6/8) | 75.0% | (6/8) | 62.5% | (5/8) | 37.5% | (3/8) |
| IDC ER(−) | 88.2% | (15/17) | 88.2% | (15/17) | 76.5% | (13/17) | 64.7% | (11/17) |
| IDC ER(−)/PR(−) | 75.0% | (6/8) | 75.0% | (6/8) | 75.0% | (6/8) | 75.0% | (6/8) |
| IDC Her2-neu(+) | 82.6% | (19/23) | 82.6% | (19/23) | 73.9% | (17/23) | 52.2% | (12/23) |
| IDC Her2-neu(−) | 88.9% | (8/9) | 88.9% | (8/9) | 88.9% | (8/9) | 88.9% | (8/9) |
| IDC PR(+) | 72.0% | (18/25) | 72.0% | (18/25) | 60.0% | (15/25) | 40.0% | (10/25) |
| IDC PR(−) | 78.9% | (15/19) | 78.9% | (15/19) | 73.7% | (14/19) | 57.9% | (11/19) |
| IDC Stage I | 50.0% | (9/18) | 44.4% | (8/18) | 33.3% | (6/18) | 22.2% | (4/18) |
| IDC Stage II | 75.0% | (51/68) | 69.1% | (47/68) | 60.3% | (41/68) | 50.0% | (34/68) |
| IDC Stage III | 71.4% | (10/14) | 71.4% | (10/14) | 57.1% | (8/14) | 50.0% | (7/14) |
| IDC Stage IV | 80.0% | (4/5) | 60.0% | (3/5) | 20.0% | (1/5) | 20.0% | (1/5) |
| IDC p53(+) | 85.7% | (6/7) | 85.7% | (6/7) | 85.7% | (6/7) | 71.4% | (5/7) |
| IDC p53(−) | 81.3% | (13/16) | 81.3% | (13/16) | 75.0% | (12/16) | 56.3% | (9/16) |

### Conclusions

The expression of PARP1 in infiltrating duct carcinoma is significantly elevated compared to normals. FIGS. 5a and 5b show that despite tills finding, not all IDC samples are over expressed. This wider distribution and shift towards higher expression in the IDC group indicates that about 70% of IDC may have PARP1 expression above the 95% upper confidence limit of the normal population. This finding supports findings previously observed by BiPar. Further analysis into various subgroups of IDC samples reveals that the percentage of IDC observed to have elevated PARP1 expression increases to 88% to 89% if their ER status is negative or if their Her2-neu status is negative. The percentage of PR negative samples above the Normal 95% UCL, 79%, is less pronounced but still elevated.

This suggests that any therapies targeting over expression of PARP1 may be more effective in cases where the ER, PR, or Her2-neu tests are negative.

In summary:
1. PARP1 expression is higher in infiltrating duct carcinoma than in normal breast tissue.
2. The percentage of tumor observed in the histopathology slides does not appear to be an important factor in measuring PARP1 expression.
3. The presence one outlier in the IDC group may indicate the existence of abnormally high expression in a small percentage of individuals.
4. Certain subtypes of infiltrating duct carcinoma appear to exhibit higher expression levels than other subtypes. In particular, the (−) subtypes for ER, Her2-neu, and PR showed higher percentages of samples above the Normal UCLs than their respective (+) subtypes.

### Discussion and Interpretation

The results of this study are consistent with increased PARP1 expression in breast infiltrating duct carcinoma. If over-expression of PARP1 in IDC is defined as a level greater than the 95% upper confidence limit of expression in normal breast tissue, then approximately two-thirds of infiltrating duct carcinomas overexpress PARP1. If PARP1 over-expression defines increased responsiveness to PARP1 inhibition, then the results imply that a substantial fraction of IDC's would be rational candidates for therapy with PARP1 inhibitors. Furthermore, in the estrogen receptor negative and Her2-neu negative IDC subsets, the fraction of PARP1 over-expressing tumors was even higher than in the entire IDC population, suggesting that (1) it may be advantageous to concentrate on specific types of PARP1 over-expressing tumors in clinical trials using standard laboratory assays or to assess differential responses to therapy, and (2) PARP1 inhibition may be a rational approach for cancers that do not qualify for antiestrogen or anti-Her2-neu therapies.

### Example 3

### Tissue Expression of PARP1 in Ovarian Cancer and Normal Ovary

### Study Design

Normal ovary and cancerous ovary samples were selected from the BioExpress® System that were members of sample sets defined for the ASCENTA® System. It should be noted that any cancerous sample may be represented in more than one subtype grouping. An example is shown in Table H for 10 selected ovary samples and their membership in multiple subtypes. For instance, sample GID 8757 is classified into the endometrioid type of cancer as well as its respective age, CA125 status, and stage subtypes. Some subtypes are exclusive of each other while others are not, yielding a full classification system for any individual sample.

TABLE H
Example of Subtype Classifications for Selected Ovary Samples

| | Normal | | | Endometrioid, | Endometrioid, | Endometrioid, | Endometrioid, | Endometrioid, |
|---|---|---|---|---|---|---|---|---|
| Genomics ID | Ovary | Clear Cell | Endometrioid | Over 45 yrs | Under 45 yrs | Elevated CA125 | Stage I | Stage III |
| 4051 | | Y | | | | | | |
| 9357 | | Y | | | | | | |
| 7473 | | Y | | | | | | |
| 31852 | | Y | | | | | | |
| 15133 | | Y | | | | | | |
| 12007 | | Y | | | | | | |
| 7389 | | | Y | Y | | | | |
| 8757 | | | Y | Y | | Y | Y | |
| 2619 | | | Y | Y | | | Y | |
| 31903 | | | Y | Y | | Y | Y | |

The PARP1 gene is represented on the HG-U133A array by a single probe set with the identifier “208644_at”. All results in this report were generated based on the MASS expression signal intensities for this probe set and will be referred to as “PARP1”. In addition, the seven genes, BRCA1, BRCA2, RAD51, MRE11, p53, PARP2 and MUCIN 16, are represented on the HG-U133A/B array set by 11 informative probe sets. Three probe sets were excluded from this analysis because they were considered to be non-responsive on the array.

### Statistical Analysis

### Normal and Cancerous Summary Statistics

The normal and main cancerous sample classes were summarized by mean, standard deviation, standard error, and several upper confidence limits based on at distribution. The upper confidence limits (UCL) are similar to standard deviation statistics in that they identify specific regions of probability for observing a value. For instance, a 95% upper confidence limit is akin to a value above which one would expect by chance in 5% of samples.

In the case of the ovary normal data, the number of samples (n=88) is large enough that the t distribution closely approximates results obtained when a standard deviation only is used to set limits as summarized in Table I. For instance, the mean+2 standard deviation of the normal ovary expression intensities is 224.18, which is very similar to the 95% confidence limit of 224.15. This would not be the case for organs where the normal sample numbers are lower.

TABLE I
Summary Statistics for the Normal and Cancerous Ovary Sample Sets

| | | | | | 90% | 95% | 99% | 99.9% |
|---|---|---|---|---|---|---|---|---|
| Main Cancerous Sample Class | Number | Mean | Std Dev | Std Err | UCL | UCL | UCL | UCL |
| Normal tissue | 88 | 163.037 | 30.572 | 3.259 | 214.15 | 224.15 | 244.00 | 267.75 |
| Clear cell adenocarcinoma | 6 | 220.757 | 45.995 | 18.777 | 320.86 | 348.46 | 421.07 | 562.00 |
| Endometrioid adenocarcinoma | 13 | 302.863 | 119.713 | 33.202 | 524.28 | 573.54 | 682.33 | 839.27 |
| Granulosa cell tumor | 3 | 422.980 | 204.006 | 117.783 | 1110.83 | 1436.54 | 2760.94 | 7866.65 |
| Mucinous cystadenocarcinoma | 7 | 191.453 | 47.990 | 18.139 | 291.14 | 316.99 | 381.66 | 497.16 |
| Mullerian mixed tumor | 5 | 371.404 | 144.270 | 64.520 | 708.32 | 810.19 | 1099.04 | 1732.18 |
| Papillary serous adenocarcinoma | 64 | 357.092 | 144.994 | 18.124 | 601.03 | 649.09 | 745.21 | 861.47 |
| Serous cystadenocarcinoma | 8 | 371.234 | 104.078 | 36.797 | 580.38 | 632.27 | 757.55 | 968.22 |

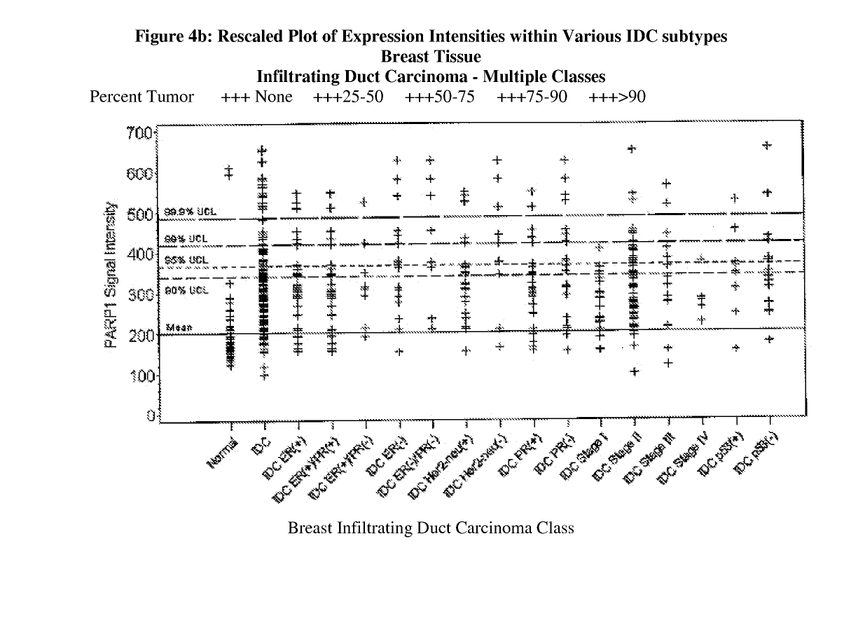

All of the ovarian cancers expressed higher mean PARP1 than normal ovary. Clear cell adenocarcinoma and mucinous cystadenocarcinoma samples expressed considerably lower PARP1 than did the other subtypes, and the variance in expression was also lower as demonstrated in FIG. 6.

Table J lists the ratio-based fold change and Student's two-tailed t-test results of the PARP1 gene as measured using the array data from Table I.

TABLE J
Comparison Statistics of Cancer Types to Normal

| | | p-value (t-test |
|---|---|---|
| | Fold Change | of Cancer |
| Main Cancerous Sample Class | (vs Normal) | Type to Normal) |
| Clear cell adenocarcinoma | 1.354 | 0.0270 |
| Endometrioid adenocarcinoma | 1.858 | 0.0012 |
| Granulosa cell tumor | 2.594 | 0.1579 |
| Mucinous cystadenocarcinoma | 1.174 | 0.1710 |
| Mullerian mixed tumor | 2.278 | 0.0319 |
| Papillary serous adenocarcinoma | 2.190 | <.0001 |
| Serous cystadenocarcinoma | 2.277 | 0.0007 |

It should be noted that while some of the fold changes are large, small sample size can yield an insignificant p-value, such as is observed for granulosa cell tumor. Alternately, papillary serous carcinoma contains a large number of samples and yields a very significant p-value, even though its ratio change is lower than what is observed for the granulosa cell tumor group. Both the size of the effect and variance-based significance need to be assessed in combination with the sample size limitations to interpret the results.

### Individual Sample Assessments

Next, individual samples from the all ovarian cancer subtypes were individually tested relative to the normal ovary sample distribution. Each was defined as exceeding the 90%, 95%, 99%, and 99.9% upper confidence limits of the normal set. None of the cancerous ovary samples were below the 90% Lower Confidence Limit of 111.92 and so LCL bounds are not presented.

FIG. 6 shows a visual summary of the results for each of the classes of ovary samples. Each symbol represents a single sample plotted according to the disease class shown on the x-axis and its PARP1 expression intensity on the y-axis. Reference lines indicating the 90%, 95%, 99%, and 99.9% Normal UCLs are plotted as horizontal dashed lines. The mean of the Normal samples is plotted as a solid horizontal reference line.

Several interpretations can be made based on FIG. 6.
The elevated expression of PARP1 in cancerous ovary samples is apparent compared to normal ovary samples.
The cancerous ovary sample expression of PARP1 exhibits a much higher degree of variation than that of the normal ovary samples.
No outliers were observed within the normal ovary sample set with respect to PARP1 expression.

Table K summarizes the percentage and numbers of samples that exceed pre-defined upper confidence limits for the ovarian cancer classes.

TABLE K
Percentages and Numbers of Samples Exceeding UCLs for Ovarian Cancer Subtypes

| | >90% UCL | | >95% UCL | | >99% UCL | | >99.9% UCL | |
|---|---|---|---|---|---|---|---|---|
| Normal | 8.0% | (7/88) | 1.1% | (1/88) | 0.0% | (0/88) | 0.0% | (0/88) |
| Papillary Serous, Stage I | 100.0% | (3/3) | 100.0% | (3/3) | 100.0% | (3/3) | 100.0% | (3/3) |
| Serous Cystadenocarcinoma | 100.0% | (8/8) | 100.0% | (8/8) | 87.5% | (7/8) | 87.5% | (7/8) |
| Granulosa Cell Tumor | 100.0% | (3/3) | 100.0% | (3/3) | 66.7% | (2/3) | 66.7% | (2/3) |
| Papillary Serous, Stage III | 100.0% | (10/10) | 90.0% | (9/10) | 90.0% | (9/10) | 80.0% | (8/10) |
| Mullerian Mixed Tumor | 100.0% | (5/5) | 80.0% | (4/5) | 80.0% | (4/5) | 60.0% | (3/5) |
| Papillary Serous, Over 45 yrs | 96.3% | (26/27) | 92.6% | (25/27) | 92.6% | (25/27) | 92.6% | (25/27) |
| Papillary Serous | 90.9% | (30/33) | 87.9% | (29/33) | 84.8% | (28/33) | 81.8% | (27/33) |
| Papillary Serous, Elevated CA125 | 88.2% | (15/17) | 88.2% | (15/17) | 88.2% | (15/17) | 88.2% | (15/17) |
| Papillary Serous Secondary | 80.6% | (25/31) | 77.4% | (24/31) | 74.2% | (23/31) | 64.5% | (20/31) |
| Endometrioid, Stage I | 71.4% | (5/7) | 57.1% | (4/7) | 57.1% | (4/7) | 57.1% | (4/7) |
| Papillary Serous, Under 45 yrs | 66.7% | (4/6) | 66.7% | (4/6) | 50.0% | (3/6) | 33.3% | (2/6) |
| Endometrioid, Over 45 yrs | 63.6% | (7/11) | 54.5% | (6/11) | 54.5% | (6/11) | 54.5% | (6/11) |
| Endometrioid | 61.5% | (8/13) | 53.8% | (7/13) | 53.8% | (7/13) | 53.8% | (7/13) |
| Endometrioid, Elevated CA125 | 60.0% | (3/5) | 60.0% | (3/5) | 60.0% | (3/5) | 60.0% | (3/5) |
| Endometrioid, Stage III | 50.0% | (1/2) | 50.0% | (1/2) | 50.0% | (1/2) | 50.0% | (1/2) |
| Endometrioid, Under 45 yrs | 50.0% | (1/2) | 50.0% | (1/2) | 50.0% | (1/2) | 50.0% | (1/2) |
| Clear Cell | 50.0% | (3/6) | 33.3% | (2/6) | 33.3% | (2/6) | 16.7% | (1/6) |
| Mucinous Cystadenocarcinoma | 14.3% | (1/7) | 14.3% | (1/7) | 14.3% | (1/7) | 14.3% | (1/7) |

Several results can be made from the summary table.

Most pathologic subtypes of ovarian cancer showed a majority of samples above the 95% UCL
Papillary serous, serous cystadenocarcinoma, granulosa cell tumor and Mullerian mixed tumor all had a similar high incidence of samples above the 95% UCL
In endometrioid adenocarcinoma about half of the samples were above the 95% UCL
In clear cell adenocarcinoma and mucinous cystadenocarcinoma one-third or less of the samples were above the 95% UCL

Clinical sub-class comparisons of PARP1 expression revealed:
Papillary serous stage I was similar to papillary serous stage III
Papillary serous elevated CA125 was similar to papillary serous

### Comparison of PARP1 to Selected Genes

PARP1 expression was correlated to the expression of other genes as measured on the HG-U133A/B array set. Correlations were based on the full set of 194 samples selected for this analysis. Table L summarizes the results of this analysis. For PARP2, more than one probe set is tiled on the HG-U133A/B array set.

TABLE L
Pearson correlations of PARP1 expression to selected probe sets

| | | Correlation with |
|---|---|---|
| Gene Symbol | Fragment | 208644_at (PARP1) |
| BRCA1 | 204531_s_at | 0.314 |
| BRCA2 | 214727_at | 0.274 |
| PARP2 | 204752_x_at | 0.048 |
| | 214086_x_at | 0.052 |
| | 215773_x_at | 0.071 |
| RAD51 | 205024_s_at | 0.488 |

The gene that correlates best with PARP1 expression is RAD51 with a pearson correlation of 0.488. PARP2 had the lowest correlations to PARP1 and was essentially uncorrelated with PARP1 expression across the set of samples considered. This low correlation was consistent for all three PARP2 probe sets evaluated.

Overall, all three PARP2 probe sets, were not significantly correlated with PARP1. All other probe sets were considered statistically significantly correlated. The level of correlation is not related to the detection rate on the array. PARP2, which has the lowest correlations, has two probe sets that are present in more than 80% of samples. RAD51, which is present in only about 5% of the samples, has the highest correlation.

In no case was a negative correlation found. Positive correlations indicate that the probe sets are changing in the same direction as PARP1. When PARP1 has low expression, such as in normal samples, the expression of these correlated genes is also expected to be low. When PARP1 has elevated expression, such as in the malignant samples, the expression of these correlated genes is expected to be elevated. All of these genes, with the exception of PARP2, appear to be markers of malignancy in Ovarian cancers and respond in a similar manner to PARP2.

### Conclusions

The expression of PARP1 in ovarian cancer samples is elevated compared to normals. FIG. 6 shows that, despite this finding, not all ovarian cancer samples exhibit this overexpression. This wider distribution and shift towards higher expression in the ovarian cancer groups indicate that ˜75% of ovarian cancers have PARP1 expression above the 95% upper confidence limit of normal ovary expression. Further analysis into various subgroups of ovarian cancer samples reveals that the percentage of ovarian cancer samples observed to have elevated PARP1 expression increases to ˜90% if they are of the subtypes papillary serous adenocarcinoma, serous cystadenocarcinoma, Mullerian mixed tumor, or granulosa cell tumor. Clear cell adenocarcinoma and mucinous cystadenocarcinoma did demonstrated elevated PARP1 in one-third or less of the samples assessed.

In summary,
1. PARP1 expression is higher in ovarian cancer than in normal ovary tissue.
2. Certain subtypes of ovarian cancer appear to exhibit higher expression levels than other subtypes. Specifically, the papillary serous adenocarcinoma, serous cystadenocarcinoma, Mullerian mixed tumor, and granulosa cell tumor samples showed higher percentages of samples above the normal UCL's than endometrioid, which, in turn, showed a higher percentage of samples above the normal UCL's than clear cell adenocarcinoma and mucinous cystadenocarcinoma.

### Discussion and Interpretation

If over-expression of PARP1 in ovarian cancer is defined as a level greater than the 95% upper confidence limit of expression in normal ovary tissue, then ˜75% of ovarian cancer samples over-express PARP1. If PARP1 over-expression defines increased responsiveness to PARP1 inhibition, then the results imply that a substantial fraction of ovarian cancers would be rational candidates for therapy with PARP1 inhibitors, in particular, the papillary serous adenocarcinoma, serous cystadenocarcinoma, Mullerian mixed tumor, and granulosa cell tumor subtypes. Clear Cell Adenocarcinoma and Mucinous Cystadenocarcinoma express much less than the other sub-types; it is possible therefore that these sub-types may be less susceptible to PARP1 inhibition.

Within the group of epithelial ovarian carcinomas (which exclude Mullerian Mixed Tumor and Granulosa Cell Tumor) it is understood that the molecular pathology is heterogeneous. High-grade serous and endometrioid carcinomas are characterized by p53 mutations, and BRCA1 and/or BRCA2 dysfunction. In contrast, low-grade serous carcinomas are characterized by KRAS or BRAF mutations and low-grade endometrioid carcinomas have mutations in PTEN and CTNNB1. Clear-cell carcinomas are characterized by mutations of TGFbetaR2, and mucinous carcinomas have KRAS mutations. (Christie M, Oehler M K. Molecular pathology of epithelial ovarian cancer. J Br Menopause Soc. 2006 Jun; 12(2):57-63.) It is possible that the variation in PARP1 expression patterns in epithelial ovarian carcinomas may reflect these mutational changes.

There was no obvious association of PARP1 expression and clinical sub-classes (based on age, CA125 level and stage) suggesting that PARP1 expression is principally related to the pathological subtype of ovarian cancers within this study. However, sample numbers for the clinical sub-classes were limited, and thus conclusions regarding them should be made cautiously.

Correlation of PARP1 expression to the genes BRCA 1, BRCA2, RAD51, and PARP2 indicated significant correlation to all except PARP2. RAD51 had the highest correlation.

### Example 4

### Gene Expression of PARP1 in Malignant and Normal Endometrium, Lung, and Prostate Tissue Samples

This project is a study of the expression of PARP1 mRNA in human normal endometrium (n=23), lung (n=122), and prostate (n=57) and various cancers of the endometrium (n=57), lung (n=101), and prostate (n=57) as measured on the Affymetrix HG-U133A/B array set.

The primary goal of the study was to define “over-expression” of PARP1 mRNA by using objective statistical thresholds based on PARP1 expression in the normal tissue samples, and then to identify and characterize cancer samples that exceed those statistical thresholds. The secondary goal of the study was to correlate PARP1 expression in the same samples to the expression of all other genes tiled on the HG-U133 A/B array set in order to identify genes with similar (or opposite) expression characteristics.

The expression of PARP1 in cancer was generally elevated compared to normals. PARP1 expression was above the 95% upper confidence limit of the normal population (“over-expression”) in about one-quarter of all endometrial, about three-quarters of all lung, and about one-eighth of all prostate cancer samples. The Mullerian mixed tumors and the lung squamous cell carcinomas exhibited the highest incidences of elevated PARP1 expression. PARP1 expression in prostate adenocarcinoma was considerably lower than for the cancer types assessed in endometrium and lung tissues.

Correlation of PARP1 to all other genes identified genes with correlations to PARP1 as high as 80%. Among the endometrium and lung samples, a common set of genes associated with cell proliferation were identified that correlated highly (i.e. in the top 40) in both tissues.

This analysis project is an investigation of the expression of the PARP1 mRNA in human normal and cancerous endometrium, lung, and prostate samples as measured on the Affymetrix HG-U133A/B array set. Samples for this project were previously accrued and processed over arrays by Gene Logic Inc. for the purposes of construction of a therapeutically relevant gene expression database. The large number of normal and cancerous samples of each tissue type and the classification of the cancerous samples into multiple subsets of therapeutically relevant types allows for a robust analysis and interpretation of results. This analysis addresses the following objectives:

characterization of the expression of PARP1 relative to individual endometrium, lung and prostate oncology samples as compared to control samples (i.e., “normals”) from the same or medically similar tissue type.

characterization of the expression of PARP1 relative to the expression of all other genes on the HG-U133A/B array set.

### Study Design

### Materials and Methods

### Quality Control

RNA is evaluated for quality and integrity (via Agilent Bioanalyzer derived 28 s/28 s ratio and RNA integrity number (RIN)), purity (via absorbance ratio at A260/A280), and quantity (via absorbance at A260 or alternative assay (i.e. ribogreen)), Quantity and purity of cRNA synthesis product is assessed using UV absorbance. Quality of cRNA synthesis is assessed using either the Agilent Bioanalyzer or a MOPS agarose gel. Array quality is evaluated using a proprietary high throughput application by which arrays are evaluated against several strict objective standards such as 5′/3′ GAPDH ratio, signal/noise ratio. and background as well as over thirty additional metrics (e.g. outlier, vertical variance). Data generated throughout the process is managed within the quality system to ensure data integrity of the data.

### Statistical Analysis

23, 122, and 57 pathologically normal tissue samples were used to determine baseline expression of the PARP1 gene in endometroid, lung, and prostate tissue, respectively. The mean and 90%, 95%, 99%, and 99.9% upper confidence limits for an individual predicted value (UCLs) were calculated. Because we are assessing the likelihood that individual samples external to the normal set are within the baseline distribution, the prediction interval, rather than the confidence interval for the mean, was selected to estimate the expected range for future individual measurements. The prediction interval is defined by the formula, X̅±AS\sqrt{1+(1/n)}, where X̅ is the mean of the normal breast samples, S is the standard deviation of the normal samples, n is the sample size of the normal samples, and A is the 100(1−(p/2))th percentile of the Student's t-distribution with n−1 degrees of freedom. Prior knowledge of the PARP1 gene's elevated expression in oncology samples indicated a primary interest in up-regulation relative to the baseline. Therefore, lower confidence limits were not calculated.

57, 101, and 57 carcinoma samples from multiple subtypes in endometroid, lung, and prostate tissues, respectively, were assessed in the context of their respective normal expression limits. These samples were grouped into various subcategories according to well-accepted characteristics including tumor stage, smoking status, or age. Some samples were members of more than one subcategory and some were not members of any subcategory beyond the primary cancer type. Each carcinoma sample was identified as being above the 90%, 95%, 99%, or 99.9% UCLs. Pearson's correlations were calculated for 44,759 probe sets on the Affymetrix HG-U133 AB array set as compared to PARP1. Correlations were based on the set of 80, 123, and 114 samples in endometroid, lung, and prostate tissues, respectively.

All analysis was performed using SAS v8.2 for Windows (www.sas.com) and utilized MAS 5 expression intensities as calculated from the Affymetrix GeneChip® Operating System (www.affymetrix.com).

Individual normal and cancerous samples from endometrium, lung, and prostate tissues were selected. Any cancerous sample may be represented in more than one subtype grouping. An example is shown in Table M for 10 selected endometrial samples and their membership in multiple subtypes.

TABLE M
Examples of subtype classification of selected endometroid samples

| | | | | AdnCarc, | | AdnCarc, | AdnCarc, | AdnCarc, | | | | AdnCarc, |
|---|---|---|---|---|---|---|---|---|---|---|---|---|
| | | | | Endo- | | Endometrioid, | Endo- | Endo- | | | | Endo- |
| | | | | metrioid, | AdnCarc, | Obese, | metrioid, | metrioid, | AdnCarc, | AdnCarc, | | metrioid, |
| | | | AdnCarc, | No | Endo- | No | Post- | Pre- | Endo- | Endo- | Mullerian | Obese, |
| | | Malig- | Endo- | Smoking | metrioid, | Smoking | meno- | meno- | metrioid, | metrioid, | Mixed | Smoking |
| GID | Normal | nant | metrioid | Hist | Obese | Hist | pausal | pausal | Stage I | Nonobese | Tumor | Hist |
| 565 | Y | | | | | | | | | | | |
| 612 | Y | | | | | | | | | | | |
| 1109 | Y | | | | | | | | | | | |
| 1119 | Y | | | | | | | | | | | |
| 1146 | | Y | Y | Y | | | Y | | | | | |
| 1427 | | Y | Y | Y | | | | Y | | | | |
| 1638 | Y | | | | | | | | | | | |
| 1815 | Y | | | | | | | | | | | |
| 2401 | | Y | Y | Y | | | | | Y | | | |
| 2402 | | Y | Y | Y | | | Y | | Y | Y | | |

The PARP1 gene is represented on the HG-U133A array by a single probe set with the identifier “208644_at”. All results were generated based on the MAS5 expression signal intensities for this probe set and will be referred to as “PARP1”.

### Statistical Analysis—Endometrium Results

The normal and malignant sample classes were summarized by mean, standard deviation, standard error, and several upper confidence limits based on at distribution. The upper confidence limits (UCL) are similar to standard deviation statistics in that they identify specific regions of probability for observing a value. For instance, a 95% upper confidence limit is akin to a value above which one would expect by chance in 5% of samples.

Table N shows summary statistics for each of the normal and cancerous endometrium sample sets.

TABLE N
Summary statistics for the normal and cancerous endometroid sample sets

| | | | Std | Std | 90% | 95% | 99% | 99.9% |
|---|---|---|---|---|---|---|---|---|
| Group | Number | Mean | Dev | Err | UCL | UCL | UCL | UCL |
| Normal | 23 | 201.21 | 62.21 | 12.97 | 310.33 | 333.00 | 380.34 | 442.20 |
| AdnCarc, Endometrioid | 50 | 297.42 | 98.78 | 13.97 | 464.67 | 497.89 | 564.77 | 646.62 |
| AdnCarc, Endometrioid, | 40 | 286.55 | 91.55 | 14.47 | 442.71 | 474.02 | 537.53 | 616.33 |
| No Smoking Hist | | | | | | | | |
| AdnCarc, Endometrioid, | 3 | 373.40 | 76.85 | 44.37 | 632.53 | 755.23 | 1254.16 | 3177.60 |
| Nonobese | | | | | | | | |
| AdnCarc, Endometrioid, | 15 | 291.22 | 79.84 | 20.61 | 436.46 | 468.08 | 536.69 | 632.64 |
| Obese | | | | | | | | |
| AdnCarc, Endometrioid, | 9 | 260.84 | 73.44 | 24.48 | 404.79 | 439.35 | 520.58 | 651.08 |
| Obese, No Smoking Hist | | | | | | | | |
| AdnCarc, Endometrioid, | 6 | 336.79 | 71.19 | 29.06 | 491.73 | 534.44 | 646.82 | 864.94 |
| Obese, Smoking Hist | | | | | | | | |
| AdnCarc, Endometrioid, | 35 | 308.83 | 97.96 | 16.56 | 476.82 | 510.73 | 579.90 | 666.56 |
| Postmenopausal | | | | | | | | |
| AdnCarc, Endometrioid, | 3 | 250.09 | 12.48 | 7.20 | 292.15 | 312.07 | 393.07 | 705.32 |
| Premenopausal | | | | | | | | |
| AdnCarc, Endometrioid, | 6 | 336.79 | 71.19 | 29.06 | 491.73 | 534.44 | 646.82 | 864.94 |
| Smoking Hist | | | | | | | | |
| AdnCarc, Endometrioid, | 9 | 340.80 | 68.13 | 22.71 | 474.34 | 506.40 | 581.76 | 702.84 |
| Stage I | | | | | | | | |
| Mullerian Mixed Tumor | 7 | 517.86 | 185.55 | 70.13 | 903.31 | 1003.23 | 1253.26 | 1699.84 |

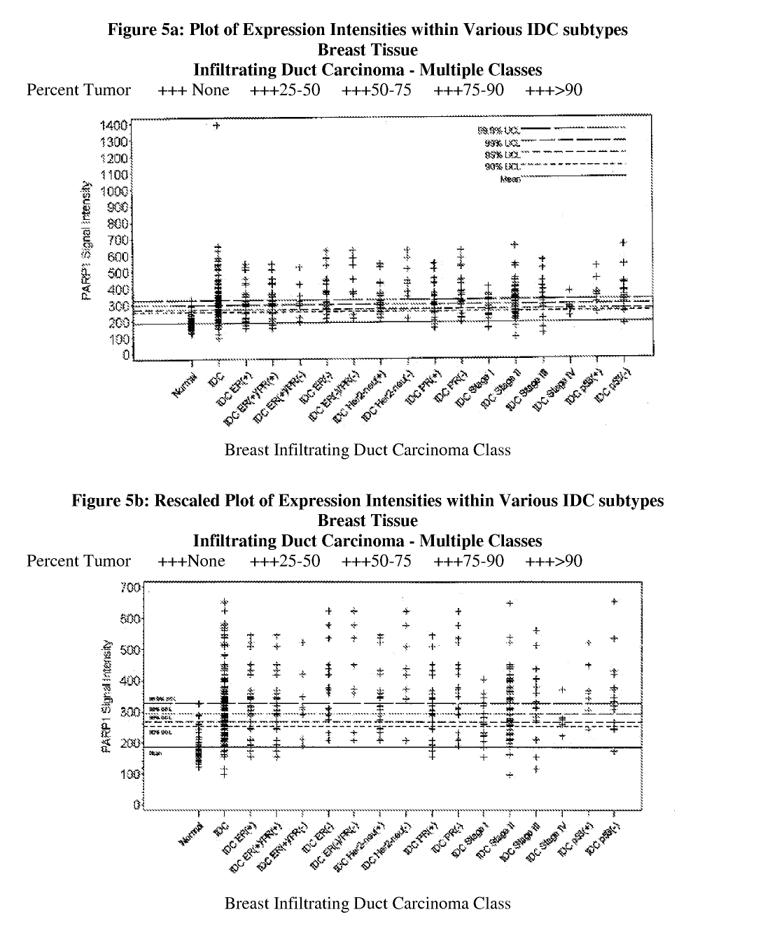

All of the endometrial cancers expressed higher mean PARP1 signal intensities than normal endometrium. The Mullerian Mixed Tumor samples expressed considerably higher PARP1 than did the other subtypes. This is shown visually in FIG. 7 below.

Table O lists the ratio-based fold change and Student's two-tailed t-test results of the PARP1 gene expression when compared to normal.

TABLE O
Comparison statistics of endometriod
cancer types to normal endometrium

| | Fold Change | t-test p-value |
|---|---|---|
| Cancer Group | (Cancer/Normal) | (vs. Normal) |
| AdnCarc, Endometrioid | 1.48 | 3.972E−06 |
| AdnCarc, Endometrioid, | 1.42 | 4.740E−05 |
| No Smoking Hist | | |
| AdnCarc, Endometrioid, | 1.86 | 5.035E−02 |
| Nonobese | | |
| AdnCarc, Endometrioid, | 1.45 | 1.088E−03 |
| Obese | | |
| AdnCarc, Endometrioid, | 1.30 | 5.109E−02 |
| Obese, No Smoking Hist | | |
| AdnCarc, Endometrioid, | 1.67 | 3.596E−03 |
| Obese, Smoking Hist | | |
| AdnCarc, Endometrioid, | 1.53 | 3.947E−06 |
| Postmenopausal | | |
| AdnCarc, Endometrioid, | 1.24 | 3.941E−03 |
| Premenopausal | | |
| AdnCarc, Endometrioid, | 1.67 | 3.596E−03 |
| Smoking Hist | | |
| AdnCarc, Endometrioid, | 1.69 | 1.172E−04 |
| Stage I | | |
| Mullerian Mixed Tumor | 2.57 | 3.721E−03 |

Next, individual samples from the all endometrial cancer subtypes were individually tested relative to the normal endometrium sample distribution. Each was defined as exceeding the 90%, 95%, 99%, and 99.9% upper confidence limits of the normal set.

FIG. 7 shows a visual summary of the results for each of the classes of endometrial samples. Each symbol represents a single sample plotted according to the disease class shown on the x-axis and its PARP1 expression intensity on the y-axis. Reference lines indicating the 90%, 95%, 99%, and 99.9% Normal UCLs are plotted as horizontal dashed lines. The mean of the Normal samples is plotted as a solid horizontal reference line.

The elevated expression of PARP1 in cancerous endometrium samples is apparent relative to normal endometrium samples. The cancerous endometrium sample expression of PARP1 exhibits a much higher degree of variation (i.e., greater spread) than that of the normal endometrium samples. No outliers were observed within the normal endometrium sample set with respect to PARP1 expression.

Table P summarizes the percentage and numbers of samples that exceed predefined upper confidence limits for the endometrium cancer classes. The table has been sorted with respect to the class with the greatest incidence of samples exceeding the 90% UCL. Therefore, the classes toward the top of the list contain the highest proportion of samples that exceed the normal threshold.

TABLE P
Percentages (counts) of samples exceeding UCLs for endometroid cancer subtypes

| | >90% UCL | | >95% UCL | | >99% UCL | | >99.9% UCL | |
|---|---|---|---|---|---|---|---|---|
| Normal | 4.3% | (1/23) | 4.3% | (1/23) | 4.3% | (1/23) | 0.0% | (0/23) |
| AdnCarc, Endometrioid, | 100.0% | (3/3) | 33.3% | (1/3) | 33.3% | (1/3) | 33.3% | (1/3) |
| Nonobese | | | | | | | | |
| Mullerian Mixed Tumor | 85.7% | (6/7) | 85.7% | (6/7) | 71.4% | (5/7) | 71.4% | (5/7) |
| AdnCarc, Endometrioid, | 83.3% | (5/6) | 50.0% | (3/6) | 16.7% | (1/6) | 0.0% | (0/6) |
| Obese, Smoking Hist | | | | | | | | |
| AdnCarc, Endometrioid, | 83.3% | (5/6) | 50.0% | (3/6) | 16.7% | (1/6) | 0.0% | (0/6) |
| Smoking Hist | | | | | | | | |
| AdnCarc, Endometrioid, | 66.7% | (6/9) | 33.3% | (3/9) | 22.2% | (2/9) | 11.1% | (1/9) |
| Stage I | | | | | | | | |
| AdnCarc, Endometrioid, | 53.3% | (8/15) | 26.7% | (4/15) | 6.7% | (1/15) | 0.0% | (0/15) |
| Obese | | | | | | | | |
| AdnCarc, Endometrioid, | 51.4% | (18/35) | 37.1% | (13/35) | 20.0% | (7/35) | 11.4% | (4/35) |
| Postmenopausal | | | | | | | | |
| AdnCarc, Endometrioid | 46.0% | (23/50) | 30.0% | (15/50) | 18.0% | (9/50) | 10.0% | (5/50) |
| AdnCarc, Endometrioid, | 40.0% | (16/40) | 25.0% | (10/40) | 15.0% | (6/40) | 7.5% | (3/40) |
| No Smoking Hist | | | | | | | | |
| AdnCarc, Endometrioid, | 33.3% | (3/9) | 11.1% | (1/9) | 0.0% | (0/9) | 0.0% | (0/9) |
| Obese, No Smoking Hist | | | | | | | | |
| AdnCarc, Endometrioid, | 0.0% | (0/3) | 0.0% | (0/3) | 0.0% | (0/3) | 0.0% | (0/3) |
| Premenopausal | | | | | | | | |

Most pathologic subtypes of endometrium cancer showed a majority of samples above the 90% UCL. Of particular note, Mullerian Mixed Tumor had the highest incidence (85.7%) of samples above the 95% UCL and remained high (71.4%) at the 99.9% UCL.

### Lung Results

The normal and malignant sample classes were summarized by mean, standard deviation, standard error, and several upper confidence limits based on at distribution. The upper confidence limits (UCL) are similar to standard deviation statistics in that they identify specific regions of probability for observing a value. For instance, a 95% upper confidence limit is akin to a value above which one would expect by chance in 5% of samples.

Table Q shows summary statistics for each of the normal and cancerous lung sample sets.

TABLE Q
Summary statistics for the normal and cancerous lung sample sets

| | | | Std | Std | 90% | 95% | 99% | 99.9% |
|---|---|---|---|---|---|---|---|---|
| Group | Number | Mean | Dev | Err | UCL | UCL | UCL | UCL |
| Normal | 122 | 162.37 | 32.85 | 2.97 | 217.03 | 227.66 | 248.68 | 273.60 |
| Adenosquamous | 3 | 209.41 | 25.20 | 14.55 | 294.36 | 334.59 | 498.17 | 1128.78 |
| Carcinoma | | | | | | | | |
| AdnCarc | 46 | 284.99 | 92.24 | 13.60 | 441.58 | 472.79 | 535.77 | 613.23 |
| AdnCarc, Smoking | 27 | 276.68 | 54.55 | 10.50 | 371.43 | 390.86 | 431.03 | 482.57 |
| Hist | | | | | | | | |
| AdnCarc, Stage I | 10 | 244.47 | 43.66 | 13.81 | 328.41 | 348.06 | 393.29 | 463.40 |
| AdnCarc, Stage II | 7 | 301.52 | 64.51 | 24.38 | 435.53 | 470.27 | 557.19 | 712.45 |
| AdnCarc, Stage III | 5 | 301.58 | 85.87 | 38.40 | 502.11 | 562.74 | 734.66 | 1111.49 |
| Large Cell | 7 | 291.08 | 122.74 | 46.39 | 546.06 | 612.16 | 777.56 | 1072.98 |
| Carcinoma | | | | | | | | |
| Large Cell | 6 | 256.71 | 90.31 | 36.87 | 453.27 | 507.46 | 650.03 | 926.74 |
| Carcinoma, Smoking | | | | | | | | |
| Hist | | | | | | | | |
| Large Cell | 4 | 356.73 | 110.50 | 55.25 | 647.46 | 749.89 | 1078.32 | 1953.37 |
| Carcinoma, Stage I | | | | | | | | |
| Neuroendocrine | 3 | 408.91 | 287.69 | 166.10 | 1378.91 | 1838.22 | 3705.88 | 10905.91 |
| Carcinoma (Non- | | | | | | | | |
| Small Cell) | | | | | | | | |
| Small Cell | 3 | 473.23 | 239.88 | 138.49 | 1282.03 | 1665.02 | 3222.30 | 9225.83 |
| Carcinoma | | | | | | | | |
| Small Cell | 3 | 473.23 | 239.88 | 138.49 | 1282.03 | 1665.02 | 3222.30 | 9225.83 |
| Carcinoma, Stage II | | | | | | | | |
| Squamous Cell | 39 | 309.53 | 103.71 | 16.61 | 486.62 | 522.16 | 594.34 | 684.05 |
| Carcinoma | | | | | | | | |
| Squamous Cell | 36 | 310.91 | 107.51 | 17.92 | 495.06 | 532.17 | 607.78 | 702.31 |
| Carcinoma, Smoking | | | | | | | | |
| Hist | | | | | | | | |
| Squamous Cell | 16 | 315.57 | 78.05 | 19.51 | 456.60 | 487.04 | 552.63 | 643.22 |
| Carcinoma, Stage I | | | | | | | | |
| Squamous Cell | 5 | 291.67 | 30.10 | 13.46 | 361.98 | 383.23 | 443.50 | 575.61 |
| Carcinoma, Stage II | | | | | | | | |
| Squamous Cell | 5 | 236.10 | 63.69 | 28.48 | 384.83 | 429.80 | 557.30 | 836.79 |
| Carcinoma, Stage | | | | | | | | |
| III | | | | | | | | |

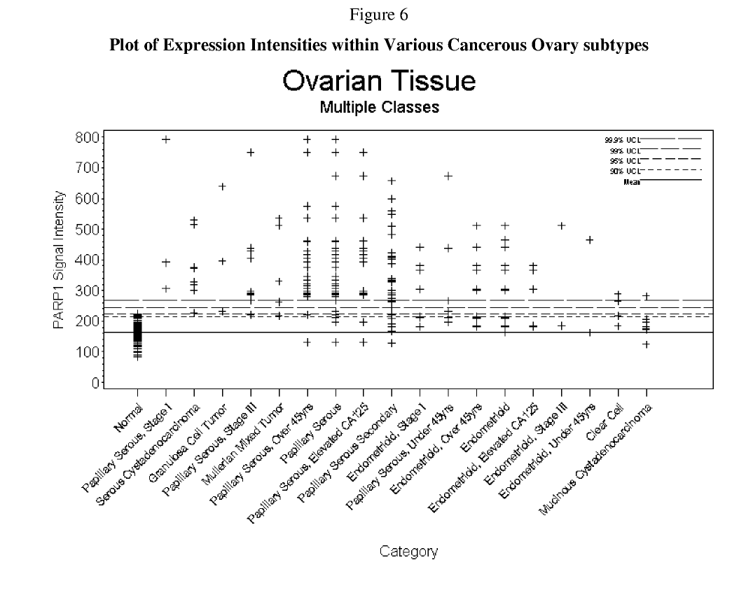

All of the lung cancers expressed higher mean PARP1 signal intensities than normal lung. This is shown visually in FIG. 8 below.

Table R lists the ratio-based fold change and Student's two-tailed t-test results of the PARP1 gene expression when compared to normal.

TABLE R
Comparison statistics of lung cancer types to normal lung

| | Fold Change | t-test p-value |
|---|---|---|
| Cancer Group | (Cancer/Normal) | (vs. Normal) |
| Adenosquamous Carcinoma | 1.29 | 7.811E−02 |
| AdnCarc | 1.76 | 1.073E−11 |
| AdnCarc, Smoking Hist | 1.70 | 1.359E−11 |
| AdnCarc, Stage I | 1.51 | 1.800E−04 |
| AdnCarc, Stage II | 1.86 | 1.176E−03 |
| AdnCarc, Stage III | 1.86 | 2.201E−02 |
| Large Cell Carcinoma | 1.79 | 3.220E−02 |
| Large Cell Carcinoma, | 1.58 | 5.062E−02 |
| Smoking Hist | | |
| Large Cell Carcinoma, | 2.20 | 3.876E−02 |
| Stage I | | |
| Neuroendocrine Carcinoma | 2.52 | 2.760E−01 |
| (Non-Small Cell) | | |
| Small Cell Carcinoma | 2.91 | 1.539E−01 |
| Small Cell Carcinoma, | 2.91 | 1.539E−01 |
| Stage II | | |
| Squamous Cell Carcinoma | 1.91 | 7.722E−11 |
| Squamous Cell Carcinoma, | 1.91 | 8.215E−10 |
| Smoking Hist | | |
| Squamous Cell Carcinoma, | 1.94 | 9.249E−07 |
| Stage I | | |
| Squamous Cell Carcinoma, | 1.80 | 4.516E−04 |
| Stage II | | |
| Squamous Cell Carcinoma, | 1.45 | 6.037E−02 |
| Stage III | | |

Next, individual samples from the all lung cancer subtypes were individually tested relative to the normal lung sample distribution. Each was defined as exceeding the 90%, 95%, 99%, and 99.9% upper confidence limits of the normal set. None of the cancerous lung samples were below the 90% Lower Confidence Limit of normals and so LCL bounds are not presented.

FIG. 8 shows a visual summary of the results for each of the classes of lung samples. Each symbol represents a single sample plotted according to the disease class shown on the x-axis and its PARP1 expression intensity on the y axis. Reference lines indicating the 90%, 95%, 99%, and 99.9% Normal UCLs are plotted as horizontal dashed lines. The mean of the Normal samples is plotted as a solid horizontal reference line. The elevated expression of PARP1 in cancerous lung samples is apparent relative to normal lung samples. The cancerous lung sample expression of PARP1 exhibits a higher degree of variation (i.e., greater spread) than that of the normal lung samples.

Table S summarizes the percentage and numbers of samples that exceed predefined upper confidence limits for the lung cancer classes. The table has been sorted with respect to the class with the greatest incidence of samples exceeding the 90% UCL. Therefore, the classes toward the top of the list contain the highest proportion of samples that exceed the normal threshold.

TABLE S
Percentages (counts) of samples exceeding UCLs for lung cancer subtypes

| | >90% UCL | | >95% UCL | | >99% UCL | | >99.9% UCL | |
|---|---|---|---|---|---|---|---|---|
| Normal | 4.9% | (6/122) | 3.3% | (4/122) | 1.6% | (2/122) | 0.0% | (0/122) |
| Small Cell Carcinoma | 100.0% | (3/3) | 100.0% | (3/3) | 100.0% | (3/3) | 100.0% | (3/3) |
| Small Cell Carcinoma, | 100.0% | (3/3) | 100.0% | (3/3) | 100.0% | (3/3) | 100.0% | (3/3) |
| Stage II | | | | | | | | |
| Large Cell Carcinoma, | 100.0% | (4/4) | 100.0% | (4/4) | 100.0% | (4/4) | 75.0% | (3/4) |
| Stage I | | | | | | | | |
| Squamous Cell Carcinoma, | 100.0% | (5/5) | 100.0% | (5/5) | 100.0% | (5/5) | 60.0% | (3/5) |
| Stage II | | | | | | | | |
| Neuroendocrine Carcinoma | 100.0% | (3/3) | 100.0% | (3/3) | 66.7% | (2/3) | 33.3% | (1/3) |
| (Non-Small Cell) | | | | | | | | |
| Squamous Cell Carcinoma, | 87.5% | (14/16) | 87.5% | (14/16) | 81.3% | (13/16) | 68.8% | (11/16) |
| Stage I | | | | | | | | |
| Squamous Cell Carcinoma | 87.2% | (34/39) | 82.1% | (32/39) | 74.4% | (29/39) | 61.5% | (24/39) |
| Squamous Cell Carcinoma, | 86.1% | (31/36) | 80.6% | (29/36) | 75.0% | (27/36) | 61.1% | (22/36) |
| Smoking Hist | | | | | | | | |
| AdnCarc, Stage II | 85.7% | (6/7) | 85.7% | (6/7) | 85.7% | (6/7) | 57.1% | (4/7) |
| AdnCarc, Smoking Hist | 85.2% | (23/27) | 85.2% | (23/27) | 74.1% | (20/27) | 40.7% | (11/27) |
| AdnCarc, Stage III | 80.0% | (4/5) | 80.0% | (4/5) | 80.0% | (4/5) | 80.0% | (4/5) |
| Squamous Cell Carcinoma, | 80.0% | (4/5) | 60.0% | (3/5) | 20.0% | (1/5) | 20.0% | (1/5) |
| Stage III | | | | | | | | |
| AdnCarc | 76.1% | (35/46) | 73.9% | (34/46) | 63.0% | (29/46) | 37.0% | (17/46) |
| Large Cell Carcinoma | 71.4% | (5/7) | 71.4% | (5/7) | 71.4% | (5/7) | 57.1% | (4/7) |
| AdnCarc, Stage I | 70.0% | (7/10) | 70.0% | (7/10) | 60.0% | (6/10) | 20.0% | (2/10) |
| Large Cell Carcinoma, | 66.7% | (4/6) | 66.7% | (4/6) | 66.7% | (4/6) | 50.0% | (3/6) |
| Smoking Hist | | | | | | | | |
| Adenosquamous Carcinoma | 33.3% | (1/3) | 33.3% | (1/3) | 0.0% | (0/3) | 0.0% | (0/3) |

### Prostate Results

Table T shows summary statistics for each of the normal and cancerous prostate sample sets.

TABLE T
Summary statistics for the normal and cancerous prostate sample sets

| | | | Std | Std | 90% | 95% | 99% | 99.9% |
|---|---|---|---|---|---|---|---|---|
| Group | Number | Mean | Dev | Err | UCL | UCL | UCL | UCL |
| Normal | 57 | 209.09 | 36.61 | 4.85 | 270.86 | 283.08 | 307.57 | 337.36 |
| AdnCarc, Age 60 and Over | 57 | 237.80 | 40.49 | 5.36 | 306.11 | 319.61 | 346.70 | 379.63 |

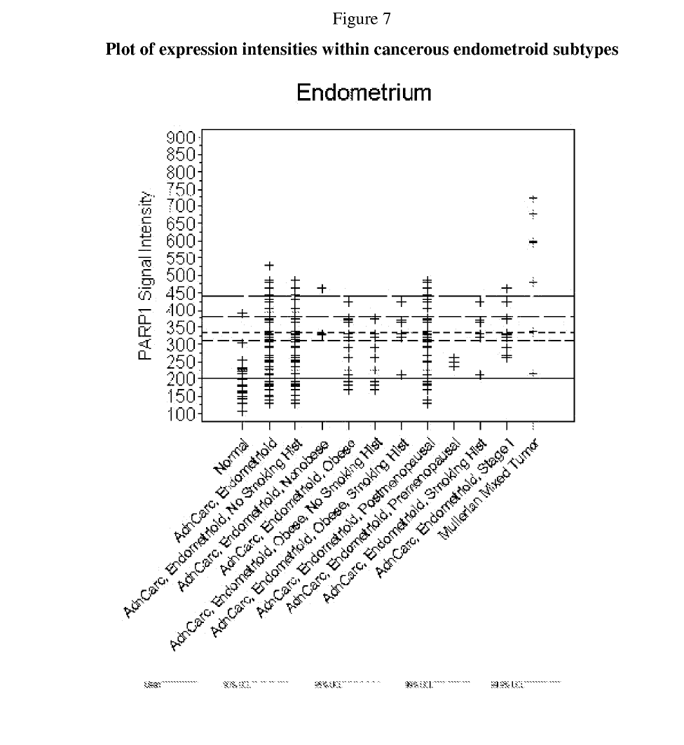

The prostate cancer group expressed a somewhat higher mean PARP1 signal intensity than the normal prostate group. This is shown visually in FIG. 9.

Table U lists the ratio-based fold change and Student's two-tailed t-test results of the PARP1 gene expression when compared to normal.

TABLE U
Comparison statistics of prostate cancer types to normal prostate

| | Fold Change | t-test p-value |
|---|---|---|
| Cancer Group | (Cancer/Normal) | (vs. Normal) |
| AdnCarc, | 1.14 | 1.273E−04 |
| Age 60 | | |
| and Over | | |

FIG. 9 shows a visual summary of the results for each of the classes of prostate samples. Each symbol represents a single sample plotted according to the disease class shown on the x-axis and its PARP1 expression intensity on the y-axis. Reference lines indicating the 90%, 95%, 99%, and 99.9% Normal UCLs are plotted as horizontal dashed lines. The mean of the Normal samples is plotted as a solid horizontal reference line. The slightly elevated expression of PARP1 in cancerous prostate samples is apparent relative to normal prostate samples. The cancerous prostate sample expression of PARP1 exhibits a similar degree of variation (i.e., equivalent spread) than that of the normal prostate samples.

Table V summarizes the percentage and numbers of samples that exceed predefined upper confidence limits for the prostate cancer class.

TABLE V
Percentages (counts) of samples exceeding
UCLs for prostate cancer subtypes

| | >90% UCL | >95% UCL | >99% UCL | >99.9% UCL |
|---|---|---|---|---|
| Normal | 7.0% (4/57) | 1.8% (1/57) | 0.0% (0/57) | 0.0% (0/57) |
| AdnCarc, | 17.5% (10/57) | 12.3% (7/57) | 7.0% (4/57) | 0.0% (0/57) |
| Age 60 | | | | |
| and Over | | | | |

The somewhat higher expression of PARP1 in Prostate Adenocarcinoma, Age 60 and Over is again reflected in slightly higher incidences of samples exceeding the 90%, 95% and 99% UCL thresholds. All samples from both the normal and cancerous groups were within the 99.9% UCL limit.

These results imply that a substantial fraction of lung and selected endometrial cancers would be rational candidates for therapy with PARP1 inhibitors, in particular, the Mullerian mixed tumor, and the squamous cell carcinomas of the lung. PARP1 expression is higher in endometrial and lung cancer than in their respective normal tissue. Certain subtypes of endometrial and lung cancer appear to exhibit higher expression levels than other subtypes. Specifically, Mullerian mixed tumor, and lung squamous cell carcinoma samples showed higher percentages of samples above the Normal UCL's than the other classes.

### Discussion and Interpretation

If over-expression of PARP1 in cancer is defined as a level greater than the 95% upper confidence limit of expression in normal tissue, then ˜37% of endometrial, ˜77% of lung, and ˜12% of prostate cancer samples over-express PARP1. The lung carcinomas have the highest rate of samples over the normal 95% UCL, but not all of the groups represent statistically significant elevations. While the endometrial carcinomas had a lower rate of samples over the normal 95% UCL, the Mullerian mixed tumor class represented the largest statistically significant fold change across all three tissues evaluated. While lung may represent the tissue type with the greatest change in PARP1 expression, the Mullerian mixed tumor class represents the most affected single class evaluated.

Accepting the premise that PARP1 over-expression defines increased responsiveness to PARP1 inhibition, these results imply that a substantial fraction of lung and selected endometrial cancers would be rational candidates for therapy with PARP1 inhibitors, in particular, the Mullerian mixed tumor, and the squamous cell carcinomas of the lung. Small cell carcinomas in lung may also eventually be found to show large increases in PARP1 expression, but the current dataset had insufficient samples to definitively measure fold change or determine statistical significance. In contrast, prostate adenocarcinoma shows only a small, though statistically significant, increase in PARP1 expression and thus may be less susceptible to PARP1 inhibition according to the premise.

### Conclusions

The expression of PARP1 in endometrial and lung cancer samples is generally elevated compared to normals. Similar signal elevation was not seen the in the prostate cancer samples evaluated. The figures show that, despite this finding, not all endometrial and lung cancer samples exhibit this overexpression. This wider distribution and shift towards higher expression in the endometrial and lung cancer groups indicate that ˜37% of endometrial and ˜77% of lung cancers have PARP1 expression above the 95% upper confidence limit of their respective normal expression. Further analysis into various subgroups of endometrial cancer samples reveals that the percentage of cancer samples observed to have elevated PARP1 expression increases to ˜86% if they are of the Mullerian mixed tumor subtype. Clear Cell Adenocarcinoma and Mucinous Cystadenocarcinoma did demonstrated elevated PARP1 in one-third or less of the samples assessed and may represent less sensitive cancer types. These findings should be further investigated and confirmed. In summary,
1. PARP1 expression is higher in endometrial and lung cancer than in their respective normal tissue.
2. Certain subtypes of endometrial and lung cancer appear to exhibit higher expression levels than other subtypes. Specifically, Mullerian mixed tumor, and lung squamous cell carcinoma samples showed higher percentages of samples above the Normal UCL's than the other classes.

### Example 5

### Monitoring PARP Expression in Tissue Samples

### Assay Description and Methods

XP™-PCR is a multiplex RT-PCR methodology that allows for the expression analysis of multiple genes in a single reaction (Quin-Rong Chen, Gordon Vansant, Kahuku Oades, Maria Pickering, Jun S. Wei, Young K. Song, Joseph Monforte, and Javed Khan: Diagnosis of the Small Round Blue Cell Tumors Using Mutliplex Polymerase Chain Reaction. Journal of Molecular Diagnostics, Vol. 9. No. 1, February 2007). A defined combination of gene specific and universal primers used in the reaction results in a series of fluorescently labeled PCR products whose size and quantity are measured using the capillary electrophoresis instrument GeXP.

### Sample Treatments

Briefly, freshly purified tissue samples will be plated in 24-well plates at 6×10⁶ cells per well. One half of the samples will be lysed immediately and the others will be quickly frozen in a dry ice and ethanol bath and stored at −80° C. for 24 hours. Total RNA from each sample will be isolated following Althea Technologies, Inc. SOP Total RNA Isolation Using Promega SV96 Kit (Cat. No. Z3505). The concentration of the RNA obtained from each sample will be obtained using 03-XP-008, RNA Quantitation Using the Quant-it Ribogreen RNA Assay Kit (Cat. No. R-11490). A portion of RNA from each sample will be adjusted to 5 ng/μL and then subjected to XP™-PCR.

### XP™-PCR

Multiplex RT-PCR will be performed using 25 ng of total RNA of each sample using a previously described protocol (Quin-Rong Chen, Gordon Vansant, Kahuku Oades, Maria Pickering, Jun S. Wei, Young K. Song, Joseph Monforte, and Javed Khan: Diagnosis of the Small Round Blue Cell Tumors Using Mutliplex Polymerase Chain Reaction. Journal of Molecular Diagnostics, Vol. 9. No. 1, February 2007). The RT reactions will be carried out as described in SOP 1′-XP-002, cDNA Production from RNA with the Applied Biosystems 9700. PCR reactions will be carried out on each cDNA according to SOP 11-XP-003, XP™-PCR with the Applied Biosystems 9700. To monitor efficiency of the RT and PCR reactions 0.24 attamoles of Kanamycin RNA will be spiked into each RT reaction. Two types of positive control RNA will be used. Other assay controls include ‘No Template Controls’ (NTC) where water instead of RNA will be added to separate reactions and ‘Reverse Transcriptase minus’ (RT-) controls where sample RNA will be subjected to the procedure without reverse transcriptase.

### Expression Analysis and Calculations

PCR reactions will be analyzed by capillary electrophoresis. The fluorescently labeled PCR reactions will be diluted, combined with Genome Lab size standard-400 (Beckman-Coulter, Part Number 608098), denatured, and loaded onto the Beckman Coulter using SOP 11-XP-004, Operation and Maintenance of the CEQ 8800 Genetic Analysis System. The data obtained from the 8800 will be analyzed with expression analysis software to generate relative expression values for each gene. The expression of each target gene relative to the expression of either cyclophilin A, GAPDH, or β-actin within the same reaction is reported as the mean of the replicate. The standard deviation and percent coefficient of variance (% CV) associated with these values will also be reported when appropriate.

### Statistical Analysis Method

The mathematical form of the ANOVA model to be used in this analysis is:

Yᵢⱼₖₗ [symbol] =μ+αᵢ+β [symbol] [symbol] +γₖ+ω [symbol] +ε [symbol] [symbol] i=1 . . . 5 j=1 . . . 4 k=1 . . . 3 l=1 . . . 3 Cov(Y [symbol] ,Y [symbol] )=σₒ²+σ [symbol] ²Cov(Y [symbol] ,Y [symbol] )=σₒ²Cov(Y [symbol] ,Y [symbol] )=0 (1)

Here Yᵢⱼₖₗ is the normalized Rfu ratio obtained in the i^{th} sample under the j^{th} dosing concentration at the k^{th} time point from the l^{th} replicate. The model parameter μ is the overall mean normalized Rfu ratio, an unknown constant, αᵢ is a fixed effect due to sample i, βⱼ is a fixed effect due to dosing concentration j, γₖ is a fixed effect due to time point k, and ωₗ₍ᵢₗₖ₎ is a random effect due to the l^{th} replicate in the i^{th} sample under j^{th} dosing concentration at k^{th} time point, which is assumed Normally distributed with mean 0 and variance σ²_{ω}. εᵢⱼₖₗ is a random error term associated with the normalized Rfu ratio from the i^{th} sample under the j^{th} dosing concentration at the k^{th} time point from the l^{th} replicate, assumed Normally distributed with mean 0 and variance σ_{ε}².

lme function in nlme package in R will be used to analyze the data with respect to the model above. The overall dosing effect (H₀: β₁=β₂=β₃=β₄=β₅=0 versus H₁: At least one βᵢ is different) will be tested in F-test for each gene.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 6

### PARP Expression in Syngenic Samples Using O-RT-PCR

### Assay Description and Methods

XP™-PCR is a multiplex RT-PCR methodology that allows for the expression analysis of multiple genes in a single reaction (Kahn et al., 2007). A defined combination of gene specific and universal primers used in the reaction results in a series of fluorescently labeled PCR products whose size and quantity are measured using the capillary electrophoresis instrument GeXP.

### XP™-PCR

Multiplex RT-PCR was performed using 25 ng of total RNA of each sample using a previously described protocol (Khan et al., 2007). The RT reactions were carried out as described in SOP 11-XP-002, cDNA Production from RNA with the Applied Biosystems 9700. PCR reactions were carried out on each cDNA according to SOP 11-XP-003, XP™-PCR with the Applied Biosystems 9700. To monitor efficiency of the RT and PCR reactions 0.24 attamoles of Kanamycin RNA was spiked into each RT reaction. A positive control RNA was used and is detailed below in the Assay Discussion section. Other assay controls included ‘No Template Controls’ (NTC) where water instead of RNA was added to separate reactions and ‘Reverse Transcriptase minus’ (RT-) controls where sample RNA was subjected to the procedure without reverse transcriptase.

### Expression Analysis and Calculations

PCR reactions were analyzed by capillary electrophoresis. The fluorescently labeled PCR reactions were diluted, combined with Genome Lab size standard-400 (Beckman-Coulter, Part Number 608098), denatured, and loaded onto the Beckman Coulter using SOP 11-XP-004, Operation and Maintenance of the CEQ 8800 Genetic Analysis System. The data obtained from the 8800 was analyzed with our proprietary expression analysis software to generate relative expression values for each gene. The expression of each target gene relative to the expression of glucuronidase beta (GUSB) within the same reaction is reported as the mean of the replicate. The standard deviation and percent coefficient of variance (% CV) associated with these values are also reported when appropriate.

### Sample Description

Frozen human breast and lung tissues were obtain during surgery as a syngenic pair on dry ice. They consisted of a tumor sample and a normal sample from each of studied individuals.

### Sample RNA Extraction:

RNA was extracted from each sample using a RiboPure™ RNA isolation kit from Ambion Cat. # 1924). To insure that the samples would be thawed only under RNase denaturing conditions, each frozen sample was placed on a new sample collection tray on top of dry ice. Using a new razor blade for each sample, an approximately 100 mg piece of lung tissue and 200 mg piece of breast tissue was cut and immediately placed into a labeled tube containing the TRI Reagent and two ceramic beads. The samples were then homogenized using a Qiagen Laboratory Vibration Mill Type MM300 for 2 minutes at 20 MHz. The orientation of the mixer mill sample block was then reversed and the samples were homogenized for another 2 minutes. The RNA was then isolated from the homogenate following the RiboPure™ protocol supplied with the kit.

Following isolation, each sample of RNA was subjected to a DNase reaction following SOP 3-XP-001 DNase I Treatment of RNA to remove any residual sample DNA.

Immediately following the DNase heat inactivation step of the DNase reaction, the ribonuclease inhibitor SUPERase-In (Ambion, Cat. No. AM2696) was added to each sample at a final concentration of 1 U/μL.

### RNA Quantitation:

The concentration of the RNA was determined using the RiboGreen RNA Quantitation Kit (Invitrogen, Cat. No. R11490) and by following SOP 3-EQ-031 Wallac Victor2 1420 Multilabel Counter.

### Sample RNA Quality:

A sample of RNA from each sample was analyzed on an Agilent Bioanalyzer following Althea Technology's SOP 11-XP-001 Operation of Agilent 2100 Bioanalyzer. The results are shown in Appendix I.

### Sample Requirements:

Triplicate definition: Each sample of RNA was assayed in three separate XP™-PCR reactions.

RT-PCR Reaction Sample Requirements:
25 ng of total RNA was utilized in each reaction.

### XP™-PCR:

RT-PCR Controls:
The reverse transcription controls for the presence of DNA contamination in the RNA (RT minus) were negative.

The PCR controls for DNA contamination in the reagents (no template control) were negative.

Positive Control:
The human positive control RNA that was used in the assay was Ambion Human Reference RNA (HUR), (Ambion, custom order).

1. A method of identifying a treatment for a PARP mediated disease comprising identifying a level of PARP in a sample from a subject and making a decision regarding treatment of said PARP mediated disease, wherein said treatment decision is made based on said level of PARP.

2. (canceled)

3. A method of treating a disease with a PARP modulator comprising identifying a level of PARP in a sample from a subject; making a decision regarding treatment of a disease with a PARP modulator, said decision being based on said level of PARP; and treating said disease in said subject with said PARP modulator, said treatment being based on said treatment decision.

4-14. (canceled)

15. The method of any of claim 1 or 3 wherein said PARP mediated disease is selected from the group consisting of cancer, inflammation, metabolic disease, CVS disease, CNS disease, disorder of hematolymphoid system, disorder of endocrine and neuroendocrine, disorder of urinary tract, disorder of respiratory system, disorder of female genital system, and disorder of male genital system.

16. The method of claim 15 wherein said cancer is selected from the group consisting of colon adenocarcinoma, esophagus adenocarcinoma, liver hepatocellular carcinoma, squamous cell carcinoma, pancreas adenocarcinoma, islet cell tumor, rectum adenocarcinoma, gastrointestinal stromal tumor, stomach adenocarcinoma, adrenal cortical carcinoma, follicular carcinoma, papillary carcinoma, breast cancer, ductal carcinoma, lobular carcinoma, intraductal carcinoma, mucinous carcinoma, phyllodes tumor, ovarian adenocarcinoma, endometrium adenocarcinoma, granulose cell tumor, mucinous cystadenocarcinoma, cervix adenocarcinoma, vulva squamous cell carcinoma, basal cell carcinoma, prostate adenocarcinoma, giant cell tumor of bone, bone osteosarcoma, larynx carcinoma, lung adenocarcinoma, kidney carcinoma, urinary bladder carcinoma, Wilm's tumor, and lymphoma.

17. The method of claim 15 wherein said inflammation is selected from the group consisting of Non-Hodgkin's lymphoma, Wegener's granulomatosis, Hashimoto's thyroiditis, hepatocellular carcinoma, chronic pancreatitis, rheumatoid arthritis, reactive lymphoid hyperplasia, osteoarthritis, ulcerative colitis, and papillary carcinoma.

18. The method of claim 15 wherein said metabolic disease is diabetes or obesity.

19. The method of claim 15 wherein said CVS disease is selected from the group consisting of atherosclerosis, coronary artery disease, granulomatous myocarditis, chronic myocarditis, myocardial infarction, and primary hypertrophic cardiomyopathy.

20. The method of claim 15 wherein said CNS disease is selected from the group consisting of Alzheimer's disease, cocaine abuse, schizophrenia, and Parkinson's disease.

21. The method of claim 15 wherein said disorder of hematolymphoid system is selected from the group consisting of Non-Hodgkin's lymphoma, chronic lymphocyte leukemia, and reactive lymphoid hyperplasia.

22. The method of claim 15 wherein said disorder of endocrine and neuroendocrine disorder is selected from the group consisting of nodular hyperplasia, Hashimoto's thyroiditis, islet cell tumor, and papillary carcinoma.

23. The method of claim 15 wherein said disorder of urinary tract is selected from the group consisting of renal cell carcinoma, transitional cell carcinoma, and Wilm's tumor.

24. The method of claim 15 wherein said disorder of respiratory system is selected from the group consisting of adenosquamous carcinoma, squamous cell carcinoma, and large cell carcinoma.

25. The method of claim 15 wherein said disorder of female genital system is selected from the group consisting of adenocarcinoma, leiomyoma, mucinous cystadenocarcinoma, and serous cystadenocarcinoma.

26. The method of claim 15 wherein said disorder of male genital system is selected from the group consisting of prostate cancer, benign nodular hyperplasia, and seminoma.

27. The method of any of claim 1 or 3 wherein said PARP modulator is 4-iodo, 3-nitro benzamide.

28. A computer-readable medium suitable for transmission of a result of an analysis of a sample comprising an information regarding a disease in a subject treatable with a PARP modulator; said information being derived by identifying a level of PARP in said sample from said subject; and making a decision based on said level of PARP regarding treating said disease by said PARP modulators.

29. (canceled)

30. A method of identifying a breast cancer treatable with a PARP inhibitor comprising identifying a level of PARP in a sample from a subject and making a decision based on said level of PARP regarding whether said breast cancer is treatable with said PARP inhibitor.

31. A method of treating a breast cancer in a subject with a PARP inhibitor comprising identifying a level of PARP in a sample from said subject; making a decision based on said level of PARP regarding whether said breast cancer is treatable with said PARP inhibitor; and treating said breast cancer with said PARP inhibitor.

32-35. (canceled)

36. A method of classifying a breast tumor in a subject comprising identifying a level of PARP in a tumor sample from said subject and making a decision regarding treating said tumor with a PARP modulator, wherein said decision is made based on said level of PARP.

37. A method of treating a breast tumor in a subject comprising identifying a level of PARP in a sample from said subject; making a decision based on said level of PARP regarding treating said tumor with a PARP modulator; and treating said tumor in said subject with said PARP modulator.

38-47. (canceled)

48. A method of identifying a breast tumor treatable with a PARP inhibitor comprising identifying a level of PARP in a sample from a subject and making a decision based on said level of PARP regarding treatment of said breast tumor with said PARP inhibitor.

49. A method of treating a breast tumor in a subject by PARP inhibitor comprising identifying a level of PARP in a sample from said subject; making a decision based on said level of PARP regarding treating said breast tumor with a PARP inhibitor, and treating said breast tumor with said PARP inhibitor.

50-52. (canceled)

53. A method of treating a cancer in a subject comprising identifying a presence or absence of ER, Her2-neu, and PR in a cancer sample from said subject and treating said cancer with a PARP inhibitor, wherein said treatment is performed if said cancer sample is negative for ER, Her2-neu, and/or PR.

54. A method of identifying a PARP mediated disease or a stage of a PARP mediated disease treatable with a PARP modulator comprising identifying a level of PARP in a sample from a subject and determining whether said level of PARP is above a predetermined level thereby determining that said PARP mediated disease is to be treated with a PARP modulator.

55. A method of treating a disease by administration of a PARP modulator to a patient comprising identifying a level of PARP in a sample from said patient; determining whether said level of PARP is above a predetermined level thereby determining that said PARP mediated disease is to be treated with a PARP modulator and treating said disease in said subject by administering said PARP modulator to said patient.

56-57. (canceled)

58. A computer-readable medium suitable for transmission of a result of an analysis of a sample wherein the medium comprises an information regarding a disease in a subject treatable with a PARP modulator; said information being derived by identifying a level of PARP in said sample from said subject; and determining whether said level of PARP is above a predetermined level thereby determining that said PARP mediated disease is to be treated with a PARP modulator.

59. (canceled)

60. A method of identifying a breast cancer treatable with a PARP inhibitor comprising identifying a level of PARP in a sample from a subject; and determining whether said level of PARP is above a predetermined level thereby determining that said breast cancer is treatable with a PARP modulator.

61. A method of treating a breast cancer in a patient with a PARP inhibitor comprising identifying a level of PARP in a sample from said subject; determining whether said level of PARP is above a predetermined level thereby determining that said breast cancer is treatable with a PARP inhibitor; and treating said breast cancer by administering said PARP inhibitor to said patient.

62-64. (canceled)

65. A method of classifying a breast tumor in a patient comprising identifying a level of PARP in a tumor sample from said patient and determining whether said level of PARP is above a predetermined level thereby classifying said breast tumor as treatable with a PARP modulator.

66. A method of treating a breast tumor in a subject comprising identifying a level of PARP in a sample from said subject; determining whether said level of PARP is above a predetermined level thereby determining that said breast tumor is treatable with a PARP modulator and treating said tumor in said patient with said PARP modulator.

67-69. (canceled)

70. A method of identifying a breast tumor treatable with a PARP inhibitor comprising identifying a level of PARP in a sample from a patient; determining whether said level of PARP is above a predetermined level thereby identifying said breast tumor as treatable with a PARP inhibitor.

71. A method of treating a breast tumor in a patient with a PARP inhibitor comprising identifying a level of PARP in a sample from said patient; determining whether said level of PARP is above a predetermined level thereby determining that said breast tumor is treatable with a PARP inhibitor and treating said breast tumor by administering said PARP inhibitor to said patient

72-73. (canceled)

74. A method of treating a cancer in a patient comprising determining whether ER, Her2-neu, and/or PR are present in a cancer sample from said patient and treating said cancer with a PARP inhibitor when ER, Her2-neu, and/or PR are not present in said sample from said patient.

75. A method of selecting a subject for therapy with the PARP inhibitor comprising:
measuring a level of PARP in a biological sample collected from the subject prior to administration of the PARP inhibitor, determining that the PARP level in the sample is higher than a predetermined value and selecting the subject for therapy with the PARP inhibitor.

76. A method of treating a subject with a PARP inhibitor comprising:
measuring a level of PARP in a biological sample collected from the subject prior to administration of the PARP inhibitor, determining that the PARP level in the sample is higher than a predetermined value and administering to the subject the PARP inhibitor.

77. A method of assessing response to treatment in a subject undergoing therapy with a PARP inhibitor the method comprising: measuring the PARP level in the subject at least a first and a second point in time to produce at least a first level of PARP and a second level of PARP, wherein a decrease in the second level of PARP compared to the first level of PARP is indicative of positive response to treatment.

78. A method for treating a patient whose condition results in an elevated PARP level, wherein a PARP level of a patient sample is higher than a pre-determined PARP level, the method comprising, administering a therapeutically effective amount of a PARP inhibitor.

79. A method of identifying a treatment for a PARP mediated disease comprising identifying a level of PARP in a plurality of samples from a population, and making a decision regarding treatment of said PARP mediated disease, wherein said treatment decision is made based on said level of PARP.

80-104. (canceled)

105. A computer readable medium suitable for transmission of a result of an analysis of a plurality of samples from a population regarding a disease treatable with at least one PARP modulator; said information being derived by identifying a level of PARP in each of said plurality of samples, and making a decision based on said level of PARP regarding treating said disease by said PARP modulator.

106. (canceled)

107. A method of identifying a breast cancer treatable with a PARP inhibitor comprising identifying a level of PARP in a plurality of samples from a population, and making a decision based on said level of PARP in each of said plurality of samples regarding whether said breast cancer is treatable with said PARP inhibitor.

108. A method of treating a breast cancer in a subject with a PARP inhibitor comprising identifying a level of PARP in a sample from said subject; making a decision based on said level of PARP to determine whether said breast cancer is treatable with a PARP inhibitor, wherein said level of PARP from said subject is compared to a level of PARP in a plurality of samples from a population with breast cancer; and treating said breast cancer with said PARP inhibitor if the PARP level from said subject is comparable to the level of PARP in the plurality of samples from the population with breast cancer.

109-112. (canceled)

113. A method of classifying a breast tumor comprising identifying a level of PARP in a plurality of tumor samples from a population and making a decision regarding treating said tumor with a PARP modulator, wherein said decision is made based on said level of PARP.

114. A method of treating a breast tumor in a subject with a PARP inhibitor comprising identifying a level of PARP in a sample from said subject; making a decision based on said level of PARP to determine whether said breast tumor is treatable with a PARP inhibitor, wherein said level of PARP from said subject is compared to a level of PARP in a plurality of samples from a population with a breast tumor; and treating said breast tumor with said PARP inhibitor if the PARP level from said subject is comparable to the level of PARP in the plurality of samples from said population.

115-124. (canceled)

125. A method of identifying a breast tumor treatable with a PARP inhibitor comprising identifying a level of PARP in a plurality of samples from a population and making a decision based on said level of PARP regarding treatment of said breast tumor with said PARP inhibitor.

126-128. (canceled)

129. A method of identifying a breast cancer treatable with a PARP inhibitor comprising identifying a presence or absence of ER, Her2-neu, and PR in a sample in a plurality of samples from a population with cancer, and making a decision based on said level of PARP regarding treatment of said breast cancer with said PARP inhibitor and said presence or absence of ER, Her2-neu and PR in said plurality of samples.

130. A method of classifying an ovarian tumor comprising identifying a level of PARP in a plurality of tumor samples from a population and making a decision regarding treating said tumor with a PARP modulator, wherein said decision is made based on said level of PARP.

131. A method of treating an ovarian tumor in a subject with a PARP inhibitor comprising identifying a level of PARP in a sample from said subject; making a decision based on said level of PARP to determine whether said ovarian tumor is treatable with a PARP inhibitor, wherein said level of PARP from said subject is compared to a level of PARP in a plurality of samples from a population with a ovarian tumor; and treating said ovarian tumor with said PARP inhibitor if the PARP level from said subject is comparable to the level of PARP in the plurality of samples from said population.

132-140. (canceled)

141. A method of identifying an ovarian tumor treatable with a PARP inhibitor comprising identifying a level of PARP in a plurality of samples from a population and making a decision based on said level of PARP regarding treatment of said ovarian tumor with said PARP inhibitor.

142. A method of identifying an ovarian cancer treatable with a PARP inhibitor comprising identifying a level of PARP in a plurality of samples from a population, and making a decision based on said level of PARP in each of said plurality of samples regarding whether said ovarian cancer is treatable with said PARP inhibitor.

143. A method of treating an ovarian cancer in a subject with a PARP inhibitor comprising identifying a level of PARP in a sample from said subject; making a decision based on said level of PARP to determine whether said ovarian cancer is treatable with a PARP inhibitor, wherein said level of PARP from said subject is compared to a level of PARP in a plurality of samples from a population with ovarian cancer; and treating said ovarian cancer with said PARP inhibitor if the PARP level from said subject is comparable to the level of PARP in the plurality of samples from the population with ovarian cancer.

144. (canceled)

145. A method of classifying an endometrial tumor comprising identifying a level of PARP in a plurality of tumor samples from a population and making a decision regarding treating said tumor with a PARP modulator, wherein said decision is made based on said level of PARP.

146. A method of treating an endometiral tumor in a subject with a PARP inhibitor comprising identifying a level of PARP in a sample from said subject; making a decision based on said level of PARP to determine whether said endometrial tumor is treatable with a PARP inhibitor, wherein said level of PARP from said subject is compared to a level of PARP in a plurality of samples from a population with an endometrial tumor; and treating said endometrial tumor with said PARP inhibitor if the PARP level from said subject is comparable to the level of PARP in the plurality of samples from said population.

147-155. (canceled)

156. A method of identifying an endometrial tumor treatable with a PARP inhibitor comprising identifying a level of PARP in a plurality of samples from a population and making a decision based on said level of PARP regarding treatment of said endometrial tumor with said PARP inhibitor.

157. A method of identifying an endometrial cancer treatable with a PARP inhibitor comprising identifying a level of PARP in a plurality of samples from a population, and making a decision based on said level of PARP in each of said plurality of samples regarding whether said endometrial cancer is treatable with said PARP inhibitor.

158. A method of treating an endometrial cancer in a subject with a PARP inhibitor comprising identifying a level of PARP in a sample from said subject; making a decision based on said level of PARP to determine whether said endometrial cancer is treatable with a PARP inhibitor, wherein said level of PARP from said subject is compared to a level of PARP in a plurality of samples from a population with endometrial cancer; and treating said endometrial cancer with said PARP inhibitor if the PARP level from said subject is comparable to the level of PARP in the plurality of samples from the population with endometrial cancer.

159. (canceled)

160. A method of classifying a lung tumor comprising identifying a level of PARP in a plurality of tumor samples from a population and making a decision regarding treating said tumor with a PARP modulator, wherein said decision is made based on said level of PARP.

161. A method of treating a lung tumor in a subject with a PARP inhibitor comprising identifying a level of PARP in a sample from said subject; making a decision based on said level of PARP to determine whether said lung tumor is treatable with a PARP inhibitor, wherein said level of PARP from said subject is compared to a level of PARP in a plurality of samples from a population with a lung tumor; and treating said lung tumor with said PARP inhibitor if the PARP level from said subject is comparable to the level of PARP in the plurality of samples from said population.

162-170. (canceled)

171. A method of identifying a lung tumor treatable with a PARP inhibitor comprising identifying a level of PARP in a plurality of samples from a population and making a decision based on said level of PARP regarding treatment of said lung tumor with said PARP inhibitor.

172. A method of identifying a lung cancer treatable with a PARP inhibitor comprising identifying a level of PARP in a plurality of samples from a population, and making a decision based on said level of PARP in each of said plurality of samples regarding whether said lung cancer is treatable with said PARP inhibitor.

173. A method of treating a lung cancer in a subject with a PARP inhibitor comprising identifying a level of PARP in a sample from said subject; making a decision based on said level of PARP to determine whether said lung cancer is treatable with a PARP inhibitor, wherein said level of PARP from said subject is compared to a level of PARP in a plurality of samples from a population with lung cancer; and treating said lung cancer with said PARP inhibitor if the PARP level from said subject is comparable to the level of PARP in the plurality of samples from the population with lung cancer.

174. (canceled)

175. A method of identifying a PARP mediated disease or a stage of a PARP mediated disease treatable with a PARP modulator comprising identifying a level of PARP in a plurality of samples from a population and determining whether said level of PARP is above a predetermined level thereby determining that said PARP mediated disease is to be treated with a PARP modulator.

176. A method of treating a disease by administration of a PARP modulator to a patient comprising identifying a level of PARP in a plurality of samples from a population with said disease; determining whether said level of PARP in the plurality of samples from the population is above a predetermined level thereby determining that said PARP mediated disease is to be treated with a PARP modulator, identifying a level of PARP in said patient and comparing said level of PARP from said patient with the level of PARP from the plurality of samples, and treating said disease in said subject by administering said PARP modulator to said patient if the PARP level from said patient is comparable to the PARP level from said plurality of samples from the population.

177-178. (canceled)

179. A computer-readable medium suitable for transmission of a result of an analysis of a sample wherein the medium comprises an information regarding a disease in a subject treatable with a PARP modulator; said information being derived by identifying a level of PARP in a plurality of samples from a population with said disease; and determining whether said level of PARP is above a predetermined level thereby determining that said PARP mediated disease is to be treated with a PARP modulator.

180. (canceled)

181. A method of identifying a disease treatable with a PARP inhibitor comprising identifying a level of PARP in a plurality of samples from a population with said disease; and determining whether said level of PARP is above a predetermined level thereby determining that said disease is treatable with a PARP modulator.

182-184. (canceled)

185. A method of classifying a disease in a patient comprising identifying a level of PARP in a plurality of tumor samples from a population with said disease and determining whether said level of PARP in the plurality of tumor samples is above a predetermined level thereby classifying said disease as treatable with a PARP modulator.

186-188. (canceled)

189. A method of selecting a subject for therapy with a PARP inhibitor comprising:
measuring a level of PARP in a biological sample collected from the subject prior to administration of the PARP inhibitor; comparing the level of PARP from the subject to a level of PARP from a plurality of samples from a population with a disease; determining that the PARP level in the sample is higher than a predetermined value; and selecting the subject for therapy with the PARP inhibitor.

190-192. (canceled)

193. A method of treating a subject with a PARP inhibitor comprising:
measuring a level of PARP in a biological sample collected from the subject prior to administration of the PARP inhibitor; comparing the level of PARP from the subject to a level of PARP from a plurality of samples from a population with a disease; determining that the PARP level in the sample is higher than a predetermined value; and administering to the subject the PARP inhibitor.

194-196. (canceled)

197. A method of treating a breast cancer in a subject comprising identifying a presence or absence of estrogen receptor (“ER”), progesterone receptor (“PR”), and human epidermal growth factor 2 receptor (“Her2-neu”) in a breast cancer sample from said subject and treating said breast cancer with a PARP inhibitor, wherein said treatment is performed if said breast cancer sample is negative for ER, Her2-neu, and PR.

198. The method of claim 197, wherein said PARP inhibitor is a compound of formula (Ia): 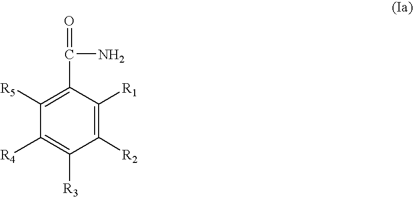
wherein R₁, R₂, R₃, R₄, and R₅ are, independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen, at least one of the five substituents is always nitro, and at least one substituent positioned adjacent to a nitro is always iodo, or a pharmaceutically acceptable salt thereof.

199. The method of claim 197, wherein said PARP inhibitor is 4-iodo-3-nitrobenzamide or a pharmaceutically acceptable salt thereof.

200. The method of claim 197, wherein said PARP inhibitor is selected from the group consisting of benzamide, quinolone, isoquinolone, benzopyrone, methyl 3,5-diiodo-4-(4′-methoxyphenoxy)benzoate, and methyl-3,5-diiodo-4-(4′-methoxy-3′,5′-diiodo-phenoxy)benzoate, cyclic benzamide, benzimidazole and indole, or a pharmaceutically acceptable salt thereof.

201. The method of claim 197, wherein said subject is deficient in BRCA gene.

202. The method of claim 197, wherein said subject has down-regulated BRCA gene.

203. The method of claim 197, wherein said sample is a tumor sample.

204. The method of claim 197, wherein said cancer is an infiltrating duct carcinoma.

205. The method of claim 197, wherein said treatment further comprises surgery or radiation therapy.

206. The method of claims 197, wherein said treatment is selected from the group consisting of oral administration, transmucosal administration, buccal administration, nasal administration, inhalation, parental administration, intravenous, subcutaneous, intramuscular, sublingual, transdermal administration, and rectal administration.
